(19) United States
(12) Patent Application Publication
(10) Pub. No.: US 2026/0327631 A1
(43) Pub. Date: Oct. 22, 2026

(54) MODIFIED MINI-NUCLEOSOME CORE PROTEINS AND USE IN NUCLEIC ACID DELIVERY

(71) Applicant: Adarsha Koirala, Framingham, MA (US)

(72) Inventor: Adarsha Koirala, Framingham, MA (US)

(21) Appl. No.: 19/570,015

(22) Filed: Mar. 17, 2026

Related U.S. Application Data
(62) Division of application No. 17/918,469, filed on Apr. 12, 2021, now Pat. No. 12,606,833.
(60) Provisional application No. 63/009,124, filed on Apr. 13, 2020.

(51) Int. Cl. C12N 15/62; A61K 38/00; A61K 48/00; C12N 15/87

(52) U.S. Cl. CPC C12N 15/62; A61K 48/0025; A61K 48/005; C12N 15/87; A61K 38/00; C07K 2319/033; C07K 2319/09; C07K 2319/35; C07K 2319/80

(57) ABSTRACT

The present disclosure provides compositions and methods relating to modified mini-nucleosome core proteins and/or delivery of nucleic acids. In particular, the present disclosure includes, among other things, non-viral proteinaceous vehicles for delivery of nucleic acids. In various embodiments, non-viral proteinaceous vehicles provided herein include (a) a nucleic acid binding domain; (b) a targeting domain; (c) a nucleic acid release domain; and, optionally, (d) further domains including, e.g., one or more of a stability domain, an oligomerization domain, and/or a linker domain. In various embodiments, the proteinaceous vehicles include one or more modified residues.

### CROSS-REFERENCE TO RELATED APPLICATIONS

This application is a divisional of U.S. application Ser. No. 17/918,469, filed on Oct. 12, 2022, which is a national stage application based on International Application No. PCT/US21/26917, filed on Apr. 12, 2021, which claims the benefit of U.S. Provisional Application No. 63/009,124, filed on Apr. 13, 2020, the content of each of which is hereby incorporated by reference in its entirety.

### SEQUENCE LISTING

The instant application contains a Sequence Listing which has been submitted electronically in XML format and is hereby incorporated by reference in its entirety. Said XML copy, created on Jun. 10, 2026, is named ASO-00202_SL.xml and is 337,038 bytes in size.

### BACKGROUND

AAV vectors are considered the current gold standard of gene therapy and have shown promise in diverse clinical trials, including clinical trials for, e.g., retinal gene therapy and systemic gene therapy in liver, CNS, and/or other tissues. With the regulatory approval of at least three different gene therapies, the field is poised for many more, so patients can access these life-changing treatments. However, despite being the industry's gold standard, AAV vectors have certain limitations. Improved and/or alternative nucleic acid delivery technologies are needed.

### SUMMARY

The present disclosure provides compositions and methods relating to, among other things, polypeptides that are capable of associating with nucleic acid molecules, wherein the polypeptides include at least one modified amino acid. In various embodiments, the polypeptides can be used for, e.g., delivering the nucleic acid molecules to a subject in need of gene therapy. Accordingly, the present disclosure includes, among other things, modified polypeptides capable of associating with nucleic acid molecules, as well as compositions including modified polypeptides disclosed herein together with associated nucleic acid molecules. The present disclosure contemplates, without wishing to be bound by any particular scientific theory, that association of a nucleic acid molecule with a modified polypeptide disclosed herein can facilitate delivery of the nucleic acid to a target cell, subject, or other system.

In particular, the present disclosure includes, among other things, “modified mini-nucleosome core proteins” for delivery of nucleic acids. In various embodiments, a mini-nucleosome core protein of the present disclosure can include (a) a nucleic acid binding domain (“NABD”); (b) a nucleic acid release domain; and, optionally, (c) further domains including, e.g., one or more of a targeting domain, a stability domain, an oligomerization domain, and/or a linker domain. In various embodiments, a mini-nucleosome core protein of the present disclosure can include (a) a nucleic acid binding domain (“NABD”); (b) a nucleic acid release domain; (c) a targeting domain; and, optionally, (d) further domains including, e.g., one or more of a stability domain, an oligomerization domain, and/or a linker domain. In various embodiments, a mini-nucleosome core protein of the present disclosure can be a modified mini-nucleosome core protein that includes (a) a nucleic acid binding domain (“NABD”); (b) a nucleic acid release domain; (c) a modified amino acid residue; and, optionally, (d) further domains including, e.g., one or more of a targeting domain, a stability domain, an oligomerization domain, and/or a linker domain. In various embodiments, a mini-nucleosome core protein of the present disclosure can be a modified mini-nucleosome core protein that includes (a) a nucleic acid binding domain (“NABD”); (b) a nucleic acid release domain; (c) a targeting domain; (d) a modified amino acid residue; and, optionally, (e) further domains including, e.g., one or more of a stability domain, an oligomerization domain, and/or a linker domain. As disclosed in herein, a modified a mini-nucleosome core protein can be referred to and/or described as a mini-nucleosome core protein and/or as a protein that is or includes a mini-nucleosome core protein having at least one modified amino acid residue. Accordingly, for clarity, all references, express or implied, to “mini-nucleosome core proteins” in the present disclosure include and encompass modified mini-nucleosome core proteins of the present disclosure. One or more mini-nucleosome core proteins associated with a nucleic acid cargo can be referred to as a “loaded mini-nucleosome.” Because a loaded mini-nucleosome that is for delivery of a nucleic acid to a target is non-viral, a mini-nucleosome is an example of a non-viral vehicle for nucleic acid delivery.

In particular, the present disclosure includes, among other things, “modified mini-nucleosome core proteins” for delivery of nucleic acids. In various embodiments, a mini-nucleosome core protein of the present disclosure can include (a) a nucleic acid binding domain (“NABD”); (b) a targeting domain; and, optionally, (c) further domains including, e.g., one or more of a nucleic acid release domain, a stability domain, an oligomerization domain, and/or a linker domain. In various embodiments, a mini-nucleosome core protein of the present disclosure can include (a) a nucleic acid binding domain (“NABD”); (b) a targeting domain; (c) a nucleic acid release domain; and, optionally, (d) further domains including, e.g., one or more of a stability domain, an oligomerization domain, and/or a linker domain. In various embodiments, a mini-nucleosome core protein of the present disclosure can be a modified mini-nucleosome core protein that includes (a) a nucleic acid binding domain (“NABD”); (b) a targeting domain; (c) a modified amino acid residue; and, optionally, (d) further domains including, e.g., one or more of a nucleic acid release domain, a stability domain, an oligomerization domain, and/or a linker domain. In various embodiments, a mini-nucleosome core protein of the present disclosure can be a modified mini-nucleosome core protein that includes (a) a nucleic acid binding domain (“NABD”); (b) a targeting domain; (c) a nucleic acid release domain; (d) a modified amino acid residue; and, optionally, (e) further domains including, e.g., one or more of a stability domain, an oligomerization domain, and/or a linker domain. As disclosed in herein, a modified a mini-nucleosome core protein can be referred to and/or described as a mini-nucleosome core protein and/or as a protein that is or includes a mini-nucleosome core protein having at least one modified amino acid residue. Accordingly, for clarity, all references, express or implied, to “mini-nucleosome core proteins” in the present disclosure include and encompass modified mini-nucleosome core proteins of the present disclosure. One or more mini-nucleosome core proteins associated with a nucleic acid cargo can be referred to as a “loaded mini-nucleosome.” Because a loaded mini-nucleosome that is for delivery of a nucleic acid to a target is non-viral, a mini-nucleosome is an example of a non-viral vehicle for nucleic acid delivery.

The present disclosure includes the recognition that at least some modified mini-nucleosome core proteins of the present disclosure have advantageous properties including, without limitation, increased bioavailability; increased half-life; increased stability; decreased degradation; increased binding affinity, e.g., for target cells; increased uptake, e.g., by target cells; improved blood-brain barrier penetration; reduced accumulation, e.g., in target cells or tissues; reduced aggregation; reduced precipitation; reduced thermal denaturation; and/or reduced kinetic denaturation, e.g., as set forth herein.

The present disclosure includes the recognition that at least certain compositions and methods described herein remedy one or more deficiencies associated with AAV vectors, including that:

1) AAV is associated with a payload limitation of 4.5 kb DNA length, which limitation prevents use of AAV in treatment of diseases caused at least in part by deficiency in expression of a gene product typically encoded by a nucleic acid larger than 4 kb (for example genes like CFTR, HTT, F8, DMD, ABCA4 etc. cannot fit into AAV vectors) (Lai Y. et al, 2010).

2) AAV has been known to integrate at low percentage and/or in a site-non-specific manner (Smith R. H., 2008). Random or site-non-specific integration may be deleterious if integration can or does disrupt a tumor suppressor gene or gene important for cellular functions.

3) Depending on the serotype of AAV, 25-70% of humans have preexisting neutralizing antibodies to AAV which means, they would be less likely to benefit for AAV therapy (Fitzpatrick Z., et al 2018).

4) Multiple treatments with AAV are highly unlikely to be effective because once a patient is injected, the patient produces a high number of antibodies against the virus. For some diseases where cellular turnover is high (e.g., in the turnover of liver cells or airway epithelial cells) multiple treatments maybe needed. Thus, due to increased antibodies against AAVs following a first treatment, the same vector may not be useful in follow-up treatments or doses.

5) Effective treatment of some diseases may require delivery of an enormous payload of particles administered by intravenous injection in order to transduce cells in vivo. A high dose of AAV comes with its own toxicities, which are well documented (Hinderer C. et al, 2018).

6) Most diseases are also associated with multi-organ defects and AAV may not be applied to various organs in the same body. One application at one site will raise antibodies and thus may block transduction at other locations in the body when injected in a subsequent treatment or dose.

Due at least in part to the deficiencies of AAV discussed above, there is a dire need for alternatives to AAV. In at least certain embodiments, non-viral vectors disclosed herein overcome one or more of the deficiencies of AAV discussed above.

Moreover, prior non-viral vectors are also associated with several barriers to therapeutic efficacy including: i) low transfection/transduction efficiency (Guerra-Crespo M et al, 2003) ii) low particle stability in blood, body fluids and other tissues (Barua and Mitragotri, 2014); iii) low cell entry via receptor-mediated endocytosis or cell fusion; iv) low stability in, and low escape from, endosomal and lysosomal compartments; v) low diffusion rate in the cytoplasm; vi) low nuclear pore transit; and vii) low release of DNA to permit biological function in the nucleus (Zabner J. et al, 1995). Several publications have documented inability or low efficiency of prior non-viral vectors to transfect post-mitotic cells (Wilke M. et al, 1996). Certain prior non-viral vectors lack longevity of expression and/or produce low amount of proteins that are not therapeutic enough and cannot be targeted to specific cell types in an efficient manner.

Thus, despite state-of-the-art research in the field of non-viral vectors, many prior non-viral vectors are not optimal for clinical use. Certain characteristics of at least certain embodiments discussed herein that contribute to, among other things, clinical utility, can include, without limitation:

Size and molecular weight: Many prior non-viral vectors that carry DNA molecule have a size of 10-200 nm in diameter (Konstan M. W. et. al, 2004). Their molecular weights can be greater than 300 kDa or greater than 500 kDa. The present disclosure provides, among other things, non-viral proteinaceous vehicles, and/or loaded mini-nucleosomes, that are <20 nm in diameter and have a molecular weight of <500 kDa. In particular embodiments, a non-viral proteinaceous vehicles, and/or loaded mini-nucleosomes, disclosed herein can pass into the nucleus more efficiently, perhaps, by passive diffusion, at least in part because a typical nuclear pore is only 20 nm in diameter, such that <20 nm size may allow passage.

Stability in body fluids: Many prior non-viral vectors are degraded in body fluids like blood or CSF before they can be delivered to target cells (Barua and Mitragotri, 2014). The present disclosure, provides, among other things, non-viral proteinaceous vehicles, and/or loaded mini-nucleosomes, that are physiologically stable and/or have properties that allow them to be stable in blood and/or other body fluids until and after entry into a target cell. At least one goal for these particles to safely reach the nucleus of desired cells.

Release of particles in nucleus: Many prior non-viral vectors have a very short life time because most release associated nucleic acids before entering target cells, and the remainder release associated nucleic acids in the cytoplasm, where delivered DNA encounters nucleases that destroys DNA (Zabner. J. et al, 1995). Certain prior vectors that make it into the cell nucleus and provide expression levels are very low, if they express at all. The present disclosure also recognizes, among other things, that it can be beneficial to release associated nucleic acids at a slow rate, instead of all at once, which may allow for longevity of expression.

Cell type specificity: Prior non-viral vectors are not targeted to specific cell types are associated with reduced levels of transduction and thus, reduced expression. The present disclosure provides, among other things, non-viral vectors optimized for cell-type specificity. Certain means of engineering cell-type specificity are described, e.g., in Templeton and Senzer, 2011.

Taken together, there is a tremendous need for nucleic acid delivery technologies that provide effective levels of expression for a desired duration, are non-immunogenic and non-toxic, and have less limited payload capacity. Moreover, the need for millions of patients of Huntington, Stargardt. Duchenne muscular dystrophy, Cystic Fibrosis, and other conditions treatable by gene therapy clearly presents a need for technology that can help treat these patients.

The present disclosure provides safe and efficacious non-viral proteinaceous vehicles (“mini-nucleosome core proteins”), and loaded mini-nucleosomes, for delivery of nucleic acids. The present disclosure further recognizes that in at least some instances modified mini-nucleosome core proteins of the present disclosure can have advantageous properties including, without limitation, increased bioavailability; increased half-life; increased stability; decreased degradation; increased binding affinity, e.g., for target cells; increased uptake, e.g., by target cells; improved blood-brain barrier penetration; reduced accumulation, e.g., in target cells or tissues; reduced aggregation; reduced precipitation; reduced thermal denaturation; and/or reduced kinetic denaturation, e.g., as set forth herein.

In various embodiments, a mini-nucleosome core protein is associated with one or more nucleic acids. As disclosed herein a mini-nucleosome core protein associated with one or more nucleic acids can be referred to as a “loaded mini-nucleosome.”

In various embodiments, a mini-nucleosome core protein includes a nucleic acid release domain that targets a loaded mini-nucleosome to one or more specific cell types for delivery and/or targeted expression of a nucleic acid, such as a gene, in or to one or more specific cell types.

In various embodiments, a mini-nucleosome core protein composition (e.g., a composition including one or more loaded mini-nucleosomes) can be titered and/or administered either once or repeatedly based on need. Furthermore, in various embodiments, a mini-nucleosome core protein or mini-nucleosome composition (e.g., a composition including one or more loaded mini-nucleosomes) is non-immunogenic and non-toxic.

Mini-nucleosome core proteins disclosed herein can, in certain embodiments, utilize principles applicable to macromolecule uptake, viral entry into cells, nucleosome formation in eukaryotic cells, cleavage of certain proteins at certain location in the cells, etc.

Various embodiments of the compositions and methods provided herein include domains that facilitate one or more of enhanced stability, targeting to specific cell types, and enhanced longevity of expression by slow nucleic acid release.

In various embodiments, a mini-nucleosome core protein and/or a mini-nucleosome is stable in body fluids and/or include domains that allow and/or target release in or to the nucleus. In at least a first aspect, the present disclosure provides an engineered polypeptide including a nucleic acid binding domain and a nucleic acid release domain, where one or more amino acids of the engineered polypeptide is a modified amino acid, optionally where the modification includes at least one of: (i) phosphorylation; (ii) sulfation; (iii) glycosylation; (iv) prenylation; (v) methylation; (vi) sialylation; (vii) lipidation and/or lipoylation; (viii) acetylation; (ix) hydroxylation; (x) palmitoylation; (xi) mannosylation; (xii) myristoylation; (xiii) fucosylation; (xiv) pegylation; and/or (xv) any combination thereof. In certain embodiments, the engineered polypeptide includes a targeting domain.

In at least a further aspect, the present disclosure provides an engineered polypeptide including a nucleic acid binding domain and a targeting domain, where one or more amino acids of the engineered polypeptide is a modified amino acid, optionally where the modification includes at least one of: (i) phosphorylation; (ii) sulfation; (iii) glycosylation; (iv) prenylation; (v) methylation; (vi) sialylation; (vii) lipidation and/or lipoylation; (viii) acetylation; (ix) hydroxylation; (x) palmitoylation; (xi) mannosylation; (xii) myristoylation; (xiii) fucosylation; (xiv) pegylation; and/or (xv) any combination thereof, where engineered polypeptide optionally further includes a nucleic acid release domain.

In at least certain embodiments of various aspects of the present disclosure, each of two or more amino acids of an engineered polypeptide is a modified amino acid. In some embodiments, at least one of the modified amino acids includes a modification chain including two or more modifications selected from: (i) phosphorylation; (ii) sulfation; (iii) glycosylation; (iv) prenylation; (v) methylation; (vi) sialylation; (vii) lipidation and/or lipoylation; (viii) acetylation; (ix) hydroxylation; (x) palmitoylation; (xi) mannosylation; (xii) myristoylation; (xiii) fucosylation; (xiv) pegylation; and (xv) any combination thereof. In some embodiments, the modification increases the stability, half-life, and/or bioavailability of the engineered polypeptide. In some embodiments, the modification increases the affinity and/or avidity of the engineered polypeptide with a binding partner, optionally where the binding partner is a receptor, cell, or cell membrane. In some embodiments, the modification increases the affinity or avidity of the engineered polypeptide with a nucleic acid. In some embodiments, the modification decreases precipitation and/or aggregation of the engineered polypeptide.

In at least certain embodiments of various aspects of the present disclosure, a nucleic acid binding domain is derived from a histone polypeptide sequence. In some embodiments, the nucleic acid binding domain is or includes the amino acid sequence KRHRK. In some embodiments, the nucleic acid binding domain is or includes an amino acid sequence that includes KRHRK, RRRRR, RRLARR, KKAKAAAKPKK, KKDGKKRKR, KKKLK, KKRIRK, RKKSK, KKPKK, or a combination thereof. In some embodiments, the nucleic acid binding domain is a modified nucleic acid binding domain in that the nucleic acid binding domain includes one or more modified amino acids.

In at least certain embodiments of various aspects of the present disclosure, a targeting domain is a targeting domain having the sequence of any one of SEQ ID NOs: 397-422, where the targeting domain is phosphorylated. In at least certain embodiments of various aspects of the present disclosure, a targeting domain is a targeting domain having the sequence of any one of SEQ ID NOs: 423-428, where the targeting domain is sulfated. In at least certain embodiments of various aspects of the present disclosure, a targeting domain is a targeting domain having the sequence of any one of SEQ ID NOs: 429-434, where the targeting domain is glycosylated.

In at least certain embodiments of various aspects of the present disclosure, a targeting domain is a targeting domain having the sequence of any one of SEQ ID NOs: 435-440, where the targeting domain is prenylated. In at least certain embodiments of various aspects of the present disclosure, a targeting domain is a targeting domain having the sequence of any one of SEQ ID NOs: 441-446, where the targeting domain is methylated. In at least certain embodiments of various aspects of the present disclosure, a targeting domain is a targeting domain having the sequence of any one of SEQ ID NOs: 447-459, where the targeting domain is sialylated. In at least certain embodiments of various aspects of the present disclosure, a targeting domain is a cell attachment targeting domain, a beta galactose binding domain, a fucose binding domain, a heparin binding domain, a sialic acid binding domain, a glycoprotein binding domain, a carbohydrate binding domain, a lysophosphatidic acid binding domain, a cAMP binding domain, a hyaluronan binding domain, a chondroitin sulfate binding domain, an integrin binding domain, a nucleolin binding domain, a collagen binding domain, a clathrin binding domain, a Fc receptor binding domain, an actin binding domain, an endocytosis motif, a nuclear localization signal, or a combination thereof.

In at least certain embodiments of various aspects of the present disclosure, a targeting domain is a cell attachment targeting domain. In some embodiments, the cell attachment targeting domain is or includes an amino acid sequence that includes WGREERQ, NTQIH, WNNKTPH, TPH, VNRWS, XBBBXXBX, ARKKAAKA, QRR, SRR, WEPSRPFPVD, HRRTRKAPKRIRLPHIR, KRTGQYKLGSKTGPGQK, KKTK, KLRSQLVKK, RRRCGQKKK, BX(7)B, RIQNLLKITNLRIKFVK, KKEKDIMKKTI, KGE, RGD, RGDS, TTVVNPKYEGK, ERMSQIKRLLS, WRHRARS, GFOGER, LFDLM, WGREERQ, QSTEKRG, LPNTG, or a combination thereof.

In at least certain embodiments of various aspects of the present disclosure, a targeting domain is an internalization domain. In some embodiments, the internalization domain is or includes an amino acid sequence that includes FXDXF, PPSY, FEDNFVP, YIRV, YADW, YTQV, KKRPKP, SSDDE, RRASS, (YXXL)2, LPLTG, LAFTG, or a combination thereof.

In at least certain embodiments of various aspects of the present disclosure, a targeting domain is a cell-type specific targeting domain. In some embodiments, the cell-type specific targeting domain is or includes an amino acid sequence that includes ASSLNIA, KKEEEKKEEEKKEEE, LIFHKEQ, KFNKPFVFLI, QPEHSST, EYHHYNK, NGR, GEKGEP, KTKKK, KALKKK, KGKKK, CSVTCG, LRE, YKYNLNGRES, YRSL, KGGK7, KKKQYTSIHHG, KDEL, LADQDYTKTA, or a combination thereof.

In at least certain embodiments of various aspects of the present disclosure, a targeting domain is a modified targeting domain in that the targeting domain includes one or more modified amino acids. In some embodiments, the nucleic acid release domain is or includes an amino acid sequence that includes GRKKRRQRRRPQ, KRH, KSVKKRSVSEIQ, NRRKKRAL, KFERQ, VRGP, NKDS, NRDN, ANNR, or a combination thereof.

In at least certain embodiments of various aspects of the present disclosure, a the nucleic acid release domain is a modified nucleic acid release domain in that the nucleic acid release domain includes one or more modified amino acids.

In at least certain embodiments of various aspects of the present disclosure, an engineered polypeptide further includes a poly-arginine domain. In some embodiments, the poly-arginine domain is a modified poly-arginine domain in that the poly-arginine domain includes one or more modified amino acids.

In at least certain embodiments of various aspects of the present disclosure, an engineered polypeptide further includes a nuclear internalization signal or a nuclear import machinery binding domain. In some embodiments, the nuclear internalization signal or nuclear import machinery binding domain is or includes an amino acid sequence that includes KKKYKLK, KKRKLE, TRSK, HRKRKR, NKRKRK, AEKSKKK, RKSK, KRVK, KRK, LQQTPLHLAVI, RRPR, PRPR, RPPP, RKKRKGK, PAAKRVKLD, KLKIKRPVK, PKKKRKV, QRKRQK, DSPE, FQVT, QSTEKRG, RQGLID, Cyclic RKKH, or a combination thereof. In some embodiments, the nuclear internalization signal or a nuclear import machinery binding domain is a modified nuclear internalization signal or a nuclear import machinery binding domain in that the nuclear internalization signal or a nuclear import machinery binding domain includes one or more modified amino acids.

In at least certain embodiments of various aspects of the present disclosure, an engineered polypeptide further includes a stability domain. In some embodiments, the stability domain is or includes an amino acid sequence that includes YTRF, GDAY, LLEE, RKKRRQRRR, YKSL, YENF, FQDL, YIGSR, IKVAV, or a combination thereof. In some embodiments, the stability domain is a modified stability domain in that the stability domain includes one or more modified amino acids.

In at least certain embodiments of various aspects of the present disclosure, an engineered polypeptide further includes an oligomerization domain. In some embodiments, the oligomerization domain is selected from the oligomerization domains of Table 11, optionally where the oligomerization domain is positioned at the C-terminus of the engineered polypeptide. In some embodiments, the oligomerization domain is a modified oligomerization domain in that the oligomerization domain includes one or more modified amino acids.

In at least certain embodiments of various aspects of the present disclosure, an engineered polypeptide further includes a linker. In some embodiments, the linker is a linker according to any one of SEQ ID NOs: 154-250. In some embodiments, the linker is a modified linker in that the linker includes one or more modified amino acids.

In some embodiments, one or more amino acids of an engineered polypeptide is a phosphorylated amino acid. In some embodiments, the phosphorylated amino acid is a serine, threonine, or tyrosine amino acid. In some embodiments, the phosphorylated amino acid is present in a linker domain or targeting domain.

In some embodiments, one or more amino acids of an engineered polypeptide is a sulfated amino acid. In some embodiments, the sulfated amino acid is a serine, threonine, or tyrosine amino acid. In some embodiments, the sulfated amino acid is present in a linker domain or targeting domain.

In some embodiments, one or more amino acids of an engineered polypeptide is an acetylated amino acid. In some embodiments, the acetylated amino acid is a lysine amino acid. In some embodiments, the acetylated amino acid is present in a linker domain or targeting domain.

In some embodiments, one or more amino acids of an engineered polypeptide is a mannosylated amino acid. In some embodiments, the mannosylated amino acid is a serine amino acid. In some embodiments, the mannosylated amino acid is present in a linker domain or targeting domain.

In at least one further aspect, the present disclosure provides a polynucleotide that encodes the amino acid sequence of an engineered polypeptide of the present disclosure, such as a DNA or RNA polynucleotide. In at least one further aspect, the present disclosure provides a vector including polynucleotide of the present disclosure. In at least one further aspect, the present disclosure provides a cell including an engineered polypeptide of the present disclosure or a vector of the present disclosure.

In at least one further aspect, the present disclosure provides method of making an engineered polypeptide of the present disclosure, the method including expressing a polynucleotide of the present disclosure in a cell. In some embodiments, the method further includes isolating the engineered polypeptide from the cell.

In at least one further aspect, the present disclosure provides a composition including: (i) at least one polynucleotide, and (ii) at least one engineered polypeptide of the present disclosure. In some embodiments, the at least one polynucleotide is or includes DNA or RNA. In some embodiments, the at least one polynucleotide includes a nucleotide sequence encoding a polypeptide. In some embodiments, the at least one polynucleotide is or includes mRNA. In some embodiments, the at least one polynucleotide includes an inhibitory RNA. In some embodiments, the inhibitory RNA is a gRNA, siRNA, miRNA, or shRNA. In some embodiment, the composition includes at least two engineered polypeptides of the present disclosure, where a first engineered polypeptide of present disclosure is able to oligomerize with a second engineered polypeptide of the present disclosure. In some embodiments, the ratio of polynucleotides to engineered polypeptides is between 1:1 and 1:2,000. In some embodiments, the ratio of polynucleotides to engineered polypeptides is between 1:1 and 1:1,000, between 1:1 and 1:500, between 1:1 and 1:200, between 1:1 and 1:100, between 1:1 and 1:50, between 1:3 and 1:1,000, between 1:3 and 1:500, between 1:3 and 1:200, between 1:3 and 1:100, or between 1:3 and 1:50. In some embodiments, the ratio of polynucleotides to engineered polypeptides is between 1:200 and 1:2,000, between 1:200 and 1:1000, or between 1:200 and 1:500. In some embodiments, the composition includes a pharmaceutical carrier.

In at least one further aspect, the present disclosure provides a method that includes administering a composition of the present disclosure to a system, where the system is a cell, tissue, or subject. In some embodiments, after administration, the modification increases the stability, half-life, and/or bioavailability of the composition in the system. In some embodiments, after administration, the modification increases the affinity or avidity of the composition with a binding partner in the system, optionally where the binding partner is a receptor, cell, or cell membrane. In some embodiments, after administration, the modification decreases precipitation and/or aggregation of the composition in the system. In some embodiments, after administration, the modification increases the rate at which the composition enters one or more cells in the system. In some embodiments, after administration, the modification increases delivery of the composition to one or more cells in the system. In some embodiments, after administration, the modification increases delivery of the nucleic acid of the composition to one or more cells in the system. In some embodiments, the system is a mammalian subject and, after administration, the modification decreases accumulation of the composition in liver. In some embodiments, the system is a mammalian subject and, after administration, the modification increases the amount of composition that crosses the blood-brain barrier.

In some embodiments, the present disclosure provides a method that includes administering a composition of the present disclosure including at least one polynucleotide and at least one engineered polypeptide to a cell, tissue, or subject, wherein one or more amino acids of the engineered polypeptide is a phosphorylated amino acid. In some embodiments, the phosphorylated amino acid is a serine, threonine, or tyrosine amino acid. In some embodiments, the phosphorylated amino acid is present in a linker domain or targeting domain. In some embodiments, the composition is delivered to cells of the central nervous system (CNS).

In some embodiments, the composition is delivered to CNS neurons. In some embodiments, the composition is delivered to CNS astrocytes, microglia, oligodendrocytes, or glia. In some embodiments, the composition is delivered to spinal cord cells, optionally wherein the spina cord cells are spinal cord neurons or spinal cord glial cells. In some embodiments, the polynucleotide encodes an expression product that is expressed in cells to which the composition is delivered. In some embodiments, the subject is a mammalian subject and the administration is intrathecal, intracranial, or intra-cisterna magna.

In some embodiments, the present disclosure provides a method that includes administering a composition of the present disclosure including at least one polynucleotide and at least one engineered polypeptide to a cell, tissue, or subject, wherein one or more amino acids of the engineered polypeptide is a sulfated amino acid. In some embodiments, the sulfated amino acid is a serine, threonine, or tyrosine amino acid. In some embodiments, the sulfated amino acid is present in a linker domain or targeting domain. In some embodiments, the composition is delivered to cells of the central nervous system (CNS). In some embodiments, the composition is delivered to CNS neurons. In some embodiments, the composition is delivered to CNS astrocytes, microglia, oligodendrocytes, or glia. In some embodiments, the composition is delivered to spinal cord cells, optionally wherein the spina cord cells are spinal cord neurons or spinal cord glial cells. In some embodiments, the polynucleotide encodes an expression product that is expressed in cells to which the composition is delivered. In some embodiments, the subject is a mammalian subject and the administration is intrathecal, intracranial, or intra-cisterna magna.

In some embodiments, the present disclosure provides a method that includes administering a composition of the present disclosure including at least one polynucleotide and at least one engineered polypeptide o a cell, tissue, or subject, wherein one or more amino acids of the engineered polypeptide is an acetylated amino acid. In some embodiments, the acetylated amino acid is a lysine amino acid. In some embodiments, the acetylated amino acid is present in a linker domain or targeting domain. In some embodiments, the composition is delivered to CNS neurons. In some embodiments, the composition is delivered to retinal cells. In some embodiments, the composition is delivered to retinal neurons, optionally wherein the retinal neurons include one or more of photoreceptors, bipolar cells, retinal ganglion cells, horizontal cells, and amacrine cells. In some embodiments, the composition is delivered to photoreceptor cells, optionally wherein the photoreceptor cells include one or both of rods and cones. In some embodiments, the polynucleotide encodes an expression product that is expressed in cells to which the composition is delivered. In some embodiments, the subject is a mammalian subject and the administration is intravitreal, suprachoroidal, or subretinal.

In some embodiments, the present disclosure provides a method that includes administering a composition of the present disclosure including at least one polynucleotide and at least one engineered polypeptide to a cell, tissue, or subject, wherein one or more amino acids of the engineered polypeptide is a mannosylated amino acid. In some embodiments, the mannosylated amino acid is a serine amino acid. In some embodiments, the mannosylated amino acid is present in a linker domain or targeting domain. In some embodiments, the composition is delivered to CNS neurons. In some embodiments, the composition is delivered to retinal cells. In some embodiments, the composition is delivered to retinal neurons, optionally wherein the retinal neurons include one or more of photoreceptors, bipolar cells, retinal ganglion cells, horizontal cells, and amacrine cells. In some embodiments, the composition is delivered to photoreceptor cells include one or both of rods and cones. In some embodiments, the polynucleotide encodes an expression product that is expressed in cells to which the composition is delivered. In some embodiments, the subject is a mammalian subject and the administration is intravitreal, suprachoroidal, or subretinal.

In at least one further aspect, the present disclosure provides a method of condensing a polynucleotide, including contacting the polynucleotide with a polypeptide of the present disclosure.

In at least one further aspect, the present disclosure provides a method of neutralizing the charge of a polynucleotide, including contacting the polynucleotide with a polypeptide of the present disclosure.

In at least one further aspect, the present disclosure provides a composition including an engineered polypeptide and at least one nucleic acid, the engineered polypeptide including a nucleic acid binding domain, a targeting domain, and a nucleic acid release domain, where one or more amino acids of the engineered polypeptide is a modified amino acid, optionally where the modification includes at least one of: (i) phosphorylation; (ii) sulfation; (iii) glycosylation; (iv) prenylation; (v) methylation; (vi) sialylation; (vii) lipidation and/or lipoylation; (viii) acetylation; (ix) hydroxylation; (x) palmitoylation; (xi) mannosylation; (xii) myristoylation; (xiii) fucosylation; (xiv) pegylation; and/or (xv) any combination thereof, optionally where the composition is for use in delivering a nucleic acid to a subject or system.

In at least one further aspect, the present disclosure provides a composition including an engineered polypeptide and at least one nucleic acid, the engineered polypeptide including a nucleic acid binding domain and a targeting domain, where one or more amino acids of the engineered polypeptide is a modified amino acid, optionally where the modification includes at least one of: (i) phosphorylation; (ii) sulfation; (iii) glycosylation; (iv) prenylation; (v) methylation; (vi) sialylation; (vii) lipidation and/or lipoylation; (viii) acetylation; (ix) hydroxylation; (x) palmitoylation; (xi) mannosylation; (xii) myristoylation; (xiii) fucosylation; (xiv) pegylation; and/or (xv) any combination thereof, optionally where the composition is for use in delivering a nucleic acid to a subject or system. In some embodiments, the engineered polypeptide comprises a targeting domain.

In at least one aspect, the present disclosure provides an engineered polypeptide that includes a nucleic acid binding domain and a targeting domain, which engineered polypeptide can be a mini-nucleosome core protein. A loaded mini-nucleosome can be or provide a non-viral vector that includes an engineered polypeptide (e.g., a mini-nucleosome core protein) as described herein and at least one nucleic acid molecule as provided herein or otherwise known in the art.

In some embodiments, an engineered polypeptide (e.g., a mini-nucleosome core protein) that is or includes a nucleic acid binding domain is derived from a histone polypeptide sequence and/or a nucleic acid binding domain that is or includes the amino acid sequence KRHRK. In certain embodiments, an engineered polypeptide of the present disclosure includes a nucleic acid binding domain that is or includes an amino acid sequence that includes KRHRK, RRRRR, RRLARR, KKAKAAAKPKK, KKDGKKRKR, KKKLK, KKRIRK, RKKSK, KKPKK, or a combination thereof, but not limited to it.

In some embodiments, an engineered polypeptide of the present disclosure includes a nucleic acid binding domain derived from any histone protein sequence or those described in Table 3 or a combination of the sequences described herein but not limited to it. These nucleic acid binding domains may be derived from various human proteins or other organisms. One skilled in the art may contemplate modifying or engineering the “NABD” with changes to the amino acid sequence. One skilled in the art may also contemplate placing the “NABD” in reverse sequence or by switching amino acid positions within the domain or adding posttranslational modifications to amino acids.

In some embodiments, an engineered polypeptide of the present disclosure includes a targeting domain that is a cell attachment targeting domain, a beta galactose binding domain, a fucose binding domain, a heparin binding domain, a sialic acid binding domain, a glycoprotein binding domain, a carbohydrate binding domain, a lysophosphatidic acid binding domain, a cAMP binding domain, a hyaluronan binding domain, a chondroitin sulfate binding domain, an integrin binding domain, a nucleolin binding domain, a collagen binding domain, a clathrin binding domain, a Fc receptor binding domain, an actin binding domain, an endocytosis motif, a nuclear localization signal, or a combination thereof but not limited to it. Some examples of those domain are described in Table 5 but is not limited to these. These domains may be derived from any human proteins or other organisms. One skilled in the art may contemplate modifying or engineering the targeting domain with changes to the amino acid sequence. One skilled in the art may also contemplate placing the targeting domain in reverse sequence or by switching amino acid positions within the domain or adding posttranslational modifications to amino acids.

In some embodiments, an engineered polypeptide of the present disclosure includes a targeting domain that is an internalization domain wherein the internalization domain is or includes an amino acid sequence that includes FXDXF, PPSY, FEDNFVP, YIRV, YADW, YTQV, KKRPKP. SSDDE. RRASS. (YXXL)2, LPLTG, LAFTG, or a combination thereof but not limited to it. These domains may be derived from human proteins or other organisms. One skilled in the art may contemplate modifying or engineering the internalization domain with changes to the amino acid sequence. One skilled in the art may also contemplate placing the internalization domain in reverse sequence or by switching amino acid positions within the domain or adding posttranslational modifications to amino acids.

Those of skill in the art will appreciate that, as used in protein sequences throughout the present specification, an “X” can refer to any amino acid unless otherwise specified. Thus, unless otherwise specified, an “X” is a placeholder for a single amino acid, which position could be filled by any single amino acid known to those of skill in the art.

In some embodiments, an engineered polypeptide of the present disclosure includes a cell attachment targeting domain that is or includes an amino acid sequence selected from WGREERQ, NTQIH, WNNKTPH, TPH, VNRWS, XBBBXXBX, ARKKAAKA, QRR, SRR, WEPSRPFPVD, HRRTRKAPKRIRLPHIR, KRTGQYKLGSKTGPGQK, KKTK, KLRSQLVKK, RRRCGQKKK, BX(7)B, RIQNLLKITNLRIKFVK, KKEKDIMKKTI, KGE, RGD, RGDS. TTVVNPKYEGK, ERMSQIKRLLS, WRHRARS, GFOGER, LFDLM, WGREERQ, QSTEKRG, LPNTG, and a combination thereof, where X can be any amino acid, but not limited to it.

In some embodiments, an engineered polypeptide of the present disclosure includes a targeting domain that is an internalization domain cell-type specific targeting domain wherein the cell-type specific targeting domain is or includes an amino acid sequence that includes ASSLNIA, KKEEEKKEEEKKEEE, LIFHKEQ, KFNKPFVFLI, QPEHSST, EYHHYNK, NGR, GEKGEP, KTKKK, KALKKK, KGKKK. CSVTCG, LRE, YKYNLNGRES, YRSL, KGGK7, KKKQYTSIHHG, KDEL, LADQDYTKTA, or a combination thereof but not limited to it. These domains may be derived from human proteins or other organisms. One skilled in the art may contemplate modifying or engineering the targeting domain with changes to the amino acid sequence. One skilled in the art may also contemplate placing the targeting domain in reverse sequence or by switching amino acid positions within the domain or adding posttranslational modifications to amino acids.

In some embodiments, an engineered polypeptide of the present disclosure includes a poly-arginine domain with varying length or multiple poly-arginine domains throughout the polypeptide sequence.

In some embodiments, an engineered polypeptide of the present disclosure includes a nuclear internalization signal or a nuclear import machinery binding domain. The engineered polypeptide, the nuclear internalization signal or a nuclear import machinery binding domain can be or include an amino acid sequence that includes KKKYKLK, KKRKLE, TRSK, HRKRKR, NKRKRK, AEKSKKK, RKSK, KRVK, KRK, LQQTPLHLAVI, RRPR, PRPR, RPPP, RKKRKGK. PAAKRVKLD, KLKIKRPVK, PKKKRKV, QRKRQK, DSPE, FQVT, QSTEKRG, RQGLID, Cyclic RKKH, or a combination thereof but not limited to it. These domains may be derived from human proteins or other organisms. One skilled in the art may contemplate modifying or engineering the nuclear internalization signal with changes to the amino acid sequence. One skilled in the art may also contemplate placing the nuclear internalization signal in reverse sequence or by switching amino acid positions within the domain or adding posttranslational modifications to amino acids.

In some embodiments, an engineered polypeptide of the present disclosure includes a nucleic acid release domain. The nucleic acid release domain is or includes an amino acid sequence that includes GRKKRRQRRRPQ, KRH, KSVKKRSVSEIQ, NRRKKRAL, KFERQ, VRGP, NKDS, NRDN, ANNR, or a combination thereof but not limited to it. These domains may be derived from various proteins that are substrates of peptidases, enzymes or other proteins found in humans or other organisms. Some nucleic acid release domains may also be derived from autolysis sites of various proteins. One skilled in the art may contemplate modifying or engineering the nucleic acid release domain with changes to the amino acid sequence. One skilled in the art may also contemplate placing the nucleic acid release signal in reverse sequence or by switching amino acid positions within the domain or adding posttranslational modifications to amino acids.

In some embodiments, an engineered polypeptide of the present disclosure further including a stability domain. In some embodiments, an engineered polypeptide of the present disclosure can include a stability domain that is or includes an amino acid sequence that includes YTRF, GDAY, LLEE, RKKRRQRRR, YKSL, YENF, FQDL, YIGSR, IKVAV, or a combination thereof but not limited to it. These domains may be derived from human proteins or other organisms. One skilled in the art may contemplate modifying or engineering the stability domain with changes to the amino acid sequence. One skilled in the art may also contemplate placing the stability domain in reverse sequence or by switching amino acid positions within the domain or adding posttranslational modifications to amino acids.

In some embodiments, an engineered polypeptide of the present disclosure includes an oligomerization domain. In some embodiments, an engineered polypeptide of the present disclosure can include an oligomerization domain is selected from the oligomerization domains of Table 11 but not limited to it. The position of oligomerization domain is positioned at the C-terminus of an engineered polypeptide of the present disclosure or at any other locations. These domains may be derived from human proteins or other organisms. One skilled in the art may contemplate modifying or engineering the oligomerization domain with changes to the amino acid sequence. One skilled in the art may also contemplate placing the oligomerization domain in reverse sequence or by switching amino acid positions within the domain or adding posttranslational modifications to amino acids.

In some embodiments, an engineered polypeptide of the present disclosure includes a Linker. In some embodiments, an engineered polypeptide of the present disclosure can include a Linker selected, without limitation, from the exemplary domains of Table 12. The position of linker in an engineered polypeptide of the present disclosure may be in between other domains and any other locations. These Linkers may be derived from human proteins or other organisms. One skilled in the art may contemplate modifying or engineering the linker domain with changes to the amino acid sequence. One skilled in the art may also contemplate placing the linker domain in reverse sequence or by switching amino acid positions within the domain or adding posttranslational modifications to amino acids.

In various embodiments, two or more engineered polypeptides of the present disclosure can oligomerize.

In some embodiments, the present disclosure includes a composition that includes an engineered polypeptide of the present disclosure (e.g., a mini-nucleosome core protein) together with at least one polynucleotide. In some embodiments, the polypeptide is a DNA or RNA polynucleotide. In some embodiments, the polypeptide is a or includes an inhibitory RNA, wherein the inhibitory RNA is a gRNA, siRNA, miRNA, or shRNA. In various embodiments, the polypeptide(s) and polynucleotide(s) are not associated but are together in a composition, e.g., a kit or solution. In various embodiments, the polypeptide(s) and polynucleotide(s) are associated, e.g., condensed, e.g., to form a loaded mini-nucleosome. In certain embodiments, the ratio of polynucleotides to engineered polypeptides is between 1:1 and 1:2,000 or between 1:3 and 1:2,000. In certain embodiments, the ratio of polynucleotides to engineered polypeptides is between 1:1 and 1:2,000. In certain embodiments, the ratio of polynucleotides to engineered polypeptides is between 1:1 and 1:1,000, between 1:1 and 1:500, between 1:1 and 1:200, between 1:1 and 1:100, between 1:1 and 1:50. In certain embodiments, the ratio of polynucleotides to engineered polypeptides is between 1:3 and 1:1,000, between 1:3 and 1:500, between 1:3 and 1:200, between 1:3 and 1:100, or between 1:3 and 1:50. In certain embodiments, the ratio of polynucleotides to engineered polypeptides is between 1:200 and 1:2,000, between 1:200 and 1:1000, or between 1:200 and 1:500. In certain embodiments, the ratio of polynucleotides to engineered polypeptides is between 1:1 and 1:50, 1:1 and 1:40, 1:1 and 1:30, 1:1 and 1:20, 1:1 and 1:10, 1:1 and 1:5, 1:1 and 1:4, 1:1 and 1:3, or 1:1 and 1:2. One skilled in the art may also contemplate chemical modifications to the DNA or RNA molecules.

In some embodiments, a composition provided herein that includes a mini-nucleosome core protein and/or a loaded mini-nucleosome) can be administered to or contacted with a cell, tissue, or subject. The conditions of application may be in in vitro, ex vivo or in vivo. Such engineered cell may include a pharmaceutical carrier, e.g., that may be used in, or is compatible with, delivery of therapeutic materials (e.g., a composition provided herein that includes a mini-nucleosome core protein and/or a loaded mini-nucleosome) to various parts of human body for example brain, retina, gut, pancreas, lung etc. without any limitations.

In some embodiments, a method of condensing a polynucleotide may include contacting a polynucleotide with a mini-nucleosome core protein as described herein. The method may include process of neutralizing the charge of a polynucleotide or condensation of the polynucleotide into nano-sized particles, including contacting the polynucleotide with a mini-nucleosome core protein described herein.

In some embodiments, the mini-nucleosome core protein may be a branched peptide or a cyclic peptide but not limited to these characteristics. One skilled in the art may contemplate changing the characteristics of mini-nucleosome core protein to obtain enhanced tropism to various cell types.

The present disclosure further provides a polynucleotide encoding an engineered polypeptide (e.g., a mini-nucleosome core protein) as provided herein. The polynucleotide encoding the engineered polypeptide can be a DNA polynucleotide or an RNA polynucleotide. In some instances, the present disclosure provides a vector including a polynucleotide that encodes an engineered polypeptide of the present disclosure. In some embodiments, the present disclosure provides a cell that includes a polynucleotide encoding an engineered polypeptide (e.g., a mini-nucleosome core protein) as provided herein, a vector including such polynucleotide, or includes the sequence of such polynucleotide. In certain embodiments, an engineered polypeptide of the present disclosure can be isolated from one or more such cells.

In various embodiments, one or more amino acids of an engineered polypeptide of the present disclosure has at least 80% sequence identity with an amino acid sequence selected from SEQ ID NOs: 336-388 (e.g., at least 80%, 85%, 90%, 95%, 96%, 97%, 98%, 99%, or 100% sequence identity with an amino acid sequence selected from SEQ ID NOs: 336-388).

In various embodiments, one or more amino acids of an engineered polypeptide of the present disclosure (e.g., a mini-nucleosome core protein) is pegylated, acetylated, methylated, glycosylated, phosphorylated, sumoylated, amidated, lipidated, prenylated, lipoylated, alkylated, acylated, glycated, nitrosylated, sulfated, carbamylated, carbonylated, neddylated, biotinylated, or ribosylated.

### Definitions

About: The term “about,” when used herein in reference to a value, refers to a value that is similar, in context to the referenced value. In general, those skilled in the art, familiar with the context, will appreciate the relevant degree of variance encompassed by “about” in that context. For example, in some embodiments, the term “about” may encompass a range of values that within 25%, 20%, 19%, 18%, 17%, 16%, 15%, 14%, 13%, 12%, 11%, 10%, 9%, 8%, 7%, 6%, 5%, 4%, 3%, 2%, 1%, or less of the referenced value.

Administration: As used herein, the term “administration” typically refers to administration of a composition to a subject or system to achieve delivery of an agent that is, or is included in, the composition. Those of ordinary skill in the art will be aware of a variety of routes that may, in appropriate circumstances, be utilized for administration to a subject, for example a human. For example, in some embodiments, administration may be ocular, oral, parenteral, topical, etc. In some particular embodiments, administration may be bronchial (e.g., by bronchial instillation), buccal, dermal (which may be or include, for example, one or more of topical to the dermis, intradermal, interdermal, transdermal, etc.), enteral, intra-arterial, intradermal, intragastric, intramedullary, intramuscular, intranasal, intraperitoneal, intrathecal, intravenous, intraventricular, within a specific organ (e. g, intrahepatic), mucosal, nasal, oral, rectal, subcutaneous, sublingual, topical, tracheal (e.g., by intratracheal instillation), vaginal, vitreal, etc. In some embodiments, administration may involve only a single dose. In some embodiments, administration may involve application of a fixed number of doses. In some embodiments, administration may involve dosing that is intermittent (e.g., a plurality of doses separated in time) and/or periodic (e.g., individual doses separated by a common period of time) dosing. In some embodiments, administration may involve continuous dosing (e.g., perfusion) for at least a selected period of time.

Associated with: Two events or entities are “associated” with one another, as that term is used herein, if the presence, level and/or form of one is correlated with that of the other. For example, a particular entity (e.g., polypeptide, genetic signature, metabolite, microbe, etc.) is considered to be associated with a particular disease, disorder, or condition, if its presence, level and/or form correlates with incidence of and/or susceptibility to the disease, disorder, or condition (e.g., across a relevant population). In some embodiments, two or more entities are physically “associated” with one another if they interact, directly or indirectly, so that they are and/or remain in physical proximity with one another. In some embodiments, two or more entities that are physically associated with one another are covalently linked to one another; in some embodiments, two or more entities that are physically associated with one another are not covalently linked to one another but are non-covalently associated, for example by means of hydrogen bonds, van der Waals interaction, hydrophobic interactions, magnetism, and combinations thereof.

Agent: As used herein, the term “agent,” may refer to a compound, molecule, or entity of any chemical class including, for example, a small molecule, polypeptide, nucleic acid, saccharide, lipid, metal, or a combination or complex thereof. In some embodiments, the term “agent” may refer to a compound, molecule, or entity that includes a polymer. In some embodiments, the term may refer to a compound or entity that includes one or more polymeric moieties. In some embodiments, the term “agent” may refer to a compound, molecule, or entity that is substantially free of a particular polymer or polymeric moiety. In some embodiments, the term may refer to a compound, molecule, or entity that lacks or is substantially free of any polymer or polymeric moiety.

Amino acid: In its broadest sense, as used herein, “amino acid” refers to any compound and/or substance that can be incorporated into a polypeptide chain, e.g., through formation of one or more peptide bonds. In some embodiments, an amino acid has the general structure H₂N—C(H)(R)—COOH. In some embodiments, an amino acid is a naturally-occurring amino acid. In some embodiments, an amino acid is a non-natural amino acid; in some embodiments, an amino acid is a D-amino acid; in some embodiments, an amino acid is an L-amino acid. “Standard amino acid” refers to any of the twenty standard L-amino acids commonly found in naturally occurring peptides. “Nonstandard amino acid” refers to any amino acid, other than the standard amino acids, regardless of whether it is prepared synthetically or obtained from a natural source. In some embodiments, an amino acid, including a carboxy- and/or amino-terminal amino acid in a polypeptide, can contain a structural modification as compared with the general structure above. For example, in some embodiments, an amino acid may be modified by methylation, amidation, acetylation, pegylation, glycosylation, phosphorylation, and/or substitution (e.g., of the amino group, the carboxylic acid group, one or more protons, and/or the hydroxyl group) as compared with the general structure. In some embodiments, such modification may, for example, alter the circulating half-life of a polypeptide containing the modified amino acid as compared with one containing an otherwise identical unmodified amino acid. In some embodiments, such modification does not significantly alter a relevant activity of a polypeptide containing the modified amino acid, as compared with one containing an otherwise identical unmodified amino acid. As will be clear from context, in some embodiments, the term “amino acid” may be used to refer to a free amino acid; in some embodiments it may be used to refer to an amino acid residue of a polypeptide.

Between: As used herein, the term “between” refers to content that falls between indicated upper and lower, or first and second, boundaries, inclusive of the boundaries.

Corresponding to: As used herein, the term “corresponding to” may be used to designate the position/identity of a structural element in a compound or composition through comparison with an appropriate reference compound or composition. For example, in some embodiments, a monomeric residue in a polymer (e.g., an amino acid residue in a polypeptide or a nucleic acid residue in a polynucleotide) may be identified as “corresponding to” a residue in an appropriate reference polymer. For example, those of ordinary skill will appreciate that, for purposes of simplicity, residues in a polypeptide are often designated using a canonical numbering system based on a reference related polypeptide, so that an amino acid “corresponding to” a residue at position 190, for example, need not actually be the 190^{th} amino acid in a particular amino acid chain but rather corresponds to the residue found at 190 in the reference polypeptide; those of ordinary skill in the art readily appreciate how to identify “corresponding” amino acids. For example, those skilled in the art will be aware of various sequence alignment strategies, including software programs such as, for example, BLAST, CS-BLAST, CUDASW++, DIAMOND, FASTA, GGSEARCH/GLSEARCH, Genoogle, HMMER, HHpred/HHsearch, IDF, Infernal, KLAST, USEARCH, parasail, PSI-BLAST, PSI-Search, ScalaBLAST, Sequilab, SAM, SSEARCH, SWAPHI, SWAPHI-LS, SWIMM, or SWIPE that can be utilized, for example, to identify “corresponding” residues in polypeptides and/or nucleic acids in accordance with the present disclosure.

Domain: The term “domain” as used herein refers to a section or portion of an entity. In some embodiments, a “domain” is associated with a particular structural and/or functional feature of the entity so that, when the domain is physically separated from the rest of its parent entity, it substantially or entirely retains the particular structural and/or functional feature. Alternatively or additionally, a domain may be or include a portion of an entity that, when separated from that (parent) entity and linked with a different (recipient) entity, substantially retains and/or imparts on the recipient entity one or more structural and/or functional features that characterized it in the parent entity. In some embodiments, a domain is a section or portion of a molecule (e.g., a small molecule, carbohydrate, lipid, nucleic acid, or polypeptide). In some embodiments, a domain is a section of a polypeptide; in some such embodiments, a domain is characterized by a particular structural element (e.g., a particular amino acid sequence or sequence motif, α-helix character, β-sheet character, coiled-coil character, random coil character, etc.), and/or by a particular functional feature (e.g., binding activity, enzymatic activity, folding activity, signaling activity, etc.). In some embodiments, a domain is or includes a characteristic portion or characteristic sequence element.

Engineered: In general, the term “engineered” refers to the aspect of having been manipulated by the hand of man. For example, a polynucleotide is considered to be “engineered” when two or more sequences, that are not linked together in that order in nature, are manipulated by the hand of man to be directly linked to one another in the engineered polynucleotide. Those of skill in the art will appreciate that an “engineered” nucleic acid or amino acid sequence can be a recombinant nucleic acid or amino acid sequence. In some embodiments, an engineered polynucleotide includes a domain-encoding sequence regulatory sequence that is found in nature in operative association with a first sequence but not in operative association with a second sequence, is linked by the hand of man so that it is operatively associated with the second sequence. Comparably, a cell or organism is considered to be “engineered” if it has been manipulated so that its genetic information is altered (e.g., new genetic material not previously present has been introduced, for example by transformation, mating, somatic hybridization, transfection, transduction, or other mechanism, or previously present genetic material is altered or removed, for example by substitution or deletion mutation, or by mating protocols). As is common practice and is understood by those in the art, progeny of an engineered polynucleotide or cell are typically still referred to as “engineered” even though the actual manipulation was performed on a prior entity.

Gene: As used herein, the term “gene” refers to a DNA sequence that codes for a product (e.g., an RNA product and/or a polypeptide product). In some embodiments, a gene includes coding sequence (i.e., sequence that encodes a particular product); in some embodiments, a gene includes non-coding sequence. In some particular embodiments, a gene may include both coding (e.g., exonic) and non-coding (e.g., intronic) sequences. In some embodiments, a gene may include one or more regulatory elements that, for example, may control or impact one or more aspects of gene expression (e.g., a promoter). A gene can be endogenous or non-endogenous in a particular context, e.g., a cell. A gene can be a transgene.

Gene product or expression product: As used herein, the term “gene product” or “expression product” generally refers to an RNA transcribed from a gene (pre- and/or post-processing) or a polypeptide (pre- and/or post-modification) encoded by an RNA transcribed from a gene.

“Improve,” “increase,” “inhibit,” or “reduce”: As used herein, the terms “improve,” “increase,” “inhibit,” “reduce,” or grammatical equivalents thereof, indicate values that are relative to a baseline or other reference measurement. In some embodiments, an appropriate reference measurement may be or include a measurement in a particular system (e.g., in a single individual) under otherwise comparable conditions absent presence of (e.g., prior to and/or after) a particular agent or treatment, or in presence of an appropriate comparable reference agent. In some embodiments, an appropriate reference measurement may be or include a measurement in comparable system known or expected to respond in a particular way, in presence of the relevant agent or treatment.

Increase and decrease: As used herein, the terms “increase,” “decrease,” and grammatical equivalents thereof, indicate qualitative or quantitative difference from a reference.

Nucleic acid: As used herein, in its broadest sense, “nucleic acid” refers to any compound and/or substance that is or can be incorporated into an oligonucleotide chain. In some embodiments, a nucleic acid is a compound and/or substance that is or can be incorporated into an oligonucleotide chain via a phosphodiester linkage. As will be clear from context, in some embodiments, “nucleic acid” refers to an individual nucleic acid residue (e.g., a nucleotide and/or nucleoside); in some embodiments, “nucleic acid” refers to an oligonucleotide chain including individual nucleic acid residues. In some embodiments, a “nucleic acid” is or includes RNA; in some embodiments, a “nucleic acid” is or includes DNA. In some embodiments, a nucleic acid is, includes, or consists of one or more natural nucleic acid residues. In some embodiments, a nucleic acid is, includes, or consists of one or more nucleic acid analogs. In some embodiments, a nucleic acid analog differs from a nucleic acid in that it does not utilize a phosphodiester backbone. For example, in some embodiments, a nucleic acid is, includes, or consists of one or more “peptide nucleic acids”, which are known in the art and have peptide bonds instead of phosphodiester bonds in the backbone, are considered within the scope of the present disclosure. Alternatively or additionally, in some embodiments, a nucleic acid has one or more phosphorothioate and/or 5′-N-phosphoramidite linkages rather than phosphodiester bonds. In some embodiments, a nucleic acid is, includes, or consists of one or more natural nucleosides (e.g., adenosine, thymidine, guanosine, cytidine, uridine, deoxyadenosine, deoxythymidine, deoxy guanosine, and deoxycytidine). In some embodiments, a nucleic acid is, includes, or consists of one or more nucleoside analogs (e.g., 2-aminoadenosine, 2-thiothymidine, inosine, pyrrolo-pyrimidine, 3-methyl adenosine, 5-methylcytidine, C-5 propynyl-cytidine, C-5 propynyl-uridine, 2-aminoadenosine, C5-bromouridine, C5-fluorouridine, C5-iodouridine, C5-propynyl-uridine, C5-propynyl-cytidine, C5-methylcytidine, 2-aminoadenosine, 7-deazaadenosine, 7-deazaguanosine, 8-oxoadenosine, 8-oxoguanosine, 0(6)-methylguanine, 2-thiocytidine, methylated bases, intercalated bases, and combinations thereof). In some embodiments, a nucleic acid includes one or more modified sugars (e.g., 2′-fluororibose, ribose, 2′-deoxyribose, arabinose, and hexose) as compared with those in natural nucleic acids. In some embodiments, a nucleic acid has a nucleotide sequence that encodes a functional gene product such as an RNA or protein. In some embodiments, a nucleic acid includes one or more introns. In some embodiments, nucleic acids are prepared by one or more of isolation from a natural source, enzymatic synthesis by polymerization based on a complementary template (in vivo or in vitro), reproduction in a recombinant cell or system, and chemical synthesis. In some embodiments, a nucleic acid is at least 3, 4, 5, 6, 7, 8, 9, 10, 15, 20, 25, 30, 35, 40, 45, 50, 55, 60, 65, 70, 75, 80, 85, 90, 95, 100, 110, 120, 130, 140, 150, 160, 170, 180, 190, 20, 225, 250, 275, 300, 325, 350, 375, 400, 425, 450, 475, 500, 600, 700, 800, 900, 1000, 1500, 2000, 2500, 3000, 3500, 4000, 4500, 5000 or more residues long. In some embodiments, a nucleic acid is partly or wholly single stranded; in some embodiments, a nucleic acid is partly or wholly double stranded. In some embodiments a nucleic acid has a nucleotide sequence including at least one element that encodes, or is the complement of a sequence that encodes, a polypeptide. In some embodiments, a nucleic acid has enzymatic activity.

Operably linked: As used herein, “operably linked” refers to a juxtaposition where the components described are in a relationship permitting them to function in their intended manner. For example, a control element “operably linked” to a functional element is associated in such a way that expression and/or activity of the functional element is achieved under conditions compatible with the control element. In some embodiments, “operably linked” control elements are contiguous (e.g., covalently linked) with the coding elements of interest; in some embodiments, control elements act in trans to or otherwise at a from the functional element of interest.

Pharmaceutical composition: As used herein, the term “pharmaceutical composition” refers to a composition in which an active agent is formulated together with one or more pharmaceutically acceptable carriers. In some embodiments, the active agent is present in unit dose amount appropriate for administration in a therapeutic regimen that shows a statistically significant probability of achieving a predetermined therapeutic effect when administered to a relevant population. In some embodiments, a pharmaceutical composition may be specially formulated for administration in solid or liquid form, including those adapted for the following: oral administration, for example, drenches (aqueous or non-aqueous solutions or suspensions), tablets, e.g., those targeted for buccal, sublingual, and systemic absorption, boluses, powders, granules, pastes for application to the tongue; parenteral administration, for example, by subcutaneous, intramuscular, intravenous or epidural injection as, for example, a sterile solution or suspension, or sustained-release formulation; topical application, for example, as a cream, ointment, or a controlled-release patch or spray applied to the skin, lungs, or oral cavity; intravaginally or intrarectally, for example, as a pessary, cream, or foam; sublingually; ocularly; transdermally; or nasally, pulmonary, and to other mucosal surfaces.

Polypeptide: As used herein, “polypeptide” refers to any polymeric chain of amino acids. In some embodiments, a polypeptide has an amino acid sequence that occurs in nature. In some embodiments, a polypeptide has an amino acid sequence that does not occur in nature. In some embodiments, a polypeptide has an amino acid sequence that is engineered in that it is designed and/or produced through action of the hand of man. In some embodiments, a polypeptide may include or consist of natural amino acids, non-natural amino acids, or both. In some embodiments, a polypeptide may include or consist of only natural amino acids or only non-natural amino acids. In some embodiments, a polypeptide may include D-amino acids, L-amino acids, or both. In some embodiments, a polypeptide may include only D-amino acids. In some embodiments, a polypeptide may include only L-amino acids. In some embodiments, a polypeptide may include one or more pendant groups or other modifications, e.g., modifying or attached to one or more amino acid side chains, at the polypeptide's N-terminus, at the polypeptide's C-terminus, or any combination thereof. In some embodiments, such pendant groups or modifications may be selected from the group consisting of acetylation, amidation, lipidation, methylation, phosphorylation, glycosylation, glycation, sulfation, mannosylation, nitrosylation, acylation, palmitoylation, prenylation, pegylation, etc., including combinations thereof. In some embodiments, a polypeptide may be cyclic, and/or may include a cyclic portion. In some embodiments, a polypeptide is not cyclic and/or does not include any cyclic portion. In some embodiments, a polypeptide is linear. In some embodiments, a polypeptide may be or include a stapled polypeptide. In some embodiments, the term “polypeptide” may be appended to a name of a reference polypeptide, activity, or structure; in such instances, it is used herein to refer to polypeptides that share the relevant activity or structure and thus can be considered to be members of the same class or family of polypeptides. For each such class, the present specification provides and/or those skilled in the art will be aware of exemplary polypeptides within the class whose amino acid sequences and/or functions are known; in some embodiments, such exemplary polypeptides are reference polypeptides for the polypeptide class or family. In some embodiments, a member of a polypeptide class or family shows significant sequence similarity (e.g., homology) or identity with, shares a common sequence motif (e.g., a characteristic sequence element) with, and/or shares a common activity (in some embodiments at a comparable level or within a designated range) with a reference polypeptide of the class; in some embodiments with all polypeptides within the class). For example, in some embodiments, a member polypeptide shows an overall degree of sequence similarity (e.g., homology) or identity with a reference polypeptide that is at least about 30-40%, and is often greater than about 50%, 60%, 70%, 80%, 90%, 91%, 92%, 93%, 94%, 95%, 96%, 97%, 98%, 99% or more and/or includes at least one region (e.g., a conserved region that may in some embodiments be or include a characteristic sequence element) that shows very high sequence identity, often greater than 90% or even 95%, 96%, 97%, 98%, or 99%. Such a conserved region usually encompasses at least 3-4 and often up to 20 or more amino acids; in some embodiments, a conserved region encompasses at least one stretch of at least 2, 3, 4, 5, 6, 7, 8, 9, 10, 11, 12, 13, 14, 15 or more contiguous amino acids. In some embodiments, a useful polypeptide may include or consist of a fragment of a parent polypeptide. In some embodiments, a useful polypeptide as may include or consist of a plurality of fragments, each of which is found in the same parent polypeptide in a different spatial arrangement relative to one another than is found in the polypeptide of interest (e.g., fragments that are directly linked in the parent may be spatially separated in the polypeptide of interest or vice versa, and/or fragments may be present in a different order in the polypeptide of interest than in the parent), so that the polypeptide of interest is a derivative of its parent polypeptide.

Prevent or prevention: As used herein, “prevent” or “prevention,” when used in connection with the occurrence of a disease, disorder, and/or condition, refers to reducing the risk of developing the disease, disorder and/or condition and/or to delaying onset of one or more characteristics or symptoms of the disease, disorder or condition. Prevention may be considered complete when onset of a disease, disorder or condition has been delayed for a predefined period of time.

Promoter: As used herein, a “promoter” or “promoter sequence” can be a DNA regulatory region that directly or indirectly (e.g., through promoter-bound proteins or substances) participates in initiation and/or processivity of transcription of a coding sequence. A promoter may, under suitable conditions, initiate transcription of a coding sequence upon binding of one or more transcription factors and/or regulatory moieties with the promoter. A promoter that participates in initiation of transcription of a coding sequence can be “operably linked” to the coding sequence. In certain instances, a promoter can be or include a DNA regulatory region that extends from a transcription initiation site (at its 3′ terminus) to an upstream (5′ direction) position such that the sequence so designated includes one or both of a minimum number of bases or elements necessary to initiate a transcription event. A promoter may be, include, or be operably associated with or operably linked to, expression control sequences such as enhancer and repressor sequences. In some embodiments, a promoter may be inducible. In some embodiments, a promoter may be a constitutive promoter. In some embodiments, a conditional (e.g., inducible) promoter may be unidirectional or bi-directional. A promoter may be or include a sequence identical to a sequence known to occur in the genome of particular species. In some embodiments, a promoter can be or include a hybrid promoter, in which a sequence containing a transcriptional regulatory region can be obtained from one source and a sequence containing a transcription initiation region can be obtained from a second source. Systems for linking control elements to coding sequence within a transgene are well known in the art (general molecular biological and recombinant DNA techniques are described in Sambrook, Fritsch, and Maniatis, Molecular Cloning: A Laboratory Manual, Second Edition, Cold Spring Harbor Laboratory Press, Cold Spring Harbor, NY, 1989).

Recombinant: As used herein, “recombinant” is intended to refer to polypeptides that are designed, engineered, prepared, expressed, created, manufactured, and/or or isolated by recombinant means, such as polypeptides expressed using a recombinant expression vector transfected into a host cell; polypeptides isolated from a recombinant, combinatorial human polypeptide library; polypeptides isolated from an animal (e.g., a mouse, rabbit, sheep, fish, etc) that is transgenic for or otherwise has been manipulated to express a gene or genes, or gene components that encode and/or direct expression of the polypeptide or one or more component(s), portion(s), element(s), or domain(s) thereof, and/or polypeptides prepared, expressed, created or isolated by any other means that involves splicing or ligating selected nucleic acid sequence elements to one another, chemically synthesizing selected sequence elements, and/or otherwise generating a nucleic acid that encodes and/or directs expression of the polypeptide or one or more component(s), portion(s), element(s), or domain(s) thereof. In some embodiments, one or more of such selected sequence elements is found in nature. In some embodiments, one or more of such selected sequence elements is designed in silico. In some embodiments, one or more such selected sequence elements results from mutagenesis (e.g., in vivo or in vitro) of a known sequence element, e.g., from a natural or synthetic source such as, for example, in the germline of a source organism of interest (e.g., of a human, a mouse, etc).

Reference: As used herein describes a standard or control relative to which a comparison is performed. For example, in some embodiments, an agent, animal, individual, population, sample, sequence or value of interest is compared with a reference or control agent, animal, individual, population, sample, sequence or value. In some embodiments, a reference or control is tested and/or determined substantially simultaneously with the testing or determination of interest. In some embodiments, a reference or control is a historical reference or control, optionally embodied in a tangible medium. Typically, as would be understood by those skilled in the art, a reference or control is determined or characterized under comparable conditions or circumstances to those under assessment. Those skilled in the art will appreciate when sufficient similarities are present to justify reliance on and/or comparison to a particular possible reference or control.

Subject: As used herein, the term “subject” refers an organism, typically a mammal (e.g., a human, in some embodiments including prenatal human forms). In some embodiments, a subject is suffering from a relevant disease, disorder or condition. In some embodiments, a subject is susceptible to a disease, disorder, or condition. In some embodiments, a subject displays one or more symptoms or characteristics of a disease, disorder or condition. In some embodiments, a subject does not display any symptom or characteristic of a disease, disorder, or condition. In some embodiments, a subject is someone with one or more features characteristic of susceptibility to or risk of a disease, disorder, or condition. In some embodiments, a subject is a patient. In some embodiments, a subject is an individual to whom diagnosis and/or therapy is and/or has been administered.

Substantial sequence similarity: The phrase “substantial sequence similarity” is used herein to refer to a comparison between amino acid or nucleic acid sequences. As will be appreciated by those of ordinary skill in the art, two sequences are generally considered to be “substantially similar” if they contain a conservative amino acid substitution in corresponding positions. A conservative substitution is one in which an amino acid has been replaced by a non-identical residue having appropriately similar structural and/or functional characteristics. For example, as is well known by those of ordinary skill in the art, certain amino acids are typically classified as “hydrophobic” or “hydrophilic” amino acids, and/or as having “polar” or “nonpolar” side chains. Substitution of one amino acid for another of the same type may often be considered a conservative substitution. Typical amino acid categorizations are summarized in Tables 1 and 2 below:

TABLE 1

| | | | | | |
|---|---|---|---|---|---|
| Alanine | Ala | A | nonpolar | neutral | 1.8 |
| Arginine | Arg | R | polar | positive | −4.5 |
| Asparagine | Asn | N | polar | neutral | −3.5 |
| Aspartic acid | Asp | D | polar | negative | −3.5 |
| Cysteine | Cys | C | nonpolar | neutral | 2.5 |
| Glutamic acid | Glu | E | polar | negative | −3.5 |
| Glutamine | Gln | Q | polar | neutral | −3.5 |
| Glycine | Gly | G | nonpolar | neutral | −0.4 |
| Histidine | His | H | polar | positive | −3.2 |
| Isoleucine | Ile | I | nonpolar | neutral | 4.5 |
| Leucine | Leu | L | nonpolar | neutral | 3.8 |
| Lysine | Lys | K | polar | positive | −3.9 |
| Methionine | Met | M | nonpolar | neutral | 1.9 |
| Phenylalanine | Phe | F | nonpolar | neutral | 2.8 |
| Proline | Pro | P | nonpolar | neutral | −1.6 |
| Serine | Ser | S | polar | neutral | −0.8 |
| Threonine | Thr | T | polar | neutral | −0.7 |
| Tryptophan | Trp | W | nonpolar | neutral | −0.9 |
| Tyrosine | Tyr | Y | polar | neutral | −1.3 |
| Valine | Val | V | nonpolar | neutral | 4.2 |

TABLE 2

| Ambiguous Amino Acids | 3-Letter | 1-Letter |
|---|---|---|
| Asparagine or aspartic acid | Asx | B |
| Glutamine or glutamic acid | Glx | Z |
| Leucine or Isoleucine | Xle | J |
| Unspecified or unknown amino acid | Xaa | X |

As is well known in this art, amino acid or nucleic acid sequences may be compared using any of a variety of algorithms, including those available in commercial computer programs such as BLASTN for nucleotide sequences and BLASTP, gapped BLAST, and PSI-BLAST for amino acid sequences. Exemplary such programs are described in Altschul, et al., Basic local alignment search tool, J. Mol. Biol., 215(3): 403-410, 1990; Altschul, et al., Methods in Enzymology; Altschul, et al., “Gapped BLAST and PSI-BLAST: a new generation of protein database search programs,” Nucleic Acids Res. 25:3389-3402, 1997; Baxevanis, et al., Bioinformatics: A Practical Guide to the Analysis of Genes and Proteins, Wiley, 1998; and Misener, et al., (eds.), Bioinformatics Methods and Protocols (Methods in Molecular Biology, Vol. 132), Humana Press, 1999. In addition to identifying similar sequences, the programs mentioned above typically provide an indication of the degree of similarity. In some embodiments, two sequences are considered to be substantially similar if at least 50%, at least 55%, at least 60%, at least 65%, at least 70%, at least 75%, at least 80%, at least 85%, at least 90%, at least 91%, at least 92%, at least 93%, at least 94%, at least 95%, at least 96%, at least 97%, at least 98%, at least 99% or more of their corresponding residues are similar and/or identical over a relevant stretch of residues. In some embodiments, the relevant stretch is a complete sequence. In some embodiments, the relevant stretch is at least 10, at least 15, at least 20, at least 25, at least 30, at least 35, at least 40, at least 45, at least 50, at least 55, at least 60, at least 65, at least 70, at least 75, at least 80, at least 85, at least 90, at least 95, at least 100, at least 125, at least 150, at least 175, at least 200, at least 225, at least 250, at least 275, at least 300, at least 325, at least 350, at least 375, at least 400, at least 425, at least 450, at least 475, at least 500 or more residues. As would be appreciated by one of ordinary skill in the art sequences with substantial sequence similarity may be homologs of one another.

Substantial sequence identity: As used herein, the phrase “substantial sequence identity” refers to a comparison between amino acid or nucleic acid sequences. As will be appreciated by those of ordinary skill in the art, two sequences are generally considered to be “substantially identical” if they contain identical residues in corresponding positions. As is well known in this art, amino acid or nucleic acid sequences may be compared using any of a variety of algorithms, including those available in commercial computer programs such as BLASTN for nucleotide sequences and BLASTP, gapped BLAST, and PSI-BLAST for amino acid sequences. Exemplary such programs are described in Altschul et al., Basic local alignment search tool, J. Mol. Biol., 215(3): 403-410, 1990; Altschul et al., Methods in Enzymology; Altschul et al., Nucleic Acids Res. 25:3389-3402, 1997; Baxevanis et al., Bioinformatics: A Practical Guide to the Analysis of Genes and Proteins, Wiley, 1998; and Misener, et al, (eds.), Bioinformatics Methods and Protocols (Methods in Molecular Biology, Vol. 132), Humana Press, 1999. In addition to identifying identical sequences, the programs mentioned above typically provide an indication of the degree of identity. In some embodiments, two sequences are considered to be substantially identical if at least 50%, 55%, 60%, 65%, 70%, 75%, 80%, 85%, 90%, 91%, 92%, 93%, 94%, 95%, 96%, 97%, 98%, 99% or more of their corresponding residues are identical over a relevant stretch of residues. In some embodiments, the relevant stretch is a complete sequence. In some embodiments, the relevant stretch is at least 10, 15, 20, 25, 30, 35, 40, 45, 50, 55, 60, 65, 70, 75, 80, 85, 90, 95, 100, 125, 150, 175, 200, 225, 250, 275, 300, 325, 350, 375, 400, 425, 450, 475, 500 or more residues.

Therapeutic agent: As used herein, the phrase “therapeutic agent” in general refers to any agent that elicits a desired pharmacological effect when administered to an organism. In some embodiments, an agent is considered to be a therapeutic agent if it demonstrates a statistically significant effect across an appropriate population. In some embodiments, the appropriate population may be a population of model organisms. In some embodiments, an appropriate population may be defined by various criteria, such as a certain age group, gender, genetic background, preexisting clinical conditions, etc. In some embodiments, a therapeutic agent is a substance that can be used to alleviate, ameliorate, relieve, inhibit, prevent, delay onset of, reduce severity of, and/or reduce incidence of one or more symptoms or features of a disease, disorder, and/or condition. In some embodiments, a “therapeutic agent” is an agent that has been or is required to be approved by a government agency before it can be marketed for administration to humans. In some embodiments, a “therapeutic agent” is an agent for which a medical prescription is required for administration to humans.

Therapeutic regimen: A “therapeutic regimen,” as that term is used herein, refers to a dosing regimen whose administration across a relevant population may be correlated with a desired or beneficial therapeutic outcome.

Therapeutically effective amount: As used herein, is meant an amount that produces the desired effect for which it is administered. In some embodiments, the term refers to an amount that is sufficient, when administered to a population suffering from or susceptible to a disease, disorder, and/or condition in accordance with a therapeutic dosing regimen, to treat the disease, disorder, and/or condition. In some embodiments, a therapeutically effective amount is one that reduces the incidence and/or severity of, and/or delays onset of, one or more symptoms of the disease, disorder, and/or condition. Those of ordinary skill in the art will appreciate that the term “therapeutically effective amount” does not in fact require successful treatment be achieved in a particular individual. Rather, a therapeutically effective amount may be that amount that provides a particular desired pharmacological response in a significant number of subjects when administered to patients in need of such treatment. In some embodiments, reference to a therapeutically effective amount may be a reference to an amount as measured in one or more specific tissues (e.g., a tissue affected by the disease, disorder or condition) or fluids (e.g., blood, saliva, serum, sweat, tears, urine, etc.). Those of ordinary skill in the art will appreciate that, in some embodiments, a therapeutically effective amount of a particular agent or therapy may be formulated and/or administered in a single dose. In some embodiments, a therapeutically effective agent may be formulated and/or administered in a plurality of doses, for example, as part of a dosing regimen.

Treatment: As used herein, the term “treatment” (also “treat” or “treating”) refers to any administration of a therapy that partially or completely alleviates, ameliorates, relives, inhibits, delays onset of, reduces severity of, and/or reduces incidence of one or more symptoms, features, and/or causes of a particular disease, disorder, and/or condition. In some embodiments, such treatment may be of a subject who does not exhibit signs of the relevant disease, disorder and/or condition and/or of a subject who exhibits only early signs of the disease, disorder, and/or condition. Alternatively or additionally, such treatment may be of a subject who exhibits one or more established signs of the relevant disease, disorder and/or condition. In some embodiments, treatment may be of a subject who has been diagnosed as suffering from the relevant disease, disorder, and/or condition. In some embodiments, treatment may be of a subject known to have one or more susceptibility factors that are statistically correlated with increased risk of development of the relevant disease, disorder, and/or condition.

Variant: As used herein in the context of molecules, e.g., nucleic acids, proteins, or small molecules, the term “variant” refers to a molecule that shows significant structural identity with a reference molecule but differs structurally from the reference molecule, e.g., in the presence or absence or in the level of one or more chemical moieties as compared to the reference entity. In some embodiments, a variant also differs functionally from its reference molecule. In general, whether a particular molecule is properly considered to be a “variant” of a reference molecule is based on its degree of structural identity with the reference molecule. As will be appreciated by those skilled in the art, any biological or chemical reference molecule has certain characteristic structural elements. A variant, by definition, is a distinct molecule that shares one or more such characteristic structural elements but differs in at least one aspect from the reference molecule. To give but a few examples, a polypeptide may have a characteristic sequence element included of a plurality of amino acids having designated positions relative to one another in linear or three-dimensional space and/or contributing to a particular structural motif and/or biological function; a nucleic acid may have a characteristic sequence element included of a plurality of nucleotide residues having designated positions relative to on another in linear or three-dimensional space. In some embodiments, a variant polypeptide or nucleic acid may differ from a reference polypeptide or nucleic acid as a result of one or more differences in amino acid or nucleotide sequence and/or one or more differences in chemical moieties (e.g., carbohydrates, lipids, phosphate groups) that are covalently components of the polypeptide or nucleic acid (e.g., that are attached to the polypeptide or nucleic acid backbone). In some embodiments, a variant polypeptide or nucleic acid shows an overall sequence identity with a reference polypeptide or nucleic acid that is at least 85%, 86%, 87%, 88%, 89%, 90%, 91%, 92%, 93%, 94%, 95%, 96%, 97%, or 99%. In some embodiments, a variant polypeptide or nucleic acid does not share at least one characteristic sequence element with a reference polypeptide or nucleic acid. In some embodiments, a reference polypeptide or nucleic acid has one or more biological activities. In some embodiments, a variant polypeptide or nucleic acid shares one or more of the biological activities of the reference polypeptide or nucleic acid. In some embodiments, a variant polypeptide or nucleic acid lacks one or more of the biological activities of the reference polypeptide or nucleic acid. In some embodiments, a variant polypeptide or nucleic acid shows a reduced level of one or more biological activities as compared to the reference polypeptide or nucleic acid. In some embodiments, a polypeptide or nucleic acid of interest is considered to be a “variant” of a reference polypeptide or nucleic acid if it has an amino acid or nucleotide sequence that is identical to that of the reference but for a small number of sequence alterations at particular positions. Typically, fewer than about 20%, about 15%, about 10%, about 9%, about 8%, about 7%, about 6%, about 5%, about 4%, about 3%, or about 2% of the residues in a variant are substituted, inserted, or deleted, as compared to the reference. In some embodiments, a variant polypeptide or nucleic acid includes about 10, about 9, about 8, about 7, about 6, about 5, about 4, about 3, about 2, or about 1 substituted residues as compared to a reference. Often, a variant polypeptide or nucleic acid includes a very small number (e.g., fewer than about 5, about 4, about 3, about 2, or about 1) number of substituted, inserted, or deleted, functional residues (i.e., residues that participate in a particular biological activity) relative to the reference. In some embodiments, a variant polypeptide or nucleic acid includes not more than about 5, about 4, about 3, about 2, or about 1 addition or deletion, and, in some embodiments, includes no additions or deletions, as compared to the reference. In some embodiments, a variant polypeptide or nucleic acid includes fewer than about 25, about 20, about 19, about 18, about 17, about 16, about 15, about 14, about 13, about 10, about 9, about 8, about 7, about 6, and commonly fewer than about 5, about 4, about 3, or about 2 additions or deletions as compared to the reference. In some embodiments, a reference polypeptide or nucleic acid is one found in nature. In some embodiments, a reference polypeptide or nucleic acid is a human polypeptide or nucleic acid.

### BRIEF DESCRIPTION OF THE DRAWING

FIG. 1 includes panel A and panel B. Panel A is a schematic representation of how a mini-nucleosome core protein modified with PEG12, shown in Panel B, at a lysine residue can undergo a condensation reaction with a DNA molecule to produce a loaded mini-nucleosome. Each nucleic acid molecule may require several (1 to 1000) mini-nucleosome core proteins to neutralize the negative charges in the DNA to form a loaded mini-nucleosome. The schematic is intended only as a cartoon diagram, and is not intended to be representative of the actual structure of loaded mini-nucleosomes except to the extent that loaded mini-nucleosome includes nucleic acids associated with core proteins.

FIG. 2 is a chart showing data obtained from mass spectrometry analyses after the formulation of the mini-nucleosome core protein modified with PEG12 at the first lysine residue in the sequence.

FIG. 3 is a schematic representation of how a mini-nucleosome core protein modified with 1 kDa PEG at a lysine residue can undergo a condensation reaction with a DNA molecule to produce a loaded mini-nucleosome. FIG. 3 includes panel A and panel B. Panel A is a schematic representation of how a mini-nucleosome core protein modified with 1 kDa PEG, shown in Panel B, at a lysine residue can undergo a condensation reaction with a DNA molecule to produce a loaded mini-nucleosome. Each nucleic acid molecule may require several (1 to 1000) mini-nucleosome core proteins to neutralize the negative charges in the DNA to form a loaded mini-nucleosome. The schematic is intended only as a cartoon diagram, and is not intended to be representative of the actual structure of loaded mini-nucleosomes except to the extent that loaded mini-nucleosome includes nucleic acids associated with core proteins.

FIG. 4 includes panel A and panel B. Panel A is a schematic representation of how a mini-nucleosome core protein modified with 2 kDa PEG, shown in Panel B, at a lysine residue can undergo a condensation reaction with a DNA molecule to produce a loaded mini-nucleosome. Each nucleic acid molecule may require several (1 to 1000) mini-nucleosome core proteins to neutralize the negative charges in the DNA to form a loaded mini-nucleosome. The schematic is intended only as a cartoon diagram, and is not intended to be representative of the actual structure of loaded mini-nucleosomes except to the extent that loaded mini-nucleosome includes nucleic acids associated with core proteins.

FIG. 5 includes panel A and panel B. Panel A is a schematic representation of how a mini-nucleosome core protein modified with 5 kDa PEG, shown in Panel B, at a lysine residue can undergo a condensation reaction with a DNA molecule to produce a loaded mini-nucleosome. Each nucleic acid molecule may require several (1 to 1000) mini-nucleosome core proteins to neutralize the negative charges in the DNA to form a loaded mini-nucleosome. The schematic is intended only as a cartoon diagram, and is not intended to be representative of the actual structure of loaded mini-nucleosomes except to the extent that loaded mini-nucleosome includes nucleic acids associated with core proteins.

FIG. 6 includes panel A and panel B. Panel A is a schematic representation of how a mini-nucleosome core protein modified with 10 kDa PEG, shown in panel B, at a lysine residue can undergo a condensation reaction with a DNA molecule to produce a loaded mini-nucleosome. Each nucleic acid molecule may require several (1 to 1000) mini-nucleosome core proteins to neutralize the negative charges in the DNA to form a loaded mini-nucleosome. The schematic is intended only as a cartoon diagram, and is not intended to be representative of the actual structure of loaded mini-nucleosomes except to the extent that loaded mini-nucleosome includes nucleic acids associated with core proteins.

FIG. 7 is a set of images that includes panels A, B, and C, each of which presents an image from Transmission Electron Microscopy (TEM) of loaded mini-nucleosomes.

FIG. 8 is a graph showing concentration of expressed Factor 8 protein as measured by Elisa.

FIG. 9 is a set of images including panels A, B, and C, each of which is a fluorescent microscopy image that illustrates gene expression in liver tissue of proteins encoded by nucleic acids present in loaded mini-nucleosomes.

FIG. 10 is a set of images including panels A, B, C & D each of which is a fluorescent microscopy image that illustrates gene expression in mice RPE tissue of proteins encoded by nucleic acids present in loaded mini-nucleosomes. Panel A is a retinal section that demonstrates RPE specific expression. Panels B is a RPE wholemount that demonstrates RPE specific expression. Panels B and D represent untreated control samples of a retina section and RPE wholemount respectively.

FIG. 11 is a set of images including panels A, B, C & D each of which is a fluorescent microscopy image that illustrates gene expression in rat retinal tissue of proteins encoded by nucleic acids present in loaded mini-nucleosomes. Panels A and C are retinal sections that demonstrates RPE specific expression and panels B and D present plasmid injected control samples.

FIG. 12 is a set of images including panels A, B, C & D each of which is a fluorescent microscopy image that illustrates gene expression in mice retinal tissue of proteins encoded by nucleic acids present in loaded mini-nucleosomes. Panel A is a retinal section that demonstrates GFP expression in retinal neurons. Panel C is a retinal wholemount that demonstrates GFP expression in retinal photoreceptors. Panels B and D represent untreated control samples of a retinal section and RPE wholemount respectively.

FIG. 13 is a set of images including panels A, B & C each of which is a fluorescent microscopy image that illustrates gene expression in mice lung of proteins encoded by nucleic acids present in loaded mini-nucleosomes. Panel A demonstrates GFP expression in alveoli and bronchioles. Panel B demonstrates CFTR staining. Panel C is a merge for panels A and B demonstrating colocalization of GFP and CFTR staining.

FIG. 14 is a set of images including panels A, B & C each of which is a fluorescent microscopy image at higher magnification that illustrates gene expression in mice lung epithelium of proteins encoded by nucleic acids present in loaded mini-nucleosomes. Panel A demonstrates GFP expression in alveoli and bronchioles. Panel B demonstrates CFTR staining. Panel C is a merge for panels A and B demonstrating colocalization of GFP and CFTR including DAPI staining.

FIG. 15 is a set of images that illustrates gene expression in mice whole lung tissue of proteins encoded by nucleic acids present in loaded mini-nucleosomes.

FIG. 16 is a set of images including panels A, B & C that illustrates gene expression in mice brain, gut and pancreas tissue of proteins encoded by nucleic acids present in loaded mini-nucleosomes. Panel A demonstrates expression pattern in olfactory neurons. Panel B and its inset below demonstrates expression pattern in small intestine. Panel C and its inset below demonstrates expression pattern in pancreas.

FIG. 17 is a set of images including panels A, B & C that illustrates gene expression in mice tracheal tissue of proteins encoded by nucleic acids present in loaded mini-nucleosomes. Panel A demonstrates GFP expression in tracheal epithelium and inner tracheal muscle. Panel B demonstrates dystrophin staining pattern in expression in inner and outer tracheal muscle. Panel C is a merge of panel A and B that demonstrates colocalization of dystrophin staining pattern with GFP in inner tracheal muscle cells.

FIG. 18 is a set of images including panels A, B & C that illustrates gene expression in mice muscle tissue of proteins encoded by nucleic acids present in loaded mini-nucleosomes. Panel A demonstrates GFP expression in mouse muscle cells. Panel B demonstrates dystrophin staining pattern in expression in mouse muscle cells. Panel C is a merge of panel A and B that demonstrates colocalization of dystrophin staining pattern with GFP in mouse muscle cells.

FIG. 19 is a graph showing increase in concentration of expressed Factor 8 protein as measured by Elisa following a first dose and a second dose suggesting lack of neutralizing effect or in other words lack neutralizing antibody activity.

FIG. 20 includes panels A, B, C, and D. Panel A is a schematic representation of an unmodified mini-nucleosome core protein. Panel B is a schematic representation of a modified mini-nucleosome core protein, where an asparagine residue (N) is modified with an unbranched modification chain including GlcNac, GlcNac, and sialic acid. Panel C is a schematic representation of a modified mini-nucleosome core protein, where an asparagine residue (N) is modified with a branched modification chain including a trunk including GlcNac, a branch including GlcNac and sialic acid, and a branch including fucose. Panel D is a schematic representation of a modified mini-nucleosome core protein, where an asparagine residue (N) is modified with a branched modification chain including a trunk including GlcNac, a branch including fucose, and a branch including a secondary trunk including GlcNac, a secondary branch including sialic acid, and a secondary branch including mannose.

FIG. 21 includes panels A, B, C, D, E, F and G. Panel A is a schematic representation of an unbranched modification chain (C₁₄0₁₀N₁H₂₃) including GlcNac and mannose that could, e.g., modify an asparagine residue in a mini-nucleosome core protein. Panel B is a schematic representation of an unbranched modification chain (C₂₅0₁₈N₂H₄₀) including GlcNac, mannose, and fucose that could, e.g., modify an asparagine residue in a mini-nucleosome core protein. Panel C is a schematic representation of a di-antennal branched modification chain (C₃₄0₂₅N₂H₅₆) including a trunk including GlcNac, GlcNac, and Mannose and two branches, each including mannose, which modification chain could modify, e.g., an asparagine residue in a mini-nucleosome core protein. A modification chain having this structure can be referred to as low mannose. Panel D is a schematic representation of di-antennal branched modification chain (C₆₄0₄₉N₄H₁₀₆) that includes a trunk including GlcNac, a branch including fucose, and a branch including a secondary trunk including GlcNac and mannose and two secondary branches each including three mannose modifications, which modification chain could, e.g., modify an asparagine residue in a mini-nucleosome core protein. A modification chain having this structure can be referred to as high mannose. Panel E is a schematic representation of a di-antennary sialylated & fucosylated branched modification chain (C₉₀0₆₅N₆H₁₄₆) that includes a trunk including GlcNac, a branch including fucose, and a branch including a secondary trunk including GlcNac and mannose, two secondary branches each including mannose, GlcNac, mannose, and fucose, which modification chain could, e.g., modify an asparagine residue in a mini-nucleosome core protein. Panel F is a schematic representation of tri-antennary sialylated and fucosylated branched modification chain (C₁₁₅0₈₃N₈H₁₈₆) that that includes a trunk including GlcNac, a branch including fucose, and a branch including a secondary trunk including GlcNac and mannose with two secondary branches. One of the secondary branches includes mannose, GlcNac, mannose, and fucose, while the other includes a tertiary trunk including mannose, and two tertiary branches each including GlcNac, mannose, and fucose. The branched modification chain could, e.g., modify an asparagine residue in a mini-nucleosome core protein. Panel G is a schematic representation of tetra-antennary sialylated & fucosylated branched modification chain (C₁₄₀0₁₀₁N₁₀H₂₂₆) that includes a trunk including GlcNac, a branch including fucose, and a branch including a secondary trunk including GlcNac and mannose with two secondary branches. Each of the secondary branches includes a tertiary trunk including mannose, and two tertiary branches each including GlcNac, mannose, and fucose. The branched modification chain could, e.g., modify an asparagine residue in a mini-nucleosome core protein.

FIG. 22 includes panels A, B, C, D, E, F, G, H, I, and J. Panel A is a schematic representation of a GlcNac modification that could modify a serine residue of a mini-nucleosome core protein. Panel B is a schematic representation of an unbranched glycan modification chain including GalNac and galactose. Panel C is a schematic representation of a di-antennary branched glycan modification including a GalNac trunk, a GlcNac branch, and a galactose branch, which branched modification could modify a serine residue of a mini-nucleosome core protein. Panel D is a schematic representation of an unbranched modification including mannose, GlcNac, galactose, and NeuAc, which unbranched modification could modify a serine residue of a mini-nucleosome core protein. Panel E is a schematic representation of an unbranched modification including fucose, GlcNac, galactose, and sialic acid, which unbranched modification could modify a serine residue of a mini-nucleosome core protein. Panel F is a schematic representation of a di-antennary branched modification including a GalNac trunk, a galactose branch, and a branch including GlcNac, galactose, GlcNac, galactose, and NeuAc, which branched modification could modify a serine residue of a mini-nucleosome core protein. Panel G is a schematic representation of a di-antennary branched modification including a GalNac trunk, a galactose branch, and branch including a secondary trunk including GlcNac, galactose, GlcNac, and galactose with a fucose secondary branch and a NeuAc secondary branch, which branched modification could modify a serine residue of a mini-nucleosome core protein. Panel H is a schematic representation of single sugar addition that could modify a serine or threonine, which single sugar modification could be, e.g., a galactose, glucose, mannose, fucose, or sialic acid. Panel I is a schematic representation of an unmodified mini-nucleosome core protein. Panel J is a schematic representation of a modified mini-nucleosome core protein, where a serine residue is modified with an unbranched glycan modification including GalNac, GalNac, and galactose.

FIG. 23 includes panels A, B, C, D, and E. Panel A is a schematic representation of an unmodified mini-nucleosome core protein. Panel B is a schematic representation of a modified mini-nucleosome core protein, where a lysine residue (K) is modified with an acetyl group. Panel C is a schematic representation of a modified mini-nucleosome core protein, where two lysines residues (K) are each modified with an acetyl group. Panel D is a schematic representation of a modified mini-nucleosome core protein, where each of a lysine residue (K) and a valine (V) is modified with an acetyl group. Panel E is a schematic representation of a modified mini-nucleosome core protein, where each of a lysine residue (K) and an alanine (A) residue is modified with an acetyl group.

FIG. 24 is a schematic representation of an acetylated lysine.

FIG. 25 includes panels A, B, and C. Panel A is a schematic representation of an unmodified mini-nucleosome core protein. Panel B is a schematic representation of a modified mini-nucleosome core protein, where a tyrosine residue (Y) is modified with a sulfate group. Panel C is a schematic representation of a sulfated tyrosine

FIG. 26 includes panels A, B, C, and D. Panel A is a schematic representation of an unmodified mini-nucleosome core protein. Panel B is a schematic representation of a modified mini-nucleosome core protein, where a cysteine residue (C) is modified with prenyl group. Panel C is a schematic representation of a farnesyl group linked with a polypeptide. Panel D is a schematic representation of a geranylgeranyl group linked with a polypeptide.

FIG. 27 includes panels A, B, C, D, E, and F. Panel A is a schematic representation of an unmodified mini-nucleosome core protein. Panel B is a schematic representation of a modified mini-nucleosome core protein, where a serine residue (S) is modified with a phospho group. Panel C is a schematic representation of phosphotyrosine. Panel D is a schematic representation of phosphoserine. Panel E is a schematic representation of phosphothreonine. Panel F is a schematic representation of bis-phosphohistidine.

FIG. 28 includes panels A, B, C, D, and E. Panel A is a schematic representation of an unmodified mini-nucleosome core protein. Panel B is a schematic representation of a modified mini-nucleosome core protein sequence, where a lysine residue (K) is methylated. Panel C is a schematic representation of mono-methyl lysine. Panel D is a schematic representation of di-methyl lysine. Panel E is a schematic representation of tri-methyl lysine.

FIG. 29 includes panels A, B, C, and D. Panel A is a schematic representation of an unmodified mini-nucleosome core protein. Panel B is a schematic representation of a modified mini-nucleosome core protein, where a proline residue (P) is hydroxylated. Panel C is a schematic representation of 4-hydroxylated proline. Panel D is a schematic representation of 3-hydroxylated proline.

FIG. 30 includes panels A, B, C, D and E. Panel A is a schematic representation of an unmodified mini-nucleosome core protein. Panel B is a schematic representation of a modified mini-nucleosome core protein, where a lysine residue (K) is lipidated and/or lipoylated. Panel C is a schematic representation of S-myristoylated glycine. Panel D is a schematic representation of S-palmitoylated cysteine. Panel E is a schematic representation of O-palmitoylated cysteine.

FIG. 31 includes panels A and B. Panel A is an IVIS® Spectrum In Vivo Imaging System (IVIS) image of a mouse administered an unmodified mini-nucleosome core protein according to SEQ ID NO: 399 loaded with a nucleic acid payload including a gene encoding luciferase as a representative expression product. Panel B is an IVIS® Spectrum In Vivo Imaging System (IVIS) image of a mouse administered a phosphorylated mini-nucleosome according to SEQ ID NO: 399 loaded with a nucleic acid payload including a gene encoding luciferase as a representative expression product. Panel B shows that the modified mini-nucleosome core protein, but not the unmodified mini-nucleosome core protein, results in robust expression of the representative nucleic acid payload-encoded expression product (here, luciferase) in certain tissues including central nervous system cells including neurons, and including spinal cord cells and brain neurons.

FIG. 32 includes panels A and B. Panel A is an IVIS® Spectrum In Vivo Imaging System (IVIS) image of a mouse administered an unmodified mini-nucleosome core protein according to SEQ ID NO: 388 loaded with a nucleic acid payload including a gene encoding luciferase as a representative expression product. Panel B is an IVIS® Spectrum In Vivo Imaging System (IVIS) image of a mouse administered a sulfated mini-nucleosome according to SEQ ID NO: 388 loaded with a nucleic acid payload including a gene encoding luciferase as a representative expression product. Panel B shows that the modified mini-nucleosome core protein, but not the unmodified mini-nucleosome core protein, results in robust expression of the representative nucleic acid payload-encoded expression product (here, luciferase) in certain tissues including central nervous system cells including neurons, and including spinal cord cells and brain neurons.

FIG. 33 includes panels A, B, C, D and E. Panel A is an IVIS® Spectrum In Vivo Imaging System (IVIS) image of a mouse administered a sulfated mini-nucleosome according to SEQ ID NO: 388 loaded with a nucleic acid payload including a gene encoding luciferase as a representative expression product. Panel A shows a high degree of expression in certain tissues including central nervous system cells including neurons, and including spinal cord cells and brain neurons. Panels B, C, D, and E are images of a representative tissue section from the brain of an animal shown in Figure A (left), each of which represents a different imaging or overlay. Panel B shows a luciferase stain. Panel C shows an anti-NeuN (neuronal marker) antibody stain. Panel D shows a D-DAPI (nuclear) stain. Panel E shows an overlay of the luciferase, anti-NeuN antibody, and D-DAPI stains. Images demonstrate robust expression of the representative nucleic acid payload-encoded expression product (here, luciferase) in brain cells, particularly including brain neurons, when the mini-nucleosome is sulfated but not when the mini-nucleosome is unmodified.

FIG. 34 includes panels A, B, C, D, E, and F, each of which shows a retinal wholemount. Panels A, B, and C show a representative retinal wholemount from a mouse administered an unmodified mini-nucleosome core protein according to SEQ ID NO: 401 loaded with a nucleic acid payload including a gene encoding green fluorescent protein (GFP) as a representative expression product. Panel A shows native GFP fluorescence. Panel B shows an anti-GFP antibody stain. Panel C shows an overlay of native GFP fluorescence and anti-GFP antibody stain. Panels D, E, and F show a representative retinal wholemount from a mouse administered an acetylated mini-nucleosome according to SEQ ID NO: 401 loaded with a nucleic acid payload including a gene encoding GFP as a representative expression product. Panel D shows native GFP fluorescence. Panel E shows an anti-GFP antibody stain. Panel F shows an overlay of native GFP fluorescence and anti-GFP antibody stain. Images demonstrate robust expression of the representative nucleic acid payload-encoded expression product (here, GFP) in retinal cells, in particular photoreceptors, when the mini-nucleosome is acetylated but not when the mini-nucleosome is unmodified.

FIG. 35 includes panels A, B, C, and D, each of which is an image of a representative retinal wholemount from a mouse administered a mannosylated mini-nucleosome according to SEQ ID NO: 447 loaded with a nucleic acid payload including a gene encoding GFP as a representative expression product. Panel A shows native GFP fluorescence. Panel B shows an anti-GFP antibody stain. Panel C shows a peanut agglutinin (PNA; marker for photoreceptors) stain. Panel D shows an overlay of native GFP fluorescence, anti-GFP antibody stain, and PNA stain. Images demonstrate robust expression of the representative nucleic acid payload-encoded expression product (here, GFP) in retinal cells, in particular photoreceptors, when the mini-nucleosome is mannosylated. An unmodified control loaded with the same nucleic acid payload was not robustly expressed in retinal cells or photoreceptors.

### DETAILED DESCRIPTION

The present disclosure provides, among other things, methods and compositions relating to mini-nucleosome core proteins and uses thereof. Mini-nucleosome core proteins disclosed herein include, among other things, (a) a nucleic acid binding domain (NABD), (b) a targeting domain and/or (c) a nucleic acid release domain, and/or a stability domain, and/or an oligomerization domain, and/or a linker domain. Certain mini-nucleosome core proteins disclosed herein include (a) a nucleic acid binding domain (NABD); (b) a targeting domain; (c) a nucleic acid release domain; and, optionally, (d) further domains including, e.g., one or more of a stability domain, an oligomerization domain, and/or a linker domain. In various embodiments, mini-nucleosome core protein of the present disclosure is a modified mini-nucleosome core protein, i.e., a mini-nucleosome core protein that is modified in that it includes one or more modified amino acid residues, such as an amino acid residue modified to include a modification provided herein, including without limitation any of one or more of (i) phosphorylation; (ii) sulfation; (iii) glycosylation (e.g., N-glycosylation, C-glycosylation, and/or O-glycosylation); (iv) prenylation (e;g., geranylation and/or farnesylation); (v) methylation; (vi) sialylation; (vii) lipidation and/or lipoylation; (viii) acetylation; (ix) hydroxylation; (x) palmitoylation; (xi) mannosylation; (xii) myristoylation; (xiii) fucosylation; (xiv) pegylation; and/or; (xv) any combination thereof, including any number of one or more of the modifications or variants thereof, e.g., in a branched or unbranched modification chain. In various embodiments, a mini-nucleosome core protein of the present disclosure can be a modified mini-nucleosome core protein that includes (a) a nucleic acid binding domain (“NABD”); (b) a targeting domain; (c) a modified amino acid residue; and, optionally, (d) further domains including, e.g., one or more of a nucleic acid release domain, a stability domain, an oligomerization domain, and/or a linker domain. In various embodiments, a mini-nucleosome core protein of the present disclosure can be a modified mini-nucleosome core protein that includes (a) a nucleic acid binding domain (“NABD”); (b) a targeting domain; (c) a nucleic acid release domain; (d) a modified amino acid residue; and, optionally, (e) further domains including, e.g., one or more of a stability domain, an oligomerization domain, and/or a linker domain. In various embodiments, a mini-nucleosome core protein is associated with one or more nucleic acid molecules, thereby forming a loaded mini-nucleosome core protein (the mini-nucleosome core protein and associated nucleic acid molecules also referred to together herein as a loaded mini-nucleosome). In various embodiments, a loaded mini-nucleosome includes two or more mini-nucleosome core proteins and one or more nucleic acid molecules. In various embodiments, a loaded mini-nucleosome is administered to a subject in need thereof.

Polynucleotide chains typically carry phosphates with negative charge. Accordingly, positive charges in proteins such as histones help condense nucleic acids. The present disclosure appreciates that nucleic acid binding domains, derived, e.g., from histones, can be utilized in artificially constructed mini-nucleosome core proteins as a non-viral proteinaceous vector.

Most mammalian cells possess cell surface binding moieties or receptors that recognize (and/or are recognized by), bind, and internalize molecules or entities like viruses and bacteria. Various compositions and methods disclosed herein make use of such cell surface binding motifs in combination with nucleic acid binding domains and poly-Arginine domains in a mini-nucleosome core protein. In various embodiments, a mini-nucleosome core protein is capable of condensing, or participating in or facilitating the condensation of, one or more nucleic acids. In various embodiments, a mini-nucleosome core protein facilitates internalization of associated nucleic acids, e.g., in a loaded mini-nucleosome, into specific cell types, e.g., via endocytosis or via other cellular entry mechanisms. Accordingly, in various embodiments, the present disclosure includes mini-nucleosome core proteins that incorporate targeting moieties capable of binding with cell surface moieties or receptors that are naturally present on cells of a system, e.g., a system that is a human, where the cell surface moiety or receptor provides a cell entry mechanism. In various instances, the cell surface moiety or receptor is cell-type specific and thus facilitates specific delivery of nucleic acids to selected cell types.

Nucleic acid molecules can have a large negative charge, are vulnerable for degradation in body fluids, e.g., after administration to subjects, and cannot enter a cell via simple injections or exposure to the cell. That large negative charge can be neutralized by mini-nucleosome core-proteins, including modified mini-nucleosome core proteins, to form loaded mini-nucleosomes of certain shape, size, and charge and/or that are capable of entering into cells by passive diffusion or active transport. Various mini-nucleosome core proteins described herein allow proper binding, condensation and targeting of nucleic acids. These domains described herein, may be derived from human proteins or other organisms. One skilled in the art may contemplate modifying or engineering domains and/or mini-nucleosome core proteins described herein, e.g., with changes to the amino acid sequence for enhancing certain functions such as cell attachment, internalization etc. but not limited to these. One skilled in the art may also contemplate placing the domain in reverse sequence or by switching amino acid positions within the domain or adding various posttranslational modifications such as acetylation, glycation etc. to amino acids but not limited to these. Various mini-nucleosome core proteins described herein include at least one modified amino acid residue.

### Nucleic Acid Binding Domains

The present disclosure includes the recognition that positively charged domains associate with nucleic acids. The present disclosure provides nucleic acid binding domains, e.g., DNA and RNA binding domains, that can be included in a mini-nucleosome core protein. In some instances, a DNA binding domain present in a mini-nucleosome core protein is a DNA binding domain disclosed herein. In some instances, a RNA binding domain present in a mini-nucleosome core protein is a RNA binding domain disclosed herein.

In some particular instances, an NABD that is a DNA binding domain present in a mini-nucleosome core protein disclosed herein can be derived from a histone polypeptide sequence. Non-viral vectors such as DNA nanoparticles utilizing poly-lysine peptides to compact DNA into smaller particles for gene delivery (Liu G. et al, 2003) have been used, at least some instances, with no success or significant responses in treatment of diseases (Konstan M. W. et al, 2004). The present disclosure provides a significantly different approach that includes, in various embodiments, use of DNA binding domain of histones, for example the amino acid sequence KRHRK. This amino acid sequence serves two purpose—first it gives the highly positive charge that is needed to associate with nucleic acids, Secondly, it also gives stability to the mini-nucleosome core protein structure. Thirdly, the amino acid sequence KRH in this NABD also is a cleavage site for proprotein convertases thus allows efficient release of the genetic cargo in cells.

Other examples of NABDs are provided in Table 3.

A poly-arginine tract such as RRRRR can be included in a mini-nucleosome core protein to increase nucleic acid binding as well as to enhance positive charge and/or cell penetration ability of the composition. A poly-arginine tract can be present in a mini-nucleosome core protein in a position suitable to facilitate penetration of cells by the mini-nucleosome core protein and/or by loaded mini-nucleosomes including the mini-nucleosome core protein. Those of skill in the art will be aware of the methods and techniques that allow determination of such a position. Arginine interacts with phospholipids to form of bi- or multi-dentate hydrogen bonding from simultaneous association with the phosphates of more than one lipid head therefore interacts with the phosphate on a single lipid head group. Since, only arginine can form bidentate hydrogen-bonds, poly-arginines could bond with more zwitterionic and anionic lipids and therefore generate positive curvature along its contour length, thus resulting in negative Gaussian curvature (Rothbard, J. B., et al. 2005). A poly-Arginine tract may also be modified to include specifically one or more Histidine (H) amino acid (or any other amino acid) to improve stability of the mini-nucleosome core protein. Histidine (or any other amino acid) may be inserted in any position in the poly-Arginine tract as shown in Table 3. Other arginine-rich peptides such as ANTP Penetratin, and TAT have also shown similar impact on cell penetration.

The present disclosure includes the recognition that localization of a mini-nucleosome core protein to a euchromatin area of the nucleus can be facilitated by acetylation of lysines in mini-nucleosome core proteins. The mechanism of this stabilization may be related, at least in part, to mechanisms that stabilize post-translationally modified histones. Methylated histones pack more tightly. Histone methylation can be dynamic. Other post translational modifications that can be applied are: phosphorylation, glycosylation, prenylation, lipoylation, alkylation, acylation, glycation, nitrosylation, sulfation, carbamylation, carbonylation, sumoylation, neddylation, biotinylation, ribosylation etc. Modifications may not be limited to these mentioned here. Other modifications may include attachment of co-factors, co-enzymes, hydrophobic groups, hydrophilic groups, smaller chemical groups, smaller peptides etc. Such modification could also be applied to amino acids in these mini-nucleosome core proteins described herein. Nucleic acid binding domains mentioned herein, in Table 3 can be incorporated in polypeptides at any location to enhance nucleic acid binding in combination with other domains provided in Tables 4, 5, 6, 7, 9, 10, 11 and 12.

TABLE 3

| Exemplary | | | | |
|---|---|---|---|---|
| Domains | SEQ ID NO: | Name | Exemplary Utility | Reference |
| KRHRK | 1 | DNA binding | Enhanced DNA | Bottomley |
| | | domain | binding | M.J., 2004 |
| RRR, RRRR, | 2, 3, 4, 5 | Poly-Arginines: | Enhanced cell | Mishra, A. et |
| RRRRRR, (RR)X | | DNA binding | penetration | al, 2008 |
| | | domain | | |
| RRLARR | 6 | Condensing | Enhanced DNA | John P. H. |
| | | domain (part of) | binding; | Th'ng et al. |
| | | | condensation | 2005 |
| KKAKAAAKPKK | 7 | Condensing | Enhanced DNA | John P. H. |
| | | domain (part of) | binding and | Th'ng et al. |
| | | | condensation | 2005 |
| KKDGKKRKR | 8 | Condensing | Enhanced DNA | John P. H. |
| | | domain (part of) | binding and | Th'ng et al. |
| | | | condensation | 2005 |
| KKKLK | 9 | HTH motif (part | Enhanced DNA | Uniprot |
| | | of | binding | |
| KKRIRK, RKKSK | 10, 11 | RUNX1 binding | Enhanced DNA | Uniprot |
| | | (part of) | binding | |
| KKPKK | 12 | Condensing | Enhanced DNA | John P. H. |
| | | domain (part of) | binding and | Th'ng et al. |
| | | | condensation | 2005 |
| RRHRR | 13 | Nucleic acid | Enhanced nucleic | Uniprot |
| | | binding | acid binding and | |
| | | | stability | |
| RHRRR | 14 | Nucleic acid | Enhanced nucleic | Uniprot |
| | | binding | acid binding and | |
| | | | stability | |
| RRRRHR | 15 | Nucleic acid | Enhanced nucleic | Uniprot |
| | | binding | acid binding and | |
| | | | stability | |
| KRTVRK | 16 | Nucleic acid | Enhanced nucleic | Uniprot |
| | | binding | acid binding | |
| KRQRNR | 17 | Nucleic acid | Enhanced nucleic | Uniprot |
| | | binding | acid binding | |
| RVCACPGR | 18 | P53 DNA | Enhanced nucleic | Uniprot |
| | | interaction | acid binding | |
| (KKK)x | 19 | Nucleic acid | Enhanced nucleic | Uniprot |
| | | binding | acid binding | |
| DEMGLGKT | 20 | Nucleic acid | Nucleotide binding | Uniprot |
| | | binding | | |
| QRE, HLSQHLN, | 21, 22, 23, | Nucleic acid | Interaction with | Uniprot |
| KTQK, RFKW, | 24, 25, 26 | binding | DNA | |
| RVY, NRRK | | | | |
| TFF | 27 | Nucleic acid | RNA binding | Uniprot |
| | | binding | | |
| RPRGRPRKHTVTS | 28 | Nucleic acid | Enhanced nucleic | Uniprot |
| | | binding | acid binding | |

For the avoidance of doubt, the present disclosure includes modified nucleic acid binding domains, including without limitation a nucleic acid binding domain of the present disclosure in which at least one amino acid of the nucleic acid binding domain includes a modification disclosed herein.

### Targeting Domains

Mini-nucleosome core proteins disclosed herein include targeting domains that target mini-nucleosomes to one or more cells or cell types.

In some embodiments, a targeting domain of a mini-nucleosome core protein is an amino acid domain that allows attachment to and enter into one or more cells or cell types. It is to be understood that targeting domains can be specific to certain cell types but can also include domains that facilitate entry into cells generally. In general, a targeting domain of a mini-nucleosome core protein can contribute to one or more of attachment, cell-type specific binding, and internalization. A targeting domain can be, for example, a cell attachment targeting domain, beta galactose binding domain, fucose binding domain, heparin binding domain, sialic acid binding domain, glycoprotein binding domain, carbohydrate binding domain, lysophosphatidic acid binding, cAMP binding domain, hyaluronan binding domain, chondroitin sulfate binding domain, integrin binding domain, nucleolin binding domain, collagen binding domain, clathrin binding domain, Fc receptor binding domain, actin binding domain, endocytosis motif or a nuclear localization signal. In some embodiments, a targeting domain of a mini-nucleosome core protein is an amino acid domain that allows binding and entry into one or more cells or cell types and that is derived from a mammal, virus, viral particle, prion, bacteria or fungal amino acid sequence.

For the avoidance of doubt, the present disclosure includes modified targeting domains, including without limitation a targeting domain of the present disclosure in which at least one amino acid of the targeting domain includes a modification disclosed herein.

### Cell Attachment Targeting Domains

Cell attachment is a means by which a mini-nucleosome core protein, or loaded mini-nucleosome include the mini-nucleosome core protein, can adhere to cell and, in various instances, facilitate entry to into the cell. Various viruses have adhesion molecules or domains that allow binding to host cells and enhance entry into them. For example, flu virus has hemagglutinin on its surface that allows it to bind to sialic acid on the cell surface. The present disclosure provides, among other things, several such domains that allow mini-nucleosome core protein binding to sialic acid, galactose, fucose, hyaluronic acid, and chondroitin sulfate, as well as glycoproteins that enhance cell attachment for internalization. A mini-nucleosome core protein disclosed herein can include one or more cell attachment targeting domains. Cell attachment targeting domains include the domains shown in Table 4. A cell attachment targeting domain of the present disclosure can be present in a mini-nucleosome core protein at any position and/or in combination with any of one or more other domains provide herein, e.g., in Tables 3, 5, 6, 7 8, 9, 10, 11 and 12.

TABLE 4

| | | | Exemplary | |
|---|---|---|---|---|
| Exemplary Domains | SEQ ID NO: | Name | Utility | Reference |
| WGREERQ | 29 | Cell | Enhanced cell | Uniprot |
| | | attachment | surface | |
| | | site on | attachment via | |
| | | LGALS3 | beta-galactose | |
| | | | binding | |
| NTQIH & WNNKTPH | 30, 31 | CTxB domain | Enhanced cell | Uniprot |
| | | | surface | |
| | | | attachment via | |
| | | | galactose binding | |
| TPH | 32 | CTxB domain | Enhanced cell | Uniprot |
| | | | surface | |
| | | | attachment via | |
| | | | Fucose binding | |
| VNRWS | 33 | Sialic acid | Enhanced muscle | Uniprot |
| | | binding | cell surface | |
| | | domain | attachment via | |
| | | | Sialic acid | |
| | | | binding | |
| XBBBXXBX, | 34, 35 | Heparin | Enhanced cell | Cardin and |
| ARKKAAKA | | binding | surface | Weintraub, |
| | | domain | attachment via | 1989 |
| | | | Heparin binding. | |
| QRR, SRR | 36, 37 | CPC motif | Enhanced cell | Torrent M. |
| | | | surface | et. al, 2012 |
| | | | attachment via | |
| | | | Heparin binding | |
| WEPSRPFPVD | 38 | B3GAT3 | Enhanced cell | Uniprot |
| | | motif | surface | |
| | | | attachment via | |
| | | | galactose binding | |
| HRRTRKAPKRIRLPHIR | 39 | Herpes | Enhanced cell | Uniprot |
| | | glycoprotein | surface | |
| | | gD motif | attachment via | |
| | | | glycoprotein | |
| | | | binding | |
| KRTGQYKLGSKTGPGQK | 40 | Heparin | Enhanced cell | Uniprot |
| | | binding | surface | |
| | | domain in | attachment via | |
| | | FGF2 | heparin binding | |
| KKTK | 41 | Heparin | Enhanced cell | Nelson C. Di |
| | | sulfate | surface | Paolo et al, |
| | | binding | attachment via | 2007 |
| | | | heparin sulfate | |
| | | | binding domain | |
| KLRSQLVKK | 42 | Hyaluronan | Enhanced cell | Uniprot |
| | | binding motif | surface | |
| | | | attachment via | |
| | | | Hyaluronan | |
| | | | binding | |
| RRRCGQKKK | 43 | Hyaluronan | Enhanced cell | Uniprot |
| | | binding motif | surface | |
| | | | attachment via | |
| | | | Hyaluronan | |
| | | | binding | |
| BX(7)B | 44 | BX7B domain | Enhanced cell | Jean L. et al, |
| | | | surface | 2001 |
| | | | attachment via | |
| | | | Hyaluronan | |
| | | | binding | |
| RIQNLLKITNLRIKFVK | 45 | AC15 domain | Enhanced cell | Kokona |
| | | | surface | Kouzi-K. et |
| | | | attachment via | al. 1989 |
| | | | heparin binding | |
| KKEKDIMKKTI | 46 | Sgl MOTIF | Enhanced cell | Joji I. et al, |
| | | of integrin | surface | 1998 |
| | | | attachment via | |
| | | | chondroitin | |
| | | | sulfate binding | |
| | | | domain | |
| HGSRFTFHRGSM, HRPH, | 47, 48, | Lectin binding | Enhanced cell | Uniprot |
| DVAR, HFNPR, WGTE | 49, 50, | | surface | |
| | 51 | | attachment via | |
| | | | Beta-galactoside | |
| | | | binding binding | |
| | | | domain | |
| KKQFGAEC | 52 | Chondroitin | Enhanced cell | Uniprot |
| | | sulfate | surface | |
| | | binding | attachment | |
| RRPRPGTGPGRRPRPRPRP | 53 | Heparan | Enhanced cell | Uniprot |
| | | sulfate | surface | |
| | | binding | attachment | |

Cell attachment can also be achieved by domains such as RGD, RGDS etc. (D'Souza S E et al, 1991). Binding to cell surface proteins such as integrins, nucleolin, collagen, clathrins, Fc receptors also help viruses and other particles get entry to the cell. The present disclosure provides, among other things, domains that allow binding to as integrins, nucleolin, collagen, clathrins, Fc receptors for increased cellular uptake. Cell attachment targeting domains include the domains shown in Table 5. A cell attachment targeting domain provided in Table 5 can be present in a mini-nucleosome core protein at any position and/or in combination with any of one or more other domains provide herein, e.g., in Tables 3, 4, 6, 7, 8, 9, 10, 11 and 12.

TABLE 5

| | SEQ ID | | | |
|---|---|---|---|---|
| Exemplary Domains | NO: | Name | Exemplary Utility | Reference |
| KGE | 54 | Cell attachment | Enhanced cell | Maginnis |
| | | motif | attachment via | M. S. et al, |
| | | | Integrin binding | 2006 |
| RGD, RGDS | 55, 56 | Cell attachment | Can be used to block | D'Souza SE |
| | | motif | RPE transduction. | et al, 1991 |
| TTVVNPKYEGK, | 57, 58 | Betal integrin cell | Enhanced cell | Reszka A. A. |
| ERMSQIKRLLS | | attachment domain | attachment via | et al, 1992 |
| | | | Integrin binding | |
| WRHRARS | 59 | NS5B domain | Enhanced cell | Kusakawa T. |
| | | | attachment via | et al, 2007 |
| | | | Nucleolin binding | |
| GFOGER | 60 | A-domains of | Enhanced cell | Knight C. G. |
| | | Integrins | attachment via | et al, 2000 |
| | | | Collagen I and IV | |
| | | | binding to Integrins | |
| LFDLM | 61 | ENTH domain | Enhanced cell | Kalthoff et al, |
| | | | attachment via | 2002 |
| | | | Clathrin terminal | |
| | | | domain binding | |
| WGREERQ | 62 | Galactose binding | Enhanced cell | Uniprot |
| | | motif | attachment via | |
| | | | galactose binding site | |
| | | | on LGALS3 | |
| QSTEKRG | 63 | Cclec6A motif | Enhanced cell | Uniprot |
| | | | attachment via | |
| | | | association with Fc | |
| | | | receptor gamma | |
| | | | chain (FCER1G) | |
| LPNTG | 64 | LPXTG motif | Enhanced cell | Dramsi et al, |
| | | | attachment | 2008 |
| DSPE, FQVT | 65, 65 | Popeye domain | cAMP binding | Brand, T. |
| | | | | 2016 |
| QSTEKRG | 66 | CLEC6a motif | Carbohydrate | Uniprot |
| | | | binding | |
| RQGLID | 67 | domain in LPAR1 | Lysophosphatidic | Uniprot |
| | | | acid binding | |
| RKKH | 68 | Midas motif | Echo virus 1 and | Pentikainen |
| | | | integrin binding | O. et al, 1999 |
| | | | motif. Collagen | |
| | | | binding. | |
| YPK, YNQYT | 69, 70 | Sialoadhesion | Myelin associated | Kelm S. et al, |
| | | domain | glycoprotein | 1994 |
| KWNYK | 71 | Sialic acid binding | Siglec7 | Uniprot |
| | | domain | | |
| GPQSVKFKSPDQI | 72 | Adhesion domain | Cytoadherence | Uniprot |
| RVGENWWY, | 73, 74, | Chondroitin sulfate | Cell surface | Uniprot |
| RTLQAHHDR, | 75, 76, | binding | attachment | |
| RESPFSGSSR, | 77, 78, 79 | | | |
| REEIQERMR, | | | | |
| QDSSSFHHQ, | | | | |
| KKQFGAEC, | | | | |
| KRALHNAEC | | | | |
| KQKIKHVVKLK, | 80, 81 | Hyaluronic acid | Cell surface | Uniprot |
| KLRCQLAKKK | | binding | attachment | |

For the avoidance of doubt, the present disclosure includes modified cell attachment targeting domains, including without limitation a cell attachment targeting domain of the present disclosure in which at least one amino acid of the cell attachment targeting domain includes a modification disclosed herein.

### Internalization Targeting Domains

Certain domains in viral and mammalian proteins can directly impact cellular internalization. For example, domains of certain proteins, and sequential arrangement, is described in Oleson et al., 2008. For example, a PPxY-Motif is required for adenovirus entry into cells (Wodrich et al, 2010), where x could be any amino acid. Another example of an internalization targeting domain is the GTALL motif—a five-amino acid residue domain, in the carboxyl-terminus tail of leutinizing hormone (LH) receptor directs the ligand-receptor complexes from a degradative to recycling pathway (Pandey, 2009). The GTALL motif also shows sequence homology to carboxyl-terminus tetrapeptide sequence motif DSLL, which has been suggested to participate in the internalization of β-adrenergic receptors. Pandey also discusses that the clathrin-dependent cargo usually contains a short sequence motif such as YXXQ (where X could be any amino acid), recognized by adaptor protein-2 (AP-2) and may contain Asn-Pro-X-Tyr sequence (NPXY) motifs, which are recognized by the accessory clathrin adaptor proteins. Transferrin. NPXY motif has also been discussed by Kirchhausen, 1999. NPTY is also the Endocytosis motif of APP. Another example of clathrin binding domain that allows internalization is FXDXF (where X could be any amino acid) (Lene E. Oleson. 2008). Internalization targeting domains include the domains provided in Table 6.

Other features provided by the present disclosure include one or more leucine and isoleucine residues, which residues are highly hydrophobic in nature. In fact, leucine is the second most hydrophobic amino acid. In various embodiments, leucine residues can serve multiple functions in the composition of mini-nucleosome core proteins. First, the hydrophobicity of the nonpolar face of an amphipathic molecule plays an important role in stabilizing the peptide secondary structure (Chen Y. et al, 2007). Secondly, dileucine-type of signal motifs have been shown to be essential for internalization and trafficking of membrane receptors and membrane proteins into subcellular compartments. For example, GLUT4 (glucose transporter 4), LDL (low density lipoprotein); LH (leutinizing hormone), TGN (Trans-Golgi network) all have dileucine motifs that help internalization into cells. Fc receptor dileucine motif also signals for endocytosis (Wu Z. and Simister N. E., 2001). An internalization targeting domain provided in Table 4 can be present in a mini-nucleosome core protein at any position and/or in combination with any of one or more other domains provide herein e.g., in Tables 3, 4, 5, 7, 8, 9, 10, 11 and 12.

TABLE 6

| | SEQ ID | | Exemplary | |
|---|---|---|---|---|
| Exemplary Domains | NO: | Name | Utility | Reference |
| FXDXF | 82 | FXDXF-motif | Clathrin binding | Lene E. Oleson |
| | | | motif facilitates | JBC. 2008 |
| | | | internalization | |
| PPSY | 83 | PPxY-Motif | Facilitates | H Wodrich et al, |
| | | | Adenovirus | 2010 |
| | | | Entry. At the | |
| | | | end of the | |
| | | | sequence. | |
| FEDNFVP | 84 | 7-mer peptide from | Enhanced | Lene E. Oleson |
| | | amphiphysin. | Internalization | JBC. 2008 |
| YIRV, YADW, YTQV | 85, 86, 87 | Internalization | Enhanced | Zrarate et al, 2007 |
| | | motif | Internalization | |
| KKRPKP | 88 | Prion | Is sufficient to | (Sunyach, 2003). |
| | | internalization | direct | |
| | | motif | internalization. | |
| SSDDE, RRASS | 89, 90 | CcN motif | Efficient nuclear | (David A Jans. |
| | | | transport and | 1995 JBC) |
| | | | localization | |
| (YXXL)2 | 91 | Internalization | For viral entry | Inabe K et al, |
| | | motif of bovine | and | 1999 |
| | | leukemia virus | incorporation of | |
| | | | viral envelope | |
| | | | protein into | |
| | | | virions. | |
| LPLTG, LAFTG | 92, 93 | Sorting signal | Sortase | Ton-That, H., and |
| | | | dependent entry. | O. Schneewind. |
| | | | | 2003 |
| L, I, LI, IL | 94, 95, | Leucines, | Increased | Chen Y. et al, |
| | 96, 97 | Isoleucine | hydrophobicity | 2007 |
| | | | for polypeptide | |
| | | | stability | |
| LL | 98 | Dileucine | Enhanced | Wu Z. and |
| | | | cellular | Simister N. E., |
| | | | internalization | 2001 |
| KRRHPKK | 99 | Cardin-Weintraub | Heparan sulfate | Uniprot |
| | | motif | binding | |
| EPS, EPNLPEE, ND | 100, 101, | Mannose binding | Enhanced | Uniprot |
| | 102 | domian | cellular | |
| | | | internalization | |
| NFR | 103 | N-acetyl-D- | Enhanced | Uniprot |
| | | glucosamine | cellular | |
| | | binding | internalization | |
| YWV | 104 | PDZ binding | Enhanced | Uniprot |
| | | | cellular | |
| | | | internalization | |
| AICKRIPNKKPGKRT | 105 | Heparin binding | Enhanced | Uniprot |
| | | | cellular | |
| | | | internalization | |
| VAR, KIL | 106 | Receptor binding | Enhanced | Uniprot |
| | | (CXCL12) | cellular | |
| | | | internalization | |
| RCPCR, | 107, 108 | Heparin binding | Enhanced | Uniprot |
| RANVKHLKILN, | 109 | | cellular | |
| VARLKNNNRQV | | | internalization | |
| VRKKP, | 110, 111 | PDGFA binding to | Enhanced | Uniprot |
| YVRKKPKLK | | its receptor | cellular | |
| | | | internalization | |
| ISRRLI | 112 | PDGFB binding to | Enhanced | Uniprot |
| | | its receptor | cellular | |
| | | | internalization | |
| LTKRSRQ, | 113, 114 | Gag binding | Enhanced | Uniprot |
| NRKISVQRL | | | cellular | |
| | | | internalization | |
| YYKQRLI | 115 | Nucleocytoplasmic | Enhanced | Uniprot |
| | | transport | cellular | |
| | | | internalization | |

For the avoidance of doubt, the present disclosure includes modified internalization targeting domains, including without limitation an internalization targeting domain of the present disclosure in which at least one amino acid of the internalization targeting domain includes a modification disclosed herein.

### Nucleus Targeting Domains

In various embodiments, it is important that, following cellular entry, a nucleic acid cargo reaches the nucleus. Nuclear internalization signals or binding to the nuclear import machinery are key to nuclear localization. Functional eukaryotic nuclear localization signals are widespread in terminal proteins of bacteriophages (Redrejo-Rodríguez et. al, 2012). Chan and Jans have shown that polylysine by itself doesn't function as a nuclear localization signal. Thus, adding a nuclear targeting signals to enhance non-viral gene transfer is a logical approach (Chan and Jans, 1999). Location of NLS in the polypeptide is also key for its function. We have listed the NLS sequences in Table 7 for enhanced nuclear entry and provided certain preferred locations of NLS signal within mini-nucleosome core proteins for efficient nuclear entry in Table 13. Domains mentioned herein, in Table 7 can be incorporated in mini-nucleosome core protein at any location to enhance nucleic acid binding in combination with other domains provided in Table 3, 4, 5, 6, 8, 9, 10, 11 and 12.

TABLE 7

| | SEQ | | | |
|---|---|---|---|---|
| | ID | Source | Exemplary | |
| Exemplary Domains | NO: | protein | Utility | Reference |
| KKKYKLK | 116 | Gag pol | Nuclear | Uniprot |
| | | | localization | |
| | | | signal | |
| KKRKLE | 117 | LMNA | Nuclear | Uniprot |
| | | | localization | |
| | | | signal | |
| TRSK | 118 | VP22 | Nuclear | Uniprot |
| | | | localization | |
| | | | signal | |
| HRKRKR | 119 | Aprataxin | Nuclear | Uniprot |
| | | | localization | |
| | | | signal | |
| NKRKRK | 120 | SAP30L | Nuclear | Uniprot |
| | | | localization | |
| | | | signal | |
| AEKSKKK | 121 | HMGB1 | Nuclear | Uniprot |
| | | | localization | |
| | | | signal | |
| RKSK, KRVK | 122, | HIPK2 | Nuclear | Uniprot |
| | 123 | | localization | |
| | | | signal | |
| KRK | 124 | NFATC1 | Nuclear | Uniprot |
| | | | localization | |
| | | | signal | |
| LQQTPLHLAVI | 125 | NFKB | Nuclear | Uniprot |
| | | inhibitor | localization | |
| | | alpha | signal contains | |
| | | | ankyrin repeats | |
| RRPR, PRPR, RPPP | 126, | Bovine | Nuclear | Uniprot |
| | 127, | Herpes | localization | |
| | 128 | Virus | signal for | |
| | | | bovine herpes. | |
| RKKRKGK | 129 | DAG1 | (dystroglycan). | Uniprot |
| | | | In the c- | |
| | | | terminal | |
| PAAKRVKLD | 130 | c-Myc | Nuclear | Uniprot |
| | | | localization | |
| | | | signal | |
| KLKIKRPVK | 131 | TUS | Nuclear | Uniprot |
| | | | localization | |
| | | | signal | |
| PKKKRKV | 132 | SV40 | Nuclear | Uniprot |
| | | | localization | |
| | | | signal | |
| QRKRQK | 133 | NFKB | Nuclear | Uniprot |
| | | | localization | |
| | | | signal | |
| KRPR | 134 | TOPBP1 | Nuclear | Uniprot |
| | | | localization | |
| | | | signal | |
| RKRRRP | 135 | DEDD2 | Nuclear | Uniprot |
| | | | localization | |
| | | | signal | |
| KKGRRNRFK | 136 | HNF1A | Nuclear | Uniprot |
| | | | localization | |
| | | | signal | |
| RHRDRLNTELDRLASLLPFPQDVI | 137 | AHR | Nuclear | Uniprot |
| NKLDK | | | localization | |
| | | | signal | |
| KRGRKP | 138 | CBX2 | Nuclear | Uniprot |
| | | | localization | |
| | | | signal | |
| KKRAGRRIFKETR | 139 | DREBE1 | Nuclear | Uniprot |
| | | | localization | |
| | | | signal | |

For the avoidance of doubt, the present disclosure includes modified nucleus targeting domains, including without limitation a nucleus targeting domain of the present disclosure in which at least one amino acid of the nucleus targeting domain includes a modification disclosed herein.

### Cell-Type Specific Targeting Domains

In various embodiments, it is most desirable that larger concentration of the particles home into the desired cell type. This allows for increased uptake and increased expression—two favorable gene therapy output. In literature, there are very few motifs that have been discovered for such properties. Most of these come from experiments that have shown viral tropism to be different from different capsids. The present disclosure includes, in various embodiments, use of some of those defined motifs, to enhance expression in neurons, muscles, liver, lung, kidney, endothelial cells or tumor sites. Cell-type specific targeting domains include the domains shown in Table 8. A cell-type specific targeting domain of Table 8 can be present in a mini-nucleosome core protein at any position and/or in combination with any of one or more other motifs provide herein, e.g., in Tables 3, 4, 5, 6, 7, 9, 10, 11 and 12.

TABLE 8

| | SEQ ID | | |
|---|---|---|---|
| Exemplary Domains | NO: | Exemplary Utility | Reference |
| ASSLNIA | 140 | Muscle targeting | Yu C-Y. et al. 2009 |
| SKIFNTHPQSTP | 141 | Muscle targeting | Y Seow et al. 2010 |
| YKQCHKKGGHCFPKEK | 142 | Muscle targeting | Uniprot |
| LGKMDCRWKWKCCKKGSG | 143 | Muscle targeting | Uniprot |
| HGSRFTFHRGSM | 144 | Muscle targeting | Uniprot |
| KKEEEKKEEEKKEEE | 145 | Renal targeting | Wischnjow A, et al, 2016 |
| LIFHKEQ | 146 | LIVER targeting | Uniprot |
| KFNKPFVFLI | 147 | Lung targeting | Buning H. et al, 2003 |
| QPEHSST | 148 | Endothelial cell targeting | Work, L. M. et. al, 2006 |
| EYHHYNK | 149 | Vascular smooth muscle cell | Work, L. M, et. al, 2004 |
| | | targeting | |
| NGR | 150 | Tumor homing | Arap W, et. al, 1998 |
| GEKGEP | 151 | Facilitate phagocytosis by | Uniprot |
| | | monocytes | |
| KTKKK, KALKKK, KGKKK | 152, 153, | Phagocytosis of the particles. | Caberoy N. B. et al, 2010 |
| | 154 | | |
| CSVTCG | 155 | Interaction with CD36; bind to | Asch A. S., et. al 1992 |
| | | cancerous cells. | |
| LRE | 156 | Neuron targeting by enhanced | Hunter D. D. et al, 1989 |
| | | neuronal attachment. | |
| YKYNLNGRES | 157 | Lung targeting | Asokan A, et al, 2006 |
| YRSL | 158 | Basolateral targeting | Anderson E., et al, 2005 |
| KGGK7 | 159 | Actin-binding | Dahlin-Huppe K. et al., 1997 |
| KKKQYTSIHHG | 160 | Basolateral sorting | Zheng P. et al, 1998 |
| KDEL | 161 | Endosomal Reticulum targeting | Chinnapen D. J. et al, 2007 |
| LADQDYTKTA | 162 | Retrograde transport | Tervo D. G. R., et. al, 2016 |
| DDNN | 163 | Corin surface targeting | Uniprot |
| SAVTTVVN | 164 | ITGB1 interaction with | Uniprot |
| | | ITGB1BP1 | |

For the avoidance of doubt, the present disclosure includes modified cell-type specific targeting domains, including without limitation a cell-type specific targeting domain of the present disclosure in which at least one amino acid of the cell-type specific targeting domain includes a modification disclosed herein.

### Nucleic Acid Release Domains

In some embodiments, a “nucleic acid release domain” (“NARD”) of a mini-nucleosome is an amino acid domain that causes or facilitates release of a nucleic acid cargo of a loaded mini-nucleosome (e.g., release of one or more of the nucleic acids associated with a mini-nucleosome core protein of a loaded mini-nucleosome). In various embodiments, by controlling or regulating the conditions under which a nucleic acid cargo is released, a nucleic acid release domain can improve delivery a nucleic acid cargo to cells, e.g., to the cytoplasm or nucleus of cells.

It is highly desirable that nucleic acids associated with a loaded mini-nucleosome core protein (e.g., when delivered to a subject or system) do not release from a loaded mini-nucleosome prior to the loaded mini-nucleosome entering a cell (e.g., a cell of the subject or system). Within a cell, release of nucleic acid cargo in the cytoplasm or nucleus may be preferred. Various proteases and endopeptidases known in the art could cause or facilitate release of one or more nucleic acids of a loaded mini-nucleosome inside cells, e.g., causing or facilitating disassociation of one or more nucleic acids of a loaded mini-nucleosome from the mini-nucleosome core protein of the loaded mini-nucleosome. Proprotein convertases and endopeptidases are exemplary agents that cleave polypeptides at certain amino acid domains, which phenomenon is utilized herein to provide mini-nucleosome core proteins that can release an associated nucleic acid cargo upon delivery into a cell (e.g., a cell of a subject or system), e.g., into the cytoplasm or nucleus of a cell.

KRH is an exemplary cleavage domain that can be included in a mini-nucleosome core protein as a nucleic acid release domain. KRH is the cleavage site for Pcsk1 and Pcsk2. To provide one non-limiting example of a KRH cleavage site, proglucagon is post-translationally processed in a tissue-specific manner in pancreatic A cells and intestinal cells by Pcsk1 or Pcsk2.

NRRKKRAL is an exemplary cleavage domain that can be included in a mini-nucleosome core protein as a nucleic acid release domain. To provide one non-limiting example of an NRRKKRAL cleavage domain, NRRKKRAL is a Furin cleavage site of for TGFB1. Another exemplary cleavage domain is KSVKKRSVSEIQ, which is a Furin cleavage site in parathyroid hormone.

Various other cleavage domains are known in the art and can be included in a mini-nucleosome core protein as a nucleic acid release domain. As those of skill in the art will appreciate, cleavage sites can also be predicted in silico using bioinformatics platforms such as Expasy, OmicX, PROSPERous, Prop1.0, SignalP-5.0, MEROPS, CutDB, Peptide Cutter etc.

The present disclosure provides that cleavage domains of the present disclosure can be included in mini-nucleosome core proteins to cause or facilitate release of a nucleic acid cargo of a loaded mini-nucleosomes in cells (e.g., cells of a subject or system), e.g., in cytoplasm or nucleus. Domains provided herein, including those provided in Table 9, can be present in a mini-nucleosome core protein of the present disclosure (e.g., a modified mini-nucleosome core protein) at any position within the mini-nucleosome core protein. For the avoidance of doubt, a nucleic acid release domain of the present disclosure can be present in a mini-nucleosome core protein in combination with other domains provided herein, including without limitation those provided in Tables 3, 4, 5, 6, 7, 8, 10, 11 and 12. When present anywhere within a mini-nucleosome core protein of the present disclosure, a nucleic acid release domain of the present disclosure can enhance release of a nucleic acid cargo of a loaded mini-nucleosome.

TABLE 9

| | SEQ ID | | |
|---|---|---|---|
| Exemplary Domains | NO: | Exemplary Utility | Reference |
| GRKKRRQRRRPQ | 165 | Release at extracellular or | Tian and Huang |
| | | intracellular sites depending on | et al, 2011 |
| | | tissues expressing furin. | |
| KRH | 166 | Release at extracellular or | Uniprot |
| | | intracellular sites depending on | |
| | | tissues expressing Pcsk1 and Pcsk2 | |
| KSVKKRSVSEIQ | 167 | Release at extracellular or | Uniprot |
| | | intracellular sites depending on | |
| | | tissues expressing Pesk1 and Pcsk2 | |
| NRRKKRAL | 168 | Release at extracellular or | Tian and Huang |
| | | intracellular sites depending on | et al, 2011 |
| | | tissues expressing furin. | |
| KFERQ | 169 | Breakdown in the lysosomes. | Park J. S. et al., 2016 |
| VRGP | 170 | Cleavage by Thrombin | Uniprot |
| NKDS, NRDN | 171 | Cleavage by Plasmin | Uniprot |
| ANNR | 172 | Cleavage by Hementin | Uniprot |
| HL | 173 | Cleavage by MMP9 | Uniprot |
| RI, ET, GQ, RS, RD, | 174, 175, | Cleavage by autolysis | Uniprot |
| RN, RC, RG, RL, DA, | 176, 177, | | |
| RA, GS, LT, FS, GL, | 178, 179, | | |
| SA, DP, GT, GC, RQ, | 180, 181, | | |
| LS, HA | 182, 183, | | |
| | 184, 185, | | |
| | 186, 187, | | |
| | 188, 189, | | |
| | 190, 191, | | |
| | 192, 193, | | |
| | 194, 195 | | |
| FV, QH, EA, AL, LY, | 196, 197, | Cleavage by Pepsin | Uniprot |
| YL, GF, PS, RE, DP, | 198, 199, | | |
| PI, QS | 200, 201, | | |
| | 202, 203, | | |
| | 204, 205, | | |
| | 206, 207 | | |
| ND | 208 | Cleavage by BMP1 | Uniprot |

For the avoidance of doubt, the present disclosure includes modified nucleic acid release domains, including without limitation a nucleic acid release domain of the present disclosure in which at least one amino acid of the nucleic acid release domain includes a modification disclosed herein.

### Stability Domains

In some embodiments, a “stability domain” of a mini-nucleosome is an amino acid domain that allows loaded mini-nucleosomes to stay stable in bodily fluids, cytoplasm and the nucleus.

Particle stability is important for safe passage into cells and longevity of expression. There are several reasons for particles to lose stability. First, particles should be stable in blood and other bodily fluids. Secondly, particles need to safely traverse the endosomal entry and escape safely to make it out to the cytoplasm. Viral particles or recycled receptors use several domains to enter the endosome and escape it. We provide examples of mini-nucleosome core proteins that incorporate endosomal entry and escape domains to increase stability. Domains mentioned herein, in Table 10 can be incorporated in mini-nucleosome core protein preferably at the C-terminal but also at any location to enhance stability of the mini-nucleosome core protein when combined with other domains provided in Table 3, 4, 5, 6, 7, 8, 9, 11 and 12. One skilled in the art may also contemplate fluorination of hydrophobic amino acids in the peptides to provide means of increasing protein stability, enhanced assembly etc. and to strengthen ligand-receptor interactions. One skilled in the art may also contemplate other post translational modifications to amino acids in the peptides to provide means of increasing protein stability, enhanced assembly etc. and to strengthen ligand-receptor interactions.

TABLE 10

| | SEQ ID | | |
|---|---|---|---|
| Exemplary Domains | NO: | Exemplary Utility | Reference |
| YTRF | 209 | Endocytosis signal for | Pandey K. N. |
| | | Transferrin receptor | 2009 |
| GDAY | 210 | Internalization signal for | Pandey K. N. |
| | | endocytosis of NPRA | 2009 |
| LLEE | 211 | Endosomal entry of Cd209 | Uniprot |
| RKKRRQRRR | 212 | Allows for endosomal escape | Najjar K, et. al., |
| | | | 2015 |
| YKSL | 213 | Endosomal entry of Cd209 | Uniprot |
| YENF | 214 | Endosomal entry of CELC10a | Uniprot |
| FQDL | 215 | Endosomal entry of CELC10a | Uniprot |
| YIGSR | 216 | Integrin conjugation, | Graf, J et al, |
| | | increased cell attachment | 1987 |
| IKVAV | 217 | Cell Membrane Penetrating | Tashiro, K, et al |
| | | Peptide, cell attachment | 1989 |
| EFAKFE | 218 | Recycling endosomes | Uniprot |
| LLEEEQLRGLGFRQTRGYKSL | 219 | Endosomal entry of Cd209 | Uniprot |

For the avoidance of doubt, the present disclosure includes modified nucleic acid release domains, including without limitation a nucleic acid release domain of the present disclosure in which at least one amino acid of the nucleic acid release domain includes a modification disclosed herein.

### Oligomerization Domains

Oligomerization is a chemical process by which monomers associate to form multimers, including dimers and higher order macromolecular complexes. Oligomerization of proteinaceous molecules is often facilitated by domains that promote association of monomers.

In some embodiments, an “oligomerization domain” of a mini-nucleosome is an amino acid domain that allows mini-nucleosome core proteins or loaded mini-nucleosomes to associate in higher order structures such as homodimer, heterodimer, tetramer, octamers or other higher order structures. Oligomerization can reduce the size of a loaded mini-nucleosome. A multimers of mini-nucleosome core proteins can include two or more of the same mini-nucleosome core protein (e.g., two mini-nucleosome core proteins having the same amino acid sequence) and/or can include two more distinct mini-nucleosome core proteins (e.g., two mini-nucleosome core proteins having different amino acid sequences). Examples of oligomerization domains provided herein are not in any way limiting and one skilled in the art can appreciated that such domains may be recognized or identified by various methods including yeast-two hybrid screening, affinity purification coupled to mass spectrometry, text mining, or by application of artificial intelligence and machine learning. One skilled in the art can also create an inducible system of forming loaded mini-nucleosomes using an inducible homodimerization system and/or chemically induced dimerization.

In some embodiments, an oligomerization domain can include 3 or more amino acids. Oligomerization domains disclosed herein, e.g., in Table 11, can be incorporated in mini-nucleosome core protein at any position of a mini-nucleosome core protein, e.g., in combination with other domains provided herein. e.g., in Table 3, 4, 5, 6, 7, 8, 9, 10 and 12. In certain particular embodiments, an oligomerization domain is positioned at the C-terminus of a mini-nucleosome core protein.

TABLE 11

| | SEQ ID | | |
|---|---|---|---|
| Exemplary Domains | NO: | Exemplary Utility | Reference |
| LIRERTE | 220 | Dimerization | Tucker C. L., et al, 1999 |
| LVEERTQ | 221 | Dimerization | Tucker C. L., et al, 1999 |
| IITFTK | 222 | Human PTB Domain helps | Markovtsov, V et al, 2000 |
| | | dimerization | |
| ILFNK | 223 | Human PTB Domain helps | Markovtsov, V et al, 2000 |
| | | dimerization | |
| PIRTLSK | 224 | Human PTB Domain helps | Markovtsov, V et al, 2000 |
| | | dimerization | |
| YGNSPLHRFK | 225 | Human PTB Domain helps | Markovtsov, V et al, 2000 |
| | | dimerization | |
| FFQKDR | 226 | Human PTB Domain helps | Markovtsov, V et al, 2000 |
| | | dimerization | |
| KSRP | 227 | Human PTB Domain helps | Markovtsov, V et al, 2000 |
| | | dimerization | |
| YVM | 228 | GRB2 domain mediated | Uniprot |
| | | interaction | |
| YMKM | 229 | YXXL domain helps | Uniprot |
| | | oligomerization | |
| RSSSFG | 230 | Protein-protein interaction | Uniprot |
| LKIRGRER, | 231, 232 | P53 oligomerization (part of) | Uniprot |
| LKIRGRKR | | | |
| HVIFKKVSR | 233 | Heterodimerization of SAG with | Uniprot |
| | | Rho | |
| RGPRV | 234 | Polymerization of Fibrin | Uniprot |
| RANVKHLK | 235 | Polymerization of CXCL12 | Uniprot |
| YPKAG, YPRTG | 236, 237 | Dimerization of DPP-IV | Tang, H-K et. al, 2011 |

For the avoidance of doubt, the present disclosure includes modified oligomerization domains, including without limitation an oligomerization domain of the present disclosure in which at least one amino acid of the oligomerization domain includes a modification disclosed herein.

### Linkers

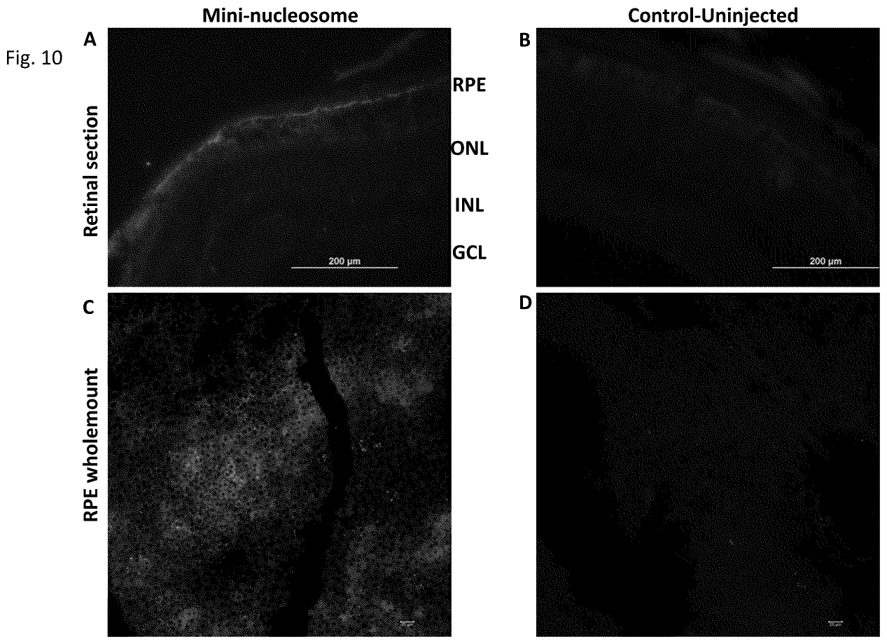

It is known in the art of creating fusion proteins that proteins can, some instances, benefit from inclusion of a linker. The present disclosure includes mini-nucleosome core proteins that include one or more linkers, e.g., between two domains of a mini-nucleosome core protein. Linkers can contribute to protein structure stability. In some cases, linkers work as a separation between domains and in others they can directly affect function of proteins. Some linkers increase stiffness thus allowing effective separation of protein domains. Linkers also may be implemented to introduce cleavage sites. Linkers have been used for these reasons in the field of protein engineering. However, in the context of non-viral gene transfer this strategy hasn't been utilized. We show here that linkers can be successfully used to engineer domains for functional purposes such as selective transduction, gene delivery and transgene expression in desired cell types (FIG. 10). In some cases, linkers separate domains and those of skill in the art will appreciate that non-functional amino acids between functional domains have been referred to in the art as spacers. For the avoidance of doubt, the term linker as used herein includes spacers.

In some embodiments, a linker sequence can include 1 or more amino acids. Linker amino acid sequences disclosed herein, e.g., in Table 12, can be incorporated in mini-nucleosome core protein between domains as shown in SEQ ID NOS: 238-335, where a linker could be a linker having any of the amino acids or amino acid sequences provided in Table 1 and 12. The linkers may contain other amino acid sequences not limited to those provided in Table 12. Linker sequences may also be generated via program called LINKER, which searches database of linker sequences using user-chosen inputs and generate output of linker sequences that fit the criteria. Threonine, serine, glycine, proline, arginine and alanine are preferred residues in natural linkers and thus, in mini-nucleosome core proteins.

TABLE 12

| | SEQ ID |
|---|---|
| Linkers | NO: |
| L | 238 |
| LL | 239 |
| GSS | 240 |
| GSSGSS | 241 |
| GGS | 242 |
| SSS | 243 |
| SSSSSS | 244 |
| GGSGG | 245 |
| GGSGGGGG | 246 |
| GGSGGHMGSGG | 247 |
| A(EAAAK)ₙA | 248 |
| (AP)ₙ | 249 |
| (KP)ₙ | 250 |
| (EP)ₙ | 251 |
| GT | 252 |
| AAGAATAA | 253 |
| GSGSGSGS | 254 |
| GGSSG | 255 |
| PP | 256 |
| WW | 257 |
| MH | 258 |
| QP | 259 |
| PL | 260 |
| CM | 261 |
| RM | 262 |
| RK | 263 |
| QR | 264 |
| HR | 265 |
| FW | 266 |
| PW | 267 |
| HR | 268 |
| DH | 269 |
| QS | 270 |
| WG | 271 |
| GM | 272 |
| KP | 273 |
| LF | 274 |
| YQ | 275 |
| RI | 276 |
| FY | 277 |
| FN | 278 |
| TA | 279 |
| HY | 280 |
| QV | 281 |
| DW | 282 |
| AW | 283 |
| YI | 284 |
| HT | 285 |
| CH | 286 |
| HP | 287 |
| TA | 288 |
| EM | 289 |
| KH | 290 |
| ML | 291 |
| AQ | 292 |
| YL | 293 |
| FI | 294 |
| KY | 295 |
| WR | 296 |
| LA | 297 |
| FS | 298 |
| AR | 299 |
| FN | 300 |
| ET | 301 |
| LW | 302 |
| NE | 303 |
| LH | 304 |
| MH | 305 |
| FY | 306 |
| PH | 307 |
| YE | 308 |
| HK | 309 |
| PW | 310 |
| HF | 311 |
| IM | 312 |
| DH | 313 |
| VH | 314 |
| DR | 315 |
| RI | 316 |
| QS | 317 |
| FC | 318 |
| GM | 319 |
| HR | 320 |
| HN | 321 |
| EC | 322 |
| VT | 323 |
| TH | 324 |
| CR | 325 |
| FQ | 326 |
| EV | 327 |
| KT | 328 |
| TD | 329 |
| SF | 330 |
| ST | 331 |
| QV | 332 |
| YK | 333 |
| NO | 334 |
| QK | 335 |

For the avoidance of doubt, the present disclosure includes a modified linker, including without limitation a linker of the present disclosure in which at least one amino acid of the linker includes a modification disclosed herein.

### Mini-Nucleosome Core Proteins

A mini-nucleosome core protein can include one or more domains provided herein.

Mini-nucleosome proteins disclosed herein include at least a positively charged amino acid sequence that contains a nucleic acid binding domain, a targeting domain and/or a nucleic acid release domain and/or a stability domain. The mini-nucleosome core protein can be sequences that have e.g., at least 65%, at least 70%, at least 75%, at least 80%, at least 85%, at least 90%, at least 95%, at least 96%, at least 97%, at least 98%, at least 99%, or 100% sequence identity with a mini-nucleosome core protein as set forth in any of SEQ ID NOs: 336-388. In various embodiments, a mini-nucleosome core protein has at least 65%, at least 70%, at least 75%, at least 80%, at least 85%, at least 90%, at least 95%, at least 96%, at least 97%, at least 98%, at least 99%, or 100% sequence identity with a mini-nucleosome core protein as set forth in one of SEQ ID NOs: 388-394, 399, 401, or 447.

In some embodiments, a mini-nucleosome core protein may contain amino acid sequence length from 10 to 100 amino acids. Amino acids, e.g., 10, 12, 15, 20, 25, 30, 35, 40, 45, 50, 55, 60, 55, 70, 75, 80, 85, 90, 95, or 100 amino acids. In certain embodiments, a mini-nucleosome core protein can have a length of, e.g., 15 to 90 amino acids, 20 to 80 amino acids, 20 to 70 amino acids, 20 to 60 amino acids, or 30 to 40 amino acids.

In certain embodiments, a mini-nucleosome core protein includes one or more domains disclosed herein and one or more amino acids that is not present in a domain disclosed herein. In certain instances, amino acids not present in a domain disclosed herein that are N-terminal or C-terminal of a domain disclosed herein can be referred to as “flanking amino acids,” and the sum of all amino acids present in a mini-nucleosome not present in any domain disclosed herein can be referred to as the “non-domain amino acids.”

In various embodiments, non-domain amino acids of a mini-nucleosome core protein can have a sequence that contributes to the charge of the mini-nucleosome core protein. In various embodiments, non-domain amino acids of a mini-nucleosome core protein include at least 10% positively charged amino acids. e.g., at least 20%, at least 30%, at least 40%, at least 50%, at least 60%, at least 70%, at least 80%, at least 90%, or 100% positively charged amino acids.

In some embodiments, at pH7, a mini-nucleosome core protein may have a total positive charge in between 10 and 100.

In some embodiments, a mini-nucleosome core protein can contain one or more nucleic acid binding domains placed at any location of the amino acid sequence. In some cases, the mini-nucleosome core protein may contain only the nucleic acid binding domains. In some cases, the mini-nucleosome core protein may contain on the nucleic acid binding domains and the poly-Arginine domains. In some cases, the mini-nucleosome core protein may contain on the nucleic acid binding domains and the targeting domains. In some cases, the mini-nucleosome core protein may contain only the poly-Arginine domains and the targeting domains. In some cases, the mini-nucleosome core protein may contain only the poly-Arginine domains, nucleic acid release domains and the targeting domains.

In some embodiments, a mini-nucleosome core protein may contain one or more poly-Arginines placed at any location of the amino acid sequence. The poly-Arginine sequence may contain 4-30 Arginines.

In some embodiments, a mini-nucleosome core protein may contain one or more targeting domains. The targeting domain may be placed at any location in the amino acid sequence of the mini-nucleosome core protein.

In some embodiments, a mini-nucleosome core protein may contain one or more nucleic acid release domains. Preferably, the nucleic acid release domains are placed in the middle of the amino acid sequence of the mini-nucleosome core protein. Preferably, the nucleic acid release domains are placed after 6 amino acids from the N-terminus or before 6 amino acids from the C-terminus.

In some embodiments, a mini-nucleosome core protein can contain one or more stability domains. Preferably, the stability domains are placed in the C-terminal of the amino acid sequence of the mini-nucleosome core protein. In some cases, the stability domains are placed in the N-terminal of the amino acid sequence of the mini-nucleosome core protein.

In some embodiments, a mini-nucleosome core protein can include one or more oligomerization domains. In certain particular embodiments, the oligomerization domains are positioned at the C-terminus of the amino acid sequence of a mini-nucleosome core protein. In some cases, the oligomerization domain is positioned at the N-terminus of the amino acid sequence of a mini-nucleosome core protein.

Thus, for the avoidance of doubt, a mini-nucleosome core protein, as set forth herein, can include (a) a nucleic acid binding domain (NABD), and (b) a targeting domain, and in some embodiments can include (a) a nucleic acid binding domain (NABD), (b) a targeting domain, and (c) a nucleic acid release domain. Those of skill in the art will appreciate from the present disclosure that a polypeptide including these components will constitute a mini-nucleosome core protein as disclosed herein, optionally subject to additional limitations set forth herein and/or including, without limitation, one or more further domains provided herein or otherwise known in the art. In some embodiments, a mini-nucleosome core protein can include a nucleic acid binding domain having at least 65% sequence identity with a nucleic acid binding domain as set forth in any of SEQ ID NOs: 1-28 (e.g., as set forth in Table 3), e.g., at least 65%, at least 70%, at least 75%, at least 80%, at least 85%, at least 90%, at least 95%, and/or that differs from a nucleic acid binding domain as set forth in any of SEQ ID NOs: 1-28 by no more than two amino acid changes (e.g., a deletion, addition, or substitution, e.g., a conservative substitution) or no more than one amino acid changes. In some embodiments, a mini-nucleosome core protein can include a targeting domain that is a cell attachment targeting domain having at least 65% sequence identity with a cell attachment targeting domain as set forth in any of SEQ ID NOs: 29-53 (e.g., as set forth in Table 4), e.g., at least 65%, at least 70%, at least 75%, at least 80%, at least 85%, at least 90%, at least 95%, and/or that differs from a cell attachment targeting domain as set forth in any of SEQ ID NOs: 29-53 by no more than two amino acid changes (e.g., a deletion, addition, or substitution, e.g., a conservative substitution) or no more than one amino acid changes. In some embodiments, a mini-nucleosome core protein can include a targeting domain that is a cell attachment targeting domain having at least 65% sequence identity with a cell attachment targeting domain as set forth in any of SEQ ID NOs: 54-81 (e.g., as set forth in Table 5), e.g., at least 65%, at least 70%, at least 75%, at least 80%, at least 85%, at least 90%, at least 95%, and/or that differs from a cell attachment targeting domain as set forth in any of SEQ ID NOs: 54-81 by no more than two amino acid changes (e.g., a deletion, addition, or substitution, e.g., a conservative substitution) or no more than one amino acid changes. In some embodiments, a mini-nucleosome core protein can include a targeting domain that is an internalization targeting domain having at least 65% sequence identity with an internalization targeting domain as set forth in any of SEQ ID NOs: 82-115 (e.g., as set forth in Table 6), e.g., at least 65%, at least 70%, at least 75%, at least 80%, at least 85%, at least 90%, at least 95%, and/or that differs from an internalization targeting domain as set forth in any of SEQ ID NOs: 82-115 by no more than two amino acid changes (e.g., a deletion, addition, or substitution, e.g., a conservative substitution) or no more than one amino acid changes. In some embodiments, a mini-nucleosome core protein can include a targeting domain that is a nucleus targeting domain having at least 65% sequence identity with a nucleus targeting domain as set forth in any of SEQ ID NOs: 116-139 (e.g., as set forth in Table 7), e.g., at least 65%, at least 70%, at least 75%, at least 80%, at least 85%, at least 90%, at least 95%, and/or that differs from a nucleus targeting domain as set forth in any of SEQ ID NOs: 116-139 by no more than two amino acid changes (e.g., a deletion, addition, or substitution, e.g., a conservative substitution) or no more than one amino acid changes. In some embodiments, a mini-nucleosome core protein can include a targeting domain that is a cell-type specific targeting domain having at least 65% sequence identity with a cell-type specific targeting domain as set forth in any of SEQ ID NOs: 140-164 (e.g., as set forth in Table 8), e.g., at least 65%, at least 70%, at least 75%, at least 80%, at least 85%, at least 90%, at least 95%, and/or that differs from a cell-type specific targeting domain as set forth in any of SEQ ID NOs: 140-164 by no more than two amino acid changes (e.g., a deletion, addition, or substitution, e.g., a conservative substitution) or no more than one amino acid changes. In some embodiments, a mini-nucleosome core protein can include a nucleic acid release domain having at least 65% sequence identity with a nucleic acid release domain as set forth in any of SEQ ID NOs: 165-208 (e.g., as set forth in Table 9), e.g., at least 65%, at least 70%, at least 75%, at least 80%, at least 85%, at least 90%, at least 95%, and/or that differs from a nucleic acid release domain as set forth in any of SEQ ID NOs: 165-208 by no more than two amino acid changes (e.g., a deletion, addition, or substitution, e.g., a conservative substitution) or no more than one amino acid changes. In some embodiments, a mini-nucleosome core protein can include a stability domain having at least 65% sequence identity with a stability domain as set forth in any of SEQ ID NOs: 209-219 (e.g., as set forth in Table 10), e.g., at least 65%, at least 70%, at least 75%, at least 80%, at least 85%, at least 90%, at least 95%, and/or that differs from a stability domain as set forth in any of SEQ ID NOs: 209-219 by no more than two amino acid changes (e.g., a deletion, addition, or substitution, e.g., a conservative substitution) or no more than one amino acid changes. In some embodiments, a mini-nucleosome core protein can include an oligomerization domain having at least 65% sequence identity with an oligomerization domain as set forth in any of SEQ ID NOs: 220-237 (e.g., as set forth in Table 11), e.g., at least 65%, at least 70%, at least 75%, at least 80%, at least 85%, at least 90%, at least 95%, and/or that differs from an oligomerization domain as set forth in any of SEQ ID NOs: 220-237 by no more than two amino acid changes (e.g., a deletion, addition, or substitution, e.g., a conservative substitution) or no more than one amino acid changes. In some embodiments, a mini-nucleosome core protein can include a linker domain having at least 65% sequence identity with a linker domain as set forth in any of SEQ ID NOs: 238-335 (e.g., as set forth in Table 12). e.g., at least 65%, at least 70%, at least 75%, at least 80%, at least 85%, at least 90%, at least 95%, and/or that differs from a linker domain as set forth in any of SEQ ID NOs: 238-335 by no more than two amino acid changes (e.g., a deletion, addition, or substitution, e.g., a conservative substitution) or no more than one amino acid changes.

Those of skill in the art that domains of a mini-nucleosome core protein provided herein can be arranged in any order, orientation, or sequence as provided herein or as will otherwise be understood from the present disclosure by those of skill in the art. For instance, those of skill in the art will appreciate the intended use of linkers. e.g., as optional sequences that can be included individually or in a tandem plurality between any pair of domains or adjacent to any domain, with or without one or more intervening amino acids not specifically disclosed herein. Thus, for example, a NABD can be C-terminal or N-terminal of a targeting domain. Additional domains provided herein, including without limitation additional NABDs or additional targeting domains, can be C-terminal or N-terminal of NABD and C-terminal or N-terminal of a targeting domain. Moreover, for each domain present in mini-nucleosome core protein, including a linker, one or more linker domains can be included C-terminal of the domain or N-terminal of the domain. Exemplary mini-nucleosome proteins are provided herein. As will be readily apparent to those of skill in the art from the present disclosure, domains provided herein are modular and can be included with their intended function in any order and/or thereby provide the mini-nucleosome with the intended utility or functionality regardless of the order in which they are present.

Those of skill in the art will further appreciate that mini-nucleosome core proteins of the present disclosure can include any number or type of modifications (e.g., posttranslational modifications) known in the art. Such modifications include, without limitation, pegylation, acetylation, methylation, glycosylation, phosphorylation, sumoylation, amidation, lipidation, and/or methylation. In various embodiments, a mini-nucleosome core protein can be pegylated.

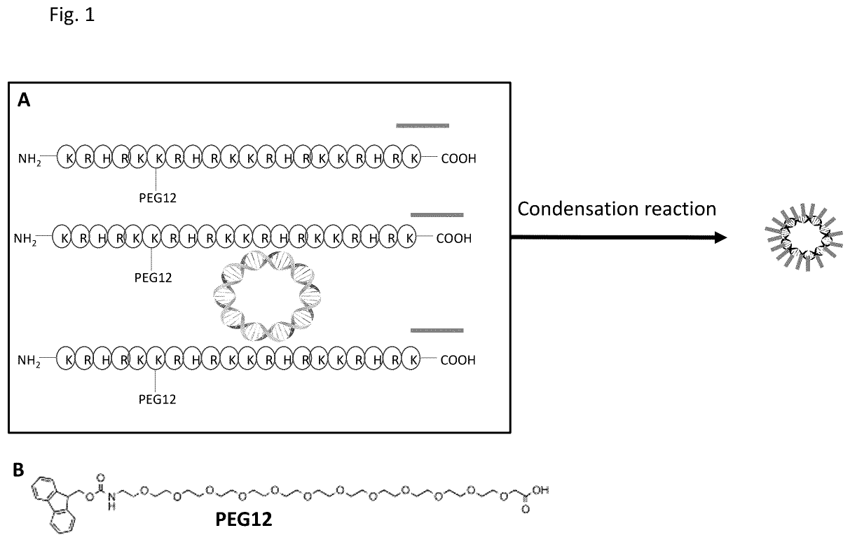

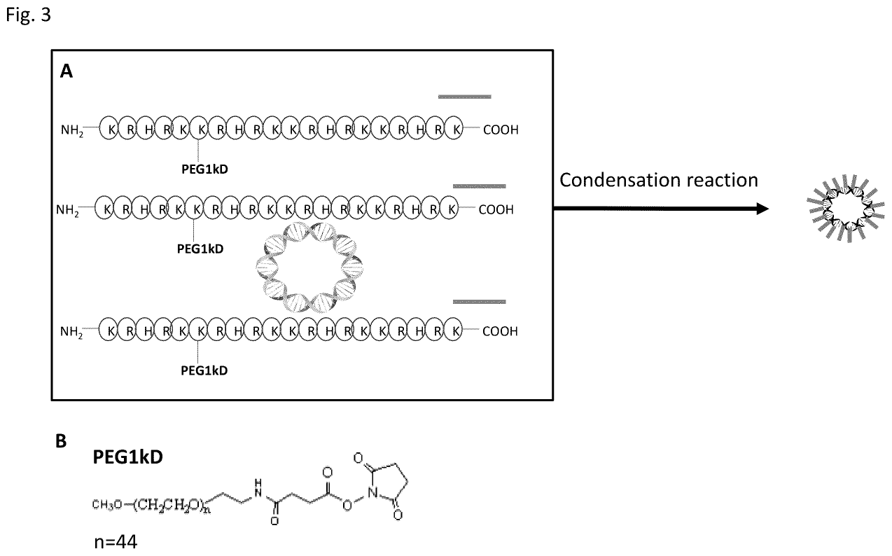

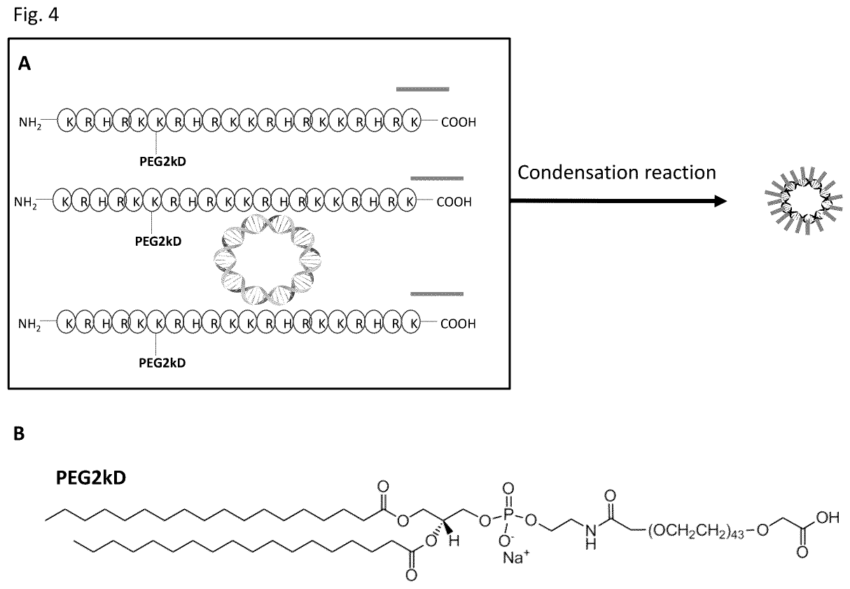

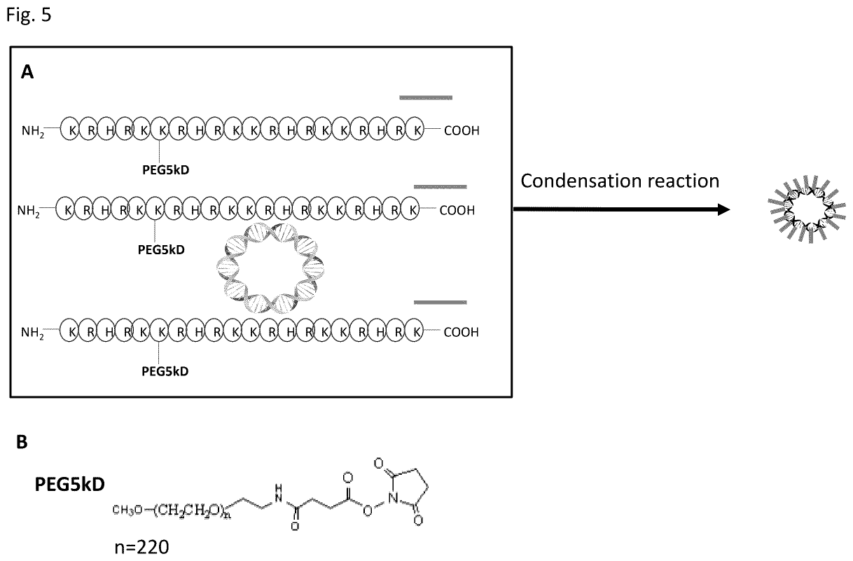

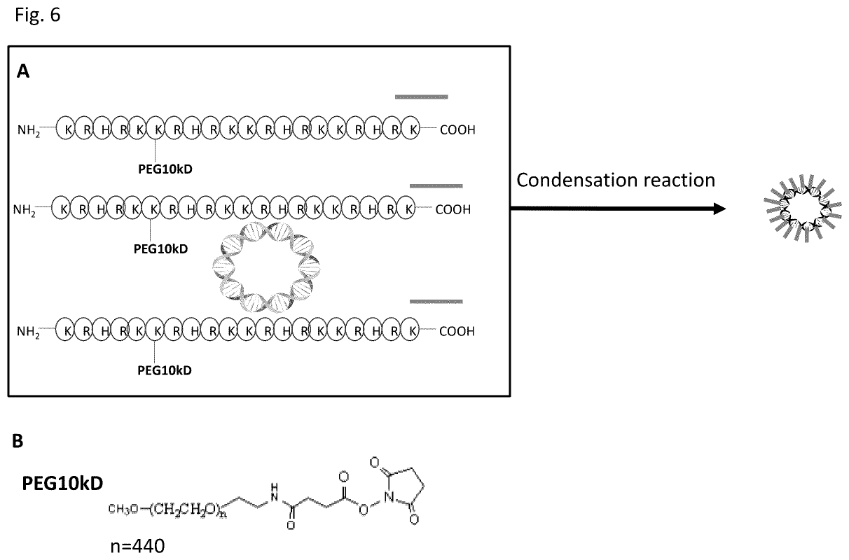

In some embodiments, a mini-nucleosome core protein is modified by association of the mini-nucleosome core protein with polyethylene glycol (PEG). PEG are nonionic, nontoxic, biocompatible and highly hydrophilic polymers. PEG is mostly used for the covalent modification of biological macromolecules and surfaces. PEG conjugation increases the apparent size of the polypeptide, thus reducing the renal filtration and altering biodistribution. PEGylation of peptides can enhance therapeutic properties due to their increased solubility (for hydrophobic peptides), prolonged half-life through reduced renal clearance, and masked antigenicity for minimum immune response in the host. PEGs of varying PEG chain lengths have been used in FDA cleared drugs with molecular weights ranging from 5-40 kDa. In FIGS. 1, 3, 4, 5 and 6, we show schematics of how PEGs of varying PEG chain lengths can be utilized to provide mini-nucleosome core proteins of varying size.

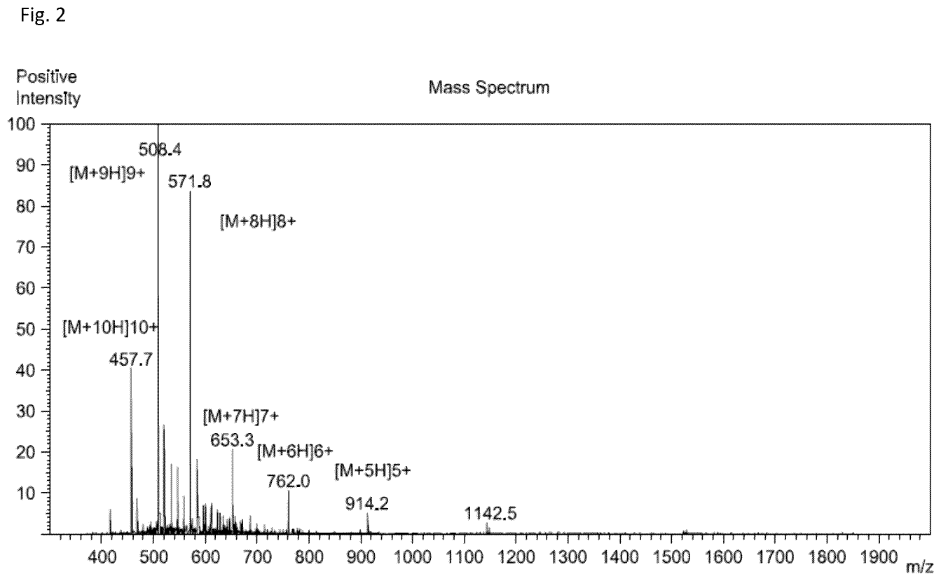

Many current particles use PEG of size 10 kDa or larger, however, a drawback to using larger PEG size is that it also increases particle size. (Feuz L. et al. 2007). The present disclosure provides, among other things, particles with varying PEG length to formulate mini-nucleosomes with varying size—preferably smaller than 20 nm in diameter. In FIG. 1, we show a minimal PEG length of 12 chains and how it can be utilized to modify amino acids in the mini-nucleosome core proteins. The final size of the loaded mini-nucleosome also depends on the PEG size used to modify the mini-nucleosome core proteins. FIG. 2 shows that by attaching PEG12, the molecular weight of the peptide increases accordingly, however doesn't change the physical characteristics such as solubility of the peptide.

In some embodiments, a mini-nucleosome core protein can have a total molecular weight between 1700 g/mol and 20000 g/mol, e.g., 1700, 1800, 1900, 2000, 2500, 3000, 3500, 4000.4500, 5000, 5500, 6000, 6500, 7000, 7500, 8000, 8500, 9000, 9500, 10000, 10500, 11000, 11500, or 20000 g/mol. In various embodiments, a mini-nucleosome core protein can have a total molecular weight between 100 Kda and 10,000 kDa, e.g. 100, 200, 500, 1000, 2,000, 3000, 5000, 8000, and 10000 kDa.

The amino acid sequence may be used in reverse or in any order. One may also contemplate changing one or non-essential amino acid in the domain to obtain same charge or other properties of the domain. For the avoidance of doubt, any mini-nucleosome core protein provided herein, including the exemplary mini-nucleosome core proteins provided in Table 13 below, can be modified at any amino acid and/or with any of one or more of the modifications provided herein.

TABLE 13

| | SEQ | Net | Number | Molecular | |
|---|---|---|---|---|---|
| Exemplary | ID | charge | of | weight | Iso-electric |
| Mini-nucleosome core protein sequences | NO. | at pH7 | residues | (g/mol) | point (pH) |
| KRHRKLREKRHRKLRRRRRLKRHRKKRHRKLREK | 336 | 22.4 | 34 | 4773.77 | 12.72 |
| KRHRKGSSLREKRHRKLRRRRRLKRHRKKRHRKLR | 337 | 22.4 | 40 | 5206.16 | 12.72 |
| EGGSK | | | | | |
| KRHRKREGSSLREKRHRKNDLRRRRRLKRHRKKRH | 338 | 21.4 | 44 | 5720.65 | 12.46 |
| RKLREGGSK | | | | | |
| KKPKKREGSSLREKRHRKNDLRRRRRLKRHRKKRH | 339 | 21.3 | 44 | 5624.6 | 12.38 |
| RKLREGGSK | | | | | |
| RRLARRGSSLREKRHRKLRRRRRLKKPKKKRHRKLR | 340 | 22.2 | 41 | 5213.23 | 12.72 |
| EGGSK | | | | | |
| KRHRKLREKRHRKLREKRHRKLKRHRKKRHRKLRE | 341 | 21.5 | 36 | 4984 | 12.48 |
| K | | | | | |
| KRHRKRILREKRHRKLREARKRHRKLKRHRKKRHR | 342 | 23.5 | 40 | 5480.61 | 12.56 |
| KLREK | | | | | |
| KRHRKKGKKKKGEKGKKKLKGKKKLRRRRRRRQR | 343 | 25.1 | 35 | 4507.55 | 12.78 |
| R | | | | | |
| KRHRKAPAPKGKKKKGEKGKKKLKGKKKLKPKPRR | 344 | 27.1 | 43 | 5294.51 | 12.79 |
| RRRRRQRR | | | | | |
| KRHRKGGSGGKGKKKKGEKGKKKLKGKKKLARRR | 345 | 25.1 | 41 | 4893.91 | 12.78 |
| RRRRQRR | | | | | |
| KRHRKLREKRHRKRRRRRRRKRHRKLREKRRQRR | 346 | 24.3 | 34 | 4906.85 | 12.84 |
| KRHRKKRHRKKRVKKKRHRKRRRRRRDSLL | 347 | 21.3 | 30 | 4141.02 | 12.86 |
| KRHRKKRHRKYQKRVKKKRHRKSSSRRRRRRDSLL | 348 | 21.3 | 35 | 4693.55 | 12.64 |
| KRHRKKKEEEKKEEEKKEEEKRRRRRRRQRRR | 349 | 12.1 | 32 | 4473.09 | 11.61 |
| KRHRKWRKKEEEKKEEEKKEEEKRIRRRRRRRQRRR | 350 | 14.1 | 36 | 5084.83 | 11.79 |
| KRHRKRGDKRHRKRRRRRKRHRKTPHKKK | 351 | 20.4 | 29 | 3964.72 | 12.82 |
| KRHRKFIRGDKRHRKRRRRRKRHRKLATPHKKK | 352 | 20.4 | 33 | 4409.28 | 12.82 |
| KRHRKRGDKRHRKRRRRRKRHRKGSSRNTPHQKKK | 353 | 22.4 | 36 | 4722.51 | 12.86 |
| K | | | | | |
| KRHRKRGDKRHRKLKRHRKRRRRKRHRKTPHKK | 354 | 22.5 | 33 | 4499.37 | 12.86 |
| KRHRKRGDKRHRKKRHRKKRHRKRGDKKTK | 355 | 19.4 | 30 | 3983.71 | 12.5 |
| KRHRKRGDKKRKKKKRGDKKRRRRRKKKPPSY | 356 | 21.1 | 32 | 4172.01 | 12.33 |
| KRHRKRKRKRKRRRRRKKKRASSLNIAKRRRR | 357 | 24.1 | 32 | 4308.23 | 13.26 |
| KRKKRKGKRLKRRREKRHRKRASSLNIAKKKK | 358 | 20.1 | 32 | 4054.95 | 12.68 |
| KRKKRRLKRKRKRRRRREKRHRKRRRQRRRKK | 359 | 27.1 | 32 | 4618.63 | 13.01 |
| KRKKRRKRKRRRRRKRHRKLRERKRRLREKK | 360 | 24.1 | 31 | 4420.4 | 12.75 |
| KRKNGRKRKRKKRHRKKKKRRRRKRHRKNGRKKK | 361 | 28.2 | 34 | 4587.61 | 13.2 |
| KRHRKWRHRARSKRHRKKKKKKRKKRKGK | 362 | 22.3 | 29 | 3902.77 | 13.03 |
| KRHRKRGDKRHRKKKKNRRKKRALRKKRKGK | 363 | 22.2 | 31 | 4047.92 | 12.73 |
| KKRKRGGKTKKKAKKALKKKKKGKKKKRRRRKKA | 364 | 28 | 38 | 4541.77 | 12.87 |
| APKK | | | | | |
| KKKAYPKALKKPKKKKKAYPKALKRRRRRKNRRK | 365 | 29.1 | 43 | 5481.83 | 12.53 |
| KRALKRHRK | | | | | |
| KTRSKKKKKRGDKKKKNRRKKRALNTQIHKKKKKA | 366 | 23.1 | 39 | 4725.78 | 12.4 |
| APKK | | | | | |
| KGKKKKGEKGKKKLKGKKKLRRRRRSPKKRRQRR | 367 | 23 | 34 | 4242.23 | 12.68 |
| KRHRKLREKRHRKLRRRRRLKRHRKKRHRKLREK | 368 | 22.4 | 34 | 4773.77 | 12.72 |
| KRHRKLREKRHRKLREKRHRKLKRHRKKRHRKLRE | 369 | 21.5 | 36 | 4984 | 12.48 |
| K | | | | | |
| KRHRKKGKKKKGEKGKKKLKGKKKLRRRRRRRQR | 370 | 25.1 | 35 | 4507.55 | 12.78 |
| R | | | | | |
| KRHRKLREKRHRKRRRRRRRKRHRKLREKRRQRR | 371 | 24.3 | 34 | 4906.85 | 12.84 |
| KRHRKKRHRKKRVKKKRHRKRRRRRRDSLL | 372 | 21.3 | 30 | 4141.02 | 12.86 |
| KRHRKKKEEEKKEEEKKEEEKRRRRRRRQRRR | 373 | 12.1 | 32 | 4473.09 | 11.61 |
| KRHRKQSKKEEEKKEEEKKEEEKNQRRRRRRRQRR | 374 | 12.1 | 36 | 4930.53 | 11.61 |
| R | | | | | |
| KRHRKRGDKRHRKRRRRRKRHRKTPHKKK | 375 | 20.4 | 29 | 3964.72 | 12.82 |
| KRHRKRGDKRHRKLKRHRKRRRRKRHRKTPHKK | 376 | 22.5 | 33 | 4499.37 | 12.86 |
| KRHRKRGDKRHRKKRHRKKRHRKRGDKKTK | 377 | 19.4 | 30 | 3983.71 | 12.5 |
| KRHRKRGDKKRKKKKRGDKKRRRRRKKKPPSY | 378 | 21.1 | 32 | 4172.01 | 12.33 |
| KRHRKGGSRGDKKRKKKKRGDSSSKKRRRRRKKKP | 379 | 21.1 | 38 | 4634.43 | 12.33 |
| PSY | | | | | |
| KRHRKRKRKRKRRRRRKKKRASSLNIAKRRRR | 380 | 24.1 | 32 | 4308.23 | 13.26 |
| KRKKRKGKRLKRRREKRHRKRASSLNIAKKKK | 381 | 20.1 | 32 | 4054.95 | 12.68 |
| KRKKRRLKRKRKRRRRREKRHRKRRRQRRRKK | 382 | 27.1 | 32 | 4618.63 | 13.01 |
| KRKKRRKRKRRRRRKRHRKLRERKRRLREKK | 383 | 24.1 | 31 | 4420.4 | pH 12.75 |
| KRKNGRKRKRKKRHRKKKKRRRRKRHRKNGRKKK | 384 | 28.2 | 34 | 4587.61 | 13.2 |
| KRKWRNGRKRKRQKRHRKKKKRARRRRKRHRKNG | 385 | 30.3 | 40 | 5422.54 | 13.26 |
| RKHKKK | | | | | |
| KRHRKWRHRARSKRHRKKKPKKRKKRKGK | 386 | 21.3 | 29 | 3871.71 | 13.03 |
| KRHRKPKPRIWRHRARSRDKRHRKKKPKKRKKRKG | 387 | 23.3 | 36 | 4734.73 | 12,78 |
| K | | | | | |
| KKKRKLRGDLKRKGSSYQPLAPAPKKKRKRGDKRK | 388 | 16.0 | 41 | 4894.87 | 11.77 |
| LFYQPL | | | | | |

For the avoidance of doubt, the present disclosure includes modified mini-nucleosome core proteins, including without limitation a mini-nucleosome core protein of the present disclosure in which at least one amino acid of the mini-nucleosome core protein includes a modification disclosed herein.

### Modified Mini-Nucleosome Core Proteins

The present disclosure provides, among other things, mini-nucleosome core proteins that include at least one modified amino acid residue. Various modifications and certain advantageous thereof are provided throughout the present disclosure. For the avoidance of doubt, the present disclosure provides that any and/or all residues of any and/or all domains, mini-nucleosome core proteins, or other polypeptides provided herein, and/or any and all residues of any portion(s) thereof, can be modified in accordance with the present disclosure of modifications. Moreover, various advantages resulting from any such amino acid modification as disclosed herein do not depend upon the specific residue within a domain, mini-nucleosome core protein, or other polypeptide that is modified, and/or the position thereof within a domain, mini-nucleosome core protein, or other polypeptide. In various embodiments, various advantages resulting from any such amino acid modification as disclosed herein are realized upon inclusion of the modification at any of one or more residues of a domain, mini-nucleosome core protein, or other polypeptide in accordance with the present disclosure.

In various embodiments, a modification of an amino acid present in a domain, mini-nucleosome core protein, or other polypeptide can be any of:
(i) phosphorylation;
(ii) sulfation;
(iii) glycosylation (e.g., N-glycosylation, C-glycosylation, and/or O-glycosylation);
(iv) prenylation (e;g., geranylation and/or farnesylation);
(v) methylation;
(vi) sialylation;
(vii) lipidation and/or lipoylation;
(viii) acetylation;
(ix) hydroxylation;
(x) palmitoylation;
(xi) mannosylation;
(xii) myristoylation;
(xiii) fucosylation;
(xiv) pegylation; and/or
(xv) any combination thereof, including any number of one or more of the modifications or variants thereof, e.g., in a branched or unbranched modification chain.

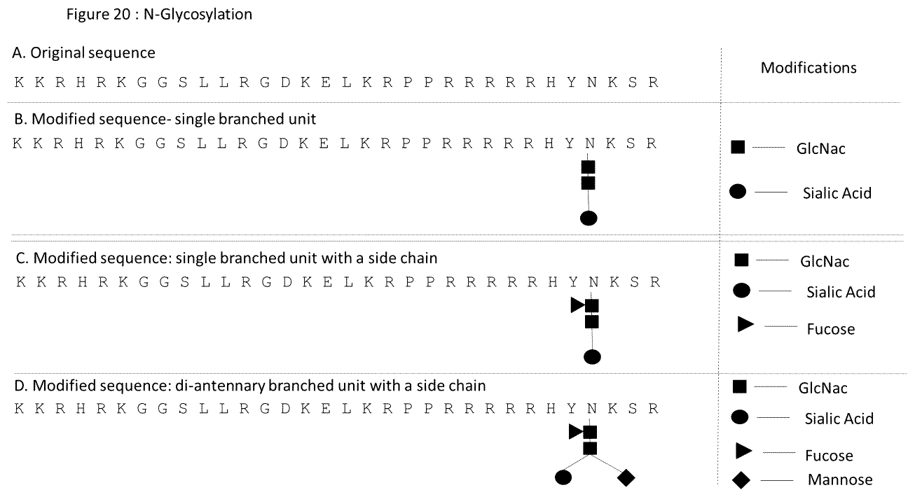

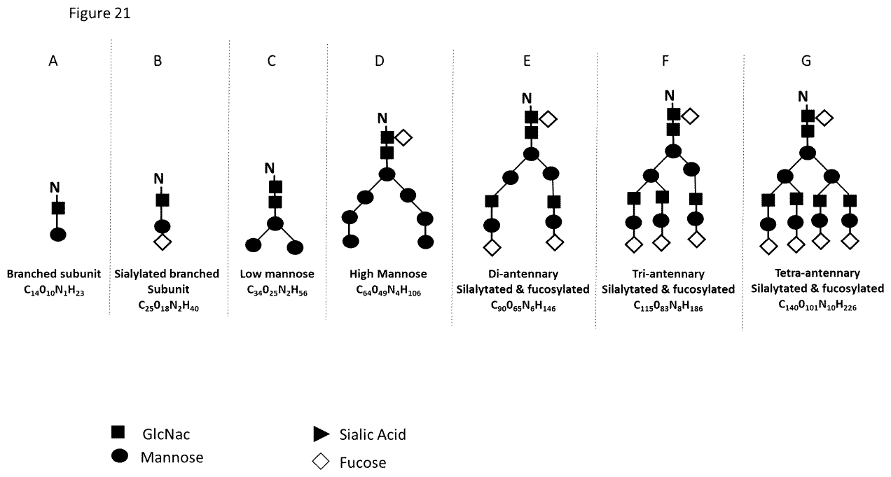

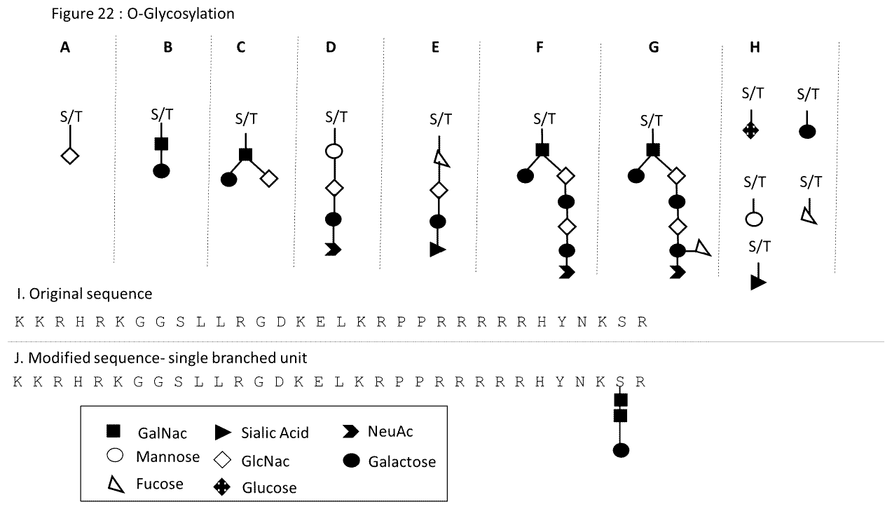

Examples of modifications and modified mini-nucleosome core proteins are found, e.g., in FIGS. 20-31. Those of skill in the art will appreciate that, even absent express disclosure of the atoms of any given chemical linkage between a modification and amino acid, or between two modifications in a modification chain, that such linkages are well known in the art of amino acids and amino acid modifications.

### Phosphorylated Mini-Nucleosome Core Proteins

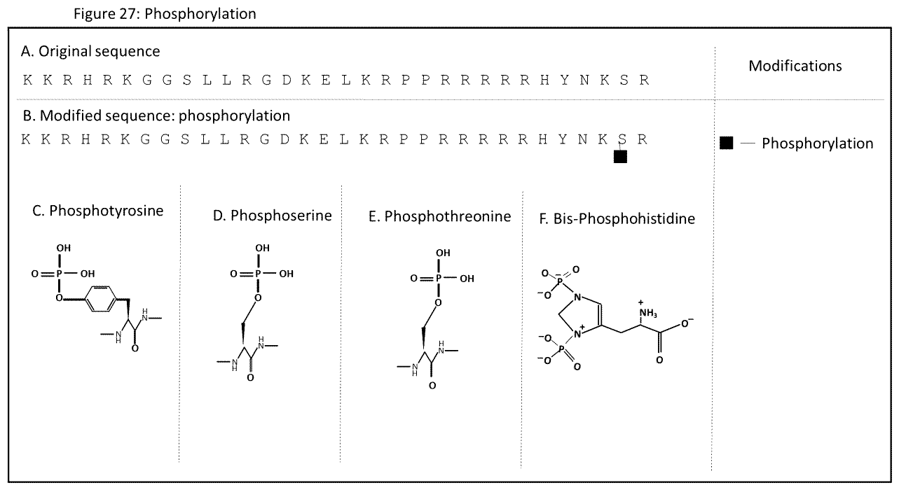

Phosphorylation typically occurs on serine, threonine, tyrosine, and/or histidine residues (S, Y, T, and/or H; see, e.g., FIG. 27). Phosphorylation includes covalent linkage of a phosphate group to an amino acid residue, such as to a hydroxyl side chain of a serine residue, a hydroxyl side chain of a threonine residue, or a phenolic side chain of a tyrosine residue. Phosphorylation is known to mediate protein functions including target binding, cellular localization, and enzymatic activity, among others. The present disclosure includes modified mini-nucleosome core proteins in which one or more threonine, tyrosine, and/or histidine residues are phosphorylated, e.g., mono-phosphorylated or bis-phosphorylated. In some embodiments, a modified mini-nucleosome core protein is phosphorylated by incorporation of protected phosphor-amino acids during polypeptide synthesis. In some embodiments, a modified mini-nucleosome core protein is phosphorylated by post-synthesis phosphorylation of residues, e.g., serine, threonine, or tyrosine residues. In general, at least certain advantages of phosphorylation have been demonstrated, e.g., by Kobayashi et. Al. 1996 (showing phosphorylation of ATF-1 increased DNA binding capabilities), Robin et. Al, 2003 (showing phosphorylation of GSTA4-4 increased targeting of GSTA4-4 to mitochondria by interaction with mitochondrial surface proteins), Rossetto D et al. 2012 (showing that phosphorylation of extra-nucleosomal histone H1, a linker Histone, increases stabilization of the nucleosome), and Anai et al, 2007 (showing a bis-phosphorylated peptide with increased binding affinity and selectivity for WW domains).

In various embodiments, phosphorylation of one more residues of a modified mini-nucleosome core protein increases the stability of the modified mini-nucleosome core protein, or loaded mini-nucleosome core proteins including the same, as compared to a reference mini-nucleosome core protein that does not include one or more of the phosphorylation modifications. In various embodiments, phosphorylation of one more residues of a modified mini-nucleosome core protein increases the half-life and/or bioavailability of the modified mini-nucleosome core protein, or loaded mini-nucleosome core proteins including the same, as compared to a reference mini-nucleosome core protein that does not include one or more of the phosphorylation modifications. In various embodiments, phosphorylation of one more residues of a modified mini-nucleosome core protein increases the affinity or avidity with a target cell or other binding partner of the modified mini-nucleosome core protein, or loaded mini-nucleosome core proteins including the same, as compared to a reference mini-nucleosome core protein that does not include one or more of the phosphorylation modifications. In various embodiments, phosphorylation of one more residues of a modified mini-nucleosome core protein increases the rate at which the modified mini-nucleosome core protein, or loaded mini-nucleosome core proteins including the same, enters one or more target cells (i.e., crosses the cell membranes of target cells) as compared to a reference mini-nucleosome core protein that does not include one or more of the phosphorylation modifications. In various embodiments, phosphorylation of one more residues of a modified mini-nucleosome core protein increases the affinity or avidity of the mini-nucleosome core protein with one or more nucleic acid cargos as compared to a reference mini-nucleosome core protein that does not include one or more of the phosphorylation modifications. Thus, in some embodiments, phosphorylation of one more residues of a modified mini-nucleosome core protein increases delivery of a mini-nucleosome core protein, loaded mini-nucleosome core protein, or nucleic acid cargo of a mini-nucleosome core protein to a target cell, tissue, or organ.

In certain particular embodiments of the present disclosure, phosphorylation of one more residues of a modified mini-nucleosome core protein decreases accumulation in liver of the modified mini-nucleosome core protein, or loaded mini-nucleosome core proteins including the same, as compared to a reference mini-nucleosome core protein that does not include one or more of the phosphorylation modifications. This is particularly advantageous in view of the empirical observation that most molecules and drug products, including adeno-associated viruses injected intravenously, accumulate in liver cells. Thus, phosphorylated mini-nucleosome core proteins of the present disclosure advantageously provide a means of nucleic acid delivery that reduces accumulation in liver of mini-nucleosome core proteins and/or loaded mini-nucleosome core proteins.

In certain particular embodiments of the present disclosure, targeting of a phosphorylated mini-nucleosome core protein, or loaded mini-nucleosome core protein including the same, to a target, target cell, or target tissue, e.g., by inclusion in the mini-nucleosome core protein of a targeting domain as provided herein, is increased in affinity, avidity, or rate as compared to a reference mini-nucleosome core protein that does not include one or more of the phosphorylation modifications. For example, phosphorylated mini-nucleosome core proteins, and loaded mini-nucleosome core proteins including the same, that are targeted to neurons, e.g., by inclusion of targeting domain for neuronal targeting, can deliver nucleic acid cargos to neurons with increased affinity, avidity, or rate as compared to a reference mini-nucleosome core protein that does not include one or more of the phosphorylation modifications.

Exemplary domains that can be included in mini-nucleosome core proteins, and which can optionally be modified by phosphorylation at one more residues, are provided in below Tables 14-16.

Table 14 includes exemplary targeting domains that target neurons, and which target neurons with greater affinity, avidity, or rate when phosphorylated at one or more underlined serine, threonine, and/or tyrosine residues.

Table 15 includes exemplary targeting domains that target muscle cells, and which target muscle cells with greater affinity, avidity, or rate when phosphorylated at one or more underlined serine, threonine, and/or tyrosine residues.

Table 16 includes exemplary targeting domains that target endothelial cells, and which target endothelial cells with greater affinity, avidity, or rate when phosphorylated at one or more underlined serine, threonine, and/or tyrosine residues.

TABLE 14

| Exemplary mini-nucleosome core | SEQ ID |
|---|---|
| protein domain | NO. |
| KKRHRKYPKKSRRSRLRNFRGDYNQYTRRRRR | 397 |
| KRKKRHRKRIRGRDVKYSYARKRHRKFQKWNYK | 398 |
| KKRHRKARRVTALREGRRHRKGERRRRRPPSY | 399 |
| KKRHRKALGSSDSLLARKRHRKKRKRKKRHRK | 400 |
| KKRHRKGSSKKRPKPRKKRHRKKRHRKKRHRKLL | 401 |
| KKRHRKRIQRRSRRGSSKHKGRDVILKKDVRKRHRK | 402 |
| KKRHRKKKDGKKRKRLLRKKHARALYIGSRKRGRKP | 403 |
| KKRHRKPPKDGEAQPKRHRKRRRRRKRHRKLRA | 404 |
| KKRKKRHRKLARGPRVARKRHRKRRRRRDRYQRL | 405 |

TABLE 15

| Exemplary mini-nucleosome core | SEQ ID |
|---|---|
| protein domain | NO. |
| KKRHRKRGFRRVSRRRGKKKEQRRERNARGKKGKRHRK | 406 |
| KKRHRKRRQPPRSISSHPLRKKRKGKTRRLRGDLRNSRR | 407 |
| KKRHRKRLRKKRKGKGSRPGSGFVKKTKQRRRRR | 408 |
| KKRHRKHRTKSGRSRIRKKRKGKRHARKKRRQRRRPPSY | 409 |
| KKRHRKKPVNRWSARNRRKKRALLRRRHYQRL | 410 |
| KKRHRKRKYKQCHKKGGHCFPKEKARRKKRKGKNEI | 411 |
| KKRHRKRIKKYRYYLKPLKKKRKKRKGKRHYLIIR | 412 |
| KKRHRKDRGRKKRRQRRRPQKPRKKRRQRRFQQI | 413 |
| KKRHRKGSSDPFRDDPFHRKRHRKKRHRKKRHRGRR | 414 |
| KKRHRKARSKTFNTHPQSTPYKRHRKRKKRKGKKRPK | 415 |
| RKKRKGKRAKRHRKKRHRKKPKNMTPYRSPPPYVPP | 416 |

TABLE 16

| Exemplary mini-nucleosome core | SEQ ID |
|---|---|
| protein domain | NO. |
| KKKRKRGDKRKRKRHRKKKRRRRLSIPPKA | 417 |
| KKKRKRGDKRKRKRHRKKKRRRRFQTPPQL | 418 |
| KKKRKRGDKRKRKRHRKKKRRRRLTPATAI | 419 |
| KKKRKRGDKRKRKRHRKKKRRRRSIGYPLP | 420 |
| KKKRKRGDKRKRKRHRKKKRRRRCLIRRTSIC | 421 |
| KKKRKRGDKRKRKRHRKKKRRRRCFFWKFRWMC | 422 |

### Sulfated Mini-Nucleosome Core Proteins

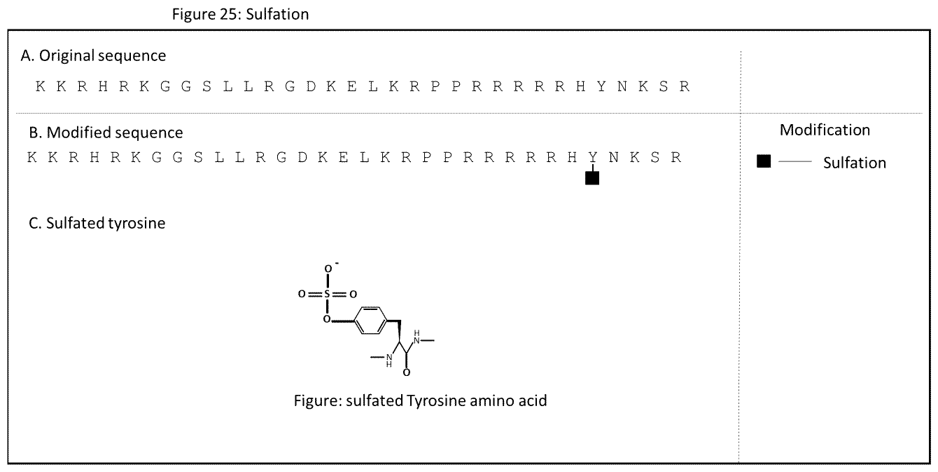

Sulfation refers to the covalent linkage of sulfate to a tyrosine (Y) residue (see, e.g., FIG. 25). In certain embodiments, an amino acid immediately N-terminal to a sulfated tyrosine in a polypeptide is an amino acid selected from E, N, S, H, V, and D, and/or an amino acid immediately C-terminal to a sulfated tyrosine in a polypeptide is an amino acid selected from E, L, D, Q, P, T, R and Y. In general, sulfation is known to increase the affinity and/or avidity of protein-protein interactions. For example, sulfation is predicted to increase selectin binding for increased update into endothelial cells, and Farzan et al. 1999 demonstrated that sulfation of CCR5 facilitates HIV entry. Proteins known to undergo sulfation include G-protein-coupled receptors, adhesion molecules, hormones, and extracellular matrix proteins. Sulfation is known to contribute to L- and P-selectin-mediated neutrophil recruitment, and leukocyte rolling (Somers et al, 2003).

In various embodiments, sulfation of one more residues of a modified mini-nucleosome core protein increases affinity or avidity with a target cell or other binding partner of the modified mini-nucleosome core protein, or loaded mini-nucleosome core proteins including the same, as compared to a reference mini-nucleosome core protein that does not include one or more of the sulfation modifications. In various embodiments, sulfation of one more residues of a modified mini-nucleosome core protein increases affinity or avidity of binding with a target receptor of the modified mini-nucleosome core protein, or loaded mini-nucleosome core proteins including the same, as compared to a reference mini-nucleosome core protein that does not include one or more of the sulfation modifications. Thus, in some embodiments, sulfation of one more residues of a modified mini-nucleosome core protein increases delivery of a mini-nucleosome core protein, loaded mini-nucleosome core protein, or nucleic acid cargo of a mini-nucleosome core protein to a target cell, tissue, or organ.

In various embodiments, sulfation of one more residues of a modified mini-nucleosome core protein increases the rate at which the modified mini-nucleosome core protein, or loaded mini-nucleosome core proteins including the same, enters one or more target cells (i.e., crosses the cell membranes of target cells) as compared to a reference mini-nucleosome core protein that does not include one or more of the sulfation modifications.

In various embodiments, sulfation of one more residues of a modified mini-nucleosome core protein increases blood-brain barrier penetration by the modified mini-nucleosome core protein, or loaded mini-nucleosome core proteins including the same, as compared to a reference mini-nucleosome core protein that does not include one or more of the sulfation modifications.

In various embodiments, sulfation of one more residues of a modified mini-nucleosome core protein increases selectivity of the modified mini-nucleosome core protein, or loaded mini-nucleosome core proteins including the same, for target receptors, and/or selectivity of internalization after receptor binding, as compared to a reference mini-nucleosome core protein that does not include one or more of the sulfation modifications.

Table 17 includes exemplary sulfated domains for inclusion in mini-nucleosome core proteins that enhance binding to endothelial cell surface markers and/or increase update of mini-nucleosome core proteins and/or loaded mini-nucleosome core proteins by endothelial cells.

TABLE 17

| Exemplary mini-nucleosome core | SEQ ID |
|---|---|
| protein domain | NO. |
| KKKRKRGDKRKRKRHRKKKRRRREYYLSIPPKA | 423 |
| KKKRKRGDKRKRKRHRKKKRRRDYRFQTPPQL | 424 |
| KKKRKRGDKRKRKRHRKKKRRRHYRLTPATAI | 425 |
| KKKRKRGDKRKRKRHRKKKRRRRVYQSIGYPLP | 426 |
| KKKRKSYRRGDKRKRKRHRKKKRRRRCLIRRTSIC | 427 |
| KKKRKEYRGDKRKRKRHRKKKRRRRCFFWKFRWMC | 428 |

### Glycosylated Mini-Nucleosome Core Proteins

Glycosylation includes, among other things, N-glycosylation, O-glycosylation, and C-glycosylation. N glycosylation, refers to covalent linkage of a glycan to a nitrogen atom of a side chain of an amino acid, typically the amide nitrogen of an asparagine (N) residue. Examples of N glycosylation include GlcNAc-β-Asn, GlcNac-α-Asn, and Glc-Asn. O-glycosylation refers to covalent linkage of a glycan to an oxygen atom of a hydroxyl side chain of an amino acid, typically an oxygen atom of a hydroxyl side chain of a serine (S) or threonine (T) residue. Examples of O-glycosylation include GlcNac-β-Ser/Thr and GalNac-α-Ser/Thr. C-glycosylation refers to covalent linkage of mannose to a carbon atom of a side chain of an amino acid, typically a carbon atom of a tryptophan (W) residue. An example of C-glycosylation is α-mannosyl tryptophan. Accordingly, in various embodiments, glycosylation can refer to one or more modifications, each of which modifications can be any of N-glycosylation, C-glycosylation, or O-glycosylation. Known functions of glycosylation modifications include contributions to protein folding and cell signaling. In general, glycosylation is also known to improve protein stability and serve as an epitope for association with binding partners.

In various embodiments, glycosylation of one more residues of a modified mini-nucleosome core protein increases the stability of the modified mini-nucleosome core protein, or loaded mini-nucleosome core proteins including the same, as compared to a reference mini-nucleosome core protein that does not include one or more of the glycosylation modifications. In some embodiments, glycosylation increases stability of the modified mini-nucleosome core protein, or loaded mini-nucleosome core proteins including the same, in that physical properties of the modified mini-nucleosome core protein, or loaded mini-nucleosome core proteins including the same, including protein structure and protein charge, are maintained and/or maintained for a longer period of time, e.g., after administration to a subject. In some embodiments, glycosylation decreases the occurrence or rate of thermal and/or kinetic denaturation of the modified mini-nucleosome core protein, or loaded mini-nucleosome core proteins including the same, as compared to a reference mini-nucleosome core protein that does not include one or more of the glycosylation modifications.

In various embodiments, glycosylation of one more residues of a modified mini-nucleosome core protein increases half-life and/or bioavailability of the modified mini-nucleosome core protein, or loaded mini-nucleosome core proteins including the same, as compared to a reference mini-nucleosome core protein that does not include one or more of the glycosylation modifications.

In various embodiments, glycosylation of one more residues of a modified mini-nucleosome core protein increases affinity or avidity with a target cell or other binding partner of the modified mini-nucleosome core protein, or loaded mini-nucleosome core proteins including the same, as compared to a reference mini-nucleosome core protein that does not include one or more of the glycosylation modifications. In various embodiments, glycosylation of one more residues of a modified mini-nucleosome core protein increases affinity or avidity of binding with a target receptor of the modified mini-nucleosome core protein, or loaded mini-nucleosome core proteins including the same, as compared to a reference mini-nucleosome core protein that does not include one or more of the glycosylation modifications. Thus, in some embodiments, glycosylation of one more residues of a modified mini-nucleosome core protein increases delivery of a mini-nucleosome core protein, loaded mini-nucleosome core protein, or nucleic acid cargo of a mini-nucleosome core protein to a target cell, tissue, or organ.

In various embodiments, glycosylation of one more residues of a modified mini-nucleosome core protein increases the rate at which the modified mini-nucleosome core protein, or loaded mini-nucleosome core proteins including the same, enters one or more target cells (i.e., crosses the cell membranes of target cells) as compared to a reference mini-nucleosome core protein that does not include one or more of the glycosylation modifications.

In various embodiments, glycosylation of one more residues of a modified mini-nucleosome core protein decreases precipitation and/or aggregation of the modified mini-nucleosome core protein, or loaded mini-nucleosome core proteins including the same, as compared to a reference mini-nucleosome core protein that does not include one or more of the glycosylation modifications.

Table 18 includes exemplary glycosylated domains, in particular galactose-modified targeting domains that target mini-nucleosome core proteins to liver.

TABLE 18

| Exemplary mini-nucleosome core | SEQ ID |
|---|---|
| protein domain | NO. |
| KKRHRKARARKKAAKARIKKAAPAKKAANRARKKH | 429 |
| KKRHRKGSSRRPRPGTGPGRRPRPRPRPRKKRNRSRQRRR | 430 |
| KKRHRKKYKQKIKHVVKLKKHRKRKRNRSIKVAV | 431 |
| KKRHRKSSSRTLQAHHDRQSNKRKRKNRSRRRRR | 432 |
| KKRHRKRNRSIHFNPRHRRRRRRDVARARAEKSKKK | 433 |
| KKRHRKNRSKKQRFRHRNRKGYRSQRGHSRGRNQNSRR | 434 |

### Prenylated Mini-Nucleosome Core Proteins

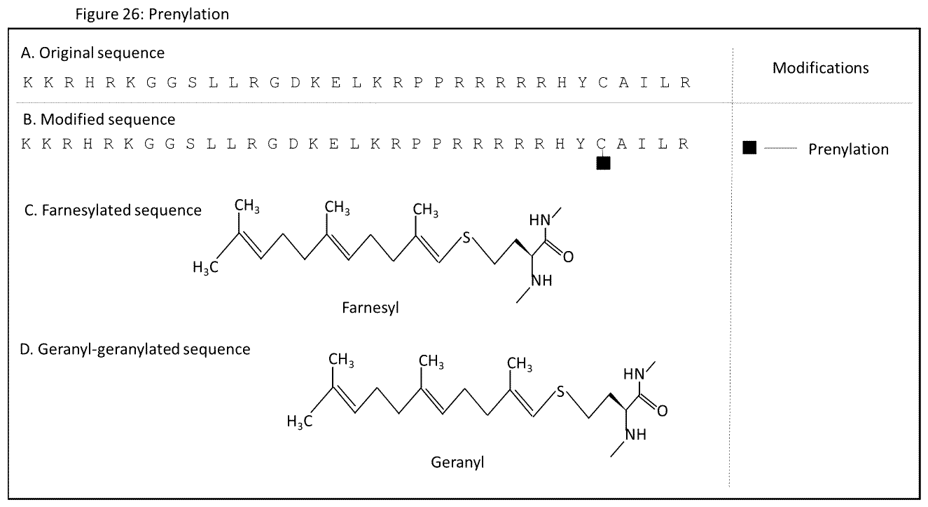

Prenylation typically occurs on cysteine residues (see, e.g., FIG. 26 for an example of a prenylated mini-nucleosome core protein). Prenylation includes covalent linkage of a lipid chain farnesyl (C15) or geranylgeranyl (C20) isoprenoid moiety to a free thiol group of a cysteine residue. Accordingly, prenylation includes modifications including farnesylation, geranylation, and geranylgeranylation. Prenylation is often found on C-terminal residues of polypeptides; in mammals, approximately 2% of proteins are prenylated at their C-terminal residues. Prenylation of a polypeptide such as a mini-nucleosome core protein significantly impacts hydrophobicity of the polypeptide. For at least that reason, prenylation of a mini-nucleosome core protein increases the strength (e.g., affinity or avidity) of interaction between a mini-nucleosome core protein and plasma membranes, facilitating efficient cellular uptake. Prenylation of a polypeptide such as a mini-nucleosome core protein also increases cell penetrating ability and/or uptake by target cells (see, e.g., Ochocki J D et al, 2011). Prenylation has also been shown to strengthen (e.g., increase the affinity or avidity of) protein-protein interactions (e.g., promoting protein-protein and protein-membrane interactions of proteins such as Ras, Rho, and Rab, etc., as reported by Gelb et al, 1998). Prenylation of proteins is also known to facilitate homing to target subcellular localizations.

In various embodiments, prenylation of one more residues of a modified mini-nucleosome core protein increases affinity or avidity with a target cell or other binding partner of the modified mini-nucleosome core protein, or loaded mini-nucleosome core proteins including the same, as compared to a reference mini-nucleosome core protein that does not include one or more of the prenylation modifications. In various embodiments, prenylation of one more residues of a modified mini-nucleosome core protein increases affinity or avidity of binding with a target receptor of the modified mini-nucleosome core protein, or loaded mini-nucleosome core proteins including the same, as compared to a reference mini-nucleosome core protein that does not include one or more of the prenylation modifications. Thus, in some embodiments, prenylation of one more residues of a modified mini-nucleosome core protein increases delivery of a mini-nucleosome core protein, loaded mini-nucleosome core protein, or nucleic acid cargo of a mini-nucleosome core protein to a target cell, tissue, or organ.

In various embodiments, prenylation of one more residues of a modified mini-nucleosome core protein increases strength (e.g., affinity or avidity) of association with cell membranes of the modified mini-nucleosome core protein, or loaded mini-nucleosome core proteins including the same, as compared to a reference mini-nucleosome core protein that does not include one or more of the prenylation modifications. In various embodiments, prenylation of one more residues of a modified mini-nucleosome core protein increases the rate at which the modified mini-nucleosome core protein, or loaded mini-nucleosome core proteins including the same, penetrates cell membranes as compared to a reference mini-nucleosome core protein that does not include one or more of the prenylation modifications. In various embodiments, prenylation of one more residues of a modified mini-nucleosome core protein increases the rate at which the modified mini-nucleosome core protein, or loaded mini-nucleosome core proteins including the same, enters one or more target cells (i.e., crosses the cell membranes of target cells) as compared to a reference mini-nucleosome core protein that does not include one or more of the prenylation modifications.

In various embodiments, prenylation of one more residues of a modified mini-nucleosome core protein increases the rate at which the modified mini-nucleosome core protein, or loaded mini-nucleosome core proteins including the same, home to target subcellular localization as compared to a reference mini-nucleosome core protein that does not include one or more of the prenylation modifications.

Table 19 includes exemplary prenylated domains, in particular prenylated targeting domains that target mini-nucleosome core proteins to liver cells and other cell types.

TABLE 19

| Exemplary mini-nucleosome core | SEQ |
|---|---|
| protein domain | ID NO. |
| KKRHRKARARKKAAKARIKKAAPAKKAARACIILRKKH | 435 |
| KKRHRKGSSRRPRPGTGPGRRPRPRPRPRKKRCASERQRRR | 436 |
| KKRHRKCIIEKYKQKIKHVVKLKKHRKRKRIKVAV | 437 |
| KKRHRKCQALSSSRTLQAHHDRQSNKRKRKRRRRR | 438 |
| KKRHRKRIHFNPRHRRRRRRCIAEDVARARAEKSKKK | 439 |
| KRHRKKKQRFRHRNRKGYRSQRGHSRGRNQNSRRCIILR | 440 |

### Methylated Mini-Nucleosome Core Proteins

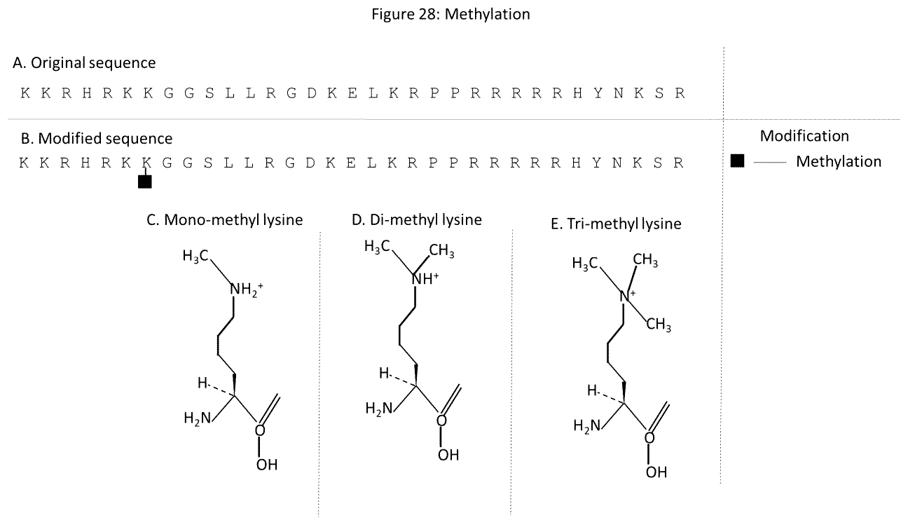

Methylation typically occurs on lysine (K) and arginine (R) residues (see, e.g., FIG. 28). Methylated residues include mono-methylated residues, di-methylated residues, and tri-methylated residues, among others. In particular, lysine residues are typically mono-methylated, di-methylated, or tri-methylated, and arginine residues are typically mono-methylated or di-methylated. Studies have demonstrated that histones methylated on certain residues cause and/or contribute to epigenetic control of gene expression. Methylation of polypeptides such as methylated mini-nucleosome core proteins increases targeting of the polypeptides to the nucleus.

In various embodiments, methylation of one more residues of a modified mini-nucleosome core protein increases the rate of delivery to the nuclei of target cells of the modified mini-nucleosome core protein, or loaded mini-nucleosome core proteins including the same, as compared to a reference mini-nucleosome core protein that does not include one or more of the methylation modifications. In various embodiments, methylation of one more residues of a modified mini-nucleosome core protein increases the rate of delivery to the nuclei of target cells of a nucleic acid cargo of a loaded mini-nucleosome core protein including the modified mini-nucleosome core protein, as compared to delivery of the nucleic acid cargo of a reference loaded mini-nucleosome core protein that includes a mini-nucleosome core protein that does not include one or more of the methylation modifications. In various embodiments, methylation of one more residues of a modified mini-nucleosome core protein increases expression in target cells of a coding sequence of a nucleic acid cargo of a loaded mini-nucleosome core protein including the modified mini-nucleosome core protein, as compared to expression of a coding sequence of a nucleic acid cargo of a reference loaded mini-nucleosome core protein that includes a mini-nucleosome core protein that does not include one or more of the methylation modifications.

Table 20 includes exemplary methylated domains, in particular methylated targeting domains that target mini-nucleosome core proteins to cell nuclei and/or increase expression of nucleic acid cargos when included in a mini-nucleosome core protein of a loaded mini-nucleosome core protein.

TABLE 20

| Exemplary mini-nucleosome core | SEQ ID |
|---|---|
| protein domain | NO. |
| KKRHRKARARKKAAKARIKKAAPAKKAARARKKH | 441 |
| KKRHRKGSSRRPRPGTGPGRRPRPRPRPRKKRRQRRR | 442 |
| KKRHRKKYKQKIKHVVKLKKHRKRKRIKVAV | 443 |
| KKRHRKSSSRTLQAHHDRQSNKRKRKRRRRR | 444 |
| KKRHRKRIHFNPRHRRRRRRDVARARAEKSKKK | 445 |
| KRHRKKKQRFRHRNRKGYRSQRGHSRGRNQNSRR | 446 |

### Sialylated Mini-Nucleosome Core Proteins

Sialylation refers to the covalent addition of sialic acid to the terminal end of a glycoprotein oligosaccharide chain. Sialylation can occur, e.g., on asparagine (N) and serine (S) residues. Sialylation can increase endothelial cell targeting and/or blood brain barrier penetration. In some instances, endothelial cell targeting provides a means of increased blood brain barrier penetration.

In various embodiments, sialylation of one more residues of a modified mini-nucleosome core protein increases blood-brain barrier penetration by the modified mini-nucleosome core protein, or loaded mini-nucleosome core proteins including the same, as compared to a reference mini-nucleosome core protein that does not include one or more of the sialylation modifications.

In various embodiments, sialylation of one more residues of a modified mini-nucleosome core protein increases strength (e.g., affinity or avidity) of association with cell membranes (e.g., endothelial cell membranes) of the modified mini-nucleosome core protein, or loaded mini-nucleosome core proteins including the same, as compared to a reference mini-nucleosome core protein that does not include one or more of the sialylation modifications. In various embodiments, sialylation of one more residues of a modified mini-nucleosome core protein increases the rate at which the modified mini-nucleosome core protein, or loaded mini-nucleosome core proteins including the same, penetrates cell membranes (e.g., endothelial cell membranes) as compared to a reference mini-nucleosome core protein that does not include one or more of the sialylation modifications. In various embodiments, sialylation of one more residues of a modified mini-nucleosome core protein increases the rate at which the modified mini-nucleosome core protein, or loaded mini-nucleosome core proteins including the same, enters one or more target cells (i.e., crosses the cell membranes of target cells), such as endothelial cells, as compared to a reference mini-nucleosome core protein that does not include one or more of the sialylation modifications.

Table 21 includes exemplary sialylated domains, in particular sialylated targeting domains that increase endothelial cell targeting and/or blood brain barrier penetration when included in a mini-nucleosome core protein.

TABLE 21

| Exemplary mini-nucleosome core | SEQ ID |
|---|---|
| protein domain | NO. |
| KKRHRKGGSLLRGEKELKRPPRRRRRKYIGSR | 447 |
| KKKRKLRGDLKRKPLISRRLIDRYQKKKRKRGDKRK | 448 |
| KKKRKLRGDLKRKSSSVRKKPGGSKKKRKRGDKRK | 449 |
| KKKRKLRGDLKRKGTQPEHSSTDHKKKRKRGDKRK | 450 |
| KKKRKRGDKRKRKRHRKKKRRRRLSIPPKA | 451 |
| KKKRKRGDKRKRKRHRKKKRRRRFQTPPQL | 452 |
| KKKRKRGDKRKRKRHRKKKRRRRNRSLTPATAI | 453 |
| KKKRKRGDKRKRKRHRKKKRRRRSIGYPLP | 454 |
| KKKRKRGDKRKRKRHRKKKRRRRNRSCLIRRTSIC | 455 |
| KKKRKRGDKRKRKRHRKKKRRRRNRSCFFWKFRWMC | 456 |
| KKKRKNRSRGDKRKRKRHRKKKRRRRIELLQARGC | 457 |
| KKKRKRGDKRKRKRHRKKKNRSRRRRREDV | 458 |
| KKKRKRGDKRKRKRHRKKKRRRRVHPKQHRGGSKGC | 459 |

Lipidated and/or Lipoylated Mini-Nucleosome Core Proteins

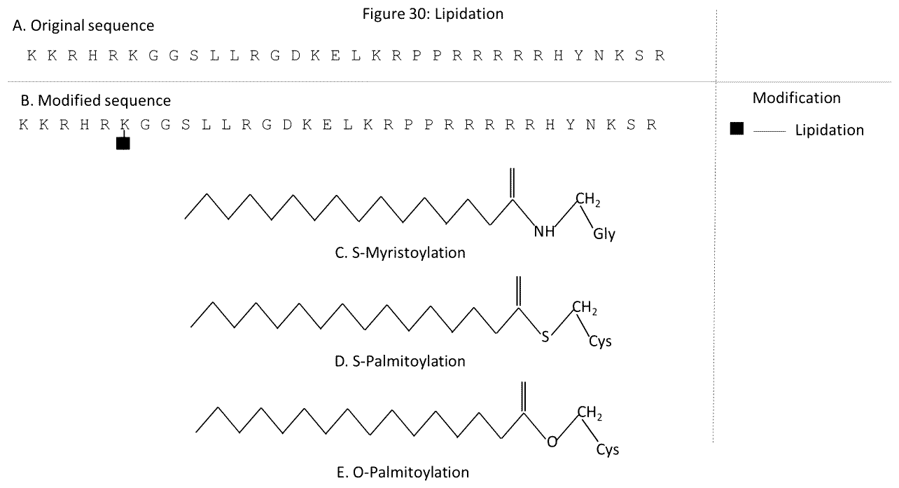

Amino acids can be covalently modified by linkage to a variety of lipids, including fatty acids, isoprenoids, and cholesterol (see, e.g., FIG. 30). Fatty acids that can be linked to amino acid residues for lipidation and/or lipoylation include caprylic acid (C8), capric acid (C10), lauric acid (C12), myristic acid (C14), palmitic acid (C16) or Stearic acid (C18). Fatty acids can be conjugated to the N-terminus of a polypeptide or to a side-chain of a lysine residue. Cysteine residues can also be covalently linked to fatty acids. Lipoylation is a form of acylation that includes linkage of an amino acid to a lipoate (C8) functional group. Lipoylation can occur, e.g., on lysine residues. In some instances, a polypeptide such as a mini-nucleosome core protein can be covalently linked to cholesterol. Cholesterol can be conjugated to a mini-nucleosome core protein at a N-terminal residue or at a C-terminal residue, and/or at a cysteine residue.

Lipidation and/or lipoylation can contribute to polypeptide localization and function. Lipidation and/or lipoylation can improve pharmacokinetic properties of a polypeptide such as a mini-nucleosome core protein, e.g., to increase half-life in circulation. Lipidation and/or lipoylation, e.g., with long-chain fatty acids, can also increase targeting of polypeptides such as a mini-nucleosome core protein (see, e.g., Hossieni et al., 2019, demonstrating that palmitoylation can significantly improve targeting in Wnt trafficking). Linkage of a mini-nucleosome core protein to cholesterol can increase membrane anchoring and thereby increase delivery to target cells.

In various embodiments, lipidation and/or lipoylation of one more residues of a modified mini-nucleosome core protein increases affinity or avidity with a target cell or other binding partner of the modified mini-nucleosome core protein, or loaded mini-nucleosome core proteins including the same, as compared to a reference mini-nucleosome core protein that does not include one or more of the lipidation and/or lipoylation modifications. In various embodiments, lipidation and/or lipoylation of one more residues of a modified mini-nucleosome core protein increases affinity or avidity of binding with a target receptor of the modified mini-nucleosome core protein, or loaded mini-nucleosome core proteins including the same, as compared to a reference mini-nucleosome core protein that does not include one or more of the lipidation and/or lipoylation modifications. Thus, in some embodiments, lipidation and/or lipoylation of one more residues of a modified mini-nucleosome core protein increases delivery of a mini-nucleosome core protein, loaded mini-nucleosome core protein, or nucleic acid cargo of a mini-nucleosome core protein to a target cell, tissue, or organ.

In various embodiments, lipidation and/or lipoylation of one more residues of a modified mini-nucleosome core protein increases half-life and/or bioavailability of the modified mini-nucleosome core protein, or loaded mini-nucleosome core proteins including the same, as compared to a reference mini-nucleosome core protein that does not include one or more of the lipidation and/or lipoylation modifications.

### Acetylated Mini-Nucleosome Core Proteins

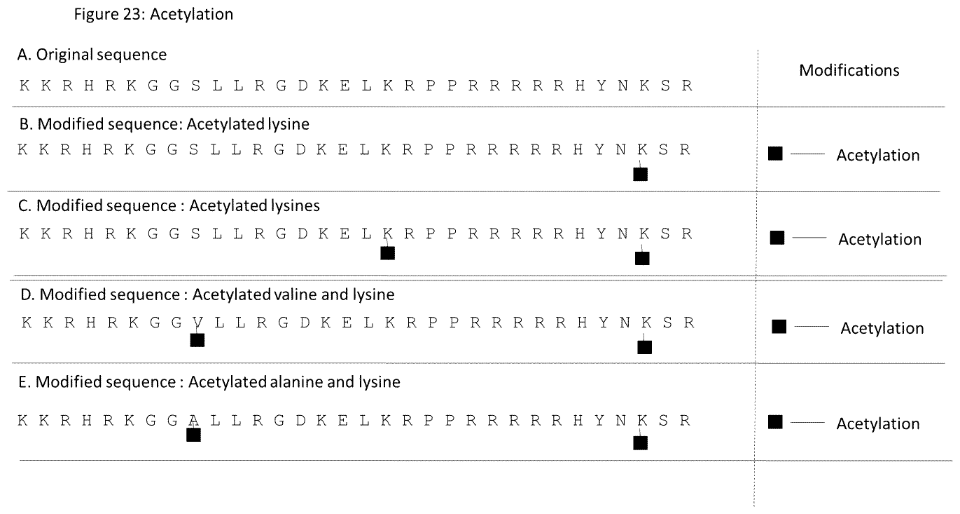

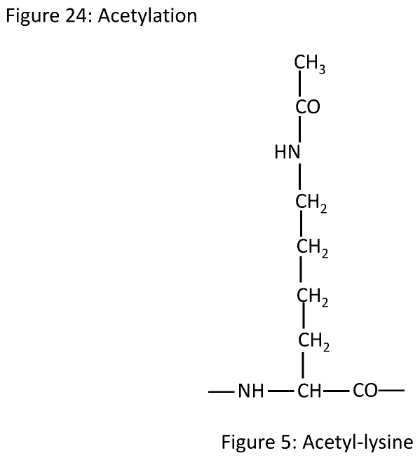

Acetylation can occur, e.g., on alanine (A), valine (V), and/or lysine (K) residues. An acetyl group can be covalently linked to an α-amino group of the N-terminus of a polypeptide or to an ε-amino group of a lysine residue (see, e.g., FIGS. 23 and 24). Acetylation refers to the substitution of hydrogen with an acetyl group in a residue of a polypeptide, such as a mini-nucleosome core protein. Acetylation can increase the stability and/or biological activity of a polypeptide, such as a mini-nucleosome core protein. For example, acetylation can increase stability of modified polypeptides by preventing N-terminal degradation. Acetylation is a reversible modification.

In various embodiments, acetylation of one more residues of a modified mini-nucleosome core protein increases the stability of the modified mini-nucleosome core protein, or loaded mini-nucleosome core proteins including the same, as compared to a reference mini-nucleosome core protein that does not include one or more of the acetylation modifications. In some embodiments, acetylation increases stability of the modified mini-nucleosome core protein, or loaded mini-nucleosome core proteins including the same, in that physical properties of the modified mini-nucleosome core protein, or loaded mini-nucleosome core proteins including the same, including protein structure and protein charge, are maintained and/or maintained for a longer period of time, e.g., after administration to a subject. In some embodiments, acetylation decreases the occurrence or rate of thermal and/or kinetic denaturation of the modified mini-nucleosome core protein, or loaded mini-nucleosome core proteins including the same, as compared to a reference mini-nucleosome core protein that does not include one or more of the acetylation modifications.

In various embodiments, acetylation of one more residues of a modified mini-nucleosome core protein increases half-life and/or bioavailability of the modified mini-nucleosome core protein, or loaded mini-nucleosome core proteins including the same, as compared to a reference mini-nucleosome core protein that does not include one or more of the acetylation modifications.

### Hydroxylated Mini-Nucleosome Core Proteins

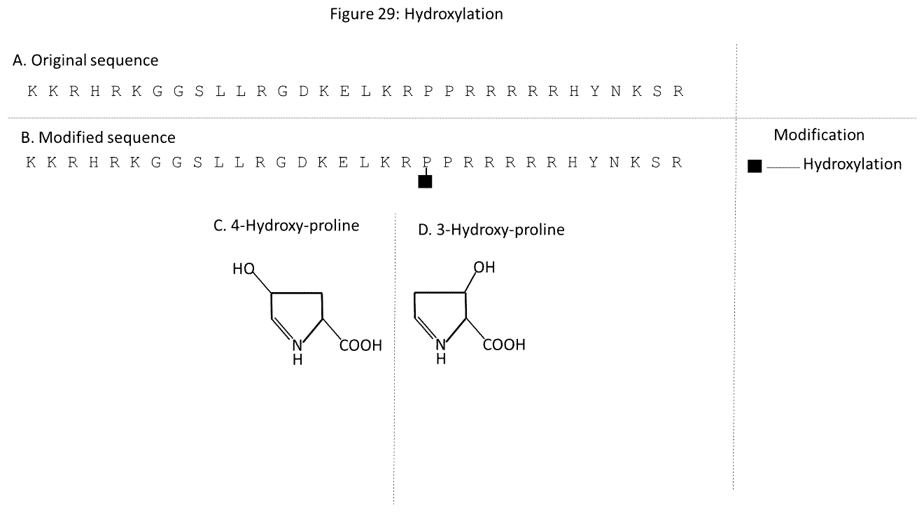

Hydroxylation can occur, e.g., on proline (P) and lysine (K) residues, as well as other amino acids including without limitation asparagine, aspartate and histidine (see, e.g., FIG. 29). Proline and/or lysine hydroxylation is associated with a variety of physiological molecules and/or processes, including certain molecules and/or process of certain pathological states. For example, proline hydroxylation can increase stability of a polypeptide such as a mini-nucleosome core protein.

In various embodiments, hydroxylation of one more residues, e.g., one or more proline residues, of a modified mini-nucleosome core protein increases the stability of the modified mini-nucleosome core protein, or loaded mini-nucleosome core proteins including the same, as compared to a reference mini-nucleosome core protein that does not include one or more of the hydroxylation modifications. In some embodiments, hydroxylation of one more residues, e.g., one or more proline residues, increases stability of the modified mini-nucleosome core protein, or loaded mini-nucleosome core proteins including the same, in that physical properties of the modified mini-nucleosome core protein, or loaded mini-nucleosome core proteins including the same, including protein structure and protein charge, are maintained and/or maintained for a longer period of time, e.g., after administration to a subject. In some embodiments, hydroxylation of one more residues, e.g., one or more proline residues, decreases the occurrence or rate of thermal and/or kinetic denaturation of the modified mini-nucleosome core protein, or loaded mini-nucleosome core proteins including the same, as compared to a reference mini-nucleosome core protein that does not include one or more of the hydroxylation modifications.

In various embodiments, hydroxylation of one more residues, e.g., one or more proline residues, of a modified mini-nucleosome core protein increases half-life and/or bioavailability of the modified mini-nucleosome core protein, or loaded mini-nucleosome core proteins including the same, as compared to a reference mini-nucleosome core protein that does not include one or more of the hydroxylation modifications.

### Palmitoylated Mini-Nucleosome Core Proteins

Palmitoylation is a lipid modification that typically occurs on cysteine I residues, and can also occur, e.g., on serine (S) and threonine (T) residues. Palmitoylation includes the covalent attachment of a fatty acid, such as palmitic acid, to a residue of a polypeptide such as a mini-nucleosome core protein. Palmitoylation can be present, e.g., on residues of membrane proteins.

In various embodiments, palmitoylation of one more residues of a modified mini-nucleosome core protein increases strength (e.g., affinity or avidity) of association with cell membranes (e.g., endothelial cell membranes) of the modified mini-nucleosome core protein, or loaded mini-nucleosome core proteins including the same, as compared to a reference mini-nucleosome core protein that does not include one or more of the palmitoylation modifications. In various embodiments, palmitoylation of one more residues of a modified mini-nucleosome core protein increases the rate at which the modified mini-nucleosome core protein, or loaded mini-nucleosome core proteins including the same, penetrates cell membranes (e.g., endothelial cell membranes) as compared to a reference mini-nucleosome core protein that does not include one or more of the palmitoylation modifications. In various embodiments, palmitoylation of one more residues of a modified mini-nucleosome core protein increases the rate at which the modified mini-nucleosome core protein, or loaded mini-nucleosome core proteins including the same, enters one or more target cells (i.e., crosses the cell membranes of target cells), such as endothelial cells, as compared to a reference mini-nucleosome core protein that does not include one or more of the palmitoylation modifications.

### Mannosylated Mini-Nucleosome Core Proteins

Mannosylation can refer to modification of an amino acid to include a mannose glycoside. Mannosylation can occur, e.g., on threonine (T) residues of a polypeptide, e.g., a mini-nucleosome core protein. To provide one particular example, mono-O-mannosyl glycans concentrate in inhibitory GABAergic neurons, and the present disclosure includes mono-O-mannosylation of mini-nucleosome core proteins to target mini-nucleosome core proteins to GABAergic neurons.

### Myristoylated Mini-Nucleosome Core Proteins

Myristoylation is a lipid modification that includes covalent linkage of a myristoyl group with an amino acid, e.g., a residue of a polypeptide such as a mini-nucleosome core protein. Myristoylation can occur, e.g., on a glycine (G) residue of a polypeptide such as a mini-nucleosome core protein. For instance, protein N-myristoylation is a lipidic modification that can be present on the alpha-amino group of an N-terminal glycine residue. Myristoylation can contribute to cellular signaling, protein-protein interaction, and targeting of proteins to endomembrane and plasma membrane systems

In various embodiments, myristoylation of one more residues of a modified mini-nucleosome core protein increases affinity or avidity with a target cell or other binding partner of the modified mini-nucleosome core protein, or loaded mini-nucleosome core proteins including the same, as compared to a reference mini-nucleosome core protein that does not include one or more of the myristoylation modifications. In various embodiments, myristoylation of one more residues of a modified mini-nucleosome core protein increases affinity or avidity of binding with a target receptor of the modified mini-nucleosome core protein, or loaded mini-nucleosome core proteins including the same, as compared to a reference mini-nucleosome core protein that does not include one or more of the myristoylation modifications. Thus, in some embodiments, myristoylation of one more residues of a modified mini-nucleosome core protein increases delivery of a mini-nucleosome core protein, loaded mini-nucleosome core protein, or nucleic acid payload of a mini-nucleosome core protein to a target cell, tissue, or organ.

In various embodiments, myristoylation of one more residues of a modified mini-nucleosome core protein increases strength (e.g., affinity or avidity) of association with cell membranes (e.g., endothelial cell membranes) of the modified mini-nucleosome core protein, or loaded mini-nucleosome core proteins including the same, as compared to a reference mini-nucleosome core protein that does not include one or more of the myristoylation modifications. In various embodiments, myristoylation of one more residues of a modified mini-nucleosome core protein increases the rate at which the modified mini-nucleosome core protein, or loaded mini-nucleosome core proteins including the same, penetrates cell membranes (e.g., endothelial cell membranes) as compared to a reference mini-nucleosome core protein that does not include one or more of the myristoylation modifications. In various embodiments, myristoylation of one more residues of a modified mini-nucleosome core protein increases the rate at which the modified mini-nucleosome core protein, or loaded mini-nucleosome core proteins including the same, enters one or more target cells (i.e., crosses the cell membranes of target cells), such as endothelial cells, as compared to a reference mini-nucleosome core protein that does not include one or more of the myristoylation modifications.

### Fucosylated Mini-Nucleosome Core Proteins

Fucosylation is a form of glycosylation that refers to the addition of fucose to an amino acid, e.g., a threonine (T) residue. Fucosylation includes attachment of a fucose residue to N-glycans, 0-glycans, and glycolipids.

### Pegylated Mini-Nucleosome Core Proteins

Pegylation (sometimes written PEGylation) includes the modification of biological molecules by covalent conjugation with polyethylene glycol (PEG), a non-toxic, non-immunogenic polymer. PEG is generally understood to be biocompatible and/or to lack immunogenicity, antigenicity, and/or toxicity. PEG is soluble in water and other organic solvents, is readily cleared from the body, and has high mobility in solution. Pegylation can impact physical and chemical properties of a polypeptide such as a mini-nucleosome core protein, including properties such as conformation, electrostatic binding, and hydrophobicity. Pegylation can cause an improvement in pharmacokinetic behavior of a polypeptide such as a mini-nucleosome core protein. Pegylation can improve drug solubility and decreases immunogenicity of a polypeptide such as a mini-nucleosome core protein. PEGylation can increases drug stability and/or retention time of a polypeptide such as a mini-nucleosome core protein in blood. Pegylation can reduce proteolysis and/or renal excretion polypeptide such as a mini-nucleosome core protein, which can reduce the therapeutically effective dose of a polypeptide such as a mini-nucleosome core protein. As those of skill in the art will appreciate, techniques for use of PEG and pegylation in combination with polypeptide agents are well known in the art. The present disclosure includes, without limitation, modified mini-nucleosome core proteins that include a pegylated amino acid alone or in combination with other modifications of the same or other amino acids of the mini-nucleosome core protein.

Modified Mini-Nucleosome Core Proteins with Multiple and/or Branched Modifications

Those of skill in the art will appreciate that a polypeptide such as a mini-nucleosome core protein can include one or more amino acids that each include one or more modifications provided herein. Accordingly, a mini-nucleosome core protein can include a plurality of modified amino acids each including the same modification, or can include a plurality of modified amino acids of which at least two include different modifications. Moreover, as those of skill in the art will further appreciate, in some embodiments a single modified amino acid can include two or more modifications (see, e.g., FIGS. 20-22). In some embodiments, a single modified amino acid can include two or more modifications, where each of the modifications is covalently linked to the canonical core structure of the amino acid at a different position thereof. In some embodiments, a single modified amino acid can include two or more modifications, where the two or more modifications are present in a modification chain (i.e., a moiety comprising two or more covalently linked modifications, which is itself covalently linked to an atom of the canonical core structure of the amino acid). Accordingly, as disclosed herein, an amino acid can be referred to as modified by a particular modification when the canonical core structure of the amino acid is directly covalently linked to the modification and/or when the canonical core structure of the amino acid is indirectly covalently linked to the modification, e.g., when the modification is present in a modification chain and is not directly linked to an atom of the canonical core structure of the amino acid.

Modification chains of the present disclosure can include a plurality of modifications. In some embodiments, a modification chain includes a plurality of linearly associated modifications, which modifications are directly or indirectly covalently linked in a single linear chain. A modification chain including a single covalently linked linear chain of directly or indirectly linked modifications can be referred to as an “unbranched” modification chain. Modification chains of the present disclosure can be “branched”, e.g., where the modification chain includes a “trunk” that is covalently linked to an atom of the canonical core structure of an amino acid, and which trunk is covalently linked to two or more branches of covalently linked atoms, each of which branches can include one or more modifications. In such branched modification chains, the trunk and two or more branches do not form a single covalently linked linear chain. As provided herein, a trunk or unbranched modification chain can include any number of modifications provided herein, e.g., 0, 1, 2, 3, 4, 5, 6, 7, 8, 9, or 10 modifications provided herein, any of which may be the same modification as any other in the trunk or chain or a different modifications from any other in the trunk or chain. As provided herein, a branch can include any number of modifications provided herein, e.g., 0, 1, 2, 3, 4, 5, 6, 7, 8, 9, or 10 modifications provided herein, any of which may be the same modification as any other in the branch or chain or a different modifications from any other in the branch or chain.

Thus, for the avoidance of doubt, a mini-nucleosome core protein, as set forth herein, can include (a) a nucleic acid binding domain (NABD), and (b) a targeting domain, and in some embodiments can include (a) a nucleic acid binding domain (NABD), (b) a targeting domain, and (c) a nucleic acid release domain, where any of the various mini-nucleosome core proteins provided herein can optionally include one or more modified amino acid residues.

### Nucleic Acid Cargos

Loaded mini-nucleosomes disclosed herein can be loaded with a nucleic acid cargo that is, e.g., RNA, DNA, or a nucleic acid analog thereof. A nucleic acid cargo can be single stranded or double stranded. A nucleic acid cargo can be linear or circular. A nucleic acid cargo can encode one or more of each of a protein, an RNA, an shRNA, an miRNA, an antibody, a nanobody, a Darpin, an Ankyrin repeat, or a polypeptide. For example, a nucleic acid cargo can be a cDNA molecule that encodes at least one functional protein. In various embodiments, a nucleic acid cargo can be an inhibitory RNA, e.g., a gRNA, siRNA, miRNA, or shRNA.

A nucleic acid cargo can encode, e.g., an RNA, protein, polypeptide, antibody, nanobody, miRNA, shRNA, gRNA, Cas9, non-coding RNA when delivered into a nucleus of any cell. Expression may not be limited to entities mentioned herein.

### Loaded Mini-Nucleosomes

A loaded mini-nucleosome of the present disclosure can include one or more mini-nucleosome core proteins of the present disclosure and one or more polynucleotides. Those of skill in the art will appreciate from the present disclosure that such loaded mini-nucleosomes can be generated from combining mini-nucleosome core proteins and polynucleotides in a variety of ways. Those of skill in the art will appreciate that, in at least one embodiment, loaded mini-nucleosome assembly will occur simply upon inclusion of one or more mini-nucleosome core proteins provided herein and one or more polynucleotides in a solution, e.g., without limitation, an aqueous solution, e.g., at a standard temperature and e.g., vortexing at a standard speed. Methods of generating loaded mini-nucleosome core proteins therefore include approaches provided herein and others that will be apparent to those of skill in the art. Those of skill in the art will appreciate that, in at least one embodiment, loaded mini-nucleosome assembly will occur upon inclusion of one or more mini-nucleosome core proteins provided herein and one or more polynucleotides in a solution, e.g., without limitation, an aqueous solution, e.g., at a standard temperature in the presence of catalysts that help enhance condensation of nucleic acids.

A loaded mini-nucleosome of the present disclosure can be at an uncondensed state and a condensed state. A loaded mini-nucleosome is in a condensed state where at least 90%, 91%, 92%, 93%, 94%, 95%, 96%, 97%, 98%, 99%, or 100% of negative charges in the nucleic acid molecule has been neutralized. A loaded mini-nucleosome is considered in an uncondensed state when less than 90% of negative charges in the nucleic acid molecule has been neutralized. Unless specified, references to mini-nucleosomes in the present disclosure encompass at least condensed and uncondensed states and, where applicable, characteristics thereof.

A mini-nucleosome can include, e.g., 1 to 10,000 mini-nucleosome core proteins. A mini-nucleosome can include, e.g., 1 to 100 nucleic acid cargo molecules.

In some embodiments, loaded mini-nucleosome can be of size between 0.5 to 50 nanometers in diameter. Mini-nucleosomes can include nucleic acid cargo molecules that can have a length of up to 50 kb while maintaining a small diameter of between 0.5 and 50 nm.

In some embodiments, loaded mini-nucleosome can have a molecular weight of between 100 and 10000 kDa, e.g., 100, 200, 500, 1000, 3000, 5.000, 8000, 10000 kDa.

In various embodiments, loaded mini-nucleosome can have a net charge of −100 to 100. In some embodiments, the zeta potential of the loaded mini-nucleosome formulation may range from −10 milliVolts to 100 millivolts. In some examples, a complex of nucleic acid cargo and mini-nucleosome core protein is condensed to a minimal size compared to the nucleic acid molecule and polypeptide molecules used to construct the mini-nucleosome particle. The final positive to negative charge ratio is approximately 1:1, thereby forming a non-charged, slightly positively charged or slightly negatively charged molecule. The final particle may form in several shapes including rod, spherical or circular but not limited to these.

In various embodiments, the mini-nucleosome core protein may be modified with one or more molecules of polyethylene glycol of molecular weight of 5 Daltons to 20 kDa. A polyethylene glycol (PEG) moiety maybe attached to any amino acid residue in the polypeptide.

In certain embodiments, a loaded mini-nucleosome includes a ratio of nucleic acid molecules to mini-nucleosome core proteins that is between 1:1 and 1:2.000 or between 1:3 and 1:2,000. In certain embodiments, a loaded mini-nucleosome includes a ratio of nucleic acid molecules to mini-nucleosome core proteins that is between 1:1 and 1:2,000. In certain embodiments, a loaded mini-nucleosome includes a ratio of nucleic acid molecules to mini-nucleosome core proteins that is between 1:1 and 1:1,000, between 1:1 and 1:500, between 1:1 and 1:200, between 1:1 and 1:100, between 1:1 and 1:50. In certain embodiments, a loaded mini-nucleosome includes a ratio of nucleic acid molecules to mini-nucleosome core proteins that is between 1:3 and 1:1,000, between 1:3 and 1:500, between 1:3 and 1:200, between 1:3 and 1:100, or between 1:3 and 1:50. In certain embodiments, a loaded mini-nucleosome includes a ratio of nucleic acid molecules to mini-nucleosome core proteins that is between 1:200 and 1:2,000, between 1:200 and 1:1000, or between 1:200 and 1:500. In certain embodiments, a loaded mini-nucleosome includes a ratio of nucleic acid molecules to mini-nucleosome core proteins that is between 1:1 and 1:50, 1:1 and 1:40, 1:1 and 1:30, 1:1 and 1:20, 1:1 and 1:10, 1:1 and 1:5, 1:1 and 1:4, 1:1 and 1:3, or 1:1 and 1:2.

In various embodiments, a loaded mini-nucleosome includes a ratio of nucleic acid molecules to mini-nucleosome core proteins that is between 1 nucleic acid molecule to 3 mini-nucleosome core proteins (1:3) and 1 nucleic acid molecule to 3,000 mini-nucleosome core proteins (1:3,000), or within any range there between.

In various embodiments, a loaded mini-nucleosome includes a ratio of nucleic acid molecules to mini-nucleosome core proteins that is between 1 nucleic acid molecule to 3 mini-nucleosome core proteins (1:3) and 1 nucleic acid molecule to 2,000 mini-nucleosome core proteins (1:2,000).

In various embodiments, a loaded mini-nucleosome includes a ratio of nucleic acid molecules to mini-nucleosome core proteins that is between 1 nucleic acid molecule to 3 mini-nucleosome core proteins (1:3) and 1 nucleic acid molecule to 1,000 mini-nucleosome core proteins (1:1,000).

In various embodiments, a loaded mini-nucleosome includes a ratio of nucleic acid molecules to mini-nucleosome core proteins that is between 1 nucleic acid molecule to 3 mini-nucleosome core proteins (1:3) and 1 nucleic acid molecule to 500 mini-nucleosome core proteins (1:500).

In various embodiments, a loaded mini-nucleosome includes a ratio of nucleic acid molecules to mini-nucleosome core proteins that is between 1 nucleic acid molecule to 3 mini-nucleosome core proteins (1:3) and 1 nucleic acid molecule to 200 mini-nucleosome core proteins (1:200).

In various embodiments, a loaded mini-nucleosome includes a ratio of nucleic acid molecules to mini-nucleosome core proteins that is between 1 nucleic acid molecule to 3 mini-nucleosome core proteins (1:3) and 1 nucleic acid molecule to 100 mini-nucleosome core proteins (1:100).

In various embodiments, a loaded mini-nucleosome includes a ratio of nucleic acid molecules to mini-nucleosome core proteins that is between 1 nucleic acid molecule to 3 mini-nucleosome core proteins (1:3) and 1 nucleic acid molecule to 50 mini-nucleosome core proteins (1:50).

In various embodiments, a loaded mini-nucleosome includes a ratio of nucleic acid molecules to mini-nucleosome core proteins that is between 1 nucleic acid molecule to 50 mini-nucleosome core proteins (1:50) and 1 nucleic acid molecule to 2,000 mini-nucleosome core proteins (1:2,000).

In various embodiments, a loaded mini-nucleosome includes a ratio of nucleic acid molecules to mini-nucleosome core proteins that is between 1 nucleic acid molecule to 50 mini-nucleosome core proteins (1:50) and 1 nucleic acid molecule to 1,000 mini-nucleosome core proteins (1:1,000).

In various embodiments, a loaded mini-nucleosome includes a ratio of nucleic acid molecules to mini-nucleosome core proteins that is between 1 nucleic acid molecule to 50 mini-nucleosome core proteins (1:50) and 1 nucleic acid molecule to 500 mini-nucleosome core proteins (1:500).

In various embodiments, a loaded mini-nucleosome includes a ratio of nucleic acid molecules to mini-nucleosome core proteins that is between 1 nucleic acid molecule to 50 mini-nucleosome core proteins (1:50) and 1 nucleic acid molecule to 200 mini-nucleosome core proteins (1:200).

In various embodiments, a loaded mini-nucleosome includes a ratio of nucleic acid molecules to mini-nucleosome core proteins that is between 1 nucleic acid molecule to 50 mini-nucleosome core proteins (1:50) and 1 nucleic acid molecule to 100 mini-nucleosome core proteins (1:100).

In various embodiments, a loaded mini-nucleosome includes a ratio of nucleic acid molecules to mini-nucleosome core proteins that is between 1 nucleic acid molecule to 200 mini-nucleosome core proteins (1:200) and 1 nucleic acid molecule to 2,000 mini-nucleosome core proteins (1:2,000).

In various embodiments, a loaded mini-nucleosome includes a ratio of nucleic acid molecules to mini-nucleosome core proteins that is between 1 nucleic acid molecule to 200 mini-nucleosome core proteins (1:200) and 1 nucleic acid molecule to 1,000 mini-nucleosome core proteins (1:1,000).

In various embodiments, a loaded mini-nucleosome includes a ratio of nucleic acid molecules to mini-nucleosome core proteins that is between 1 nucleic acid molecule to 200 mini-nucleosome core proteins (1:200) and 1 nucleic acid molecule to 500 mini-nucleosome core proteins (1:500).

The skilled artisan will appreciate that mini-nucleosome core protein molecules can be produced and/or constituted by various means, including without limitation in several different salt conditions including acetate, trifluoroacetate, bicarbonate, and chloride. Final formulation of the loaded mini-nucleosome may be constituted in normal saline, water or any other pharmaceutically acceptable buffers.

### Delivery of Loaded Mini-Nucleosomes to Target Cells or Tissues

In certain embodiments, a mini-nucleosome can deliver a nucleic acid where the target cell is the retinal pigment epithelium (RPE). For efficient gene therapy, some embodiments include delivery of a large copy number of genetic cargo such as DNA or RNA into one cell type. For example, in wet-age-related macular degeneration, expressing anti-VEGF in the RPE may provide therapeutic levels of proteins necessary for inhibiting endothelial cell proliferation and vascular leakage. We provide herein, examples of mini-nucleosomes core proteins (SEQ ID NO. 392) that allow enhanced uptake into the RPE (FIG. 10, 11).

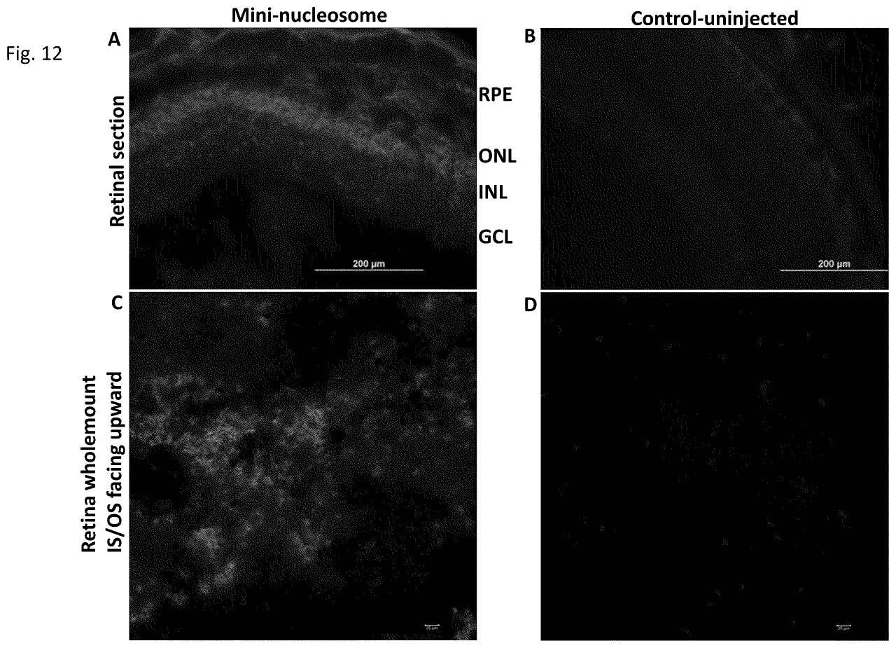

In certain embodiments, a mini-nucleosome can deliver a nucleic acid where the target cell is a neuron in the retina. It has been described that amino acid domain LRE (SEQ ID NO. 156) could be used for enhanced neuronal attachment (Dale D, et al, 1989). We have made use of such domain in a non-viral vector using a GFP construct (SEQ ID NO. 395) with mini-nucleosome core protein (SEQ ID NO. 394) to express GFP to target neuronal cells in the retina (FIG. 12). This maybe particularly useful for delivering DNA or RNA to treat retinal degeneration caused by genetic mutations in genes expressed in retinal neurons.

In various embodiments, a mini-nucleosome can deliver a nucleic acid where the target cell is for e.g. a muscle cell, a liver cell, an endothelial cell, hematopoietic stem cell, lung epithelial, cell, a pericyte, a beta cell, gut epithelial cell, a microglial cell, a macrophage cell, a neuronal cell, skin cell, a blood cell, etc. but not limited to these. Various combination of domains described herein (Table 3-12), may allow delivery of loaded mini-nucleosomes to certain target cell type for therapeutic effects in other parts of the body including brain, retina, gut, liver, lung, kidney, muscle, pancreas but not limited to it.

### Pharmaceutical Compositions

The present disclosure contemplates a “loaded mini-nucleosome therapeutic” that includes a loaded mini-nucleosome and at least one pharmaceutically acceptable carrier. Formulations of pharmaceutically acceptable carrier solutions are well-known to those skilled in the art, as is the development of suitable dosing and treatment regimens. Typically, these formulations can contain 10² genome copies or more of desired transgenes. Other factors such as solubility, bioavailability, half-life, shelf-life will be contemplated by one skilled in the art. As such, various doses and treatment regiments may be desirable. Loaded mini-nucleosome therapeutic could be used to deliver nucleotides to variety of cell types, tissue types or organs in a human body including retina, liver, CNS, gut etc. but not limited to it.

A loaded mini-nucleosome therapeutic can be formulated such that it is pharmaceutically acceptable for administration to cells or animals. Loaded mini-nucleosome therapeutic may be administered in vitro, ex vivo or in vivo. A loaded mini-nucleosome therapeutic can be administered to a subject either alone or in combination with one or more other therapeutic modalities, e.g., antibodies, steroids, vitamins, AAVs etc.

In certain instances, a loaded mini-nucleosome therapeutic can include one or more nucleic acid cargos that each or together encode one or more distinct expression products.

In certain circumstances, it will be desirable to deliver the loaded mini-nucleosome formulations in suitably formulated pharmaceutical compositions disclosed herein either by subcutaneous, intraocular, intravitreal, parenteral, intravenous, intramuscular, intrathecal, topical, oral, intraperitoneal injections, or by nasal inhalation but not limited to these techniques. Solutions of the loaded mini-nucleosome formulations may be prepared in sterile water, sterile saline and may also suitably mixed with one or more surfactants, such as pluronic acid. Dispersions may also be prepared in glycerol, liquid polyethylene glycols, and mixtures thereof. Storage preparations may contain preservatives to prevent microorganisms from growing.

A suitable means of administration of a loaded mini-nucleosome therapeutic agent can be selected based on the condition or disease to be treated and upon the age and condition of a subject. Dose and method of administration can vary depending on the weight, age, condition, and the like of a patient, and can be suitably selected as needed by those skilled in the art.

In various instances, a loaded mini-nucleosome therapeutic agent composition can be formulated to include a pharmaceutically acceptable carrier or excipient. Examples of pharmaceutically acceptable carriers include, without limitation, any and all solvents, dispersion media, coatings, antibacterial and antifungal agents, isotonic and absorption delaying agents, and the like that are physiologically compatible. Compositions of the present invention can include a pharmaceutically acceptable salt, e.g., an acid addition salt or a base addition salt.

In various embodiments, a composition including a loaded mini-nucleosome therapeutic agent as described herein, e.g., a sterile formulation for injection, can be formulated in accordance with conventional pharmaceutical practices using distilled water for injection as a vehicle. For example, physiological saline or an isotonic solution containing glucose and other supplements such as D-sorbitol, D-mannose, D-mannitol, and sodium chloride may be used as an aqueous solution for injection, optionally in combination with a suitable solubilizing agent, for example, alcohol such as ethanol and polyalcohol such as propylene glycol or polyethylene glycol, and a nonionic surfactant such as polysorbate 80™, HCO-50 and the like.

As disclosed herein, a loaded mini-nucleosome therapeutic agent composition may be in any form known in the art. Such forms include, e.g., liquid, semi-solid and solid dosage forms, such as liquid solutions (e.g., injectable and infusible solutions), dispersions or suspensions, tablets, pills, powders, liposomes and suppositories.

Selection or use of any particular form may depend, in part, on the intended mode of administration and therapeutic application. For example, compositions containing a composition intended for systemic or local delivery can be in the form of injectable or infusible solutions. Accordingly, a loaded mini-nucleosome therapeutic agent composition can be formulated for administration by a parenteral mode (e.g., intravenous, subcutaneous, intraperitoneal, or intramuscular injection). As used herein, parenteral administration refers to modes of administration other than enteral and topical administration, usually by injection, and include, without limitation, intravenous, intranasal, intraocular, pulmonary, intramuscular, intraarterial, intrathecal, intracapsular, intraorbital, intracardiac, intradermal, intrapulmonary, intraperitoneal, transtracheal, subcutaneous, subcuticular, intraarticular, subcapsular, subarachnoid, intraspinal, epidural, intracerebral, intracranial, intracarotid and intrasternal injection and infusion.

A parenteral route of administration can be, for example, administration by injection, transnasal administration, transpulmonary administration, or transcutaneous administration. Administration can be systemic or local by intravenous injection, intramuscular injection, intraperitoneal injection, subcutaneous injection.

In various embodiments, a loaded mini-nucleosome therapeutic agent composition of the present invention can be formulated as a solution, microemulsion, dispersion, liposome, or other ordered structure suitable for stable storage at high concentration. Sterile injectable solutions can be prepared by incorporating a composition described herein in the required amount in an appropriate solvent with one or a combination of ingredients enumerated above, as required, followed by filter sterilization. Generally, dispersions are prepared by incorporating a composition described herein into a sterile vehicle that contains a basic dispersion medium and the required other ingredients from those enumerated above. In the case of sterile powders for the preparation of sterile injectable solutions, methods for preparation include vacuum drying and freeze-drying that yield a powder of a composition described herein plus any additional desired ingredient (see below) from a previously sterile-filtered solution thereof. The proper fluidity of a solution can be maintained, for example, by the use of a coating such as lecithin, by the maintenance of the required particle size in the case of dispersion and by the use of surfactants. Prolonged absorption of injectable compositions can be brought about by including in the composition a reagent that delays absorption, for example, monostearate salts, and gelatin.

A loaded mini-nucleosome therapeutic agent composition can be administered parenterally in the form of an injectable formulation comprising a sterile solution or suspension in water or another pharmaceutically acceptable liquid. For example, the loaded mini-nucleosome therapeutic agent composition can be formulated by suitably combining the therapeutic molecule with pharmaceutically acceptable vehicles or media, such as sterile water and physiological saline, vegetable oil, emulsifier, suspension agent, surfactant, stabilizer, flavoring excipient, diluent, vehicle, preservative, binder, followed by mixing in a unit dose form required for generally accepted pharmaceutical practices. The amount of loaded mini-nucleosome therapeutic agent included in the pharmaceutical preparations is such that a suitable dose within the designated range is provided. Nonlimiting examples of oily liquid include sesame oil and soybean oil, and it may be combined with benzyl benzoate or benzyl alcohol as a solubilizing agent. Other items that may be included are a buffer such as a phosphate buffer, or sodium acetate buffer, a soothing agent such as procaine hydrochloride, a stabilizer such as benzyl alcohol or phenol, and an antioxidant. The formulated injection can be packaged in a suitable ampule.

In some embodiments, a loaded mini-nucleosome therapeutic agent composition can be formulated for storage at a temperature below 0° C. (e.g., −20° C. or −80° C.). In some embodiments, the composition can be formulated for storage for up to 2 years (e.g., one month, two months, three months, four months, five months, six months, seven months, eight months, nine months, 10 months, 11 months, 1 year, 1½ years, or 2 years) at 2-8° C. (e.g., 4° C.). Thus, in some embodiments, the compositions described herein are stable in storage for at least 1 year at 2-8° C. (e.g., 4° C.).

In particular instances, a loaded mini-nucleosome therapeutic agent composition can be formulated as a solution. In some embodiments, a composition can be formulated, for example, as a buffered solution at a suitable concentration and suitable for storage at 2-8° C. (e.g., 4° C.).

Compositions including a loaded mini-nucleosome therapeutic agent as described herein can be formulated in immunoliposome compositions. Such formulations can be prepared by methods known in the art. Liposomes with enhanced circulation time are disclosed in, e.g., U.S. Pat. No. 5,013,556.

In certain embodiments, compositions can be formulated with a carrier that will protect the compound against rapid release, such as a controlled release formulation, including implants and microencapsulated delivery systems. Biodegradable, biocompatible polymers can be used, such as ethylene vinyl acetate, polyanhydrides, polyglycolic acid, collagen, polyorthoesters, and polylactic acid. Many methods for the preparation of such formulations are known in the art. See, e.g., J. R. Robinson (1978) “Sustained and Controlled Release Drug Delivery Systems,” Marcel Dekker, Inc., New York.

In some embodiments, compositions can be formulated in a composition suitable for intrapulmonary administration (e.g., for administration via an inhaler or nebulizer) to a mammal such as a human. Methods for formulating such compositions are well known in the art. Dry powder inhaler formulations and suitable systems for administration of the formulations are also known in the art. Pulmonary administration may be oral and/or nasal. Examples of pharmaceutical devices for pulmonary delivery include metered dose inhalers, dry powder inhalers (DPIs), and nebulizers. For example, a composition described herein can be administered to the lungs of a subject by way of a dry powder inhaler. These inhalers are propellant-free devices that deliver dispersible and stable dry powder formulations to the lungs. Dry powder inhalers are well known in the art of medicine and include, without limitation: the TURBOHALER® (AstraZeneca; London, England) the AIR® inhaler (ALKERMES®; Cambridge, Mass.); ROTAHALER® (GlaxoSmithKline; London, England); and ECLIPSE™ (Sanofi-Aventis; Paris, France). See also, e.g., PCT Publication Nos. WO 04/026380, WO 04/024156, and WO 01/78693. DPI devices have been used for pulmonary administration of polypeptides such as insulin and growth hormone. In some embodiments, a composition described herein can be intrapulmonarily administered by way of a metered dose inhaler. These inhalers rely on a propellant to deliver a discrete dose of a compound to the lungs. Additional devices and intrapulmonary administration methods are set forth in, e.g., U.S. Patent Application Publication Nos. 20050271660 and 20090110679, the disclosures of each of which are incorporated herein by reference in their entirety.

In some embodiments, loaded mini-nucleosome therapeutic agent compositions can be formulated for delivery to the eye, e.g., in the form of a pharmaceutically acceptable solution, suspension or ointment. A preparation for use in treating an eye can be in the form of a sterile aqueous solution containing, e.g., additional ingredients such as, but not limited to, preservatives, buffers, tonicity agents, antioxidants and stabilizers, nonionic wetting or clarifying agents, and viscosity-increasing agents. A preparation as described herein can be administered topically to the eye of the subject in need of treatment (e.g., a subject afflicted with AMD) by conventional methods, e.g., in the form of drops, or by bathing the eye in a therapeutic solution, containing one or more compositions.

A variety of devices for introducing drugs into the vitreal cavity of the eye may be appropriate, in certain embodiments, for administration of a composition as described herein. For example, U.S. Publication No. 2002/0026176 describes a pharmaceutical-containing plug that can be inserted through the sclera such that it projects into the vitreous cavity to deliver the pharmaceutical agent into the vitreous cavity. In another example, U.S. Pat. No. 5,443,505 describes an implantable device for introduction into a suprachoroidal space or an avascular region for sustained release of drug into the interior of the eye. U.S. Pat. Nos. 5,773,019 and 6,001,386 each disclose an implantable drug delivery device attachable to the scleral surface of an eye. Additional methods and devices (e.g., a transscleral patch and delivery via contact lenses) for delivery of a loaded mini-nucleosome therapeutic agent to the eye are described in, e.g., Ambati and Adamis (2002) Prog Retin Eye Res 21(2):145-151; Ranta and Urtti (2006) Adv Drug Delivery Rev 58(11):1164-1181; Barocas and Balachandran (2008) Expert Opin Drug Delivery 5(1):1-10(10); Gulsen and Chauhan (2004) Invest Opthalmol Vis Sci 45:2342-2347; Kim et al. (2007) Ophthalmic Res 39:244-254; and PCT publication no. WO 04/073551, the disclosures of which are incorporated herein by reference in their entirety.

In various embodiments, subcutaneous administration can be accomplished by means of a device, such as a syringe, a prefilled syringe, an auto-injector (e.g., disposable or reusable), a pen injector, a patch injector, a wearable injector, an ambulatory syringe infusion pump with subcutaneous infusion sets, or other device for subcutaneous injection.

In some embodiments, a loaded mini-nucleosome therapeutic agent composition described herein can be therapeutically delivered to a subject by way of local administration. As used herein, “local administration” or “local delivery,” can refer to delivery that does not rely upon transport of the loaded mini-nucleosome therapeutic agent composition or loaded mini-nucleosome therapeutic agent to its intended target tissue or site via the vascular system. For example, the loaded mini-nucleosome therapeutic agent composition may be delivered by injection or implantation of the composition or agent or by injection or implantation of a device containing the composition or agent. In certain embodiments, following local administration in the vicinity of a target tissue or site, the composition or agent, or one or more components thereof, may diffuse to an intended target tissue or site that is not the site of administration.

In some embodiments, the compositions provided herein are present in unit dosage form, which unit dosage form can be suitable for self-administration. Such a unit dosage form may be provided within a container, typically, for example, a vial, cartridge, prefilled syringe or disposable pen. A doser such as the doser device described in U.S. Pat. No. 6,302,855, may also be used, for example, with an injection system as described herein.

A suitable dose of a loaded mini-nucleosome therapeutic agent composition described herein, which dose is capable of treating or preventing a disorder in a subject, can depend on a variety of factors including, e.g., the age, sex, and weight of a subject to be treated, the condition or disease to be treated, and the particular loaded mini-nucleosome therapeutic agent used. Other factors affecting the dose administered to the subject include, e.g., the type or severity of the condition or disease. Other factors can include, e.g., other medical disorders concurrently or previously affecting the subject, the general health of the subject, the genetic disposition of the subject, diet, time of administration, rate of excretion, drug combination, and any other additional therapeutics that are administered to the subject. It should also be understood that a specific dosage and treatment regimen for any particular subject can also be adjusted based upon the judgment of a medical practitioner.

A loaded mini-nucleosome therapeutic agent solution can include a therapeutically effective amount of a composition described herein. Such effective amounts can be readily determined by one of ordinary skill in the art based, in part, on the effect of the administered composition, or the combinatorial effect of the composition and one or more additional active agents, if more than one agent is used. A therapeutically effective amount can be an amount at which any toxic or detrimental effects of the composition are outweighed by therapeutically beneficial effects.

Pharmaceutical forms of loaded mini-nucleosome therapeutic formulations suitable for injection can include sterile aqueous solutions or dispersions. A formulation can be sterile and must be fluid to allow proper flow in and out of a syringe. A formulation can also be stable under the conditions of manufacture and storage. A carrier can be a solvent or dispersion medium containing, for example, water and saline or buffered aqueous solutions. Preferably, isotonic agents, for example, sugars or sodium chloride can be used in the formulations. For human administration, final preparations and compositions should meet sterility, pyrogenicity, and the general endotoxin levels, safety and purity standards as required by the US FDA and EU regulatory standards. Temperature and exposure to other proteins can alter the properties of loaded mini-nucleosomes. The final preparations and compositions must be stored at appropriate temperatures, preferably at 2-8 degree Celsius or at room temperature (20-25 degree Celsius).

In addition, one skilled in the art may also contemplate additional delivery method may be via electroporation, sonophoresis, intraosseous injections methods or by using gene gun. Vectors may also be implanted into microchips, nano-chips or nanoparticles.

In certain embodiments, the compositions described herein may be formulated in a kit. Such kits may be used for therapeutic or diagnostic purposes. The present disclosure provides, among other things, one or more compositions together with one or more pharmaceutically-acceptable excipients, carriers, diluents, adjuvants, and/or other components, as may be employed in the formulation of a composition consisting of mini-nucleosome core proteins and nucleic acids, and in the preparation of therapeutic agents for administration to a mammal, and in particularly, to a human, for one or more diseases described herein. In particular, such kits may include one or more of the disclosed mini-nucleosome core protein compositions in combination with instructions for using nucleic acids in the treatment various disorders in a mammal, and may typically include containers prepared for convenient commercial packaging.

Compositions described herein can be administered to an animal that is a mammal, e.g., a human. Compositions described herein are also applicable to animals of commercial interest, livestock, and household pets such as dogs and cats. Compositions in kits can include partially or significantly purified loaded mini-nucleosomes compositions, either alone, or in combination with one or more other ingredients or drugs for therapeutic or diagnostic use. Therapeutic kits can also be prepared that include at least one loaded mini-nucleosome component based gene therapy compositions disclosed herein and instructions for using the composition as a therapeutic agent. The container means for such kits may typically include at least one vial, test tube, flask, bottle, syringe or other container means, into which the disclosed mini-nucleosomes composition(s) may be placed, and preferably suitably aliquoted.

### Applications

Mini-nucleosomes provided herein can, in various embodiments, be characterized by small size, ability to enter cells by receptor mediated or passive diffusion processes, precision in the location of gene expression, precision in the duration of gene expression, and/or retention until release of nucleic acids in the cytoplasm of the nucleus of a target cell. Some of the desired application of the mini-nucleosome technology are described herein.

The present disclosure further includes the recognition that modified mini-nucleosomes of the present disclosure can target delivery of nucleic acid payloads to particular cells or tissues and/or to achieve expression of an expression product encoded by a nucleic acid payload in one or more particular cells or tissues. As will be readily apparent from the present disclosure, delivery and/or expression of a nucleic acid payload in a particular cell or tissue can be useful in a variety of methods and compositions that benefit from the presence of a particular expression product (e.g., a particular protein) in a particular targeted cell or tissue.

In certain embodiments, a modified mini-nucleosome of the present disclosure includes one or more phosphorylated residues (e.g., where one or more, or all, of the phosphorylated residues are selected from serine, threonine, or tyrosine residues). The present disclosure includes the recognition that the presence, rather than the specific number or position, of the phosphorylated residue mediates the ability of the modified mini-nucleosome to target delivery of nucleic acid payloads to particular cells or tissues and/or to achieve expression of an expression product encoded by a nucleic acid payload in one or more particular cells or tissues. Without wishing to be bound by any particular scientific theory, the present disclosure includes that at least certain advantages that characterize phosphorylated mini-nucleosome core proteins (e.g., targeting of particular cell or tissues) result from interaction of the modification with cell surface receptors of target cells and are therefore not sequence specific or specific to the position of any modified residue. The present disclosure specifically includes the recognition that phosphorylated mini-nucleosome core proteins can deliver a nucleic acid payload to, and/or cause expression of a nucleic acid payload in, target cells that are cells of the central nervous system (CNS). In certain embodiments, a phosphorylated mini-nucleosome core proteins can deliver a nucleic acid payload to, and/or cause expression of a nucleic acid payload in, CNS neurons. In certain embodiments, a phosphorylated mini-nucleosome core proteins can deliver a nucleic acid payload to, and/or cause expression of a nucleic acid payload in, CNS astrocytes, microglia, oligodendrocytes, or glia. In certain embodiments, a phosphorylated mini-nucleosome core protein can deliver a nucleic acid payload to, and/or cause expression of a nucleic acid payload in, spinal cord cells, e.g., spinal cord neurons or spinal cord glial cells. In various embodiments, expression of an expression product encoded by a nucleic acid payload associated with a modified mini-nucleosome core protein (e.g., a phosphorylated mini-nucleosome core protein, e.g., in a loaded mini-nucleosome) is increased as compared to expression achieved under reference (e.g., same or similar) conditions using a reference (e.g., unmodified) mini-nucleosome core protein. The present disclosure recognizes that expression in various such target cell types is significant for treatment of certain conditions that effect or are effected by target cell types, e.g., diseases of the CNS.

In certain embodiments, delivery of a nucleic acid payload and/or expression of a nucleic acid payload in target cells is facilitated by a particular route of delivery. In various embodiments in which a phosphorylated mini-nucleosome core protein delivers a nucleic acid payload to, and/or cause expression of a nucleic acid payload in, one or more target cells, cell types, or tissues disclosed herein, administration to a subject is by a route that achieves delivery to cells of the central nervous system. In various embodiments in which a phosphorylated mini-nucleosome core protein delivers a nucleic acid payload to, and/or cause expression of a nucleic acid payload in, one or more target cells, cell types, or tissues disclosed herein, administration to a subject is by a route that achieves delivery to CNS neurons. In various embodiments in which a phosphorylated mini-nucleosome core protein delivers a nucleic acid payload to, and/or cause expression of a nucleic acid payload in, one or more target cells, cell types, or tissues disclosed herein, administration to a subject is by a route that achieves delivery to CNS astrocytes, microglia, oligodendrocytes, or glia. In various embodiments in which a phosphorylated mini-nucleosome core protein delivers a nucleic acid payload to, and/or cause expression of a nucleic acid payload in, one or more target cells, cell types, or tissues disclosed herein, administration to a subject is by a route that achieves delivery to spinal cord cells, e.g., spinal cord neurons or spinal cord glial cells. Those of skill in the art will be familiar with routes of administration to particular cells, cell types, and tissues. In certain particular embodiments, administration to a subject is by injection, e.g., injection to the CNS (e.g., to a cell or tissue of the CNS). In certain particular embodiments, administration to a subject is intrathecal, intracranial, or intra-cisterna magna. In certain particular embodiments, administration to a subject is intrathecal.

In various embodiments, a phosphorylated mini-nucleosome core protein can include multiple phosphorylated residues, e.g., one, two, three, or four phosphorylated residues. In various embodiments, a phosphorylated mini-nucleosome core protein can include one or more amino acid modifications disclosed herein that is not phosphorylation. Accordingly, the present disclosure includes combinations of different modifications present in a single modified mini-nucleosome core protein. A phosphorylated amino acid of a modified mini-nucleosome can be present in any domain or at any amino acid position of a modified mini-nucleosome. A phosphorylated amino acid of a modified mini-nucleosome can be present, without limitation, in a linker domain or targeting domain.

In certain embodiments, a modified mini-nucleosome of the present disclosure includes one or more sulfated residues (e.g., where one or more, or all, of the sulfated residues are selected from serine, threonine, or tyrosine residues). The present disclosure includes the recognition that the presence, rather than the specific number or position, of the sulfated residue mediates the ability of the modified mini-nucleosome to target delivery of nucleic acid payloads to particular cells or tissues and/or to achieve expression of an expression product encoded by a nucleic acid payload in one or more particular cells or tissues. Without wishing to be bound by any particular scientific theory, the present disclosure includes that at least certain advantages that characterize sulfated mini-nucleosome core proteins (e.g., targeting of particular cell or tissues) result from interaction of the modification with cell surface receptors of target cells and are therefore not sequence specific or specific to the position of any modified residue. The present disclosure specifically includes the recognition that sulfated mini-nucleosome core proteins can deliver a nucleic acid payload to, and/or cause expression of a nucleic acid payload in, target cells that are cells of the central nervous system. In certain embodiments, a sulfated mini-nucleosome core protein can deliver a nucleic acid payload to, and/or cause expression of a nucleic acid payload in, CNS neurons. In certain embodiments, a sulfated mini-nucleosome core protein can deliver a nucleic acid payload to, and/or cause expression of a nucleic acid payload in, CNS astrocytes, microglia, oligodendrocytes, or glia. In certain embodiments, a sulfated mini-nucleosome core protein can deliver a nucleic acid payload to, and/or cause expression of a nucleic acid payload in, spinal cord cells, e.g., spinal cord neurons or spinal cord glial cells. In various embodiments, expression of an expression product encoded by a nucleic acid payload associated with a modified mini-nucleosome core protein (e.g., a sulfated mini-nucleosome core protein, e.g., in a loaded mini-nucleosome) is increased as compared to expression achieved under reference (e.g., same or similar) conditions using a reference (e.g., unmodified) mini-nucleosome core protein. The present disclosure recognizes that expression in various such target cell types is significant for treatment of certain conditions that effect or are effected by target cell types, e.g., diseases of the CNS.

In certain embodiments, delivery of a nucleic acid payload and/or expression of a nucleic acid payload in target cells is facilitated by a particular route of delivery. In various embodiments in which a sulfated mini-nucleosome core protein delivers a nucleic acid payload to, and/or cause expression of a nucleic acid payload in, one or more target cells, cell types, or tissues disclosed herein, administration to a subject is by a route that achieves delivery to cells of the central nervous system. In various embodiments in which a sulfated mini-nucleosome core protein delivers a nucleic acid payload to, and/or cause expression of a nucleic acid payload in, one or more target cells, cell types, or tissues disclosed herein, administration to a subject is by a route that achieves delivery to CNS neurons. In various embodiments in which a sulfated mini-nucleosome core protein delivers a nucleic acid payload to, and/or cause expression of a nucleic acid payload in, one or more target cells, cell types, or tissues disclosed herein, administration to a subject is by a route that achieves delivery to CNS astrocytes, microglia, oligodendrocytes, or glia. In various embodiments in which a sulfated mini-nucleosome core protein delivers a nucleic acid payload to, and/or cause expression of a nucleic acid payload in, one or more target cells, cell types, or tissues disclosed herein, administration to a subject is by a route that achieves delivery to spinal cord cells, e.g., spinal cord neurons or spinal cord glial cells. Those of skill in the art will be familiar with routes of administration to particular cells, cell types, and tissues. In certain particular embodiments, administration to a subject is by injection, e.g., injection to the CNS (e.g., to a cell or tissue of the CNS). In certain particular embodiments, administration to a subject is intrathecal, intracranial, or intra-cisterna magna. In certain particular embodiments, administration to a subject is intrathecal.

In various embodiments, a sulfated mini-nucleosome core protein can include multiple sulfated residues, e.g., one, two, three, or four sulfated residues. In various embodiments, a sulfated mini-nucleosome core protein can include one or more amino acid modifications disclosed herein that is not sulfation. Accordingly, the present disclosure includes combinations of different modifications present in a single modified mini-nucleosome core protein. A sulfated amino acid of a modified mini-nucleosome can be present in any domain or at any amino acid position of a modified mini-nucleosome. A sulfated amino acid of a modified mini-nucleosome can be present, without limitation, in a linker domain or targeting domain.

In certain embodiments, a modified mini-nucleosome of the present disclosure includes one or more acetylated residues (e.g., where one or more, or all, of the acetylated residues are lysine residues). The present disclosure includes the recognition that the presence, rather than the specific number or position, of the acetylated residue mediates the ability of the modified mini-nucleosome to target delivery of nucleic acid payloads to particular cells or tissues and/or to achieve expression of an expression product encoded by a nucleic acid payload in one or more particular cells or tissues. Without wishing to be bound by any particular scientific theory, the present disclosure includes that at least certain advantages that characterize acetylated mini-nucleosome core proteins (e.g., targeting of particular cell or tissues) result from interaction of the modification with cell surface receptors of target cells and are therefore not sequence specific or specific to the position of any modified residue. The present disclosure specifically includes the recognition that acetylated mini-nucleosome core proteins can deliver a nucleic acid payload to, and/or cause expression of a nucleic acid payload in, target cells that are CNS neurons and/or retinal cells. In certain embodiments, an acetylated mini-nucleosome core protein can deliver a nucleic acid payload to, and/or cause expression of a nucleic acid payload in, CNS neurons. In certain embodiments, an acetylated mini-nucleosome core protein can deliver a nucleic acid payload to, and/or cause expression of a nucleic acid payload in, retinal cells, e.g., retinal neurons including one or more of photoreceptors, bipolar cells, retinal ganglion cells, horizontal cells and amacrine cells). In certain embodiments, an acetylated mini-nucleosome core protein can deliver a nucleic acid payload to, and/or cause expression of a nucleic acid payload in, photoreceptor cells (e.g., rods and/or cones). In various embodiments, expression of an expression product encoded by a nucleic acid payload associated with a modified mini-nucleosome core protein (e.g., an acetylated mini-nucleosome core protein, e.g., in a loaded mini-nucleosome) is increased as compared to expression achieved under reference (e.g., same or similar) conditions using a reference (e.g., unmodified) mini-nucleosome core protein. The present disclosure recognizes that expression in various such target cell types is significant for treatment of certain conditions that effect or are effected by target cell types, e.g., diseases of the CNS, including diseases of the retina (e.g., Retinitis pigmentosa and/or Stargardt's disease).

In certain embodiments, delivery of a nucleic acid payload and/or expression of a nucleic acid payload in target cells is facilitated by a particular route of delivery. In various embodiments in which an acetylated mini-nucleosome core protein delivers a nucleic acid payload to, and/or cause expression of a nucleic acid payload in, one or more target cells, cell types, or tissues disclosed herein, administration to a subject is by a route that achieves delivery to CNS neurons and/or retinal cells. In various embodiments in which an acetylated mini-nucleosome core protein delivers a nucleic acid payload to, and/or cause expression of a nucleic acid payload in, one or more target cells, cell types, or tissues disclosed herein, administration to a subject is by a route that achieves delivery to CNS neurons. In various embodiments in which an acetylated mini-nucleosome core protein delivers a nucleic acid payload to, and/or cause expression of a nucleic acid payload in, one or more target cells, cell types, or tissues disclosed herein, administration to a subject is by a route that achieves delivery to retinal cells, e.g., retinal neurons including one or more of photoreceptors, bipolar cells, retinal ganglion cells, horizontal cells and amacrine cells). In various embodiments in which an acetylated mini-nucleosome core protein delivers a nucleic acid payload to, and/or cause expression of a nucleic acid payload in, one or more target cells, cell types, or tissues disclosed herein, administration to a subject is by a route that achieves delivery to photoreceptor cells (e.g., rods and/or cones). Those of skill in the art will be familiar with routes of administration to particular cells, cell types, and tissues. In certain particular embodiments, administration to a subject is by injection, e.g., injection to the eye (e.g., to a cell or tissue of the eye). In certain particular embodiments, administration to a subject is intravitreal, suprachoroidal, or subretinal.

In various embodiments, an acetylated mini-nucleosome core protein can include multiple acetylated residues, e.g., one, two, three, or four acetylated residues. In various embodiments, an acetylated mini-nucleosome core protein can include one or more amino acid modifications disclosed herein that is not acetylation. Accordingly, the present disclosure includes combinations of different modifications present in a single modified mini-nucleosome core protein. An acetylated amino acid of a modified mini-nucleosome can be present in any domain or at any amino acid position of a modified mini-nucleosome. An acetylated amino acid of a modified mini-nucleosome can be present, without limitation, in a linker domain or targeting domain.

In certain embodiments, a modified mini-nucleosome of the present disclosure includes one or more mannosylated residues (e.g., where one or more, or all, of the mannosylated residues are serine residues). The present disclosure includes the recognition that the presence, rather than the specific number or position, of the mannosylated residue mediates the ability of the modified mini-nucleosome to target delivery of nucleic acid payloads to particular cells or tissues and/or to achieve expression of an expression product encoded by a nucleic acid payload in one or more particular cells or tissues. Without wishing to be bound by any particular scientific theory, the present disclosure includes that at least certain advantages that characterize mannosylated mini-nucleosome core proteins (e.g., targeting of particular cell or tissues) result from interaction of the modification with cell surface receptors of target cells and are therefore not sequence specific or specific to the position of any modified residue. The present disclosure specifically includes the recognition that mannosylated mini-nucleosome core proteins can deliver a nucleic acid payload to, and/or cause expression of a nucleic acid payload in, target cells that are CNS neurons and/or retinal cells. In certain embodiments, a mannosylated mini-nucleosome core protein can deliver a nucleic acid payload to, and/or cause expression of a nucleic acid payload in, CNS neurons. In certain embodiments, a mannosylated mini-nucleosome core protein can deliver a nucleic acid payload to, and/or cause expression of a nucleic acid payload in, retinal cells, e.g., retinal neurons including one or more of photoreceptors, bipolar cells, retinal ganglion cells, horizontal cells and amacrine cells). In certain embodiments, a mannosylated mini-nucleosome core protein can deliver a nucleic acid payload to, and/or cause expression of a nucleic acid payload in, photoreceptor cells (e.g., rods and/or cones). In various embodiments, expression of an expression product encoded by a nucleic acid payload associated with a modified mini-nucleosome core protein (e.g., a mannosylated mini-nucleosome core protein, e.g., in a loaded mini-nucleosome) is increased as compared to expression achieved under reference (e.g., same or similar) conditions using a reference (e.g., unmodified) mini-nucleosome core protein. The present disclosure recognizes that expression in various such target cell types is significant for treatment of certain conditions that effect or are effected by target cell types, e.g., diseases of the CNS, including diseases of the retina (e.g., Retinitis pigmentosa and/or Stargardt's disease.

In certain embodiments, delivery of a nucleic acid payload and/or expression of a nucleic acid payload in target cells is facilitated by a particular route of delivery. In various embodiments in which a mannosylated mini-nucleosome core protein delivers a nucleic acid payload to, and/or cause expression of a nucleic acid payload in, one or more target cells, cell types, or tissues disclosed herein, administration to a subject is by a route that achieves delivery to CNS neurons and/or retinal cells. In various embodiments in which a mannosylated mini-nucleosome core protein delivers a nucleic acid payload to, and/or cause expression of a nucleic acid payload in, one or more target cells, cell types, or tissues disclosed herein, administration to a subject is by a route that achieves delivery to CNS neurons. In various embodiments in which a mannosylated mini-nucleosome core protein delivers a nucleic acid payload to, and/or cause expression of a nucleic acid payload in, one or more target cells, cell types, or tissues disclosed herein, administration to a subject is by a route that achieves delivery to retinal cells, e.g., retinal neurons including one or more of photoreceptors, bipolar cells, retinal ganglion cells, horizontal cells and amacrine cells). In various embodiments in which a mannosylated mini-nucleosome core protein delivers a nucleic acid payload to, and/or cause expression of a nucleic acid payload in, one or more target cells, cell types, or tissues disclosed herein, administration to a subject is by a route that achieves delivery to photoreceptor cells (e.g., rods and/or cones). Those of skill in the art will be familiar with routes of administration to particular cells, cell types, and tissues. In certain particular embodiments, administration to a subject is by injection, e.g., injection to the eye (e.g., to a cell or tissue of the eye). In certain particular embodiments, administration to a subject is intravitreal, suprachoroidal, or subretinal.

In various embodiments, a mannosylated mini-nucleosome core protein can include multiple mannosylated residues, e.g., one, two, three, or four mannosylated residues. In various embodiments, a mannosylated mini-nucleosome core protein can include one or more amino acid modifications disclosed herein that is not mannosylation. Accordingly, the present disclosure includes combinations of different modifications present in a single modified mini-nucleosome core protein. A mannosylated amino acid of a modified mini-nucleosome can be present in any domain or at any amino acid position of a modified mini-nucleosome. A mannosylated amino acid of a modified mini-nucleosome can be present, without limitation, in a linker domain or targeting domain.

### Gene Therapy

In various embodiments, mini-nucleosomes provided herein can be used in methods of gene therapy. The general principles of gene therapy are well known in the art and include the delivery of a polynucleotide to a subject in need thereof to provide an expression product (e.g., an mRNA, protein, or inhibitory RNA) of therapeutic value. In some embodiments, gene therapy can include gene or protein replacement therapy (e.g., enzyme replacement therapy), augmentation, or target inhibition. In various embodiments, mini-nucleosomes provided herein can be applied to rescue deleterious effects of any mutations that cause diseases including, without limitation, Cystic fibrosis, Duchenne muscular dystrophy, Stargardt's disease, Age-related macular degeneration, Huntington, Hemophilia A, Spinal muscular atrophy, Usher syndrome etc. In such diseases, a genetic mutation renders a gene nonfunctional or not available. In such cases, replacing the mutated gene by a functional copy may be beneficial to the patients. By incorporating a functional cDNA or whole gene into a loaded mini-nucleosome, and delivering it to desired cells or tissues, one may receive, in various embodiments, a therapeutic benefit.

In some embodiments, mini-nucleosomes provided herein can be applied to inhibit genes that are upregulated and disease causing. For example, P53 overexpression has been described in various diseases. In some instances, it is also beneficial to knock down genes at specific cells or tissues to downregulate genes that cause inflammation, hypoxia etc. to have therapeutic effects.

### Ex-Vivo Engineered Cells

Mini-nucleosomes of the present disclosure can be used to engineer cells ex vivo. Cells can be engineered to express therapeutics in various ways. One such cell is immune cell, e.g., T cell. Immune cells can be genetically engineered to express new proteins or receptors that may allow immune recognition of cancerous cells or other harmful cell types for killing and clearance. Such genetic engineering may be performed ex vivo. In various embodiments, mini-nucleosomes provided herein can be used in methods of genetically engineering cells ex vivo. Combination of domains provided herein, may allow loaded mini-nucleosome entry to variety of T cells and deliver a genetic cargo to the nucleus in such cells. The genetic cargo may encode and/or allow expression of chimeric receptors, knockdown of genes or other therapeutic entity. Such cells may then be infused into patients for therapy. One skilled in the art, may contemplate using loaded mini-nucleosomes for creating chimeric antigen receptor T cells (CAR T cells) for use in immunotherapy.

In some embodiments, mini-nucleosomes provided herein can be applied to engineering stem cells ex vivo to express new proteins or receptors for therapeutic purposes. Combination of domains provided herein, may allow loaded mini-nucleosome entry to variety of stem cells to deliver a genetic cargo to the nucleus/cytoplasm in such cells. The genetic cargo may allow expression of chimeric receptors, knockdown of genes or other therapeutic entity. Such cells may then be infused into patients for therapy. One skilled in the art, may contemplate using loaded mini-nucleosomes for creating chimeric stem cells or chimeric hematopoietic stem cells for use in immunotherapy.

### Gene Editing and Base Excision Repair

Gene editing, base editing and manipulation is also an applicable area for this mini-nucleosome technology described herein. Gene editing and base excision repair are state-of-the-art technologies that allow correcting a genetic mutation or editing the genes at the DNA or RNA level. Towards this application, a loaded mini-nucleosome may incorporate nucleic acids that encode for gRNA, sgRNA, spCas9, saCas9, dCas9, cytidine deaminase and several other enzymes that help cleave DNA or convert one base to another. One skilled in the art can appreciate that incorporating multiple gRNAs and Cas9 or similar editing enzymes in an AAV is a cumbersome and often inefficient process. Hence, using the method and compositions described herein, that enables easy compaction of nucleic acids onto loaded mini-nucleosomes allows incorporation of several gRNAs and even the largest of Cas9 genes to deliver to desired cells.

### Antibody Delivery

Antibodies are a class of drugs that have been life changing for millions of patients worldwide. However, one big drawback in this therapy is the requirement of repeat administration which poses immense burden to patients, physicians and caregivers. One skilled in the art can appreciate that a DNA molecule can be used to express antibodies. Mini-nucleosome technology described herein, provides an opportunity to vectorize the antibody and deliver to desired cells in the patients to create a long-term depot in their bodies to reduce the burden of multiple administration. These DNA molecules that express part or whole of antibody domains can be incorporated into loaded mini-nucleosomes to create a long term therapeutic option for patients that take antibody drugs. One skilled in the art may also vectorize and deliver other antibody like molecules such as nanobody, antibody mimetics, fusion peptides, antibody fragments, camelid or camelid single-domain antibody fragments using mini-nucleosome core proteins.

### Vaccine Delivery

Genetically engineered DNA or RNA can produce an antigen to provide a protective immunological response. Nucleic acid vaccines have several potential advantages such as wide-range immunological response over conventional vaccines. Mini-nucleosome technology described herein, can incorporate and deliver such DNA or RNA constructs to desired cells or tissues in animals including humans to protect from several viral, bacterial or parasitic infections.

### Cosmetics

Genetically engineered DNA or RNA can be developed for several cosmetic applications for example to enhance muscle mass, repair skin in burn victims, for weight loss, to improve immune function, to slow aging and many other applications. Mini-nucleosome technology described herein, can incorporate and deliver applicable DNA or RNA constructs to desired cells or tissues in animals including humans for desired cosmetic effect.

In various embodiments, the present disclosure further provides vectors relating to preventing or treating a disease in humans or other animals. A prophylactically or therapeutically effective amount of a composition could be administered via intravenous, intramuscular, intranasal, intraperitoneal, subcutaneous, intracerebral, subretinal, intravitreal, via lumbar puncture, topical, rectal, or direct delivery to local organs or tumors but not limited to these techniques. The composition includes of nucleic acid complexes, each complex consisting essentially of a single or more nucleic acid molecule and one or more mini-nucleosome core protein molecules.

The present disclosure provides, among other things, improved methods of condensing DNA, RNA and their analogs etc. for efficient delivery into human cells to treat certain diseases and/or cosmetic applications. The nucleic acid delivered may also have applications to deliver vaccines.

### EXAMPLES

### Example 1: Design and Synthesis of Mini-Nucleosome Core Proteins

This Example is representative of methods and compositions relating to mini-nucleosome core proteins. In this Example, amino acid sequences of peptides (that can condense nucleic acids into loaded-mini-nucleosomes) and their synthesis process are described.

Loaded mini-nucleosomes of the present Example are produced for efficient gene transfer and release of loaded nucleic acid cargo to various cell types. Loaded mini-nucleosomes of the present Example are designed to actively engage with cell surface via binding to cell surface proteins, to be translocated to the cytoplasm/nucleus in cells, and to allow release of the nucleic acid cargo. These characteristics can be achieved by mini-nucleosome core protein and loaded mini-nucleosomes designed based on structured protein/DNA interaction. Accordingly, the present Example includes mini-nucleosome core proteins that include one or more amino acid domains that enhance one or more of cellular attachment, enhanced uptake, enhanced stability, active transport to the nucleus of a target cell, and release via peptidases.

In the present Example, synthesized mini-nucleosome core proteins may include, without limitation, a sequence according to any one of SEQ ID NOS: 389-394, or other sequences derived from domains disclosed herein in Table 3-12, or any combination thereof. Mini-nucleosome core proteins of the present Example are peptides with net positive charge >8 at pH 7 and isoelectric point >9. For example, SEQ ID NO: 394 is a mini-nucleosome core protein sequence including multiple DNA binding domains (KRHRK) combined with multiple Neuronal attachment domains (LRE) and a poly-Arginine domain (RRRRR). In this same construct, Leucines (L) surround the poly-Arginine domain to separate charged domains with hydrophobic amino acids, enabling the cell attachment targeting domain to bind to the cell surface. In this construct, the mini-nucleosome core protein (SEQ ID NO: 394) is designed for enhanced attachment to neurons via LRE domain while the poly-Arginine domain would help cell entry. The present Example also includes mini-nucleosome core proteins with various linkers positioned between certain domains, and examples of linkers include those provided in SEQ ID NOS: 389-394. By design, KRH in SEQ ID NO: 394 also serves as a cut site for PCSK1 for enhanced release of nucleic acids. Other nucleic acid release domains or cleavage domains that could be included in mini-nucleosome core proteins include, without limitation, those described in Table 9. Domains for inclusion in mini-nucleosome core proteins can also be derived for other peptidases, including without limitation those in Table 9.

Mini-nucleosome core proteins of the present Example, including mini-nucleosome core proteins according to SEQ ID NOS: 389-394, include various combinations of sequence features that allows efficient condensation with nucleic acid molecules and delivery of loaded mini-nucleosomes to desired cell types, e.g., animal cells and tissues. In certain mini-nucleosome core proteins of the present Example, an oligomerization domain is included in a mini-nucleosome core protein in order to cause a loaded mini-nucleosome core protein formed by association of the mini-nucleosome core protein with a nucleic acid cargo to have a relatively smaller size as compared to a reference loaded mini-nucleosome core protein, e.g., as compared to a loaded mini-nucleosome including mini-nucleosome core proteins that lack the oligomerization domain(s) but otherwise are identical in amino acid sequence. Exemplary oligomerization domains include those provided in Table 11. Similarly, endosomal entry and escape signals may also be included in mini-nucleosome core proteins for enhanced stability and release.

Mini-nucleosome core proteins of the present Example can be synthesized by various methods. One method of synthesizing mini-nucleosome core proteins is peptide synthesis. Peptide synthesis allows linking of amino acids via amide bonds. For example, mini-nucleosomes core proteins can be chemically synthesized via a condensation reaction between carboxyl group of one amino acid to the amino group of the next desired amino acid, in order of the sequence of a mini-nucleosome core protein. An established method of peptide syntheses is known in the art as solid phase peptide synthesis.

Several strategies can optionally be applied to protect the amino (N-terminal) and carboxy-terminal (C-terminal) of mini-nucleosome core proteins of the present disclosure. If the mini-nucleosome core protein is lyophilized, the lyophilized peptide may contain traces of salts used during the synthesis process. Other methods of mini-nucleosome core protein production include expressing the mini-nucleosome core protein in a cell system or in vivo form DNA constructs encoding the mini-nucleosome core protein. Produced mini-nucleosome core proteins can be purified by a variety of methods known in the art. For instance, several resins may be utilized during the process. Mini-nucleosomes core proteins, in various instances of the present Example, are >90% pure. However, a less pure <90% core protein may also be used to form a loaded mini-nucleosome. Mini-nucleosomes core proteins, in various instances of the present Example, are >90% conjugated with PEG. However, a less conjugated (<90%) or non-conjugated core protein may also be used to form a loaded mini-nucleosome. Mini-nucleosomes core protein purity can be determined by high-pressure liquid chromatography (HPLC) and identity confirmed by mass spectrometry to the very least.

### Example 2. Production of Loaded Mini-Nucleosomes

This Example describes techniques relating to production of a loaded mini-nucleosome, including without limitation a loaded mini-nucleosome of Example 1. Loaded mini-nucleosomes of the present Example include a nucleic acid cargo (DNA or RNA) condensed with mini-nucleosome core proteins with net positive charges. The mini-nucleosome core protein net positive charge neutralizes negative charges of the nucleic acid cargo, resulting in nanometer sized particles. Conjugation of the said mini-nucleosomes core proteins and DNA or RNA can occur in small or large quantities. There are 2 phosphates meaning 2 negative charges associated with every base. The present Example provides that at least 90% of DNA negative charges are neutralized by a nucleosome core protein positive charge. For example, 90-95 percent of DNA negative charges need to be neutralized for efficient condensation of the nucleic acids with a mini-nucleosome core protein. Various mini-nucleosome core proteins of the present Example can include amino acid domains that enhance one or more of cellular attachment, cellular uptake, protein stability, active transport to the nucleus of a target cell, and release of nucleic acid cargo. Thus, certain mini-nucleosome core proteins provided herein can be particularly useful in certain contexts. During the process of mixing the nucleic acids and mini-nucleosomes core proteins to produce a loaded mini-nucleosome, the mixture of nucleic acids and mini-nucleosome core proteins can be mixed or vortexed between 100 rpms to 4000 rpms. In the process of conjugation of nucleic acids, certain catalysts, such as NaOH and spermidines, that enhance the condensation reaction may be added. These catalysts can be added to the reactor prior to adding the polypeptides and nucleic acids. The nucleic acids may be added in concentrations ranging from 0.1 microgram/microliter to 100 grams/liter. Mini-nucleosomes core proteins may be added at a concentration of 0.1 microgram/microliter to 100 grams/liter. The nucleic acids may be added at once or may be added gradually, e.g., steadily or in sequentially in drops to a vortexing solution. Once the mixing is over, the condensed materials, i.e., loaded mini-nucleosomes may be allowed to be equilibrated for a period of several minutes to several hours, e.g., a period of 2 minutes to a period of 6 hours, prior to purification. Dialysis may be performed to remove impurities and exchange buffers at this stage. Loaded mini-nucleosomes may be purified using several techniques. One such technique is to centrifuge the particles at high speed in a column with molecular weight cutoff parameters of 1 kiloDalton or higher. The centrifugation speed may range from 7000×g to 10,000×g depending on the sample volume. Similarly, duration of centrifugation may vary from 20 minutes at room temperature to one hour depending in sample volume. Another technique available to purify the mini-nucleosomes is dialysis. The purification technique may not be limited to these two techniques and those of skill in the art will be aware of various further purification techniques from literature that can be used to purify protein/nucleic acid complexes. Finally, the loaded mini-nucleosomes may be eluted or collected in endotoxin free water, normal saline or any other buffered solution but not limited to these. The expected recovery of DNA is ~30-70%. Loaded mini-nucleosomes may also undergo further centrifugation in molecular weight cut-off columns to further concentrate the amount of vector genome in the solution. In the present Example, the loaded mini-nucleosome is formulated to minimize the presence of endotoxin. Typical sources of endotoxin are known to include plasmids, peptide synthesis, or from materials used in the prep. Hence, endotoxin free plasmids can be used, and materials and equipment that have been scrubbed of endotoxin can be used, during preparations described in this Example.

A bioreactor can also be used to formulate loaded mini-nucleosomes for consistent mixing of the nucleic acids and peptides to produce particles for commercial and clinical use.

### Example 3: Favorable Shapes/Sizes and Formulations for Loaded Mini-Nucleosomes

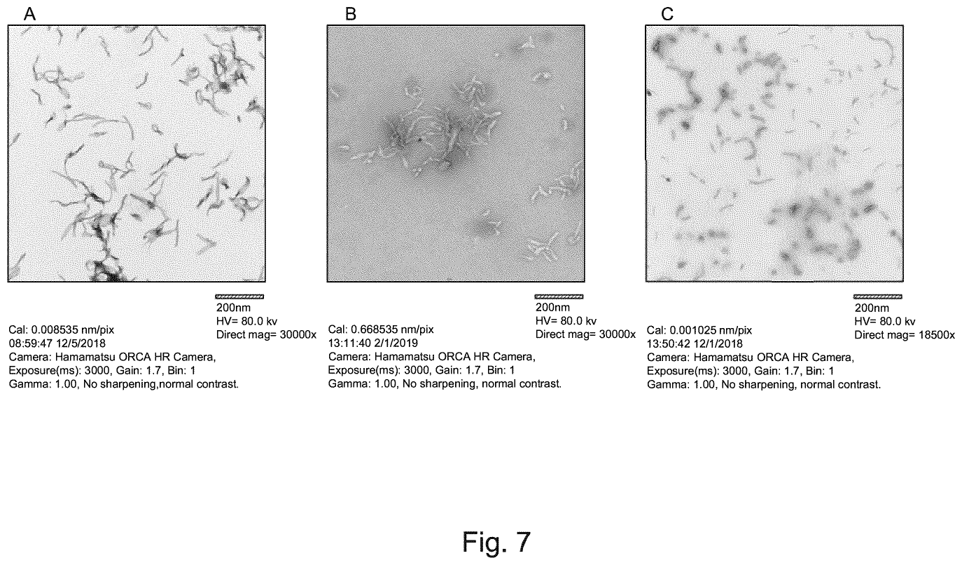

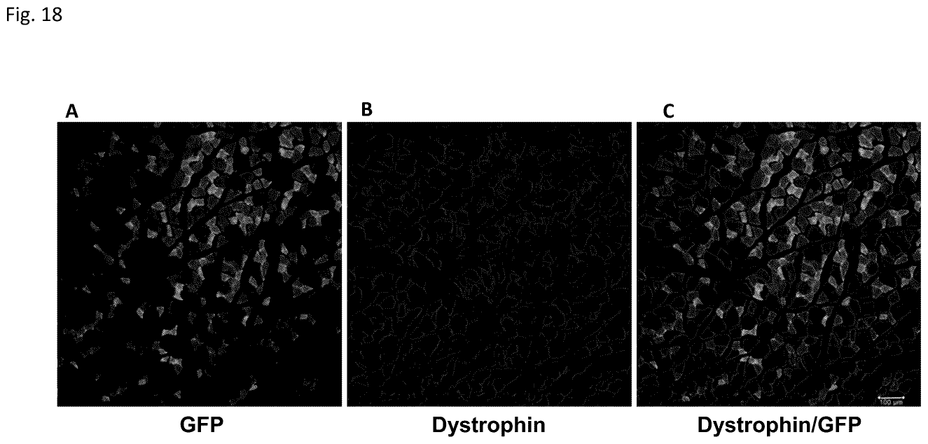

Provided in this Example are techniques to produce loaded mini-nucleosomes in various formulations, including formulations useful for administration to cells and to mammalian subjects, e.g., humans. Loaded mini-nucleosomes can be formulated to different shape and/or sizes parameters based on the mini-nucleosome core protein amino acid sequence and the buffer conditions in which the synthesis occurs. Loaded mini-nucleosomes can be formulated in different conditions, e.g., with solubility suitable for therapeutic use. Solubility of loaded mini-nucleosomes in water and/or normal saline is one means to allow non-toxic formulation of compositions for administration to patients, and to ease of delivery into patients. To form loaded mini-nucleosomes represented in FIG. 7, core proteins were synthesized by solid phase synthesis using trifluoroacetate buffers. 200 micrograms of DNA (SEQ ID NO: 396) were added to 1 milligram of lyophilized core proteins and vortexed together, and purified to produce loaded mini-nucleosomes (FIG. 7). Buffer exchange was performed and final formulation of mini-nucleosome was made in sterile, endotoxin free water. 1 microgram of each kind of mini-nucleosomes was diluted in water and then placed on grids that were stained with freshly prepared in 0.75% uranyl acetate in methanol solution for two minutes. Grids were dipped in 100% ethanol and then blotted into lens absorbent paper. The grids were then air-dried for few minutes with film side up and taken for imaging with Hammatsu ORCA HR camera (FIG. 7). The polynucleotide utilized in generating loaded mini-nucleosome core proteins of the present disclosure as a plasmid encoding luciferase, but those of skill in the art will appreciate that the present Example is broadly demonstrative of the general capacity of mini-nucleosome core proteins of the present disclosure to associate with polynucleotides and form loaded mini-nucleosomes. Luciferase plasmid is representative of nucleic acid in general, including, without limitation, plasmids, linear nucleic acids, RNA and DNA of all kinds. In other cases, e.g., RNA or DNA of other sequences or structures could be used in producing loaded mini-nucleosomes. Luciferase plasmid condensed with core protein of SEQ ID NO: 393, led to spiral/helical-shaped loaded mini-nucleosome (FIG. 7A). Luciferase plasmid condensed with core protein with SEQ ID NO: 390, led to rod/lobular shaped loaded mini-nucleosomes (FIG. 7B). A mixture of circular and rod like molecules were observed for loaded mini-nucleosome produced by condensation of luciferase plasmid with core protein SEQ ID NO: 391 (FIG. 7C). There are other buffer conditions and amino acid sequence with varying charge and iso-electric point that could produce spherical or circular loaded mini-nucleosomes. Molecules of different shapes and sizes can enhance tropism to certain cell types. Differently shaped viruses transduce different cell types more effectively. For example, the tobacco mosaic virus is a rod/helical shaped nucleocapsid structure that transduces tobacco plant cells, HIV is round or ball-shaped that infects white blood cells, and AAV2 is an icosahedral shape that transduces liver cells effectively. We observed better transduction tropism of spiral shaped mini-nucleosomes compared to rod shaped ones in muscle cells (FIG. 18). We have been able to formulate differently shaped loaded mini-nucleosomes as described herein. Distinct mini-nucleosomes can also be purified based on unique shapes and sizes.

### Example 4: Route of Administration—Intravenous (Systemic) and Application in Systemic Diseases Such as Hemophilia A

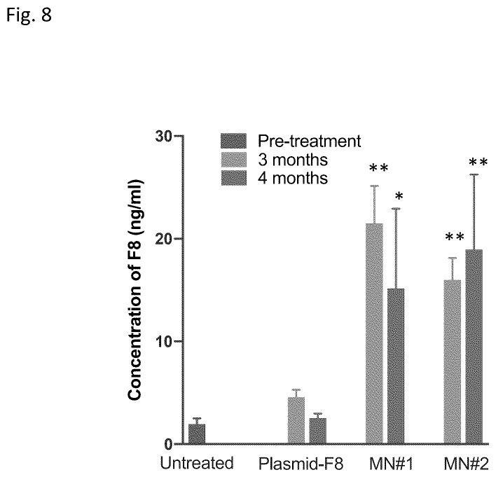

This Example demonstrates that loaded mini-nucleosomes can be delivered by intravenous routes to express proteins in the liver and other organs. Balb/c mice were restrained using standard techniques and insulin syringes were used to deliver loaded mini-nucleosomes and plasmid controls via tail vein injections. F8 expressing plasmid constructs (“F8 plasmid”; see, e.g., MN #1 and MN #2, FIG. 8) were prepared by condensation of SEQ ID NO: 390 and SEQ ID NO: 391, respectively, with F8 plasmid DNA (SEQ ID NO: 460). Plasmid sequence for GFP expressing construct is provided in SEQ ID NO: 8. In the present Example, to target loaded mini-nucleosomes to liver cells, we incorporated 2 NGR amino acid domains alongside nucleic acid binding domains (SEQ ID NO: 3). NGR domains in AAV2 have been shown to promote αVβ5 integrin binding. NGR domains are implicated in heparan sulfate binding, known as receptor for AAV2. AAV2 is known for high liver tropism. KRH amino acid motif also incorporated in these core proteins serve as a cut site for PCSK1 for enhanced release of nucleic acids. Inclusion of multiple KRH amino sequences should enhance release of loaded mini-nucleosomes. Each mouse received 40 micrograms dose of either MN #1, MN #2 or naked plasmid F8 (SEQ ID NO: 460). To test for expression of F8 protein, ~150 μl blood was collected by cheek bleed technique before (1 day prior) and after treatments (post treatment—3 days, 1 week, 2 weeks, 1 month, 3 months and 4 months). Serum was prepared from blood using standard techniques. F8 Elisa (Aviva Systems Biology) was performed according to manufacturer's instructions using 1:6 serum dilutions. Loaded mini-nucleosomes #1 (MN #1 includes SEQ ID NO: 390+F8 plasmid) and MN #2 (MN #2 includes SEQ ID NO: 391+F8 plasmid) expressed approximately six folds more F8 compared the level of F8 detected by ELISA in pre-treatment samples. MN #1 sustained significantly elevated levels of expression at 3 months and 4 months after a single injection of loaded mini-nucleosome (FIG. 8). Control mice treated with naked plasmid encoding F8 (not complexed with mini-nucleosome core proteins) did not demonstrate significant increase in F8 expression at either time points (FIG. 8).

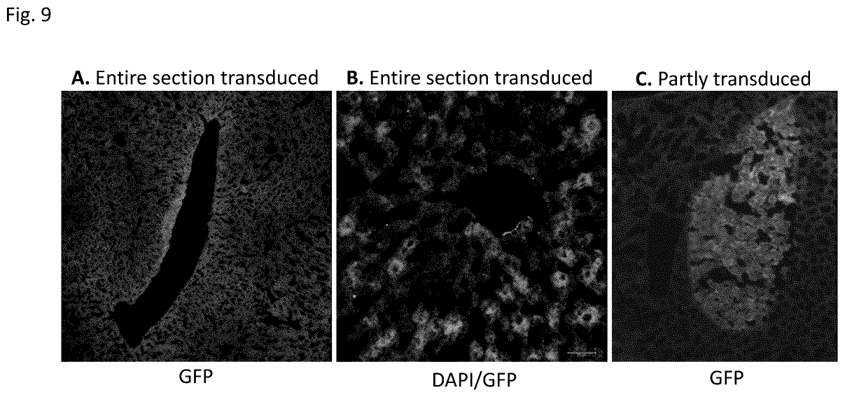

In another experiment, direct GFP fluorescence was observed in tissues collected from mice that underwent intravenous injection of loaded-mini-nucleosomes carrying GFP expressing plasmid (SEQ ID NO: 390+GFP plasmid, SEQ ID NO: 395) (FIG. 9). Briefly, mice were perfused with 1×PBS and sacrificed. Entire liver was collected following dissection. The liver tissues were fixed in 4% paraformaldehyde overnight then washed in 1×PBS, immersed in 15% sucrose for few hours and then in 30% sucrose solution overnight for cryopreservation. The tissues were then placed in a plastic vial and frozen using OCT compound for sectioning. 10-micron thick tissue sections were obtained using a cryotome. The liver sections were mounted with mounting media with or without DAPI, coverslip and sealed. Images were acquired by Leica SP5 confocal and epi-fluorescent scopes.

Results demonstrated that when delivered by intravenous route, mini-nucleosomes successfully reached liver and mini-nucleosome cargo-encoded genes were expressed in liver cells (FIG. 9). Expression in multiple liver cell types was observed. The observations of the present Example suggest that delivery of loaded mini-nucleosomes to liver is not dependent upon targeting domains. One of skill in the art, in view of the data provided in the present Examples, would understand that loaded mini-nucleosomes can be delivered to cells in kidney and spleen via intravenous delivery, since, like the liver, these organs normally function in clearance of, e.g., drugs.

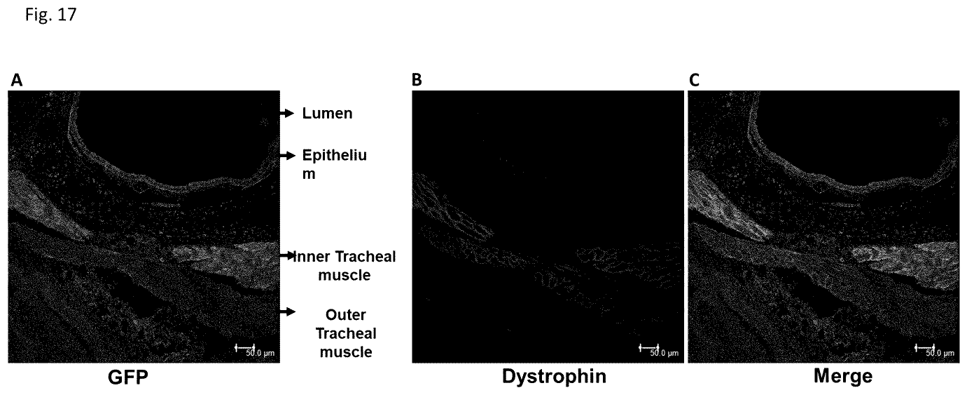

One example of a condition that, in view of the present disclosure, can be treated by use of a loaded mini-nucleosome therapeutic agent is Hemophilia A. Hemophilia A is a severe bleeding disorder caused by mutation in factor 8, a clotting factor. It is inherited in an X-lined recessive manner. It occurs in approximately 1 in 5,000 live births. Most serious implications are internal bleeding that may lead to death. Severity depends on amount of F8 circulating in the body. 75% of the hemophilia patients take a recombinant F8 product as therapy. Subjects receiving F8 therapy are repeatedly infused intravenously, leading to huge burden for patients, physicians, and caregivers over time. Currently, gene therapy trials are underway to deliver long term expression of F8 via AAVs. However, F8 is a large gene that cannot be fully incorporated in AAV. Thus, mini-F8 has been utilized to deliver functional domains of F8 to treat this disease. It is well known that mini-F8 doesn't have the same functional capability and stability as of full-length F8. Moreover, 20-40% of population already has neutralizing antibodies against AAV that will render a large population of Hemophilic patients unable to receive the AAV-based medicine. In addition, if a further treatment were to be needed after a first discontinued course of AAV treatment, AAV vectors cannot be redosed due to immunogenicity. By being able to deliver full size of F8 gene (FIG. 8) and because of its redosable nature (FIG. 17), loaded mini-nucleosomes solve these two problems of AAV gene therapy. Thus, the present disclosure provides techniques to deliver loaded mini-nucleosomes into different cell types in the systemic space such as liver, kidney, spleen etc. using intravenous mode of delivery, for use in many conditions of which Hemophilia A is exemplary.

Other systemic diseases that often stem from defects in secreted proteins could also be treated using loaded mini-nucleosomes therapeutic agents. The present Example (FIG. 8) demonstrated that loaded-mini-nucleosomes, delivered intravenously (systemic administration), produce proteins at levels higher than the therapeutic threshold which is approximately 10% of endogenous levels determined by various clinical trials demonstrating, among other things, therapeutic potential of mini-nucleosomes as therapeutic agents for treatment of, e.g., systemic diseases where a secreted protein can be expressed by variety of cell types. In some cases, expression can be restricted to certain cell types by using a cell-type specific promoter. One skilled in the art would also understand from the present disclosure that other tissues such as brain, heart, muscles etc. may also be accessed and transduced via intravenous delivery. The targeting mechanism built into the mini-nucleosome core proteins shall aid in that context.

When injected intravenously, loaded mini-nucleosomes may be delivered at a dose greater than 1e5 genome copies per kg and up to a dose of 1e25 copies per kg of body weight (e.g., at about 1e5, 1e6, 1e7, 1e8, 1e9, 1e10, 1e15, 1e20, or 1e25 copies per kg body weight, or any range there between). Volume of the material may range from 1-900 milliliters (e.g., 1, 5, 10, 20, 30, 40, 50, 100, 200, 300, 400, 500, 600, 700, 800, or 900 milliliters). The loaded mini-nucleosomes may also be administered repeatedly (e.g., a selected volume and/or number of genome copies can be administered multiple times or divided among two or more does).

### Example 5: Route of Administration—Intraocular

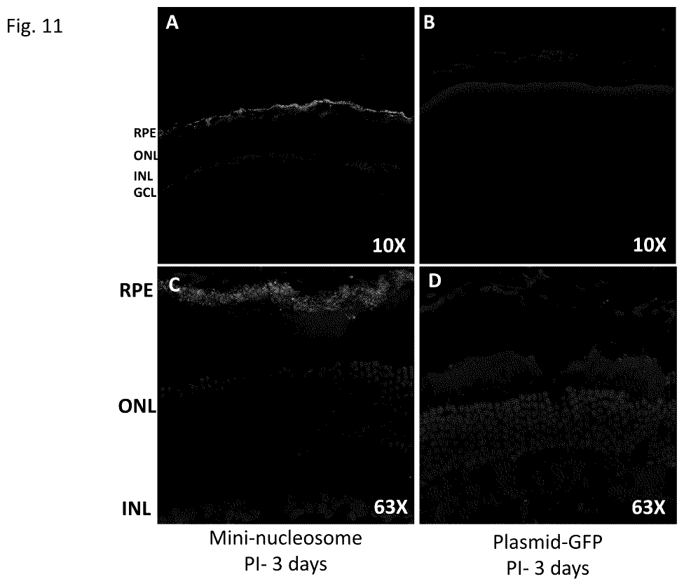

This example demonstrates that loaded mini-nucleosomes can be delivered by intra-ocular route to express proteins in the retinal pigment epithelium (RPE) or in other retinal neurons such as photoreceptors, bipolar cells and ganglion cells. In the present Example, Balb/c mice were anesthetized by IP injection with Ketamine/Xylazine (90-100 mg/kg+10 mg/kg) and positioned underneath a microscope. Mice eyes were dilated with topical Tropicamide (1%) and 1 ul of loaded mini-nucleosomes (total dose 1.5 micrograms in mice) were injected into the vitreous cavity using 32 gauge blunt needle passing through the incision made by a 25-gauge needle below the limbus. At various time points, mice were perfused with 10 ml of 1×PBS, and then sacrificed using standard techniques. Mice were enucleated and eyecups were collected and incubated in 4% paraformaldehyde overnight. The eyecups were washed with 1×PBS, then immersed in 15% sucrose for few hours and then in 30% sucrose solution overnight for cryopreservation. The eyecups were then placed in a plastic vial and frozen using OCT compound for cryo-sectioning. 10-micron thick tissue sections were obtained for staining. The retinal sections were mounted with mounting media with or without DAPI, coverslip and sealed. Images were acquired by Leica SP5. For wholemount imaging, eyecups were fixed in 4% paraformaldehyde overnight. Eyecups were washed in 1×PBS, retina was removed and the remaining eyecup or RPE wholemount was processed for staining. The RPE tissue was wholemounted with mounting media, coverslip and sealed. Images were acquired by Leica SP5. Native GFP fluorescence were observed in retina and RPE cells (FIGS. 10, 11 & 12).

To target the RPE cells, the present Example utilized a mini-nucleosome core protein (SEQ ID NO: 392) that could bind to the phagocytic proteins like MERTK. RPE are phagocytic cells, that extend their microvilli to the photoreceptor inner/outer segment junction. MERTK is expressed in those microvilli. In SEQ ID NO: 392, we incorporated the “eat me” signals as descried in Table 8. In literature, “eat me” signals are described as domains exposed in cellular debris that are primed for phagocytosis (Wei Li, Journal of Cell physiology, 2016, which is incorporated herein by reference). To the present inventor's knowledge, these “eat me” signals have never been utilized in the context of non-viral vectors before. These “eat me” signal domains have not been previously applied for non-viral vectors to target the RPE cells.

To selectively transduce photoreceptors, the present Example utilized core proteins like those of SEQ ID NO:394. SEQ ID NO:394 included a neuronal attachment element (LRE) described herein Table 8, that could allow transduction into ganglion cells, bipolar cells and photoreceptors which are all neurons in the retina (FIG. 12). This neuronal attachment domain has not been previously applied for non-viral vectors to target neurons. The present disclosure provides that this neuronal targeted vector can transduce neurons in the brain via local or systemic administration. The present disclosure further provides for targeting photoreceptor binding and internalization by incorporating lectin binding domains (described in Table 4) in mini-nucleosomes for attachment to photoreceptor extracellular matrix to enhance uptake. An integrin binding domain incorporated in the mini-nucleosome core protein (SEQ ID NO: 390) also could transduce RPE cells in rat eyes exclusively when delivered intraocular (FIG. 11). Moreover, more than one domain could be utilized to selectively transduce a plurality of diverse cell types. This core protein (SEQ ID NO: 390) with integrin binding properties may also be utilized for delivery of nucleic acids to other cell types that express high levels of αVβ5 integrin. The present disclosure further provides use of other intra-ocular injection techniques such as subretinal, suprachoroidal, intra-cameral, or topical administration to target photoreceptors, RPE, Mueller cells or other cell types in the retina.

Provided herein are techniques to deliver loaded mini-nucleosomes into different cell types in the retina using intravitreal, suprachoroidal, or subretinal mode of delivery. Diseases like retinal degeneration are mostly caused by mutations in genes expressed in the photoreceptors. Age-related macular degeneration (AMD), is a disease of retinal pigment epithelium (RPE) and choriocapillaries, that affects >10 million Americans and >100 million people worldwide, Currently, the predominant technology to deliver gene therapy vectors to photoreceptors and RPE is a surgical technique where viruses are injected subretinally into the retina. However, subretinal procedure is a complex surgery performed in the operating room by a trained Ophthalmic surgeon. There is an unmet need at least in that, in the United states, there are only a handful of surgeons trained to perform this surgery. One way to reduce the burden for patients and physicians is to develop vectors that can be injected intravitreally that can pass through the retina to transduce the photoreceptors and RPE. Intravitreal injection can be performed by all ophthalmologist in an in-patient visit. Loaded mini-nucleosome therapy solves this problem as intravitreal injections could transduce photoreceptors and RPE selectively (FIGS. 10, 11 and 12). This makes mini-nucleosomes highly suitable for treating most retinal diseases with genetic defects.

When injected intraocular, the loaded mini-nucleosomes may be delivered at a dose greater than 1e5 genome copies per eye and up to a dose of 1e25 copies per eye (e.g., at about 1e5, 1e6, 1e7, 1e8, 1e9, 1e10, 1e15, 1e20, or 1e25 copies per, or any range there between). Volume of the material may range from 10-500 microliters when injected subretinally (e.g., 1, 5, 10, 20, 30, 40, 50, 100, 200, 300, 400, or 500 microliters) and 10-250 microliters when injection is intravitreal, suprachoroidal, or intracameral (e.g., 1, 5, 10, 20, 30, 40, 50, 100, 150, 200, or 250 microliters). A loaded mini-nucleosome therapeutic agent may also be administered repeatedly (e.g., a selected volume and/or number of genome copies can be administered multiple times or divided among two or more does).

### Example 6: Route of Administration—Intranasal

This example demonstrates that loaded mini-nucleosomes can be delivered by intra-nasal route to express proteins in lung, trachea, and gut cells. In the present Example, to target epithelial cells in the lung epithelium, 2 NGR amino acid domains were included in a mini-nucleosome core protein alongside nucleic acid binding domains (see use of NGR amino acid domains SEQ ID NO: 390). To the present inventor's knowledge, NGR domains have never been utilized to create and deliver non-viral DNA/protein complexes to retinal cells as disclosed herein. NGR domains in AAV2 have been shown to promote αVβ5 integrin binding. NGR domains are implicated in heparan sulfate binding, known as receptor for AAV2.

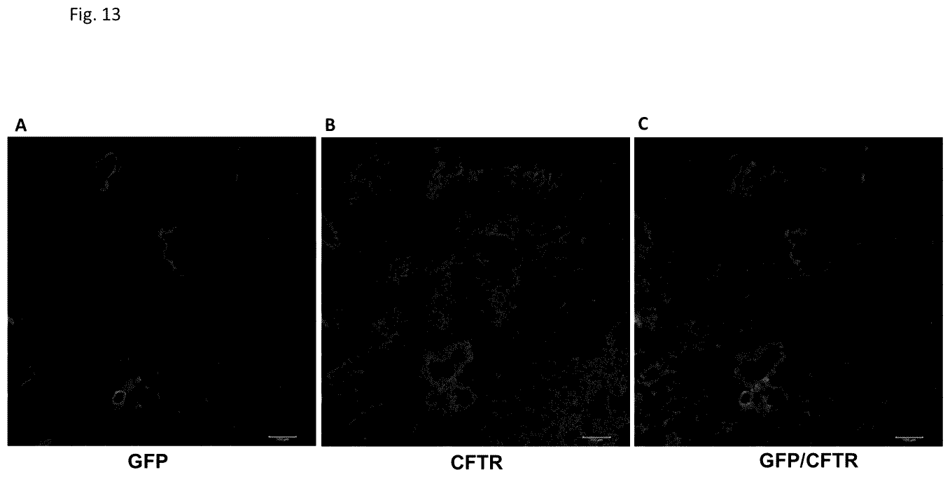

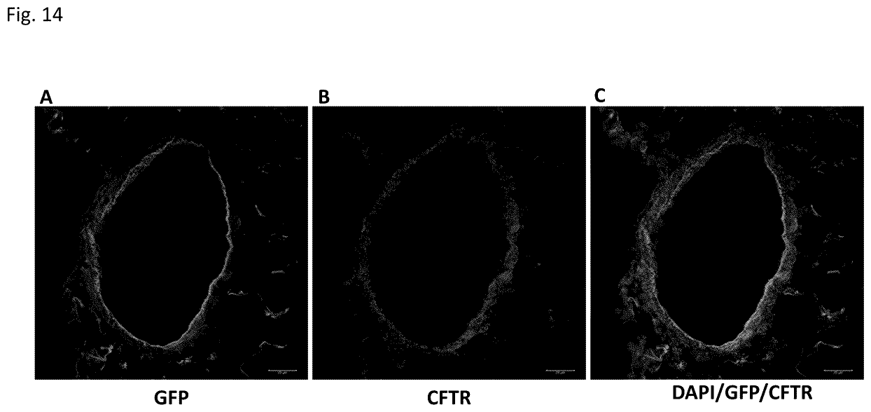

In the present Example, Balb/c mice were anesthetized by IP injection with Ketamine/Xylazine (90-100 mg/kg+10 mg/kg) and the anesthetized mice were positioned underneath a microscope for visual of the nasal area for intranasal delivery. 1 ul of loaded mini-nucleosome (SEQ ID NO: 390+GFP plasmid) solution was delivered into the nasal cavity every few seconds until 12 microliters were delivered to each nasal side. Total dose of 25 micrograms was delivered. Following sacrifice, mice lung was processed to obtain 10 micron thick sections. Sections were washed in PBS and incubated in blocking buffer (0.1% TritonX-100, 1% BSA, 3% donkey serum) for 1 hr and then incubated in CFTR antibody (prepared on blocking buffer) blocking buffer overnight at 4 degree Celsius. Next day wash in PBS 3×5 min and incubated in AlexaFlour-555 (Donkey Anti-rabbit IgG secondary) in blocking buffer at RT for 1 hour and washed in PBS 3×5 min. Mounting media was added and coverslip was applied and sealed. Native fluorescence of GFP was obtained in the 486 nm channel of Leica SP5 scope in the 486-nm wavelength and CFTR expression in the 555-nm channel. We observed loaded mini-nucleosomes expression as early as 3 days and at PI-3 months as well (FIG. 13). We observed expression in the epithelium of both alveoli and bronchioles (FIGS. 13A and 13C) depicted by sharp green fluorescence along with CFTR staining. Co-localization of CFTR and GFP (FIG. 13C) demonstrates expression of genes encoded by mini-nucleosomes in lung epithelium. Higher magnification images taken from an alveoli ring (FIGS. 14 A, B and C) also clearly exhibit bright green ring of GFP fluorescence in the epithelium together with red fluoresce in CFTR stained cells.

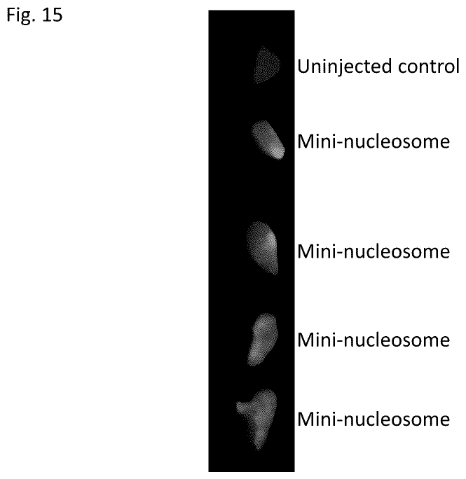

In the present Example, whole lung tissue and biodistribution via mini-nucleosome was also evaluated (FIG. 15). Whole lung tissue was extracted form mice following perfusion and sacrifice. Lung tissue was fixed in 4% PFA and washed with 1×PBS. Whole tissues were placed in the Odyssey imager for detecting GFP native fluorescence. Uninjected control did not exhibit any fluorescence (FIG. 15). Loaded mini-nucleosomes including plasmid nucleic acid cargo encoding GFP demonstrated GFP fluorescence in whole lung tissue in 5-week post injection samples (FIG. 15).

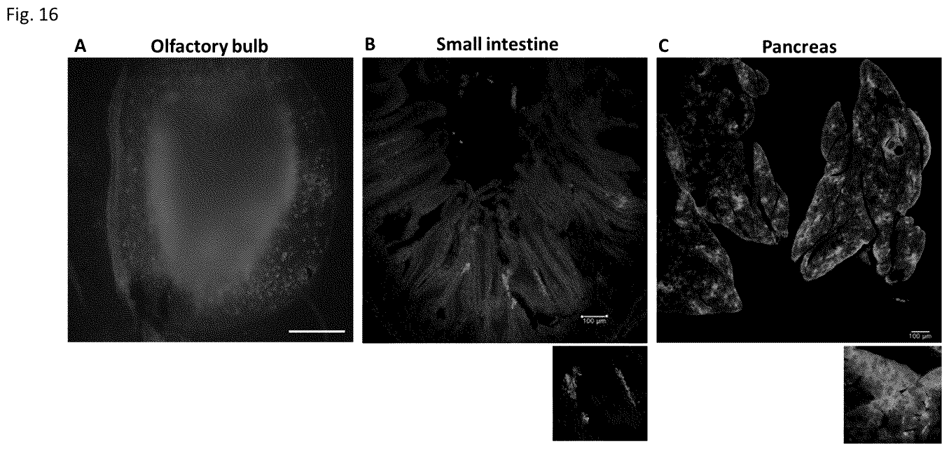

Provided herein are techniques to deliver loaded mini-nucleosomes into different cell types in tissues of the pulmonary space such as lung epithelium, and/or trachea using intranasal mode of delivery. Genetic diseases such as cystic fibrosis affect the lung and other organs. To deliver genes to the lung, the intranasal is one of the routes of choices. We observed that loaded-mini-nucleosomes when delivered intranasally, expresses proteins in the alveoli and bronchioles (FIG. 13). These are tissues that would normally express the CFTR protein implicated in cystic fibrosis. In other diseases, this route of administration can be used to produce therapeutic proteins that could alleviate other diseases. Intranasal route may also provide access to other organs such as the gut and brain (FIG. 16). Inclusion of NGR domains in the mini-nucleosome core proteins (SEQ ID NO: 390), allowed enhanced uptake and release of DNA molecules into the nucleus for high levels of sustained expression. This is evidenced in FIG. 16 by the bright green fluorescence observed from loaded-mini-nucleosomes vs no such pattern in the untreated animals (lung image in the first row in FIG. 16) at 5-weeks post treatment. We also observed transduction of expression of GFP in tracheal epithelium and tracheal muscle following intranasal delivery of loaded mini-nucleosomes (FIG. 17).

When injected intranasally, the loaded mini-nucleosomes may be delivered at a dose greater than 1e5 genome copies per kg and up to a dose of 1e25 copies per kg of body weight (e.g., at about 1e5, 1e6, 1e7, 1e8, 1e9, 1e10, 1e15, 1e20, or 1e25 copies per kg of body weight, or any range there between). Volume of the material may range from 1-200 milliliters (e.g., 1, 5, 10, 20, 30, 40, 50, 100, or 200 milliliters). The loaded mini-nucleosomes may also be administered repeatedly. The loaded mini-nucleosomes may also be delivered orally to access gut, pancreas etc.

### Example 7: Route of Administration—Intramuscular

This example demonstrates that loaded mini-nucleosomes can be delivered by intra-muscular route to express proteins in the muscle cells. Balb/c mice were anesthetized by IP injection with Ketamine/Xylazine (90-100 mg/kg+10 mg/kg) and several loaded mini-nucleosomes were injected into both leg muscle at 17.5 ug doses per leg using an insulin syringe (Total dose 35 micrograms per mice). Mice were sacrificed at various time points and leg muscle were obtained for tissue sections. Constructs that contained core proteins such as polylysine (SEQ ID NO: 393) or mini-nucleosome with other domain combinations (SEQ ID NO: 389) didn't exhibit GFP fluorescence at the 3-month time point. Surprisingly, in muscle tissue sections obtained from 3-months post injections, we observed sharp green fluorescence in skeletal muscle cells injected with loaded mini-nucleosomes with containing galactose and fucose binding domain as shown in SEQ ID NO: 391 (FIGS. 18 A, B and C). This demonstrates that some domains have a higher propensity of attachment and internalization into muscle cells and could be utilized for efficient gene transfer to muscle cells. One skilled in the art may contemplate combining such domains with other domains known for muscle tropism.

To validate muscle specificity of expression of genes encoded by the nucleic acid cargo, we utilized dystrophin immunolabeling as an endogenous secondary marker. Regions of sharp green fluorescence (panel A) encircled by red fluorescence (panel B; merged in panel C) of Dystrophin staining clearly demonstrates that loaded mini-nucleosomes injected intramuscularly can deliver genes to muscle cells (FIG. 18). Native fluorescence of GFP was obtained in the 486-nm channel of Leica SP5 scope. Dystrophin in red is the RFP channel (555-nm). Untransduced muscle cells in figure also serve as internal control for differentiation between GFP signal and autofluorescence.

Provided herein are techniques to deliver loaded mini-nucleosomes into muscle cells by intramuscular mode of delivery. Many genetic muscular dystrophies lead to atrophy of the muscle cells. To deliver functional genes to these muscle cells, intramuscular route provides direct routes of administration. We demonstrated the muscle tropism and ability of loaded-mini-nucleosomes to express genes in the skeletal muscle cells (FIG. 18). Expression was observed in muscle cells as early as day 2 after delivery. Provided herein are muscle-tropic domains that could enhance vector uptake and gene expression, however is not limited to it. We also observed that spiral shaped loaded mini-nucleosomes delivered via intramuscular route, transduce muscle cells effectively and for longer durations—in this case 3 months (FIG. 18) compared to lobular shaped molecule (data not shown). The shape of vectors has not been described before in the context of delivering genes to the muscle cells. One skilled in the art may contemplate, utilizing other structures for increased cell tropism for muscle cells. Overall, the expression of GFP in dystrophin expressing muscle cells demonstrates the ability of loaded mini-nucleosomes to rescue diseases like Duchenne muscular dystrophy or other muscular dystrophies. Muscle tropism may also be enhanced by inclusion of other domains described in Table 4. Muscle tropism may also be achieved by intravenous delivery.

When injected via intramuscular route, the loaded mini-nucleosomes may be delivered at a dose greater than 1e5 genome copies per kg and up to a dose of 1e25 copies per kg of body weight (e.g., at about 1e5, 1e6, 1e7, 1e8, 1e9, 1e10, 1e15, 1e20, or 1e25 copies per kg body weight, or any range there between). Volume of the material may range from 1-900 milliliters (e.g., 1, 5, 10, 20, 30, 40, 50, 100, 200, 300, 400, 500, 600, 700, 800, or 900 milliliters). The loaded mini-nucleosomes may also be administered repeatedly (e.g., a selected volume and/or number of genome copies can be administered multiple times or divided among two or more does). The loaded mini-nucleosomes may also be administered intravenously to access muscle cells.

### Example 8: Loaded Mini-Nucleosomes are Redosable

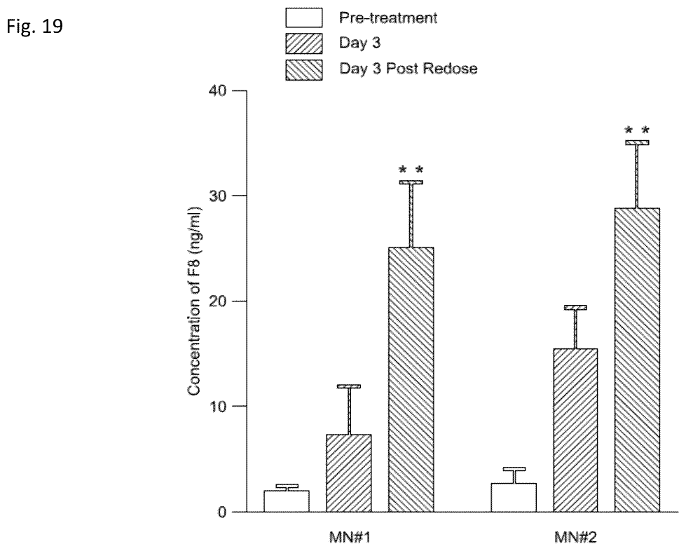

This example demonstrates that mini-nucleosomes can be re-administered without any neutralizing effect on the expression of proteins (FIG. 19). Balb/c mice were simply restrained using standard restraining techniques and Insulin syringe were used to deliver the loaded mini-nucleosomes MN #1 (SEQ ID NO: 390+F8 plasmid), and MN #2 (SEQ ID NO: 391+F8 plasmid, SEQ ID NO: 393) via tail vein injection. Each mouse received 20 micrograms 1^{st} dose and 40 micrograms 2^{nd} dose (30 days after 1^{st} dose). Serum were collected by cheek bleed technique at day 3 post 1^{st} and 2^{nd} doses. ~150 ul blood were collected each time and serum was collected from blood using standard techniques. F8 Elisa was performed to determine expression levels of F8 in serum in Balb/c mice following intravenous delivery of loaded mini-nucleosomes. F8 Elisa was performed according to manufacturer's (Aviva Systems Biology) instructions. 1:6 serum dilutions were made for all assays. We observed that when delivered a second time, there was no neutralizing effect in the expression levels, as evidenced by increase in protein levels of F8 (FIG. 19).

Provided herein are examples of mini-nucleosome core proteins and loaded mini-nucleosome that can be delivered repeatedly to boost expression levels of desired proteins. Redosability is a very important feature for any drug that may require repeat administration. In gene therapy, currently one of the most undesirable features of viral vectors is the inability to re-administer drug products. Viral vector once injected into the patient leads to formation of neutralizing antibodies. This causes immunogenicity and inexpressibility when they are administered the second time. We show here that, mini-nucleosome mediated gene delivery solves this problem. The non-immunogenic nature of mini-nucleosome is engineered in by design: by combining self-peptides or human derived amino acid sequences and enhanced by pegylation. In literature, pegylated proteins have been shown to evade the immune system. In this case, in mice, lack of immunogenicity for artificial human derived core proteins, further validates the case for pegylation. This redosability feature will allow multiple treatments to patients when needed. In case of diminishing expression levels over time, this redosable feature will allow repeat treatment to boost the expression to desired levels. This piece of data also shows that in some patients that need multi-organ injections, mini-nucleosome mediated gene transfer will be most desirable. One skilled in the art may also contemplate repeat dosing via many other routes of administration such as topical, oral, vaginal, intraperitoneal, intraocular, intrathecal, intracerebral, subcutaneous etc. or via encapsulation in liposomes or other synthetic materials.

Repeat doses may be delivered at a concentration greater than 1e5 genome copies per kg and up to a dose of 1e25 copies per kg of body weight (e.g., at about 1e5, 1e6, 1e7, 1e8, 1e9, 1e10, 1e15, 1e20, or 1e25 copies per kg body weight, or any range there between). Volume of the material may range from 1-900 milliliters (e.g., 1, 5, 10, 20, 30, 40, 50, 100, 200, 300, 400, 500, 600, 700, 800, or 900 milliliters). The loaded mini-nucleosomes may also be administered repeatedly (e.g., a selected volume and/or number of genome copies can be administered multiple times or divided among two or more does).

### Example 9: General Techniques

This example describes general techniques for cloning, delivery of min-nucleosomes into cells. Some of the cloning techniques that can be applied to constructing these vectors may include—synthesis of transgene constructs, TOPO PCR cloning, blunt end cloning, seamless cloning, long fragment cloning, restriction enzyme digestion and ligation but not limited to these techniques. DNA or RNA molecules may express one or more expression markers such as GFP, YFP and Luciferase but not limited to it. DNA or RNA molecules may express one or more therapeutic RNA or proteins but not limited to it.

Loaded mini-nucleosomes can be tested for their function and characterized in vitro by expressing them in HEK cells or other animal cell lines. Ability of synthesized and/or purified loaded mini-nucleosomes to transduce hematopoietic stem cells or differentiated peripheral blood mononuclear cells can be assayed by exposing the cells to the loaded mini-nucleosomes in culture. Loaded mini-nucleosomes can also be tested for their function and ability to form chimeric T cells in vitro by exposure to mini-nucleosomes or via techniques of transfection, or other physical methods for insertions. Loaded mini-nucleosomes can be tested for their function and characterized in vivo by delivering in mice or any other animal models but not limited to it.

### Example 10: Phosphorylated Mini-Nucleosome Core Proteins Deliver Nucleic Acid Payloads to Neurons and Central Nervous System Cells

The present Example demonstrates that a mini-nucleosome core protein including one or more residues modified by phosphorylation is particularly advantageous at least for expression of a nucleic acid payload (i.e., expression of an expression product encoded by a nucleic acid payload) in certain cells or tissues. For example, a mini-nucleosome core protein including one or more residues modified by phosphorylation can be particularly advantageous for expression of a nucleic acid payload in central nervous system cells including neurons, and particularly including spinal cord cells and brain neurons.

Modified mini-nucleosome core proteins were prepared by phosphorylation of a mini-nucleosome core protein having an amino acid sequence according to SEQ ID NO: 399. In particular, a threonine residue at position 11 of the mini-nucleosome core protein was phosphorylated. This residue is positioned in a linker domain (VT) of the mini-nucleosome core protein. However, the present disclosure provides that the presence of the modification, rather than the particular position of the modification within the mini-nucleosome core protein, determines the characteristics of the mini-nucleosome core proteins disclosed herein. Without wishing to be bound by any particular scientific theory, the present Example includes the recognition that modifications of mini-nucleosome core proteins according the present Example can interact with cell surface receptors, improving delivery of loaded mini-nucleosomes to certain target cells. Accordingly, the present disclosure provides that phosphorylation of any amino acid residue(s) of a mini-nucleosome core protein, in particular a serine, threonine, or tyrosine, would provide equivalent advantages and characteristics as phosphorylation of the particular residue of the particular mini-nucleosome core protein modified in the present Example. Unmodified mini-nucleosome core proteins according to the same amino acid sequence were also included in the present example as a control.

Mini-nucleosome core proteins of the present Example were loaded with a nucleic acid payload that included a gene encoding luciferase. In the present Example, luciferase is representative of protein expression generally, in that expression of luciferase is indicative of the ability of a mini-nucleosome core protein modified as disclosed herein and discussed in the present Example to successfully delivery any nucleic acid payload for expression of any expression product. Loaded mini-nucleosomes of the present Example were administered intrathecally to one or more 9-10 week old Balb/c mice (3e10 gc/mouse; 10 ul volume).

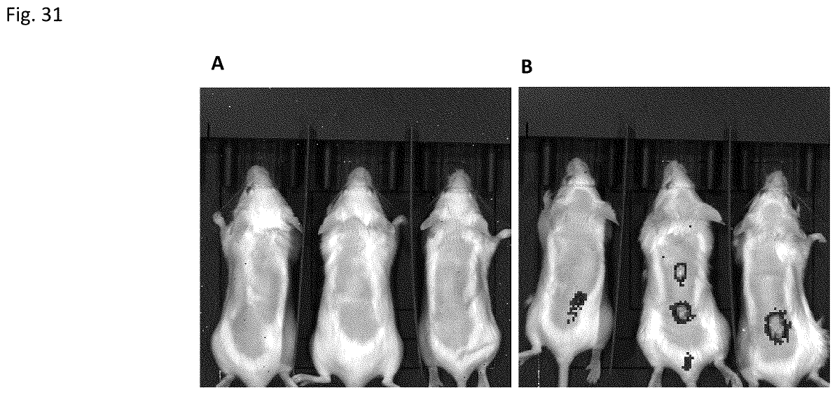

Results are shown in FIG. 31. On day 11 post dose administration, mice were dosed with luciferin at 150 mg/kg (60 mg/mL) via intraperitoneal (IP) injection at 2.5 ml/kg. At ~15 minutes post each luciferin administration (±5%), all animals underwent an IVIS imaging session. Panel A of FIG. 31 shows results using loaded mini-nucleosomes including an unmodified mini-nucleosome core protein, while Panel B of FIG. 31 shows results using loaded mini-nucleosomes including a phosphorylated mini-nucleosome core protein. All mini-nucleosome core proteins were loaded with a nucleic acid payload including a gene encoding luciferase. Panel B shows that the modified mini-nucleosome core protein, but not the unmodified mini-nucleosome core protein results in robust expression of the representative nucleic acid payload-encoded expression product (here, luciferase) in certain tissues including central nervous system cells including neurons, and including spinal cord cells and brain neurons. Expression levels observed in the present Example were highly unexpected. Detection of expression by IVIS imaging requires a very high level of expression, and the present data therefore reflects remarkable efficiency of both cellular uptake and payload expression. Levels of expression dramatically exceed those that would be expected for a non-viral vector.

### Example 11: Sulfated Mini-Nucleosome Core Proteins Deliver Nucleic Acid Payloads to Neurons and Central Nervous System Cells

The present Example demonstrates that a mini-nucleosome core protein including one or more residues modified by sulfation is particularly advantageous at least for expression of a nucleic acid payload (i.e., expression of an expression product encoded by a nucleic acid payload) in certain cells or tissues. For example, a mini-nucleosome core protein including one or more residues modified by sulfation can be particularly advantageous for expression of a nucleic acid payload in central nervous system cells including neurons, and particularly including spinal cord cells and brain neurons.

Modified mini-nucleosome core proteins were prepared by sulfation of a mini-nucleosome core protein having an amino acid sequence according to SEQ ID NO: 388. In particular, a tyrosine residue at position 38 of the mini-nucleosome core protein was sulfated. This residue is positioned in a targeting domain (FYQPL) of the mini-nucleosome core protein. However, the present disclosure provides that the presence of the modification, rather than the particular position of the modification within the mini-nucleosome core protein, determines the characteristics of the mini-nucleosome core proteins disclosed herein. Without wishing to be bound by any particular scientific theory, the present Example includes the recognition that modifications of mini-nucleosome core proteins according the present Example can interact with cell surface receptors, improving delivery of loaded mini-nucleosomes to certain target cells. Accordingly, the present disclosure provides that sulfation of any amino acid residue(s) of a mini-nucleosome core protein, in particular a serine, threonine, or tyrosine, would provide equivalent advantages and characteristics as sulfation of the particular residue of the particular mini-nucleosome core protein modified in the present Example. Unmodified mini-nucleosome core proteins according to the same amino acid sequence were also included in the present example as a control.

Mini-nucleosome core proteins of the present Example were loaded with a nucleic acid payload that included a gene encoding luciferase. In the present Example, luciferase is representative of protein expression generally, in that expression of luciferase is indicative of the ability of a mini-nucleosome core protein modified as disclosed herein and discussed in the present Example to successfully delivery any nucleic acid payload for expression of any expression product. Loaded mini-nucleosomes of the present Example were administered intrathecally to one or more 9-10 week old Balb/c mice (3e10 gc/mouse; 10 ul volume).

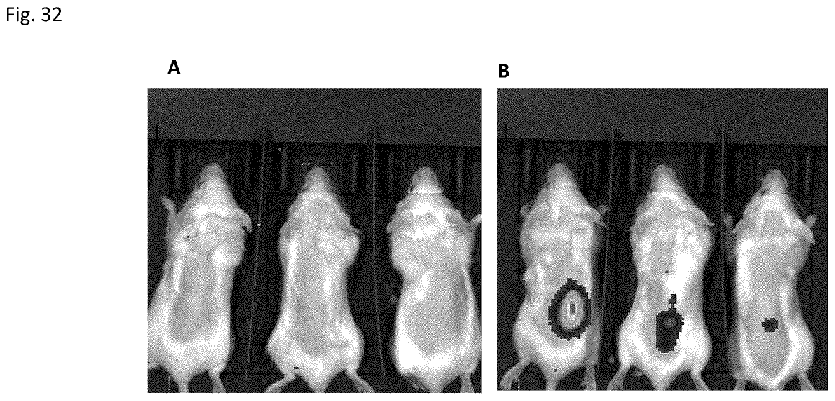

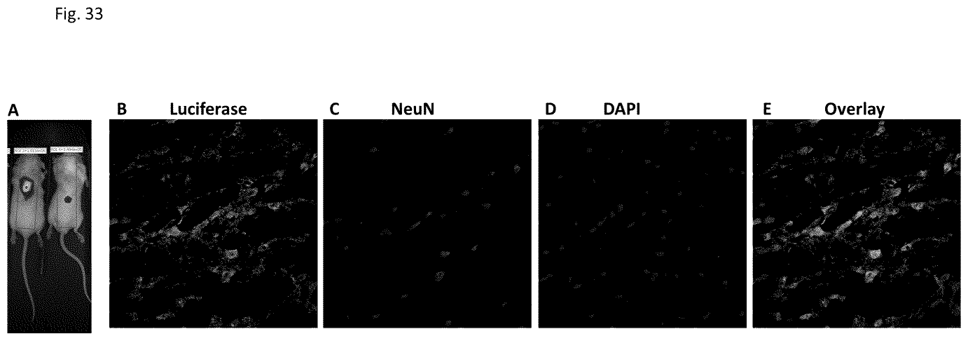

Results are shown in FIGS. 32 and 33. On day 11 post dose administration, mice were dosed with luciferin at 150 mg/kg (60 mg/mL) via intraperitoneal (IP) injection at 2.5 ml/kg. At ~15 minutes post each luciferin administration (t 5%), all animals underwent an IVIS imaging session. Panel A of FIG. 32 shows results using loaded mini-nucleosomes including an unmodified mini-nucleosome core protein, while Panel B of FIG. 32 shows results using loaded mini-nucleosomes including a sulfated mini-nucleosome core protein. All mini-nucleosome core proteins were loaded with a nucleic acid payload including a gene encoding luciferase. Panel B of FIG. 32 shows that the modified mini-nucleosome core protein, but not the unmodified mini-nucleosome core protein results in robust expression of the representative nucleic acid payload-encoded expression product (here, luciferase) in certain tissues including central nervous system cells including neurons, and including spinal cord cells and brain neurons. FIG. 33 includes brain tissue sections of a mouse administered a sulfated mini-nucleosome core protein loaded with the luciferase-encoding payload, which mouse showed a high degree of expression in certain tissues including central nervous system cells including neurons, and including spinal cord cells and brain neurons (Panel A). Panels B, C, and D respectively show luciferase, anti-NeuN antibody, and D-DAPI stains, with an overlay of these stains shown in Panel E. Images of FIG. 33 demonstrate robust expression of the representative nucleic acid payload-encoded expression product (here, luciferase) in brain cells, particularly including brain neurons, when the mini-nucleosome is sulfated but not when the mini-nucleosome is unmodified. Expression levels observed in the present Example were highly unexpected. Detection of expression by IVIS imaging requires a very high level of expression, and the present data therefore reflects remarkable efficiency of both cellular uptake and payload expression. Levels of expression dramatically exceed those that would be expected for a non-viral vector. Moreover, it was unexpected that sulfation would increase expression in brain cells, particularly including brain neurons.

### Example 12: Acetylated Mini-Nucleosome Core Proteins Deliver Nucleic Acid Payloads to Neurons and Central Nervous System Cells

The present example demonstrates that a mini-nucleosome core protein including one or more residues modified by acetylation is particularly advantageous at least for expression of a nucleic acid payload (i.e., expression of an expression product encoded by a nucleic acid payload) in certain cells or tissues. For example a mini-nucleosome core protein including one or more residues modified by acetylation can be particularly advantageous for expression of a nucleic acid payload in retinal cells, in particular photoreceptors

Modified mini-nucleosome core proteins were prepared by acetylation of a mini-nucleosome core protein having an amino acid sequence according to SEQ ID NO: 401. In particular, a lysine residue at position 10 of the mini-nucleosome core protein was acetylated. This residue is positioned in a targeting domain (KKRPKP) of the mini-nucleosome core protein. However, the present disclosure provides that the presence of the modification, rather than the particular position of the modification within the mini-nucleosome core protein, determines the characteristics of the mini-nucleosome core proteins disclosed herein. Without wishing to be bound by any particular scientific theory, the present Example includes the recognition that modifications of mini-nucleosome core proteins according the present Example can interact with cell surface receptors, improving delivery of loaded mini-nucleosomes to certain target cells. Accordingly, the present disclosure provides that acetylation of any amino acid residue(s) of a mini-nucleosome core protein, in particular a lysine, would provide equivalent advantages and characteristics as acetylation of the particular residue of the particular mini-nucleosome core protein modified in the present Example. Unmodified mini-nucleosome core proteins according to the same amino acid sequence were also included in the present example as a control.

Mini-nucleosome core proteins of the present Example were loaded with a nucleic acid payload that included a gene encoding GFP. In the present Example, GFP is representative of protein expression generally, in that expression of GFP is indicative of the ability of a mini-nucleosome core protein modified as disclosed herein and discussed in the present Example to successfully delivery any nucleic acid payload for expression of any expression product. Loaded mini-nucleosomes of the present Example were administered intravitreally to one or more mice. For intravitreal injections, mice were anesthetized by IP injection with Ketamine/Xylazine (90-100 mg/kg+10 mg/kg), a target pupil was dilated under a microscope using topical Tropicamide (1%), and an incision/insertion was made on the sclera ~1 mm below the limbus using a 30-gauge needle. 1 μl of loaded mini-nucleosome was injected into the vitreous cavity using 32 gauge blunt needle passing through the incision made by the 25-gauge needle.

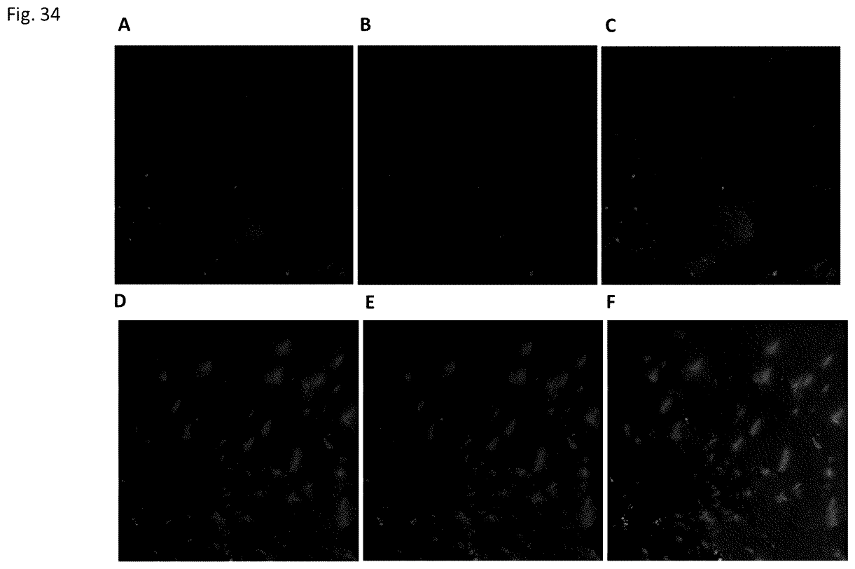

Results are shown in FIG. 34. Eyes were enucleated at 4 weeks post injection and placed in 4% PFA for fixation. Lenses were removed from the eyes by dissection and the retinal wholemount was dissected from eyecup. Retinal wholemounts were hydrated with 1×PBS, blocked using 2% BSA and stained with GFP antibodies coupled to Alexa-flour 555 at room temperature for 1 hr and washed with 1×PBS 4 times, 5 minutes each. Images were obtained using Leica SP5 confocal microscope.

Panels A-C of FIG. 34 show results using loaded mini-nucleosomes including an unmodified mini-nucleosome core protein, while Panel D-F of FIG. 34 shows results using loaded mini-nucleosomes including an acetylated mini-nucleosome core protein. All mini-nucleosome core proteins were loaded with a nucleic acid payload including a gene encoding GFP. Images demonstrate robust expression of the representative nucleic acid payload-encoded expression product (here, GFP) in retinal cells, in particular photoreceptors, when the mini-nucleosome is acetylated but not when the mini-nucleosome is unmodified. Expression levels observed in the present Example were highly unexpected. Detection of expression by IVIS imaging requires a very high level of expression, and the present data therefore reflects remarkable efficiency of both cellular uptake and payload expression. Levels of expression dramatically exceed those that would be expected for a non-viral vector. Moreover, it was unexpected that acetylation would increase expression in retinal cells, in particular photoreceptors.

### Example 13: Phosphorylated Mini-Nucleosome Core Proteins Deliver Nucleic Acid Payloads to Neurons and Central Nervous System Cells

The present Example demonstrates that a mini-nucleosome core protein including one or more residues modified by mannosylation is particularly advantageous at least for expression of a nucleic acid payload (i.e., expression of an expression product encoded by a nucleic acid payload) in certain cells or tissues. For example, a mini-nucleosome core protein including one or more residues modified by mannosylation can be particularly advantageous for expression of a nucleic acid payload in retinal cells, in particular photoreceptors.

Modified mini-nucleosome core proteins were prepared by mannosylation of a mini-nucleosome core protein having an amino acid sequence according to SEQ ID NO: 447. In particular, a serine residue at position 9 of the mini-nucleosome core protein was mannosylated. This residue is positioned in a linker domain (GGS) of the mini-nucleosome core protein. However, the present disclosure provides that the presence of the modification, rather than the particular position of the modification within the mini-nucleosome core protein, determines the characteristics of the mini-nucleosome core proteins disclosed herein. Without wishing to be bound by any particular scientific theory, the present Example includes the recognition that modifications of mini-nucleosome core proteins according the present Example can interact with cell surface receptors, improving delivery of loaded mini-nucleosomes to certain target cells. Accordingly, the present disclosure provides that mannosylation of any amino acid residue(s) of a mini-nucleosome core protein, in particular a serine, would provide equivalent advantages and characteristics as mannosylation of the particular residue of the particular mini-nucleosome core protein modified in the present Example. Unmodified mini-nucleosome core proteins according to the same amino acid sequence were also included in the present example as a control.

Mini-nucleosome core proteins of the present Example were loaded with a nucleic acid payload that included a gene encoding GFP. In the present Example, GFP is representative of protein expression generally, in that expression of GFP is indicative of the ability of a mini-nucleosome core protein modified as disclosed herein and discussed in the present Example to successfully delivery any nucleic acid payload for expression of any expression product. Loaded mini-nucleosomes of the present Example were administered subretinally to one or more mice. For subretinal injections, mice were anesthetized by IP injection with Ketamine/Xylazine (90-100 mg/kg+10 mg/kg), a target pupil was dilated under a microscope using topical Tropicamide (1%), and an incision/insertion was made on the sclera ~1 mm below the limbus using a 30-gauge needle. 1 μl of loaded mini-nucleosome was injected into the vitreous cavity using 32 gauge blunt needle passing through the incision made by the 25-gauge needle behind the retina. A micropump controlled injector was utilized for this procedure.

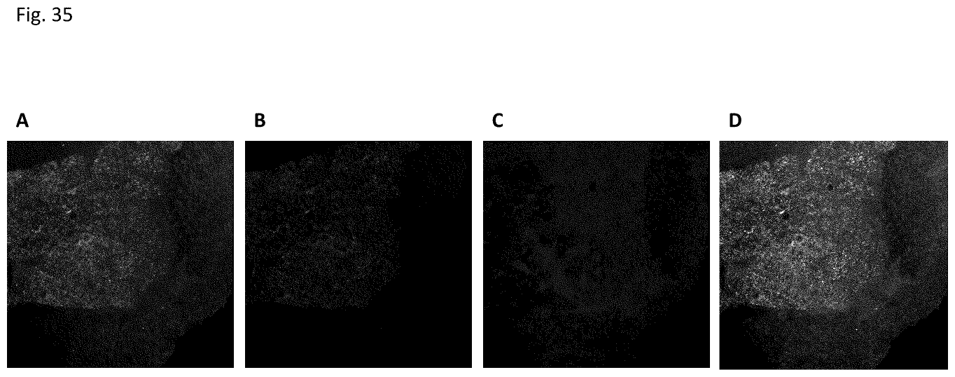

Results are shown in FIG. 35. Eyes were enucleated at 2 weeks post injection and placed in 4% PFA for fixation. Lenses were removed from the eyes by dissection and the retinal wholemount was dissected from eyecup. Retinal wholemounts were hydrated with 1×PBS, blocked using 2% BSA and stained with GFP antibodies coupled to Alexa-flour 555 at room temperature for 1 hour and washed with 1×PBS 4 times, 5 minutes each. Images were obtained using Leica SP5 confocal microscope.

FIG. 35, panels A-D, show a representative retinal wholemount from a mouse administered loaded mini-nucleosomes including mannosylated mini-nucleosome core protein. Images demonstrate robust expression of the representative nucleic acid payload-encoded expression product (here, GFP) in retinal cells, in particular photoreceptors, when the mini-nucleosome is mannosylated. An unmodified control loaded with the same nucleic acid payload was not robustly expressed in retinal cells or photoreceptors. Expression levels observed in the present Example were highly unexpected. Detection of expression by IVIS imaging requires a very high level of expression, and the present data therefore reflects remarkable efficiency of both cellular uptake and payload expression. Levels of expression dramatically exceed those that would be expected for a non-viral vector.

### CERTAIN SEQUENCES

SEQ ID NO: 394
KKRHRK-[LINKER]-LRE-[LINKER]KRHRKLRRRRRLKRHRKKRHRK-[LINKER]-
LRE-[LINKER]-K
(where [LINKER] could be any amino acid sequence described in Table 12 but not
limited to it)
SEQ ID NO: 389
KKKRHRKRKRKRKRRRRKKK-[LINKER]-ASSLNIAK-[LINKER]-RRRR
(where [LINKER] could be any amino acid sequence described in Table 12 but not limited to it)
SEQ ID NO: 390
KKKRK-[LINKER]-NGR-[LINKER]-KRKRKKRHRKKKKRRRRKRHRK-[LINKER]-
NGR-[LINKER]-KKK
(where [LINKER] could be any amino acid sequence described in Table 12 but not limited to it)
SEQ ID NO: 391
KKKRHRKKKKK-[LINKER]-RGD-[LINKER]-KKKK-[LINKER]-NTQIH-[LINKER]-
RRRRR-[LINKER]-TPH-[LINKER]-KK
(where [LINKER] could be any amino acid sequence described in Table 12 but not
limited to it)
SEQ ID NO: 392
KKKRK-[LINKER]-KTKKK-[LINKER]-AK-[LINKER]-KALKKK-[LINKER]-
KKGKKKKRRRRKAAPKK
(where [LINKER] could be any amino acid sequence described in Table 12 but not
limited to it)
SEQ ID NO: 393
CKKKKKKKKKKKKKKKKKKKKKKKKKKKKKK
CBA-F8 plasmid
SEQ ID NO: 460
TCGCGCGTTTCGGTGATCGAGGTGAGCCCCACGTTCTGCTTCACTCTCCCCATCTCCC
CCCCCTCCCCACCCCCAATTTTGTATTTATTTATTTTTTAATTATTTTGTGCAGCGATG
GGGGCGGGGGGGGGGGGGGGGCGCGCGCCAGGCGGGGCGGGGCGGGGCGAGGGG
CGGGGCGGGGCGAGGCGGAGAGGTGCGGCGGCAGCCAATCAGAGCGGCGCGCTCC
GAAAGTTTCCTTTTATGGCGAGGCGGCGGCGGCGGCGGCCCTATAAAAAGCGAAGC
GCGCGGCGGGCGGGAGTCGCTGCGACGCTGCCTTCGCCCCGTGCCCCGCTCCGCCGC
CGCCTCGCGCCGCCCGCCCCGGCTCTGACTGACCGCGTTACTCCCACAGGTGAGCGG
GCGGGACGGCCCTTCTCCTCCGGGCTGTAATTAGCGCTTGGTTTAATGACGGCTTGT
TTCTTTTCTGTGGCTGCGTGAAAGCCTTGAGGGGCTCCGGGAGGGCCCTTTGTGCGG
GGGGGAGCGGCTCGGGGGGTGCGTGCGTGTGTGTGTGCGTGGGGAGCGCCGCGTGC
GGCCCGCGCTGCCCGGCGGCTGTGAGCGCTGCGGGCGCGGCGCGGGGCTTTGTGCG
CTCCGCAGTGTGCGCGAGGGGAGCGCGGCCGGGGGCGGTGCCCCGCGGTGCGGGGG
GGGCTGCGAGGGGAACAAAGGCTGCGTGCGGGGTGTGTGCGTGGGGGGGTGAGCA
GGGGGTGTGGGCGCGGCGGTCGGGCTGTAACCCCCCCCTGCACCCCCCTCCCCGAGT
TGCTGAGCACGGCCCGGCTTCGGGTGCGGGGCTCCGTACGGGGCGTGGCGCGGGGC
TCGCCGTGCCGGGCGGGGGGTGGCGGCAGGTGGGGGTGCCGGGCGGGGCGGGGCC
GCCTCGGGCCGGGGAGGGCTCGGGGGAGGGGCGCGGCGGCCCCCGGAGCGCCGGC
GGCTGTCGAGGCGCGGCGAGCCGCAGCCATTGCCTTTTATGGTAATCGTGCGAGAG
GGCGCAGGGACTTCCTTTGTCCCAAATCTGTGCGGAGCCGAAATCTGGGAGGCGCC
GCCGCACCCCCTCTAGCGGGCGCGGGGCGAAGCGGTGCGGCGCCGGCAGGAAGGA
AATGGGCGGGGAGGGCCTTCGTGCGTCGCCGCGCCGCCGTCCCCTTCTCCCTCTCCA
GCCTCGGGGCTGTCCGCGGGGGGACGGCTGCCTTCGGGGGGGACGGGGCAGGGCGG
GGTTCGGCTTCTGGCGTGTGACCGGCGGCTCTAGAGCCTCTGCTAACCATGTTCATG
CCTTCTTCTTTTTCCTACAGCTCCTGGGCAACGTGCTGGTTATTGTGCTGTCTCATCAT
TTTGGCAAAACCGGTCTCGAAGGCCTGCAGGCGGCCGCCGCCACCGCCACCATGCA
AATAGCACTCTTCGCTTGCTTCTTTCTGAGCCTTTTCAATTTCTGCTCTAGTGCCATCA
GAAGATACTACCTTGGTGCAGTGGAATTGTCCTGGAACTATATTCAGAGTGATCTGC
TCAGTGTGCTGCATACAGACTCAAGATTTCTTCCTAGAATGTCAACATCTTTTCCATT
CAACACCTCCATCATGTATAAAAAGACTGTGTTTGTAGAGTACAAGGACCAGCTTTT
CAACATTGCCAAGCCCAGGCCACCCTGGATGGGTTTGCTAGGTCCTACCATTTGGAC
TGAGGTTCATGACACAGTGGTCATTACACTTAAAAACATGGCTTCTCATCCTGTCAG
TCTTCATGCTGTTGGTGTGTCCTACTGGAAAGCTTCTGAGGGAGATGAATATGAAGA
TCAGACAAGCCAAATGGAGAAGGAAGATGATAAAGTTTTCCCTGGTGAAAGTCATA
CTTATGTTTGGCAAGTCCTGAAAGAGAATGGTCCAATGGCCTCTGACCCTCCATGTC
TCACTTACTCATATATGTCTCATGTGGATCTGGTGAAAGATTTGAATTCAGGCCTCAT
TGGAGCTCTGCTAGTATGTAAAGAAGGCAGTCTCTCCAAAGAAAGAACACAGATGT
TGTACCAATTTGTACTGCTTTTTGCTGTATTTGATGAAGGGAAGAGCTGGCACTCAG
AAACAAACGACTCTTATACACAGTCTATGGATTCTGCATCTGCTAGAGACTGGCCTA
AAATGCACACAGTCAATGGCTATGTAAACAGGTCTCTTCCAGGTCTGATTGGATGCC
ATAGGAAATCAGTCTACTGGCACGTGATTGGAATGGGCACCACTCCTGAAATACACT
CAATATTCCTCGAAGGTCACACATTTTTTGTGAGGAACCACCGTCAAGCTTCATTGG
AGATATCACCAATAACTTTCCTTACTGCTCAAACACTCTTGATAGATCTTGGGCAGTT
CCTACTATTTTGTCATATCTCTTCCCATAAACATGATGGCATGGAAGCTTATGTCAAA
GTAGATAGCTGCCCTGAGGAATCCCAATGGCAAAAGAAAAATAATAATGAGGAAAT
GGAAGATTATGATGATGATCTTTATTCAGAAATGGATATGTTCACATTGGATTATGA
CAGCTCTCCTTTTATCCAAATTCGCTCGGTTGCTAAAAAGTACCCTAAAACTTGGAT
ACATTATATTTCTGCTGAGGAGGAAGACTGGGACTATGCACCTTCAGTTCCTACCTC
GGATAATGGAAGTTATAAAAGCCAGTATCTGAGCAATGGTCCTCATCGGATTGGTA
GGAAATATAAAAAAGTCAGATTTATAGCATACACAGATGAAACCTTTAAGACTCGT
GAAACTATTCAGCATGAATCAGGACTCTTGGGACCTTTACTTTATGGAGAAGTTGGA
GACACACTGTTGATTATTTTTAAGAATCAAGCAAGCCGACCATATAACATTTACCCT
CATGGAATCACTGATGTCAGTCCTCTACATGCAAGGAGATTGCCAAGAGGTATAAA
GCACGTGAAGGATTTGCCAATTCATCCAGGAGAGATATTCAAGTACAAGTGGACAG
TTACAGTAGAAGATGGACCAACTAAATCAGATCCACGGTGCCTGACCCGCTATTATT
CAAGTTTCATTAACCCTGAGAGAGATCTAGCTTCAGGACTGATTGGCCCTCTTCTCA
TCTGCTACAAAGAATCTGTAGATCAAAGGGGAAACCAGATGATGTCAGACAAAAGA
AATGTCATCCTGTTTTCTATATTTGATGAGAACCAAAGCTGGTACATCACAGAGAAC
ATGCAACGCTTCCTCCCCAATGCAGCTAAAACACAGCCCCAGGACCCTGGGTTCCAG
GCCTCCAACATCATGCACAGCATCAATGGCTATGTTTTTGATAGCTTGGAGTTGACA
GTTTGTTTGCATGAGGTGGCATACTGGCACATTCTCAGTGTTGGAGCACAGACAGAC
TTCTTATCTATCTTCTTCTCTGGATATACTTTCAAACACAAAATGGTCTATGAAGATA
CACTTACCCTGTTCCCATTCTCAGGAGAAACTGTCTTTATGTCGATGGAAAACCCAG
GTCTATGGGTCTTGGGGTGTCATAATTCAGACTTTCGGAAGAGAGGTATGACAGCAT
TGCTGAAAGTTTCTAGTTGTGACAAGAGCACTAGTGATTATTATGAAGAAATATATG
AAGATATTCCAACACAGTTGGTGAATGAGAACAATGTCATTGATCCCAGAAGCTTCT
TCCAGAATACAAATCATCCTAATACTAGGAAAAAGAAATTCAAAGATTCCACAATT
CCAAAAAATGATATGGAGAAGATTGAGCCTCAGTTTGAAGAGATAGCAGAGATGCT
TAAAGTACAGAGTGTCTCAGTTAGTGACATGTTGATGCTCTTGGGACAGAGTCATCC
TACTCCACATGGCTTATTTTTATCAGATGGCCAAGAAGCCATCTATGAGGCTATTCA
TGATGATCATTCACCAAATGCAATAGACAGCAATGAAGGCCCATCTAAAGTGACCC
AACTCAGGCCAGAATCCCATCACAGTGAGAAAATAGTATTTACTCCTCAGCCCGGCC
TCCAGTTAAGATCCAATAAAAGTTTGGAGACAACTATAGAAGTAAAGTGGAAGAAA
CTTGGTTTGCAAGTTTCTAGTTTGCCAAGTAATCTAATGACTACAACAATTCTGTCAG
ACAATTTGAAAGCAACTTTTGAAAAGACAGATTCTTCAGGATTTCCAGATATGCCAG
TTCACTCTAGTAGTAAATTAAGTACTACTGCATTTGGTAAGAAAGCATATTCCCTTGT
TGGGTCTCATGTACCTTTAAACGTGAGTGAAGAAAATAGTGATTCCAACATATTGGA
TTCAACTTTAATGTATAGTCAAGAAAGTTTACCAAGAGATAATATATTATCAATGGA
GAATGATAGATTACTCAGAGAGAAGAGGTTTCATGGAATTGCTTTATTGACCAAAG
ATAATACTTTATTCAAAGACAATGTCTCCTTAATGAAAACAAACAAAACATATAATC
ATTCAACAACTAATGAAAAACTACACACTGAGAGCCCAACATCAATTGAGAATAGT
ACAACAGACTTGCAAGATGCCATATTAAAGGTCAATAGTGAGATTCAAGAAGTAAC
AGCTTTGATTCATGATGGAACACTTTTAGGCAAAAATTCTACATATTTGAGACTAAA
CCATATGCTAAATAGAACTACCTCAACAAAAAATAAAGACATATTTCATAGAAAAG
ATGAAGATCCTATTCCACAAGATGAAGAGAATACAATCATGCCATTTTCCAAGATGT
TGTTCTTGTCAGAATCTTCAAATTGGTTTAAAAAGACCAATGGAAATAATTCCTTGA
ACTCTGAGCAAGAACATAGTCCAAAGCAATTAGTATATTTAATGTTTAAAAAATATG
TAAAAAATCAAAGTTTCTTGTCAGAGAAAAATAAAGTCACAGTAGAACAGGATGGA
TTTACAAAGAACATAGGACTTAAAGACATGGCTTTTCCACATAATATGAGCATATTT
CTTACCACTTTGTCTAACGTACATGAAAATGGTAGGCACAATCAAGAAAAAAATATT
CAGGAAGAGATAGAGAAGGAAGCACTAATTGAAGAGAAAGTAGTTTTGCCCCAGGT
GCACGAAGCAACTGGCTCTAAGAATTTCTTGAAAGACATATTGATACTAGGCACTAG
GCAAAATATAAGTTTATATGAAGTACATGTACCAGTACTTCAAAACATCACATCAAT
AAACAATTCAACAAATACAGTACAGATTCACATGGAGCATTTCTTTAAAAGAAGGA
AGGACAAGGAAACAAATTCAGAAGGCTTGGTAAATAAAACCAGAGAAATGGTAAA
AAACTATCCAAGCCAGAAGAATATTACTACTCAACGTAGTAAACGGGCTTTGGGAC
AATTCAGACTGTCAACTCAATGGCTTAAAACCATAAACTGTTCAACACAGTGTATCA
TTAAACAGATAGACCACAGCAAGGAAATGAAAAAGTTCATTACTAAATCTTCCTTAT
CAGATTCTTCTGTGATTAAAAGCACCACTCAGACAAATAGTTCTGACTCACACATTG
TAAAAACATCAGCATTTCCACCAATAGATCTCAAAAGGAGTCCATTCCAAAACAAA
TTTTCTCATGTTCAAGCATCATCCTACATTTATGACTTTAAGACAAAAAGTTCAAGA
ATTCAAGAAAGCAATAATTTCTTAAAAGAAACCAAAATAAATAACCCTTCTTTAGCC
ATTCTACCATGGAATATGTTCATAGATCAAGGAAAATTTACCTCCCCAGGGAAAAGT
AACACAAACTCAGTCACATATAAGAAACGTGAGAACATTATTTTCTTGAAACCAACT
TTGCCTGAAGAATCTGGCAAAATTGAATTGCTTCCTCAAGTTTCCATTCAAGAGGAA
GAAATTTTACCTACAGAAACTAGCCATGGATCTCCTGGACACTTGAATCTCATGAAA
GAGGTCTTTCTTCAGAAAATACAGGGGCCTACTAAATGGAATAAAGCAAAGAGGCA
TGGAGAAAGTATAAAAGGTAAAACAGAGAGCTCTAAAAATACTCGCTCAAAACTGC
TAAATCATCATGCTTGGGATTATCATTATGCTGCACAGATACCAAAAGATATGTGGA
AATCCAAAGAGAAGTCACCAGAAATTATATCCATTAAGCAAGAGGACACCATTTTG
TCTCTGAGGCCTCATGGAAACAGTCATTCAATAGGGGCAAATGAGAAACAAAATTG
GCCTCAAAGAGAAACCACTTGGGTAAAGCAAGGCCAAACTCAAAGGACATGCTCTC
AAATCCCACCAGTGTTGAAACGACATCAAAGGGAACTTAGTGCTTTTCAATCAGAAC
AAGAAGCAACTGACTATGATGATGCCATCACCATTGAAACAATCGAGGATTTTGAC
ATTTACAGTGAGGACATAAAGCAAGGTCCCCGCAGCTTTCAACAGAAAACAAGGCA
CTATTTTATTGCAGCTGTGGAACGACTCTGGGACTATGGGATGAGTACATCTCATGT
TCTACGAAATAGGTATCAAAGTGACAATGTACCTCAGTTCAAGAAAGTAGTTTTCCA
GGAATTTACTGATGGCTCCTTTAGTCAGCCCTTATATCGTGGAGAATTAAATGAACA
CCTGGGGTTGTTGGGCCCATATATAAGAGCAGAAGTTGAAGACAACATTATGGTAA
CTTTCAAAAACCAGGCCTCCCGTCCCTACTCCTTCTATTCTAGCCTCATTTCTTATAA
AGAAGATCAGAGAGGAGAAGAACCTAGAAGAAACTTTGTCAAGCCTAATGAAACC
AAAATTTATTTTTGGAAAGTACAACATCATATGGCACCCACAGAAGATGAGTTTGAC
TGCAAGGCCTGGGCTTATTTCTCTGATGTTGATCTTGAAAGAGATATGCACTCGGGA
TTAATTGGACCCCTTCTGATTTGCCACGCGAACACACTGAATCCTGCTCATGGGAGA
CAAGTGTCAGTACAGGAATTTGCTCTGCTTTTCACTATCTTTGATGAGACCAAGAGC
TGGTACTTCACTGAAAACGTGAAAAGGAACTGCAAGACACCCTGCAATTTCCAGAT
GGAAGACCCCACTTTGAAAGAGAATTATCGCTTCCATGCAATCAATGGTTATGTAAT
GGATACCCTACCAGGCTTAGTAATGGCTCAAGATCAAAGGATTCGATGGTATCTTCT
CAGCATGGGCAACAATGAGAACATCCAATCTATTCATTTCAGTGGACATGTTTTCAC
TGTACGGAAAAAAGAGGAGTATAAAATGGCAGTGTACAACCTCTACCCAGGTGTTT
TTGAGACTCTGGAAATGATACCATCCAGAGCTGGAATATGGCGAGTAGAATGCCTT
ATTGGCGAGCACTTACAGGCTGGGATGAGCACTCTTTTTCTGGTGTACAGCAAGCAG
TGTCAGATTCCTCTTGGAATGGCTTCTGGAAGCATCCGTGATTTCCAGATTACAGCTT
CAGGACATTATGGACAGTGGGCCCCAAACCTGGCAAGACTTCATTATTCCGGATCAA
TCAATGCCTGGAGTACCAAGGAGCCCTTTTCTTGGATCAAGGTAGATCTGTTGGCAC
CAATGATTGTTCATGGCATCAAGACTCAGGGTGCTCGTCAGAAATTTTCCAGCCTTT
ATATCTCTCAATTTATCATCATGTATAGCCTGGATGGGAAGAAGTGGCTGAGTTATC
AAGGAAATTCCACTGGAACCTTAATGGTTTTCTTTGGCAATGTGGACTCATCTGGGA
TTAAGCATAATAGTTTTAATCCTCCAATTATTGCTCGATATATCCGTTTGCACCCCAC
TCATTCTAGCATCCGTAGTACTCTTCGCATGGAGTTGATGGGCTGTGATTTAAACAG
TTGCAGCATACCATTGGGAATGGAAAGTAAAGTAATATCAGATACACAAATCACTG
CCTCATCCTACTTCACCAACATGTTTGCTACTTGGTCTCCTTCACAAGCTCGACTTCA
CCTCCAGGGAAGGACTAATGCCTGGCGACCTCAGGTGAATGATCCAAAACAATGGT
TGCAAGTGGACTTACAAAAGACAATGAAAGTCACTGGAATAATAACCCAGGGAGTG
AAATCTCTCTTTACCAGCATGTTTGTGAAAGAGTTCCTTATTTCCAGCAGTCAAGATG
GCCATCACTGGACTCAAATTTTATACAATGGCAAGGTAAAGGTTTTTCAGGGGAATC
AGGACTCATCCACACCTATGATGAATTCTCTAGACCCACCATTACTCACTCGCTATC
TTCGAATTCACCCCCAGATCTGGGAGCACCAAATTGCTCTGAGGCTTGAGATTCTAG
GATGTGAGGCCCAGCAGCAATACTGACCATGGCCCAACTTGTTTATTGCAGCTTATA
ATGGTTACAAATAAAGCAATAGCATCACAAATTTCACAAATAAAGCATTTTTTTCAC
TGCATTCTAGTTGTGGTTTGTCCAAACTCATCAATGTATCTTATCATGTCTGGATCTC
GTTAACTCGAGGGATCCATCGATGTCGACTGCAGAGGCCTGCATGCAAGCTTGGTGT
AATCATGGTCATAGCTGTTTCCTGTGTGAAATTGTTATCCGCTCACAATTCCACACAA
CATACGAGCCGGAAGCATAAAGTGTAAAGCCTGGGGTGCCTAATGAGTGAGCTAAC
TCACATTAATTGCGTTGCGCTCACTGCCCGCTTTCCAGTCGGGAAACCTGTCGTGCC
AGCTGCATTAATGAATCGGCCAACGCGCGGGGAGAGGCGGTTTGCGTATTGGGCGC
TCTTCCGCTTCCTCGCTCACTGACTCGCTGCGCTCGGTCGTTCGGCTGCGGCGAGCG
GTATCAGCTCACTCAAAGGCGGTAATACGGTTATCCACAGAATCAGGGGATAACGC
AGGAAAGAACATGTGAGCAAAAGGCCAGCAAAAGGCCAGGAACCGTAAAAAGGCC
GCGTTGCTGGCGTTTTTCCATAGGCTCCGCCCCCCTGACGAGCATCACAAAAATCGA
CGCTCAAGTCAGAGGTGGCGAAACCCGACAGGACTATAAAGATACCAGGCGTTTCC
CCCTGGAAGCTCCCTCGTGCGCTCTCCTGTTCCGACCCTGCCGCTTACCGGATACCTG
TCCGCCTTTCTCCCTTCGGGAAGCGTGGCGCTTTCTCATAGCTCACGCTGTAGGTATC
TCAGTTCGGTGTAGGTCGTTCGCTCCAAGCTGGGCTGTGTGCACGAACCCCCCGTTC
AGCCCGACCGCTGCGCCTTATCCGGTAACTATCGTCTTGAGTCCAACCCGGTAAGAC
ACGACTTATCGCCACTGGCAGCAGCCACTGGTAACAGGATTAGCAGAGCGAGGTAT
GTAGGCGGTGCTACAGAGTTCTTGAAGTGGTGGCCTAACTACGGCTACACTAGAAG
AACAGTATTTGGTATCTGCGCTCTGCTGAAGCCAGTTACCTTCGGAAAAAGAGTTGG
TAGCTCTTGATCCGGCAAACAAACCACCGCTGGTAGCGGTGGTTTTTTTGTTTGCAA
GCAGCAGATTACGCGCAGAAAAAAAGGATCTCAAGAAGATCCTTTGATCTTTTCTAC
GGGGTCTGACGCTCAGTGGAACGAAAACTCACGTTAAGGGATTTTGGTCATGAGATT
ATCAAAAAGGATCTTCACCTAGATCCTTTTAAATTAAAAATGAAGTTTTAAATCAAG
CCCAATCTGAATAATGTTACAACCAATTAACCAATTCTGATTAGAAAAACTCATCGA
GCATCAAATGAAACTGCAATTTATTCATATCAGGATTATCAATACCATATTTTTGAA
AAAGCCGTTTCTGTAATGAAGGAGAAAACTCACCGAGGCAGTTCCATAGGATGGCA
AGATCCTGGTATCGGTCTGCGATTCCGACTCGTCCAACATCAATACAACCTATTAAT
TTCCCCTCGTCAAAAATAAGGTTATCAAGTGAGAAATCACCATGAGTGACGACTGA
ATCCGGTGAGAATGGCAAAAGTTTATGCATTTCTTTCCAGACTTGTTCAACAGGCCA
GCCATTACGCTCGTCATCAAAATCACTCGCATCAACCAAACCGTTATTCATTCGTGA
TTGCGCCTGAGCGAGACGAAATACGCGATCGCTGTTAAAAGGACAATTACAAACAG
GAATCGAATGCAACCGGCGCAGGAACACTGCCAGCGCATCAACAATATTTTCACCT
GAATCAGGATATTCTTCTAATACCTGGAATGCTGTTTTTCCGGGGATCGCAGTGGTG
AGTAACCATGCATCATCAGGAGTACGGATAAAATGCTTGATGGTCGGAAGAGGCAT
AAATTCCGTCAGCCAGTTTAGTCTGACCATCTCATCTGTAACATCATTGGCAACGCT
ACCTTTGCCATGTTTCAGAAACAACTCTGGCGCATCGGGCTTCCCATACAAGCGATA
GATTGTCGCACCTGATTGCCCGACATTATCGCGAGCCCATTTATACCCATATAAATC
AGCATCCATGTTGGAATTTAATCGCGGCCTCGACGTTTCCCGTTGAATATGGCTCAT
AACACCCCTTGTATTACTGTTTATGTAAGCAGACAGTTTTATTGTTCATGATGATATA
TTTTTATCTTGTGCAATGTAACATCAGAGATTTTGAGACACGGGCCAGAGCTGCA
CBA-GFP plasmid
SEQ ID NO: 395
TCGCGCGTTTCGGTGATGACGGTCGAGGTGAGCCCCACGTTCTGCTTCACTCTCCCC
ATCTCCCCCCCCTCCCCACCCCCAATTTTGTATTTATTTATTTTTTAATTATTTTGTGC
AGCGATGGGGGCGGGGGGGGGGGGGGGGCGCGCGCCAGGCGGGGCGGGGCGGGG
CGAGGGGGGGGCGGGGCGAGGCGGAGAGGTGCGGCGGCAGCCAATCAGAGCGGC
GCGCTCCGAAAGTTTCCTTTTATGGCGAGGCGGCGGCGGCGGCGGCCCTATAAAAA
GCGAAGCGCGCGGCGGGCGGGAGTCGCTGCGACGCTGCCTTCGCCCCGTGCCCCGC
TCCGCCGCCGCCTCGCGCCGCCCGCCCCGGCTCTGACTGACCGCGTTACTCCCACAG
GTGAGCGGGCGGGACGGCCCTTCTCCTCCGGGCTGTAATTAGCGCTTGGTTTAATGA
CGGCTTGTTTCTTTTCTGTGGCTGCGTGAAAGCCTTGAGGGGCTCCGGGAGGGCCCT
TTGTGCGGGGGGGAGCGGCTCGGGGGGTGCGTGCGTGTGTGTGTGCGTGGGGAGCG
CCGCGTGCGGCCCGCGCTGCCCGGCGGCTGTGAGCGCTGCGGGCGCGGCGCGGGGC
TTTGTGCGCTCCGCAGTGTGCGCGAGGGGAGCGCGGCCGGGGGCGGTGCCCCGCGG
TGCGGGGGGGGCTGCGAGGGGAACAAAGGCTGCGTGCGGGGTGTGTGCGTGGGGG
GGTGAGCAGGGGGTGTGGGCGCGGCGGTCGGGCTGTAACCCCCCCCTGCACCCCCC
TCCCCGAGTTGCTGAGCACGGCCCGGCTTCGGGTGCGGGGCTCCGTACGGGGCGTG
GCGCGGGGCTCGCCGTGCCGGGCGGGGGGTGGCGGCAGGTGGGGGTGCCGGGCGG
GGCGGGGCCGCCTCGGGCCGGGGAGGGCTCGGGGGAGGGGCGCGGCGGCCCCCGG
AGCGCCGGCGGCTGTCGAGGCGCGGCGAGCCGCAGCCATTGCCTTTTATGGTAATC
GTGCGAGAGGGCGCAGGGACTTCCTTTGTCCCAAATCTGTGCGGAGCCGAAATCTG
GGAGGCGCCGCCGCACCCCCTCTAGCGGGCGCGGGGCGAAGCGGTGCGGCGCCGGC
AGGAAGGAAATGGGCGGGGAGGGCCTTCGTGCGTCGCCGCGCCGCCGTCCCCTTCT
CCCTCTCCAGCCTCGGGGCTGTCCGCGGGGGGACGGCTGCCTTCGGGGGGGACGGG
GCAGGGCGGGGTTCGGCTTCTGGCGTGTGACCGGCGGCTCTAGAGCCTCTGCTAACC
ATGTTCATGCCTTCTTCTTTTTCCTACAGCTCCTGGGCAACGTGCTGGTTATTGTGCT
GTCTCATCATTTTGGCAAAACCGGTCTCGAAGGCCTGCAGGCGGCCGCCGCCACCGC
CACCATGGTGAGCAAGGGCGAGGAGCTGTTCACCGGGGTGGTGCCCATCCTGGTCG
AGCTGGACGGCGACGTAAACGGCCACAAGTTCAGCGTGTCCGGCGAGGGCGAGGGC
GATGCCACCTACGGCAAGCTGACCCTGAAGTTCATCTGCACCACCGGCAAGCTGCCC
GTGCCCTGGCCCACCCTCGTGACCACCCTGACCTACGGCGTGCAGTGCTTCAGCCGC
TACCCCGACCACATGAAGCAGCACGACTTCTTCAAGTCCGCCATGCCCGAAGGCTAC
GTCCAGGAGCGCACCATCTTCTTCAAGGACGACGGCAACTACAAGACCCGCGCCGA
GGTGAAGTTCGAGGGCGACACCCTGGTGAACCGCATCGAGCTGAAGGGCATCGACT
TCAAGGAGGACGGCAACATCCTGGGGCACAAGCTGGAGTACAACTACAACAGCCAC
AACGTCTATATCATGGCCGACAAGCAGAAGAACGGCATCAAGGTGAACTTCAAGAT
CCGCCACAACATCGAGGACGGCAGCGTGCAGCTCGCCGACCACTACCAGCAGAACA
CCCCCATCGGCGACGGCCCCGTGCTGCTGCCCGACAACCACTACCTGAGCACCCAGT
CCGCCCTGAGCAAAGACCCCAACGAGAAGCGCGATCACATGGTCCTGCTGGAGTTC
GTGACCGCCGCCGGGATCACTCTCGGCATGGACGAGCTGTACAAGTAATCCATGGC
CCAACTTGTTTATTGCAGCTTATAATGGTTACAAATAAAGCAATAGCATCACAAATT
TCACAAATAAAGCATTTTTTTCACTGCATTCTAGTTGTGGTTTGTCCAAACTCATCAA
TGTATCTTATCATGTCTGGATCTCGTTAACTCGAGGGATCCATCGATGTCGACTGCA
GAGGCCTGCATGCAAGCTTGGTGTAATCATGGTCATAGCTGTTTCCTGTGTGAAATT
GTTATCCGCTCACAATTCCACACAACATACGAGCCGGAAGCATAAAGTGTAAAGCC
TGGGGTGCCTAATGAGTGAGCTAACTCACATTAATTGCGTTGCGCTCACTGCCCGCT
TTCCAGTCGGGAAACCTGTCGTGCCAGCTGCATTAATGAATCGGCCAACGCGCGGG
GAGAGGCGGTTTGCGTATTGGGCGCTCTTCCGCTTCCTCGCTCACTGACTCGCTGCG
CTCGGTCGTTCGGCTGCGGCGAGCGGTATCAGCTCACTCAAAGGCGGTAATACGGTT
ATCCACAGAATCAGGGGATAACGCAGGAAAGAACATGTGAGCAAAAGGCCAGCAA
AAGGCCAGGAACCGTAAAAAGGCCGCGTTGCTGGCGTTTTTCCATAGGCTCCGCCCC
CCTGACGAGCATCACAAAAATCGACGCTCAAGTCAGAGGTGGCGAAACCCGACAGG
ACTATAAAGATACCAGGCGTTTCCCCCTGGAAGCTCCCTCGTGCGCTCTCCTGTTCC
GACCCTGCCGCTTACCGGATACCTGTCCGCCTTTCTCCCTTCGGGAAGCGTGGCGCT
TTCTCATAGCTCACGCTGTAGGTATCTCAGTTCGGTGTAGGTCGTTCGCTCCAAGCTG
GGCTGTGTGCACGAACCCCCCGTTCAGCCCGACCGCTGCGCCTTATCCGGTAACTAT
CGTCTTGAGTCCAACCCGGTAAGACACGACTTATCGCCACTGGCAGCAGCCACTGGT
AACAGGATTAGCAGAGCGAGGTATGTAGGCGGTGCTACAGAGTTCTTGAAGTGGTG
GCCTAACTACGGCTACACTAGAAGAACAGTATTTGGTATCTGCGCTCTGCTGAAGCC
AGTTACCTTCGGAAAAAGAGTTGGTAGCTCTTGATCCGGCAAACAAACCACCGCTG
GTAGCGGTGGTTTTTTTGTTTGCAAGCAGCAGATTACGCGCAGAAAAAAAGGATCTC
AAGAAGATCCTTTGATCTTTTCTACGGGGTCTGACGCTCAGTGGAACGAAAACTCAC
GTTAAGGGATTTTGGTCATGAGATTATCAAAAAGGATCTTCACCTAGATCCTTTTAA
ATTAAAAATGAAGTTTTAAATCAAGCCCAATCTGAATAATGTTACAACCAATTAACC
AATTCTGATTAGAAAAACTCATCGAGCATCAAATGAAACTGCAATTTATTCATATCA
GGATTATCAATACCATATTTTTGAAAAAGCCGTTTCTGTAATGAAGGAGAAAACTCA
CCGAGGCAGTTCCATAGGATGGCAAGATCCTGGTATCGGTCTGCGATTCCGACTCGT
CCAACATCAATACAACCTATTAATTTCCCCTCGTCAAAAATAAGGTTATCAAGTGAG
AAATCACCATGAGTGACGACTGAATCCGGTGAGAATGGCAAAAGTTTATGCATTTCT
TTCCAGACTTGTTCAACAGGCCAGCCATTACGCTCGTCATCAAAATCACTCGCATCA
ACCAAACCGTTATTCATTCGTGATTGCGCCTGAGCGAGACGAAATACGCGATCGCTG
TTAAAAGGACAATTACAAACAGGAATCGAATGCAACCGGCGCAGGAACACTGCCAG
CGCATCAACAATATTTTCACCTGAATCAGGATATTCTTCTAATACCTGGAATGCTGTT
TTTCCGGGGATCGCAGTGGTGAGTAACCATGCATCATCAGGAGTACGGATAAAATG
CTTGATGGTCGGAAGAGGCATAAATTCCGTCAGCCAGTTTAGTCTGACCATCTCATC
TGTAACATCATTGGCAACGCTACCTTTGCCATGTTTCAGAAACAACTCTGGCGCATC
GGGCTTCCCATACAAGCGATAGATTGTCGCACCTGATTGCCCGACATTATCGCGAGC
CCATTTATACCCATATAAATCAGCATCCATGTTGGAATTTAATCGCGGCCTCGACGT
TTCCCGTTGAATATGGCTCATAACACCCCTTGTATTACTGTTTATGTAAGCAGACAGT
TTTATTGTTCATGATGATATATTTTTATCTTGTGCAATGTAACATCAGAGATTTTGAG
ACACGGGCCAGAGCTGCA
CBA-Luciferase plasmid
SEQ ID NO: 396
TCGCGCGTTTCGGTGATGACGGTCGAGGTGAGCCCCACGTTCTGCTTCACTCTCCCC
ATCTCCCCCCCCTCCCCACCCCCAATTTTGTATTTATTTATTTTTTAATTATTTTGTGC
AGCGATGGGGGCGGGGGGGGGGGGGGGGCGCGCGCCAGGCGGGGGGGGCGGGG
CGAGGGGCGGGGCGGGGCGAGGCGGAGAGGTGCGGCGGCAGCCAATCAGAGCGGC
GCGCTCCGAAAGTTTCCTTTTATGGCGAGGCGGCGGCGGCGGCGGCCCTATAAAAA
GCGAAGCGCGCGGCGGGCGGGAGTCGCTGCGACGCTGCCTTCGCCCCGTGCCCCGC
TCCGCCGCCGCCTCGCGCCGCCCGCCCCGGCTCTGACTGACCGCGTTACTCCCACAG
GTGAGCGGGCGGGACGGCCCTTCTCCTCCGGGCTGTAATTAGCGCTTGGTTTAATGA
CGGCTTGTTTCTTTTCTGTGGCTGCGTGAAAGCCTTGAGGGGCTCCGGGAGGGCCCT
TTGTGCGGGGGGGAGCGGCTCGGGGGGTGCGTGCGTGTGTGTGTGCGTGGGGAGCG
CCGCGTGCGGCCCGCGCTGCCCGGCGGCTGTGAGCGCTGCGGGCGCGGCGCGGGGC
TTTGTGCGCTCCGCAGTGTGCGCGAGGGGAGCGCGGCCGGGGGCGGTGCCCCGCGG
TGCGGGGGGGGCTGCGAGGGGAACAAAGGCTGCGTGCGGGGTGTGTGCGTGGGGG
GGTGAGCAGGGGGTGTGGGCGCGGCGGTCGGGCTGTAACCCCCCCCTGCACCCCCC
TCCCCGAGTTGCTGAGCACGGCCCGGCTTCGGGTGCGGGGCTCCGTACGGGGCGTG
GCGCGGGGCTCGCCGTGCCGGGCGGGGGGTGGCGGCAGGTGGGGGTGCCGGGCGG
GGCGGGGCCGCCTCGGGCCGGGGAGGGCTCGGGGGAGGGGCGCGGCGGCCCCCGG
AGCGCCGGCGGCTGTCGAGGCGCGGCGAGCCGCAGCCATTGCCTTTTATGGTAATC
GTGCGAGAGGGCGCAGGGACTTCCTTTGTCCCAAATCTGTGCGGAGCCGAAATCTG
GGAGGCGCCGCCGCACCCCCTCTAGCGGGCGCGGGGCGAAGCGGTGCGGCGCCGGC
AGGAAGGAAATGGGCGGGGAGGGCCTTCGTGCGTCGCCGCGCCGCCGTCCCCTTCT
CCCTCTCCAGCCTCGGGGCTGTCCGCGGGGGGACGGCTGCCTTCGGGGGGGACGGG
GCAGGGCGGGGTTCGGCTTCTGGCGTGTGACCGGCGGCTCTAGAGCCTCTGCTAACC
ATGTTCATGCCTTCTTCTTTTTCCTACAGCTCCTGGGCAACGTGCTGGTTATTGTGCT
GTCTCATCATTTTGGCAAAACCGGTCTCGAAGGCCTGCAGGCGGCCGCCGCCACCGC
CACCATGGAAGACGCCAAAAACATAAAGAAAGGCCCGGCGCCATTCTATCCGCTGG
AAGATGGAACCGCTGGAGAGCAACTGCATAAGGCTATGAAGAGATACGCCCTGGTT
CCTGGAACAATTGCTTTTACAGATGCACATATCGAGGTGGACATCACTTACGCTGAG
TACTTCGAAATGTCCGTTCGGTTGGCAGAAGCTATGAAACGATATGGGCTGAATACA
AATCACAGAATCGTCGTATGCAGTGAAAACTCTCTTCAATTCTTTATGCCGGTGTTG
GGCGCGTTATTTATCGGAGTTGCAGTTGCGCCCGCGAACGACATTTATAATGAACGT
GAATTGCTCAACAGTATGGGCATTTCGCAGCCTACCGTGGTGTTCGTTTCCAAAAAG
GGGTTGCAAAAAATTTTGAACGTGCAAAAAAAGCTCCCAATCATCCAAAAAATTAT
TATCATGGATTCTAAAACGGATTACCAGGGATTTCAGTCGATGTACACGTTCGTCAC
ATCTCATCTACCTCCCGGTTTTAATGAATACGATTTTGTGCCAGAGTCCTTCGATAGG
GACAAGACAATTGCACTGATCATGAACTCCTCTGGATCTACTGGTCTGCCTAAAGGT
GTCGCTCTGCCTCATAGAACTGCCTGCGTGAGATTCTCGCATGCCAGAGATCCTATT
TTTGGCAATCAAATCATTCCGGATACTGCGATTTTAAGTGTTGTTCCATTCCATCACG
GTTTTGGAATGTTTACTACACTCGGATATTTGATATGTGGATTTCGAGTCGTCTTAAT
GTATAGATTTGAAGAAGAGCTGTTTCTGAGGAGCCTTCAGGATTACAAGATTCAAAG
TGCGCTGCTGGTGCCAACCCTATTCTCCTTCTTCGCCAAAAGCACTCTGATTGACAA
ATACGATTTATCTAATTTACACGAAATTGCTTCTGGTGGCGCTCCCCTCTCTAAGGAA
GTCGGGGAAGCGGTTGCCAAGAGGTTCCATCTGCCAGGTATCAGGCAAGGATATGG
GCTCACTGAGACTACATCAGCTATTCTGATTACACCCGAGGGGGATGATAAACCGG
GCGCGGTCGGTAAAGTTGTTCCATTTTTTGAAGCGAAGGTTGTGGATCTGGATACCG
GGAAAACGCTGGGCGTTAATCAAAGAGGCGAACTGTGTGTGAGAGGTCCTATGATT
ATGTCCGGTTATGTAAACAATCCGGAAGCGACCAACGCCTTGATTGACAAGGATGG
ATGGCTACATTCTGGAGACATAGCTTACTGGGACGAAGACGAACACTTCTTCATCGT
TGACCGCCTGAAGTCTCTGATTAAGTACAAAGGCTATCAGGTGGCTCCCGCTGAATT
GGAATCCATCTTGCTCCAACACCCCAACATCTTCGACGCAGGTGTCGCAGGTCTTCC
CGACGATGACGCCGGTGAACTTCCCGCCGCCGTTGTTGTTTTGGAGCACGGAAAGAC
GATGACGGAAAAAGAGATCGTGGATTACGTCGCCAGTCAAGTAACAACCGCGAAAA
AGTTGCGCGGAGGAGTTGTGTTTGTGGACGAAGTACCGAAAGGTCTTACCGGAAAA
CTCGACGCAAGAAAAATCAGAGAGATCCTCATAAAGGCCAAGAAGGGCGGAAAGA
TCGCCGTGTAATCCATGGCCCAACTTGTTTATTGCAGCTTATAATGGTTACAAATAA
AGCAATAGCATCACAAATTTCACAAATAAAGCATTTTTTTCACTGCATTCTAGTTGT
GGTTTGTCCAAACTCATCAATGTATCTTATCATGTCTGGATCTCGTTAACTCGAGGGA
TCCATCGATGTCGACTGCAGAGGCCTGCATGCAAGCTTGGTGTAATCATGGTCATAG
CTGTTTCCTGTGTGAAATTGTTATCCGCTCACAATTCCACACAACATACGAGCCGGA
AGCATAAAGTGTAAAGCCTGGGGTGCCTAATGAGTGAGCTAACTCACATTAATTGC
GTTGCGCTCACTGCCCGCTTTCCAGTCGGGAAACCTGTCGTGCCAGCTGCATTAATG
AATCGGCCAACGCGCGGGGAGAGGCGGTTTGCGTATTGGGCGCTCTTCCGCTTCCTC
GCTCACTGACTCGCTGCGCTCGGTCGTTCGGCTGCGGCGAGCGGTATCAGCTCACTC
AAAGGCGGTAATACGGTTATCCACAGAATCAGGGGATAACGCAGGAAAGAACATGT
GAGCAAAAGGCCAGCAAAAGGCCAGGAACCGTAAAAAGGCCGCGTTGCTGGCGTTT
TTCCATAGGCTCCGCCCCCCTGACGAGCATCACAAAAATCGACGCTCAAGTCAGAG
GTGGCGAAACCCGACAGGACTATAAAGATACCAGGCGTTTCCCCCTGGAAGCTCCC
TCGTGCGCTCTCCTGTTCCGACCCTGCCGCTTACCGGATACCTGTCCGCCTTTCTCCC
TTCGGGAAGCGTGGCGCTTTCTCATAGCTCACGCTGTAGGTATCTCAGTTCGGTGTA
GGTCGTTCGCTCCAAGCTGGGCTGTGTGCACGAACCCCCCGTTCAGCCCGACCGCTG
CGCCTTATCCGGTAACTATCGTCTTGAGTCCAACCCGGTAAGACACGACTTATCGCC
ACTGGCAGCAGCCACTGGTAACAGGATTAGCAGAGCGAGGTATGTAGGCGGTGCTA
CAGAGTTCTTGAAGTGGTGGCCTAACTACGGCTACACTAGAAGAACAGTATTTGGTA
TCTGCGCTCTGCTGAAGCCAGTTACCTTCGGAAAAAGAGTTGGTAGCTCTTGATCCG
GCAAACAAACCACCGCTGGTAGCGGTGGTTTTTTTGTTTGCAAGCAGCAGATTACGC
GCAGAAAAAAAGGATCTCAAGAAGATCCTTTGATCTTTTCTACGGGGTCTGACGCTC
AGTGGAACGAAAACTCACGTTAAGGGATTTTGGTCATGAGATTATCAAAAAGGATC
TTCACCTAGATCCTTTTAAATTAAAAATGAAGTTTTAAATCAAGCCCAATCTGAATA
ATGTTACAACCAATTAACCAATTCTGATTAGAAAAACTCATCGAGCATCAAATGAAA
CTGCAATTTATTCATATCAGGATTATCAATACCATATTTTTGAAAAAGCCGTTTCTGT
AATGAAGGAGAAAACTCACCGAGGCAGTTCCATAGGATGGCAAGATCCTGGTATCG
GTCTGCGATTCCGACTCGTCCAACATCAATACAACCTATTAATTTCCCCTCGTCAAA
AATAAGGTTATCAAGTGAGAAATCACCATGAGTGACGACTGAATCCGGTGAGAATG
GCAAAAGTTTATGCATTTCTTTCCAGACTTGTTCAACAGGCCAGCCATTACGCTCGTC
ATCAAAATCACTCGCATCAACCAAACCGTTATTCATTCGTGATTGCGCCTGAGCGAG
ACGAAATACGCGATCGCTGTTAAAAGGACAATTACAAACAGGAATCGAATGCAACC
GGCGCAGGAACACTGCCAGCGCATCAACAATATTTTCACCTGAATCAGGATATTCTT
CTAATACCTGGAATGCTGTTTTTCCGGGGATCGCAGTGGTGAGTAACCATGCATCAT
CAGGAGTACGGATAAAATGCTTGATGGTCGGAAGAGGCATAAATTCCGTCAGCCAG
TTTAGTCTGACCATCTCATCTGTAACATCATTGGCAACGCTACCTTTGCCATGTTTCA
GAAACAACTCTGGCGCATCGGGCTTCCCATACAAGCGATAGATTGTCGCACCTGATT
GCCCGACATTATCGCGAGCCCATTTATACCCATATAAATCAGCATCCATGTTGGAAT
TTAATCGCGGCCTCGACGTTTCCCGTTGAATATGGCTCATAACACCCCTTGTATTACT
GTTTATGTAAGCAGACAGTTTTATTGTTCATGATGATATATTTTTATCTTGTGCAATG
TAACATCAGAGATTTTGAGACACGGGCCAGAGCTGCA

### INCORPORATION BY REFERENCE

The publications and patents referenced in this application have been incorporated in their entirety.

### Non-Patent Literature

1. Lai, Y, Yue, Y and Duan, D Evidence for the failure of adeno-associated virus serotype 5 to package a viral genome ≥8.2 kb. (2010). Mol Ther 18: 75-79.
2. Smith R. H. Adeno-associated virus integration: virus versus vector. Gene Ther. 2008; 15:817-822.
3. Fitzpatrick Z., Leborgne C, Barbon E., et al. Influence of Pre-existing Anti-capsid Neutralizing and Binding Antibodies on AAV Vector Transduction. Mol Ther Methods Clin Dev. 2018 Jun. 15; 9: 119-129.
4. Guerra-Crespo M, Charli J L, Rosales-Garcia V H, Pedraza-Alva G, Perez-Martinez L. Polyethylenimine improves the transfection efficiency of primary cultures of post-mitotic rat fetal hypothalamic neurons. J Neurosci Methods. 2003; 127(2):179-92.
5. Sutapa Barua and Samir Mitragotri. Challenges associated with Penetration of Nanoparticles across Cell and Tissue Barriers: A Review of Current Status and Future Prospects. Nano today. 2014. 9(2): 223-243.
6. Zabner, J., Fasbender, A. J., Moninger, T., Poellinger, D. A., and Welsh, M. J. Cellular and molecular barriers to gene transfer by a cationic lipid. J. Biol. Chem. (1995) 270:18997-19007.
7. Templeton N S, Senzer N (2011) Optimization of Non-Viral Gene Therapeutics Using Bilamellar Invaginated Vesicles. J Genet Syndr Gene Ther S5:002
8. Wilke, M., Fortunati, E., van den Broek, M., Hoogeveen, A. T., and Scholte, B. J. Efficacy of a peptide-based gene delivery system depends on mitotic activity. Gene Ther. (1996) 3:1133-1142.
9. Ge Liu, DeShan Li, Murali K Pasumarthy, et al. 2003. Nanoparticles of Compacted DNA Transfect Postmitotic Cells. The Journal of Biological Chemistry. Vol. 278, No. 35, Issue of August 29, pp. 32578-32586
10. Michael W. Konstan, Pamela B. D., Jefferey S. W., Kathleen A. H., Robert C. S., Laura J. H. M., Tomasz H. K., Susannah L. H., Tamara L. F., Christopher R. G., Sharon M. O., Jennifer M. P., Osman M., Assem G. Z., Robert C. M., and Mark J. C. Compacted DNA Nanoparticles Administered to the Nasal Mucosa of Cystic Fibrosis Subjects Are Safe and Demonstrate Partial to Complete Cystic Fibrosis Transmembrane Regulator Reconstitution. 2004. Human Gene Therapy. 15:1255-1269
11. D'Souza S E, Ginsberg M H, Plow E F. Arginyl-glycyl-aspartic acid (RGD): a cell adhesion motif. Trends Biochem Sci. 1991 July; 16(7):246-50.
12. Christian Hinderer, Nathan Katz, Elizabeth L. Buza, Cecilia Dyer, Tamara Goode, Peter Bell, Laura K. Richman, and James M. Wilson. Severe Toxicity in Nonhuman Primates and Piglets Following High-Dose Intravenous Administration of an Adeno-Associated Virus Vector Expressing Human SMN. 2018. Human Gene Therapy. Vol 29. No 3.
13. Wodrich H, Henaff D, Jammart B, Segura-Morales C, Seelmeir S, et al. (2010) A Capsid-Encoded PPxY-Motif Facilitates Adenovirus Entry. PLoS Pathog 6(3):e1000808.
14. Kailash N. Pandey. Functional roles of short sequence motifs in the endocytosis of membrane receptors. Frontiers in Bioscience 14, 5339-5360, Jun. 1, 2009
15. Claire Sunyach, Angela Jen, Juelin Deng, Kathleen T. Fitzgerald, Yveline Frobert, Jacques Grassi, Mary W. McCaffrey, Roger Morris. The mechanism of internalization of glycosylphosphatidylinositol-anchored prion protein. The EMBO Journal Vol. 22 No. 14. pp. 3591±3601, 2003
16. Modesto Redrejo-Rodrfguez, Daniel Mufloz-Espfn, Isabel Holguera, Mario Mencfa, and Margarita Salas. Functional eukaryotic nuclear localization signals are widespread in terminal proteins of bacteriophages. PNAS. 2012. Vol 109. No 45. 18482-18487.
17. Chee Kai Chan and David A Jans. Enhancement of Polylysine-Mediated Transferrinfection by Nuclear Localization Sequences: Polylysine Does Not Function as a Nuclear Localization Sequence. Human Gene Therapy. Vol 10. No 10. 1999.
18. Jans D A, Moll T, Nasmyth K, Jans P. Cyclin-dependent kinase site-regulated signal-dependent nuclear localization of the SW15 yeast transcription factor in mammalian cells. J Biol Chem. 1995 Jul. 21; 270(29):17064-7.
19. Kirchhausen T, 1999. Adaptors for clathrin-mediated traffic. Annu Rev Cell Dev. 1999; 15:705-32.
20. Stephanie VandeVondele Janos Voros, Jeffrey A. Hubbell. RGD-Grafted Poly-L-lysine-graft (polyethylene glycol) Copolymers Block Non-specific Protein Adsorption While Promoting Cell Adhesion. Biotechnology and Bioengineering, Vol. 82, No. 7, 2003
21. L. Feuz et al.: Small-angle neutron scattering of PLL grafted PEG molecular brushes. Eur. Phys. J. E 23, 237-245 (2007).
22. Sun Tian, Qingsheng Huang, Ying Fang, Jianhua Wu. (2011) FurinDB: a database of 20-residue furin cleavage site motifs, substrates and their associated drugs. International Journal of Molecular Sciences., 12, 1060-1065.
23. Najjar K, Erazo-Oliveras A, Pellois J. Delivery of proteins, peptides or cell-impermeable small molecules into live cells by incubation with the endosomolytic reagent of TAT. J Vis Exp. 2015; 103
24. Tashiro K, Sephel G. C., Weeks B., Sasaki, M., Martin, G. R., Kleinman, H. K. et al. 1989. A synthetic peptide containing the IKVAVA sequence form the A chain of Laminin mediates cell attachment, migration and neurite growth. J. Biol Chem. 264, 16174-16182.
25. Graf, J., Iwamoto, Y., Sasaki, M., Martin, G. R., Kleinman, H. K., Robey, F. A., et al. 1987. Identification of the major epithelial-cell attachment site (yigsr) in the bl-chain of Laminin. J. Invest. Dermatol., 88, 491.
26. Mishra, A., Gordon, V., Yang, L., Coridan, R. and Wong, G. (2008) HIV TAT forms pores in membranes by inducing saddle-splay curvature: potential role of bidentate hydrogen bonding. Angew. Chem., Int. Ed. 47, 2986-2989.
27. Rothbard, J. B., Jessop, T. C. and Wender, P. A. (2005) Adaptive translocation: the
28. role of hydrogen bonding and membrane potential in the uptake of guanidinium-rich transporters into cells. Adv. Drug Deliv. Rev. 57, 495-504.
29. Yuxin Chen, Michael T. Guamieri Adriana I. Vasil, Michael L. Vasil, Colin T. Mant, and Robert S. Hodges. Role of Peptide Hydrophobicity in the Mechanism of Action of-Helical Antimicrobial Peptides. 2007. Antimicrobial Agents and Chemotherapy, April 2007, p. 1398-1406
30. Wu Z, Simister N E. Tryptophan- and dileucine-based endocytosis signals in the neonatal Fc receptor. J Biol Chem. 2001. eb 16; 276(7):5240-7. Epub 2000 Nov. 28.
31. John P. H. Th'ng, Rohyun Sung, Ming Ye Michael J. Hendzel. H1 family histones in the nucleus control of binding and localization by the C-terminal domain. J. Biol. Chem. 2005; 280:27809-27814
32. Cardin A D, Weintraub H J (1989) Molecular modeling of protein-glycosamino-glycan interactions. Arteriosclerosis 9: 21-32.
33. Torrent M, Nogue s MV, Andreu D, Boix E (2012) The “CPC Clip Motif”: A Conserved Structural Signature for Heparin-Binding Proteins. PLoS ONE 7(8): e42692. doi:10.1371/journal.pone.0042692
34. Nelson C. Di Paolo, Oleksandr Kalyuzhniy, and Dmitry M. Shayakhmetov. Fiber Shaft-Chimeric Adenovirus Vectors Lacking the KKTK Motif Efficiently Infect Liver Cells In Vivo. Journal of Virology, November 2007, p. 12249-12259
35. Laetitia Jean, Charlotte Mizon, William J. Larsen, Jacques Mizon and Jean-Philippe Salier. Unmasking a hyaluronan-binding site of the BX7B type in the H3 heavy chain of the inter-a-inhibitor family. Eur. J. Biochem. 268, 544±553 (2001)
36. Kokona Kouzi-Koliakos, George G. Koliakos, EffieC. Tsilibary, Leo T. Furcht S, and Aristidis S. Charonis. Mapping of Three Major Heparin-binding Sites on Laminin and Identification of a Novel Heparin-binding Site on theB1 Chain. The Journal of Biological Chemistry. 1989. Vol 264. No 30.
37. Joji lida, Alexandra M. L. Meijne, Theodore R. Oegema, Jr., Ted A. Yednock, Nicholas L. Kovach, Leo T. Furcht, and James B. McCarthy. A Role of Chondroitin Sulfate Glycosaminoglycan Binding Site in a4p IIntegrin-mediated Melanoma Cell Adhesion. The Journal of Biological Chemistry273, 5955-5962.
38. Melissa S. Maginnis, J. Craig Forrest, Sarah A. Kopecky-Bromberg, S. Kent Dickeson, Samuel A. Santoro, Mary M. Zutter, Glen R. Nemerow, Jeffrey M. Bergelson, and Terence S. Dermody. Beta1 Integrin Mediates Internalization of Mammalian Reovirus. Journal of Virology, March 2006, p. 2760-277
39. Alfred A. Reszka, Yokichi Hayashi, and Alan E Horwitz. Identification of Amino Acid Sequences in the Integrin/31 Cytoplasmic Domain Implicated in Cytoskeletal Association. The Journal of CeU Biology, Volume 117, Number 6, June 1992 1321-1330
40. Kusakawa T, Simakami T, Kaneko S, Yoshioka K, Murakami S. Functional interaction of hepatitis C Virus NS5B with Nucleolin GAR domain. J Biochemistry. 2007. June 141(6) 917-27
41. C. Graham Knight, Laurence F. Morton, Anthony R. Peachey, Danny S. Tuckwell, Richard W. Famdale, and Michael J. Barnes. The Collagen-binding A-domains of Integrins α1β1 and α2β1 Recognize the Same Specific Amino Acid Sequence, GFOGER, in Native (Triple-helical) Collagens. The Journal of Biological Chemistry. 2000. Vol 275. No. 1
42. Kalthoff C, Alves J, Urbanke C, Knorr R, Ungewickell E J. (2002). Unusual structural organization of the endocytic proteins AP180 and epsin 1. J Biol Chem 277: 8209-8216
43. Igor Beitia Ortiz de Zarate, Lilia Cantero-Aguilar, Magalie Longo, Clarisse Berlioz-Torrent, and Flore Rozenberg. Contribution of Endocytic Motifs in the Cytoplasmic Tail of Herpes Simplex Virus Type 1 Glycoprotein B to Virus Replication and Cell-Cell Fusion. Journal of Virology, December 2007, p. 13889-13903
44. Shaynoor Dramsi, Sophie Magnet, Sophie Davison, Michel Arthur. Covalent attachment of proteins to peptidoglycan. FEMS Microbiol Rev32 (2008)307-320
45. Olli Pentikainen, Anna-Marja Hoffren, Johanna Ivaska, Jarmo Kapyla, Tommi Nyronen, Jyrki Heino, and Mark S. Johnson. “RKKH” Peptides from the Snake Venom Metalloproteinase of Bothrops jararaca Bind Near the Metal Ion-dependent Adhesion Site of the Human Integrin a2 I-domain. The Journal of Biological Chemistry. 274, 31493-31505.
46. Thomas Brand. The Popeye Domain Containing Genes and Their Function as cAMP Effector Proteins in Striated Muscle. J. Cardiovasc. Dev. Dis. 2018, 5, 18
47. Asch A S, Silbiger S, Heimer E, Nachman R L. Thrombospondin sequence motif (CSVTCG) is responsible for CD36 binding. Biochemical and biophysical research communications. Feb. 14 1992; 182(3):1208-1217.
48. Nora B Caberoy, Yixiong Zhou1 and Wei Li. Tubby and tubby-like protein 1 are new MerTK ligands for phagocytosis. The EMBO Journal (2010) 29, 3898-3910
49. Chi-Yi Yu, Zhenhua Yuan, Zhongren Cao, Bing Wang, Chunping Qiao, Juan Li, Xiao Xiao. A muscle-targeting peptide displayed on AAV2 improves muscle tropism upon systemic delivery. Gene Ther. 2009 August; 16(8): 953-962
50. H Buning, MU Ried, L Perabo, F M Gerner, NA Huttner, J Enssle and M Hallekn. Receptor targeting of adeno-associated virus vectors. Gene Therapy (2003) 10, 1142-1151.
51. Wischnjow A, Sarko D, Janzer M, Kaufman C, Beijer B, Brings S, Haberkorn U, Larbig G, Kubelbeck A, Mier W. Bioconjugate Chem. 2016; 27:1050-1057.
52. Lorraine M. Work, Hildegard Buning, Ela Hunt, Stuart A. Nicklin, Laura Denby, Nicola Britton, Kristen Leike, Margarete Odenthal, Uta Drebber, Michael Hallek, and Andrew H. Baker. Vascular Bed-Targeted in Vivo Gene Delivery Using Tropism-Modified Adeno-associated Viruses. Molecular Therapy. Vol. 13, No. 4, April 2006
53. Lorraine M. Work, Stuart A. Nicklin, Nick J. R. Brain, Kate L Dishart, Dan J. Von Seggern, Michael Hallek, Hildegard Buning and Andrew H. Baker. Development of Efficient Viral Vectors Selective for Vascular Smooth Muscle Cells. Molecular Therapy Vol. 9, No. 2, February 2004
54. Wadih Arap, Renata Pasqualini, Erkki Ruoslahti. Cancer Treatment by Targeted Drug Delivery to Tumor Vasculature in a Mouse Model. Science. 16 Jan. 1998: Vol. 279, Issue 5349, pp. 377-380
55. Dale D. Hunter, Brenda E. Porter, Joseph W. Mock, Steven R Adams, John R Merlie, and Joshua R. Sanes. Primary Sequence of a Motor Neuron-Selective Adhesive Site in the Synaptic Basal Lamina Protein S-Laminin. Cell, Vol. 59, 905-913, Dec. 1, 1989,
56. Eric Anderson, Sandra Maday, Jeff Sfakianos, Michael Hull, Bettina Winckler, David Sheff, Heike Fölsch, and Ira Mellman. Transcytosis of NgCAM in epithelial cells reflects differential signal recognition on the endocytic and secretory pathways. The Journal of Cell Biology, Vol. 170, No. 4, Aug. 15, 2005 595-605
57. Matthew J. Bottomley. Structures of protein domains that create or recognize histone modifications., EMBO reports 5, 464-469 (2004).
58. Dahlin-Huppe K, Berglund E O., Ranscht B, Stallcup W B. Mutational analysis of the L1 neuronal cell adhesion molecule identifies membrane-proximal amino acids of the cytoplasmic domain that are required for cytoskeletal anchorage. Mol Cell Neurosci. 1997; 9(2):144-56.
59. P Zheng, J Eastman, S V Pol, and S W. Pimplikar. PAT1, a microtubule-interacting protein, recognizes the basolateral sorting signal of amyloid precursor protein Proc. Natl. Acad. Sci. USA. Vol. 95, pp. 14745-14750, December 1998
60. Daniel J.-F. Chinnapen, Himani Chinnapen, David Saslowsky, and Wayne I. Lencer. Rafting with cholera toxin: endocytosis and tra/cking from plasma membrane to ER. FEMS Microbiol Lett. 2007 January; 266(2): 129-137.
61. D. Gowanlock R. Tervo, Bum-Yeol Hwang, Sarada Viswanathan, Loren L. Looger, David V. Schaffer, Alla Y. Karpova. A Designer AAV Variant Permits Efficient Retrograde Access to Projection Neurons. 2016, Neuron 92, 372-382
62. K Inabe, M Nishizawa, S Tajima, K Ikuta, and Y Aida. The YXXL sequences of a transmembrane protein of bovine leukemia virus are required for viral entry and incorporation of viral envelope protein into virions. J. Virol. 1999 February; 73(2):1293-301.
63. Ton-That, H., and O. Schneewind. 2003. Assembly of pili on the surface of C. diphtheriae. Mol. Microbiol.50:1429-1438.
64. Aravind Asokan, Julie B. Hamra, Lakshmanan Govindasamy, Mavis Agbandje-McKenna, and Richard J. Samulski. Adeno-Associated Virus Type 2 Contains an Integrin alpha 5 betal Binding Domain Essential for Viral Cell Entry. Journal of Virology, September 2006, p. 8961-8969
65. Ji-Seon Park, Dong-Hou Kim, Seung-Yong Yoon. Regulation of amyloid precursor protein processing by its KFERQ motif. BMB Rep. 2016; 49(6): 337-342
66. Kobayashi M, Shimomura A, Hagiwara M, Kawakami K. Phosphorylation of ATF-1 enhancesits DNA binding and transcription of the Na,K-ATPase alpha 1 subunit gene promoter. Nucleic Acids Res. 1997; 25(4):877-882. doi:10.1093/nar/25.4.877
67. Marie-Anne Robin, Subbuswamy K. Prabu, Haider Raza, Hindupur K. Anandatheerthavarada, and Narayan G. Avadhani. Phosphorylation Enhances Mitochondrial Targeting of GSTA4-4 through Increased Affinity for Binding to Cytoplasmic Hsp70. THE JOURNAL OF BIOLOGICAL CHEMISTRY. Vol. 278, No. 21, Issue of May 23, pp. 18960-18970, 2003.
68. Rossetto D, Avvakumov N, C6W6 J. Histone phosphorylation: a chromatin modification involved in diverse nuclear events. Epigenetics. 2012; 7(10):1098-1108. doi:10.4161/epi.21975
69. Ojida, A., Mito-Oka, Y., Inoue, M. A., & Hamachi, I. (2002). First artificial receptors and chemosensors toward phosphorylated peptide in aqueous solution. Journal of the American Chemical Society, 124(22), 6256-6258.
70. Anai, T., Nakata, E., Koshi, Y., Ojida, A., & Hamachi, I. (2007). Design of a hybrid biosensor for enhanced phosphopeptide recognition based on a phosphoprotein binding domain coupled with a fluorescent chemosensor. Journal of the American Chemical Society, 129(19), 6232-6239.
71. Somers W S1, Tang J, Shaw G D, Camphausen R T. Insights into the molecular basis of leukocyte tethering and rolling revealed by structures of P- and E-selectin bound to SLe(X) and PSGL-1. Cell. 2000 Oct. 27; 103(3):467-79.
72. Farzan M., Mirzabekov T., Kolchinsky P., Wyatt R., Cayabyab M., Gerard N. P., Gerard C., Sodroski J., Choe H. Tyrosine sulfation of the amino terminus of CCR5 facilitates HIV-1 entry.” Cell 96:667-676(1999)
73. Gelb, M. H., Scholten, J. D., & Sebolt-Leopold, J. S. (1998). Protein prenylation: from discovery to prospects for cancer treatment. Current opinion in chemical biology, 2(1), 40-48.
74. Xie, F., Li, P., Gong, J., Zhang, J., & Ma, J. (2015). The bisphosphonate zoledronic acid effectively targets lung cancer cells by inhibition of protein prenylation. Biochemical and biophysical research communications, 467(4), 664-669.
75. Ochocki J D, Mullen D G, Wattenberg E V, Distefano M D. Evaluation of a cell penetrating prenylated peptide lacking an intrinsic fluorophore via in situ click reaction. Bioorg Med Chem Lett. 2011; 21(17):4998-5001. doi:10.1016/j.bmcl.2011.04.138
76. Hosseini V, Dani C, Geranmayeh M H, Mohammadzadeh F, Nazari Soltan Ahmad S, Darabi M. Wnt lipidation: Roles in trafficking, modulation, and function. J Cell Physiol. 2019; 234(6):8040-8054. doi:10.1002/jcp.27570
77. Walsh, C. T., Gameau-Tsodikova, S., & Gatto Jr, G. J. (2005). Protein posttranslational modifications: the chemistry of proteome diversifications. Angewandte Chemie International Edition, 44(45), 7342-7372.
78. Shi, S. P., Chen, X., Xu, H. D., & Qiu, J. D. (2015). PredHydroxy: computational prediction of protein hydroxylation site locations based on the primary structure. Molecular BioSystems, 11(3), 819-825.
79. Bartels M F, Winterhalter P R, Yu J, Liu Y, Lommel M, Mohrlen F, et al. (2016) Protein OMannosylation in the Murine Brain: Occurrence of Mono-O-Mannosyl Glycans and Identification of New Substrates. PLoS ONE 11(11): e0166119.

| | Publication | | | |
|---|---|---|---|---|
| Publication number | date | Author | Assignee | Title |
| U S. Pat. No. 6,506,890 B1 | Jan. 14, 2003 | Mark J. C | Mark J | Method of nucleic acid compaction |
| | | et al | Cooper et al | |
| US20100203627 A1 | Aug. 12, 2010 | Mark J. C | Copernicus | Long term in vivo transgene |
| | | et al | Therapeutics | expression |
| US20110035819 A1 | Feb. 10, 2011 | Mark J. C | Copernicus | Codon optimized CFTR |
| | | et al | Therapeutics | |
| WO2011017313 A1 | Feb. 10, 2011 | Mark J | Copernicus | Method of administering non-viral |
| | | Cooper | Therapeutics | nucleic acid vectors to the eye |
| U.S. Pat. No. 9,486,540 (B2) | Nov. 8, 2016 | Harmon | Copernicus | Methods for delivery to the central |
| 2016 Nov. 8 | | Bredan, | Therapeutics | nervous system of nucleic acid |
| | | and | | nanoparticles to treat central |
| | | Waszczak | | nervous system disorders |
| | | Barbara | | |
| WO2008137066 (A1) | Nov. 13, 2008 | Naash | Univ | Use of compacted nucleic acids |
| | | Muna I, | Oklahoma, | nanoparticles in non-viral |
| | | and | Copernicus | treatments of ocular diseases. |
| | | Mark J. C | Therapeutics | |
| WO1997030731A2 | Aug. 28, 1997 | Lollo | Immune | Method of preparing |
| | | Charles P. | Response | polynucleotide-carrier complexes |
| | | et. al. | Corp Inc. | for delivery to cells |
| WO1998046274A2 | Oct. 22, 1998 | Burgess | Avanti Polar | Cationic polymers for nucleic |
| | | Stephen | Lipids Inc, | Lipids, Inc. acid transfection |
| | | W et. al. | UAB | |
| | | | Research | |
| | | | Foundation | |
| EP1031626A1 | Aug. 30, 2000 | Erbacher | Qiagen | Method for stabilising and/or |
| | | Christoph | | GmbH isolating nucleic acids |
| | | et al. | | |
| US2014134232 (A1) | May 15, 2014 | Boulikas | Regulon Inc. | Encapsulation of |
| | | Teni | | Plasmid DNA (Lipogenes(TM)) |
| | | | | and Therapeutic Agents with |
| | | | | Nuclear Localization |
| | | | | Signal/Fusogenic Peptide |
| | | | | Conjugates into |
| | | | | Targeted Liposome Complexes |
| U.S. Pat. No. 5,844,107 | Dec. 1, 1998 | Richard, | Case | Compacted nucleic acids and their |
| | | WH. et. al. | Western | delivery to cells |
| | | | Reserve Uni | |
| U.S. Pat. No. 5,166,320 | Nov. 24, 1992 | Wu G. Y | Univ | Carrier system and method for the |
| | | and Wu C. | Connecticut | introduction of genes into |
| | | H. | | mammalian cells |
| US2017258933 (A1) | Sep. 14, 2017 | Jean- | Texas A & | Compositions and methods for the |
| | | Phillipe P. | M Univ Sys | delivery of molecules into live |
| | | | | cells |
| US2017057997 (A1) | Mar. 2, 2017 | Je-Min C. | IUCF-HYU | 1. Cell penetrating peptide and |
| | | et al. | | method for delivering biologically |
| | | | | active substance using same |

1-123. (canceled)

124. An engineered polypeptide comprising: a modified amino acid, wherein the amino acid modification comprises phosphorylation, and wherein the engineered polypeptide comprises an amino acid sequence having at least 90% sequence identity with SEQ ID NO: 399.

125. A composition comprising:
(i) at least one polynucleotide, and
(ii) at least one engineered polypeptide of claim 124.

126. A polynucleotide that encodes the amino acid sequence of an engineered polypeptide of claim 124.

127. The engineered polypeptide of claim 124, wherein the engineered polypeptide comprises SEQ ID NO: 399.

128. A composition comprising:
(i) at least one polynucleotide, and
(ii) at least one engineered polypeptide of claim 127.

129. A polynucleotide that encodes the amino acid sequence of an engineered polypeptide of claim 127.


SEQUENCE LISTING (488 sequences)

SEQ ID NO: 1 - protein, 5 aa, synthetic construct
KRHRK

SEQ ID NO: 2 - skipped

SEQ ID NO: 3 - protein, 4 aa, synthetic construct
RRRR

SEQ ID NO: 4 - protein, 6 aa, synthetic construct
RRRRRR

SEQ ID NO: 5 - skipped

SEQ ID NO: 6 - protein, 6 aa, synthetic construct
RRLARR

SEQ ID NO: 7 - protein, 11 aa, synthetic construct
KKAKAAAKPKK

SEQ ID NO: 8 - protein, 9 aa, synthetic construct
KKDGKKRKR

SEQ ID NO: 9 - protein, 5 aa, synthetic construct
KKKLK

SEQ ID NO: 10 - protein, 6 aa, synthetic construct
KKRIRK

SEQ ID NO: 11 - protein, 5 aa, synthetic construct
RKKSK

SEQ ID NO: 12 - protein, 5 aa, synthetic construct
KKPKK

SEQ ID NO: 13 - protein, 5 aa, synthetic construct
RRHRR

SEQ ID NO: 14 - protein, 5 aa, synthetic construct
RHRRR

SEQ ID NO: 15 - protein, 6 aa, synthetic construct
RRRRHR

SEQ ID NO: 16 - protein, 6 aa, synthetic construct
KRTVRK

SEQ ID NO: 17 - protein, 6 aa, synthetic construct
KRQRNR

SEQ ID NO: 18 - protein, 8 aa, synthetic construct
RVCACPGR

SEQ ID NO: 19 - skipped

SEQ ID NO: 20 - protein, 8 aa, synthetic construct
DEMGLGKT

SEQ ID NO: 21 - skipped

SEQ ID NO: 22 - protein, 7 aa, synthetic construct
HLSQHLN

SEQ ID NO: 23 - protein, 4 aa, synthetic construct
KTQK

SEQ ID NO: 24 - protein, 4 aa, synthetic construct
RFKW

SEQ ID NO: 25 - skipped

SEQ ID NO: 26 - protein, 4 aa, synthetic construct
NRRK

SEQ ID NO: 27 - skipped

SEQ ID NO: 28 - protein, 13 aa, synthetic construct
RPRGRPRKHTVTS

SEQ ID NO: 29 - protein, 7 aa, synthetic construct
WGREERQ

SEQ ID NO: 30 - protein, 5 aa, synthetic construct
NTQIH

SEQ ID NO: 31 - protein, 7 aa, synthetic construct
WNNKTPH

SEQ ID NO: 32 - skipped

SEQ ID NO: 33 - protein, 5 aa, synthetic construct
VNRWS

SEQ ID NO: 34 - skipped

SEQ ID NO: 35 - protein, 8 aa, synthetic construct
ARKKAAKA

SEQ ID NO: 36-37 - skipped (2 entries)

SEQ ID NO: 38 - protein, 10 aa, synthetic construct
WEPSRPFPVD

SEQ ID NO: 39 - protein, 17 aa, synthetic construct
HRRTRKAPKRIRLPHIR

SEQ ID NO: 40 - protein, 17 aa, synthetic construct
KRTGQYKLGSKTGPGQK

SEQ ID NO: 41 - protein, 4 aa, synthetic construct
KKTK

SEQ ID NO: 42 - protein, 9 aa, synthetic construct
KLRSQLVKK

SEQ ID NO: 43 - protein, 9 aa, synthetic construct
RRRCGQKKK

SEQ ID NO: 44 - skipped

SEQ ID NO: 45 - protein, 17 aa, synthetic construct
RIQNLLKITNLRIKFVK

SEQ ID NO: 46 - protein, 11 aa, synthetic construct
KKEKDIMKKTI

SEQ ID NO: 47 - protein, 12 aa, synthetic construct
HGSRFTFHRGSM

SEQ ID NO: 48 - protein, 4 aa, synthetic construct
HRPH

SEQ ID NO: 49 - protein, 4 aa, synthetic construct
DVAR

SEQ ID NO: 50 - protein, 5 aa, synthetic construct
HFNPR

SEQ ID NO: 51 - protein, 4 aa, synthetic construct
WGTE

SEQ ID NO: 52 - protein, 8 aa, synthetic construct
KKQFGAEC

SEQ ID NO: 53 - protein, 19 aa, synthetic construct
RRPRPGTGPGRRPRPRPRP

SEQ ID NO: 54-55 - skipped (2 entries)

SEQ ID NO: 56 - protein, 4 aa, synthetic construct
RGDS

SEQ ID NO: 57 - protein, 11 aa, synthetic construct
TTVVNPKYEGK

SEQ ID NO: 58 - protein, 11 aa, synthetic construct
ERMSQIKRLLS

SEQ ID NO: 59 - protein, 7 aa, synthetic construct
WRHRARS

SEQ ID NO: 60 - protein, 6 aa, synthetic construct
  SITE 3: note = Hydroxyproline
GFPGER

SEQ ID NO: 61 - protein, 5 aa, synthetic construct
LFDLM

SEQ ID NO: 62 - protein, 7 aa, synthetic construct
WGREERQ

SEQ ID NO: 63 - protein, 7 aa, synthetic construct
QSTEKRG

SEQ ID NO: 64 - protein, 5 aa, synthetic construct
LPNTG

SEQ ID NO: 65 - protein, 4 aa, synthetic construct
DSPE

SEQ ID NO: 66 - protein, 7 aa, synthetic construct
QSTEKRG

SEQ ID NO: 67 - protein, 6 aa, synthetic construct
RQGLID

SEQ ID NO: 68 - protein, 4 aa, synthetic construct
RKKH

SEQ ID NO: 69 - skipped

SEQ ID NO: 70 - protein, 5 aa, synthetic construct
YNQYT

SEQ ID NO: 71 - protein, 5 aa, synthetic construct
KWNYK

SEQ ID NO: 72 - protein, 13 aa, synthetic construct
GPQSVKFKSPDQI

SEQ ID NO: 73 - protein, 8 aa, synthetic construct
RVGENWWY

SEQ ID NO: 74 - protein, 9 aa, synthetic construct
RTLQAHHDR

SEQ ID NO: 75 - protein, 10 aa, synthetic construct
RESPFSGSSR

SEQ ID NO: 76 - protein, 9 aa, synthetic construct
REEIQERMR

SEQ ID NO: 77 - protein, 9 aa, synthetic construct
QDSSSFHHQ

SEQ ID NO: 78 - protein, 8 aa, synthetic construct
KKQFGAEC

SEQ ID NO: 79 - protein, 9 aa, synthetic construct
KRALHNAEC

SEQ ID NO: 80 - protein, 11 aa, synthetic construct
KQKIKHVVKLK

SEQ ID NO: 81 - protein, 10 aa, synthetic construct
KLRCQLAKKK

SEQ ID NO: 82 - skipped

SEQ ID NO: 83 - protein, 4 aa, synthetic construct
PPSY

SEQ ID NO: 84 - protein, 7 aa, synthetic construct
FEDNFVP

SEQ ID NO: 85 - protein, 4 aa, synthetic construct
YIRV

SEQ ID NO: 86 - protein, 4 aa, synthetic construct
YADW

SEQ ID NO: 87 - protein, 4 aa, synthetic construct
YTQV

SEQ ID NO: 88 - protein, 6 aa, synthetic construct
KKRPKP

SEQ ID NO: 89 - protein, 5 aa, synthetic construct
SSDDE

SEQ ID NO: 90 - protein, 5 aa, synthetic construct
RRASS

SEQ ID NO: 91 - protein, 8 aa, synthetic construct
  VARIANT 2..3: note = Any amino acid
  VARIANT 6..7: note = Any amino acid
YXXLYXXL

SEQ ID NO: 92 - protein, 5 aa, synthetic construct
LPLTG

SEQ ID NO: 93 - protein, 5 aa, synthetic construct
LAFTG

SEQ ID NO: 94-98 - skipped (5 entries)

SEQ ID NO: 99 - protein, 7 aa, synthetic construct
KRRHPKK

SEQ ID NO: 100 - skipped

SEQ ID NO: 101 - protein, 7 aa, synthetic construct
EPNLPEE

SEQ ID NO: 102-104 - skipped (3 entries)

SEQ ID NO: 105 - protein, 15 aa, synthetic construct
AICKRIPNKKPGKRT

SEQ ID NO: 106 - skipped

SEQ ID NO: 107 - protein, 5 aa, synthetic construct
RCPCR

SEQ ID NO: 108 - protein, 11 aa, synthetic construct
RANVKHLKILN

SEQ ID NO: 109 - protein, 11 aa, synthetic construct
VARLKNNNRQV

SEQ ID NO: 110 - protein, 5 aa, synthetic construct
VRKKP

SEQ ID NO: 111 - protein, 9 aa, synthetic construct
YVRKKPKLK

SEQ ID NO: 112 - protein, 6 aa, synthetic construct
ISRRLI

SEQ ID NO: 113 - protein, 7 aa, synthetic construct
LTKRSRQ

SEQ ID NO: 114 - protein, 9 aa, synthetic construct
NRKISVQRL

SEQ ID NO: 115 - protein, 7 aa, synthetic construct
YYKQRLI

SEQ ID NO: 116 - protein, 7 aa, synthetic construct
KKKYKLK

SEQ ID NO: 117 - protein, 6 aa, synthetic construct
KKRKLE

SEQ ID NO: 118 - protein, 4 aa, synthetic construct
TRSK

SEQ ID NO: 119 - protein, 6 aa, synthetic construct
HRKRKR

SEQ ID NO: 120 - protein, 6 aa, synthetic construct
NKRKRK

SEQ ID NO: 121 - protein, 7 aa, synthetic construct
AEKSKKK

SEQ ID NO: 122 - protein, 4 aa, synthetic construct
RKSK

SEQ ID NO: 123 - protein, 4 aa, synthetic construct
KRVK

SEQ ID NO: 124 - skipped

SEQ ID NO: 125 - protein, 11 aa, synthetic construct
LQQTPLHLAVI

SEQ ID NO: 126 - protein, 4 aa, synthetic construct
RRPR

SEQ ID NO: 127 - protein, 4 aa, synthetic construct
PRPR

SEQ ID NO: 128 - protein, 4 aa, synthetic construct
RPPP

SEQ ID NO: 129 - protein, 7 aa, synthetic construct
RKKRKGK

SEQ ID NO: 130 - protein, 9 aa, synthetic construct
PAAKRVKLD

SEQ ID NO: 131 - protein, 9 aa, synthetic construct
KLKIKRPVK

SEQ ID NO: 132 - protein, 7 aa, synthetic construct
PKKKRKV

SEQ ID NO: 133 - protein, 6 aa, synthetic construct
QRKRQK

SEQ ID NO: 134 - protein, 4 aa, synthetic construct
KRPR

SEQ ID NO: 135 - protein, 6 aa, synthetic construct
RKRRRP

SEQ ID NO: 136 - protein, 9 aa, synthetic construct
KKGRRNRFK

SEQ ID NO: 137 - protein, 29 aa, synthetic construct
RHRDRLNTELDRLASLLPFPQDVINKLDK

SEQ ID NO: 138 - protein, 6 aa, synthetic construct
KRGRKP

SEQ ID NO: 139 - protein, 13 aa, synthetic construct
KKRAGRRIFKETR

SEQ ID NO: 140 - protein, 7 aa, synthetic construct
ASSLNIA

SEQ ID NO: 141 - protein, 12 aa, synthetic construct
SKTFNTHPQSTP

SEQ ID NO: 142 - protein, 16 aa, synthetic construct
YKQCHKKGGHCFPKEK

SEQ ID NO: 143 - protein, 18 aa, synthetic construct
LGKMDCRWKWKCCKKGSG

SEQ ID NO: 144 - protein, 12 aa, synthetic construct
HGSRFTFHRGSM

SEQ ID NO: 145 - protein, 15 aa, synthetic construct
KKEEEKKEEEKKEEE

SEQ ID NO: 146 - protein, 7 aa, synthetic construct
LIFHKEQ

SEQ ID NO: 147 - protein, 10 aa, synthetic construct
KFNKPFVFLI

SEQ ID NO: 148 - protein, 7 aa, synthetic construct
QPEHSST

SEQ ID NO: 149 - protein, 7 aa, synthetic construct
EYHHYNK

SEQ ID NO: 150 - skipped

SEQ ID NO: 151 - protein, 6 aa, synthetic construct
GEKGEP

SEQ ID NO: 152 - protein, 5 aa, synthetic construct
KTKKK

SEQ ID NO: 153 - protein, 6 aa, synthetic construct
KALKKK

SEQ ID NO: 154 - protein, 5 aa, synthetic construct
KGKKK

SEQ ID NO: 155 - protein, 6 aa, synthetic construct
CSVTCG

SEQ ID NO: 156 - skipped

SEQ ID NO: 157 - protein, 10 aa, synthetic construct
YKYNLNGRES

SEQ ID NO: 158 - protein, 4 aa, synthetic construct
YRSL

SEQ ID NO: 159 - protein, 10 aa, synthetic construct
KGGKKKKKKK

SEQ ID NO: 160 - protein, 11 aa, synthetic construct
KKKQYTSIHHG

SEQ ID NO: 161 - protein, 4 aa, synthetic construct
KDEL

SEQ ID NO: 162 - protein, 10 aa, synthetic construct
LADQDYTKTA

SEQ ID NO: 163 - protein, 4 aa, synthetic construct
DDNN

SEQ ID NO: 164 - protein, 8 aa, synthetic construct
SAVTTVVN

SEQ ID NO: 165 - protein, 12 aa, synthetic construct
GRKKRRQRRRPQ

SEQ ID NO: 166 - skipped

SEQ ID NO: 167 - protein, 12 aa, synthetic construct
KSVKKRSVSEIQ

SEQ ID NO: 168 - protein, 8 aa, synthetic construct
NRRKKRAL

SEQ ID NO: 169 - protein, 5 aa, synthetic construct
KFERQ

SEQ ID NO: 170 - protein, 4 aa, synthetic construct
VRGP

SEQ ID NO: 171 - protein, 4 aa, synthetic construct
NKDS

SEQ ID NO: 172 - protein, 4 aa, synthetic construct
ANNR

SEQ ID NO: 173-208 - skipped (36 entries)

SEQ ID NO: 209 - protein, 4 aa, synthetic construct
YTRF

SEQ ID NO: 210 - protein, 4 aa, synthetic construct
GDAY

SEQ ID NO: 211 - protein, 4 aa, synthetic construct
LLEE

SEQ ID NO: 212 - protein, 9 aa, synthetic construct
RKKRRQRRR

SEQ ID NO: 213 - protein, 4 aa, synthetic construct
YKSL

SEQ ID NO: 214 - protein, 4 aa, synthetic construct
YENF

SEQ ID NO: 215 - protein, 4 aa, synthetic construct
FQDL

SEQ ID NO: 216 - protein, 5 aa, synthetic construct
YIGSR

SEQ ID NO: 217 - protein, 5 aa, synthetic construct
IKVAV

SEQ ID NO: 218 - protein, 6 aa, synthetic construct
EFAKFE

SEQ ID NO: 219 - protein, 21 aa, synthetic construct
LLEEEQLRGLGFRQTRGYKSL

SEQ ID NO: 220 - protein, 7 aa, synthetic construct
LIRERTE

SEQ ID NO: 221 - protein, 7 aa, synthetic construct
LVEERTQ

SEQ ID NO: 222 - protein, 6 aa, synthetic construct
IITFTK

SEQ ID NO: 223 - protein, 5 aa, synthetic construct
ILFNK

SEQ ID NO: 224 - protein, 7 aa, synthetic construct
PIRTLSK

SEQ ID NO: 225 - protein, 10 aa, synthetic construct
YGNSPLHRFK

SEQ ID NO: 226 - protein, 6 aa, synthetic construct
FFQKDR

SEQ ID NO: 227 - protein, 4 aa, synthetic construct
KSRP

SEQ ID NO: 228 - skipped

SEQ ID NO: 229 - protein, 4 aa, synthetic construct
YMKM

SEQ ID NO: 230 - protein, 6 aa, synthetic construct
RSSSFG

SEQ ID NO: 231 - protein, 8 aa, synthetic construct
LKIRGRER

SEQ ID NO: 232 - protein, 8 aa, synthetic construct
LKIRGRKR

SEQ ID NO: 233 - protein, 9 aa, synthetic construct
HVIFKKVSR

SEQ ID NO: 234 - protein, 5 aa, synthetic construct
RGPRV

SEQ ID NO: 235 - protein, 8 aa, synthetic construct
RANVKHLK

SEQ ID NO: 236 - protein, 5 aa, synthetic construct
YPKAG

SEQ ID NO: 237 - protein, 5 aa, synthetic construct
YPRTG

SEQ ID NO: 238-240 - skipped (3 entries)

SEQ ID NO: 241 - protein, 6 aa, synthetic construct
GSSGSS

SEQ ID NO: 242-243 - skipped (2 entries)

SEQ ID NO: 244 - protein, 6 aa, synthetic construct
SSSSSS

SEQ ID NO: 245 - protein, 5 aa, synthetic construct
GGSGG

SEQ ID NO: 246 - protein, 8 aa, synthetic construct
GGSGGGGG

SEQ ID NO: 247 - protein, 11 aa, synthetic construct
GGSGGHMGSGG

SEQ ID NO: 248 - protein, 7 aa, synthetic construct
AEAAAKA

SEQ ID NO: 249-252 - skipped (4 entries)

SEQ ID NO: 253 - protein, 8 aa, synthetic construct
AAGAATAA

SEQ ID NO: 254 - protein, 8 aa, synthetic construct
GSGSGSGS

SEQ ID NO: 255 - protein, 5 aa, synthetic construct
GGSSG

SEQ ID NO: 256-335 - skipped (80 entries)

SEQ ID NO: 336 - protein, 34 aa, synthetic construct
KRHRKLREKRHRKLRRRRRLKRHRKKRHRKLREK

SEQ ID NO: 337 - protein, 40 aa, synthetic construct
KRHRKGSSLREKRHRKLRRRRRLKRHRKKRHRKLREGGSK

SEQ ID NO: 338 - protein, 44 aa, synthetic construct
KRHRKREGSSLREKRHRKNDLRRRRRLKRHRKKRHRKLREGGSK

SEQ ID NO: 339 - protein, 44 aa, synthetic construct
KKPKKREGSSLREKRHRKNDLRRRRRLKRHRKKRHRKLREGGSK

SEQ ID NO: 340 - protein, 41 aa, synthetic construct
RRLARRGSSLREKRHRKLRRRRRLKKPKKKRHRKLREGGSK

SEQ ID NO: 341 - protein, 36 aa, synthetic construct
KRHRKLREKRHRKLREKRHRKLKRHRKKRHRKLREK

SEQ ID NO: 342 - protein, 40 aa, synthetic construct
KRHRKRILREKRHRKLREARKRHRKLKRHRKKRHRKLREK

SEQ ID NO: 343 - protein, 35 aa, synthetic construct
KRHRKKGKKKKGEKGKKKLKGKKKLRRRRRRRQRR

SEQ ID NO: 344 - protein, 43 aa, synthetic construct
KRHRKAPAPKGKKKKGEKGKKKLKGKKKLKPKPRRRRRRRQRR

SEQ ID NO: 345 - protein, 41 aa, synthetic construct
KRHRKGGSGGKGKKKKGEKGKKKLKGKKKLARRRRRRRQRR

SEQ ID NO: 346 - protein, 34 aa, synthetic construct
KRHRKLREKRHRKRRRRRRRKRHRKLREKRRQRR

SEQ ID NO: 347 - protein, 30 aa, synthetic construct
KRHRKKRHRKKRVKKKRHRKRRRRRRDSLL

SEQ ID NO: 348 - protein, 35 aa, synthetic construct
KRHRKKRHRKYQKRVKKKRHRKSSSRRRRRRDSLL

SEQ ID NO: 349 - protein, 32 aa, synthetic construct
KRHRKKKEEEKKEEEKKEEEKRRRRRRRQRRR

SEQ ID NO: 350 - protein, 36 aa, synthetic construct
KRHRKWRKKEEEKKEEEKKEEEKRIRRRRRRRQRRR

SEQ ID NO: 351 - protein, 29 aa, synthetic construct
KRHRKRGDKRHRKRRRRRKRHRKTPHKKK

SEQ ID NO: 352 - protein, 33 aa, synthetic construct
KRHRKFIRGDKRHRKRRRRRKRHRKLATPHKKK

SEQ ID NO: 353 - protein, 36 aa, synthetic construct
KRHRKRGDKRHRKRRRRRKRHRKGSSRNTPHQKKKK

SEQ ID NO: 354 - protein, 33 aa, synthetic construct
KRHRKRGDKRHRKLKRHRKRRRRKRHRKTPHKK

SEQ ID NO: 355 - protein, 30 aa, synthetic construct
KRHRKRGDKRHRKKRHRKKRHRKRGDKKTK

SEQ ID NO: 356 - protein, 32 aa, synthetic construct
KRHRKRGDKKRKKKKRGDKKRRRRRKKKPPSY

SEQ ID NO: 357 - protein, 32 aa, synthetic construct
KRHRKRKRKRKRRRRRKKKRASSLNIAKRRRR

SEQ ID NO: 358 - protein, 32 aa, synthetic construct
KRKKRKGKRLKRRREKRHRKRASSLNIAKKKK

SEQ ID NO: 359 - protein, 32 aa, synthetic construct
KRKKRRLKRKRKRRRRREKRHRKRRRQRRRKK

SEQ ID NO: 360 - protein, 31 aa, synthetic construct
KRKKRRKRKRRRRRKRHRKLRERKRRLREKK

SEQ ID NO: 361 - protein, 34 aa, synthetic construct
KRKNGRKRKRKKRHRKKKKRRRRKRHRKNGRKKK

SEQ ID NO: 362 - protein, 29 aa, synthetic construct
KRHRKWRHRARSKRHRKKKKKKRKKRKGK

SEQ ID NO: 363 - protein, 31 aa, synthetic construct
KRHRKRGDKRHRKKKKNRRKKRALRKKRKGK

SEQ ID NO: 364 - protein, 38 aa, synthetic construct
KKRKRGGKTKKKAKKALKKKKKGKKKKRRRRKKAAPKK

SEQ ID NO: 365 - protein, 43 aa, synthetic construct
KKKAYPKALKKPKKKKKAYPKALKRRRRRKNRRKKRALKRHRK

SEQ ID NO: 366 - protein, 39 aa, synthetic construct
KTRSKKKKKRGDKKKKNRRKKRALNTQIHKKKKKAAPKK

SEQ ID NO: 367 - protein, 34 aa, synthetic construct
KGKKKKGEKGKKKLKGKKKLRRRRRSPKKRRQRR

SEQ ID NO: 368 - protein, 34 aa, synthetic construct; identical to SEQ ID NO: 336

SEQ ID NO: 369 - protein, 36 aa, synthetic construct; identical to SEQ ID NO: 341

SEQ ID NO: 370 - protein, 35 aa, synthetic construct; identical to SEQ ID NO: 343

SEQ ID NO: 371 - protein, 34 aa, synthetic construct; identical to SEQ ID NO: 346

SEQ ID NO: 372 - protein, 30 aa, synthetic construct; identical to SEQ ID NO: 347

SEQ ID NO: 373 - protein, 32 aa, synthetic construct; identical to SEQ ID NO: 349

SEQ ID NO: 374 - protein, 36 aa, synthetic construct
KRHRKQSKKEEEKKEEEKKEEEKNQRRRRRRRQRRR

SEQ ID NO: 375 - protein, 29 aa, synthetic construct
KRHRKRGDKRHRKRRRRRKRHRKTPHKKK

SEQ ID NO: 376 - protein, 33 aa, synthetic construct; identical to SEQ ID NO: 354

SEQ ID NO: 377 - protein, 30 aa, synthetic construct; identical to SEQ ID NO: 355

SEQ ID NO: 378 - protein, 32 aa, synthetic construct; identical to SEQ ID NO: 356

SEQ ID NO: 379 - protein, 38 aa, synthetic construct
KRHRKGGSRGDKKRKKKKRGDSSSKKRRRRRKKKPPSY

SEQ ID NO: 380 - protein, 32 aa, synthetic construct; identical to SEQ ID NO: 357

SEQ ID NO: 381 - protein, 32 aa, synthetic construct; identical to SEQ ID NO: 358

SEQ ID NO: 382 - protein, 32 aa, synthetic construct; identical to SEQ ID NO: 359

SEQ ID NO: 383 - protein, 31 aa, synthetic construct; identical to SEQ ID NO: 360

SEQ ID NO: 384 - protein, 34 aa, synthetic construct; identical to SEQ ID NO: 361

SEQ ID NO: 385 - protein, 40 aa, synthetic construct
KRKWRNGRKRKRQKRHRKKKKRARRRRKRHRKNGRKHKKK

SEQ ID NO: 386 - protein, 29 aa, synthetic construct
KRHRKWRHRARSKRHRKKKPKKRKKRKGK

SEQ ID NO: 387 - protein, 36 aa, synthetic construct
KRHRKPKPRIWRHRARSRDKRHRKKKPKKRKKRKGK

SEQ ID NO: 388 - protein, 41 aa, synthetic construct
KKKRKLRGDLKRKGSSYQPLAPAPKKKRKRGDKRKLFYQPL

SEQ ID NO: 389-392 - skipped (4 entries)

SEQ ID NO: 393 - protein, 31 aa, synthetic construct
CKKKKKKKKKKKKKKKKKKKKKKKKKKKKKK

SEQ ID NO: 394 - skipped

SEQ ID NO: 395 - DNA, 4431 bp, synthetic construct
  source 1..4431: mol_type = other DNA
tcgcgcgtttcggtgatgacggtcgaggtgagccccacgttctgcttcactctccccatctcccccccctccccacccccaattttgtatttatttattttttaattattttgtgcagcgatgggggcggggggggggggggggcgcgcgccaggcggggcggggcggggcgaggggcggggcggggcgaggcggagaggtgcggcggcagccaatcagagcggcgcgctccgaaagtttccttttatggcgaggcggcggcggcggcggccctataaaaagcgaagcgcgcggcgggcgggagtcgctgcgacgctgccttcgccccgtgccccgctccgccgccgcctcgcgccgcccgccccggctctgactgaccgcgttactcccacaggtgagcgggcgggacggcccttctcctccgggctgtaattagcgcttggtttaatgacggcttgtttcttttctgtggctgcgtgaaagccttgaggggctccgggagggccctttgtgcgggggggagcggctcggggggtgcgtgcgtgtgtgtgtgcgtggggagcgccgcgtgcggcccgcgctgcccggcggctgtgagcgctgcgggcgcggcgcggggctttgtgcgctccgcagtgtgcgcgaggggagcgcggccgggggcggtgccccgcggtgcggggggggctgcgaggggaacaaaggctgcgtgcggggtgtgtgcgtgggggggtgagcagggggtgtgggcgcggcggtcgggctgtaacccccccctgcacccccctccccgagttgctgagcacggcccggcttcgggtgcggggctccgtacggggcgtggcgcggggctcgccgtgccgggcggggggtggcggcaggtgggggtgccgggcggggcggggccgcctcgggccggggagggctcgggggaggggcgcggcggcccccggagcgccggcggctgtcgaggcgcggcgagccgcagccattgccttttatggtaatcgtgcgagagggcgcagggacttcctttgtcccaaatctgtgcggagccgaaatctgggaggcgccgccgcaccccctctagcgggcgcggggcgaagcggtgcggcgccggcaggaaggaaatgggcggggagggccttcgtgcgtcgccgcgccgccgtccccttctccctctccagcctcggggctgtccgcggggggacggctgccttcgggggggacggggcagggcggggttcggcttctggcgtgtgaccggcggctctagagcctctgctaaccatgttcatgccttcttctttttcctacagctcctgggcaacgtgctggttattgtgctgtctcatcattttggcaaaaccggtctcgaaggcctgcaggcggccgccgccaccgccaccatggtgagcaagggcgaggagctgttcaccggggtggtgcccatcctggtcgagctggacggcgacgtaaacggccacaagttcagcgtgtccggcgagggcgagggcgatgccacctacggcaagctgaccctgaagttcatctgcaccaccggcaagctgcccgtgccctggcccaccctcgtgaccaccctgacctacggcgtgcagtgcttcagccgctaccccgaccacatgaagcagcacgacttcttcaagtccgccatgcccgaaggctacgtccaggagcgcaccatcttcttcaaggacgacggcaactacaagacccgcgccgaggtgaagttcgagggcgacaccctggtgaaccgcatcgagctgaagggcatcgacttcaaggaggacggcaacatcctggggcacaagctggagtacaactacaacagccacaacgtctatatcatggccgacaagcagaagaacggcatcaaggtgaacttcaagatccgccacaacatcgaggacggcagcgtgcagctcgccgaccactaccagcagaacacccccatcggcgacggccccgtgctgctgcccgacaaccactacctgagcacccagtccgccctgagcaaagaccccaacgagaagcgcgatcacatggtcctgctggagttcgtgaccgccgccgggatcactctcggcatggacgagctgtacaagtaatccatggcccaacttgtttattgcagcttataatggttacaaataaagcaatagcatcacaaatttcacaaataaagcatttttttcactgcattctagttgtggtttgtccaaactcatcaatgtatcttatcatgtctggatctcgttaactcgagggatccatcgatgtcgactgcagaggcctgcatgcaagcttggtgtaatcatggtcatagctgtttcctgtgtgaaattgttatccgctcacaattccacacaacatacgagccggaagcataaagtgtaaagcctggggtgcctaatgagtgagctaactcacattaattgcgttgcgctcactgcccgctttccagtcgggaaacctgtcgtgccagctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaagcccaatctgaataatgttacaaccaattaaccaattctgattagaaaaactcatcgagcatcaaatgaaactgcaatttattcatatcaggattatcaataccatatttttgaaaaagccgtttctgtaatgaaggagaaaactcaccgaggcagttccataggatggcaagatcctggtatcggtctgcgattccgactcgtccaacatcaatacaacctattaatttcccctcgtcaaaaataaggttatcaagtgagaaatcaccatgagtgacgactgaatccggtgagaatggcaaaagtttatgcatttctttccagacttgttcaacaggccagccattacgctcgtcatcaaaatcactcgcatcaaccaaaccgttattcattcgtgattgcgcctgagcgagacgaaatacgcgatcgctgttaaaaggacaattacaaacaggaatcgaatgcaaccggcgcaggaacactgccagcgcatcaacaatattttcacctgaatcaggatattcttctaatacctggaatgctgtttttccggggatcgcagtggtgagtaaccatgcatcatcaggagtacggataaaatgcttgatggtcggaagaggcataaattccgtcagccagtttagtctgaccatctcatctgtaacatcattggcaacgctacctttgccatgtttcagaaacaactctggcgcatcgggcttcccatacaagcgatagattgtcgcacctgattgcccgacattatcgcgagcccatttatacccatataaatcagcatccatgttggaatttaatcgcggcctcgacgtttcccgttgaatatggctcataacaccccttgtattactgtttatgtaagcagacagttttattgttcatgatgatatatttttatcttgtgcaatgtaacatcagagattttgagacacgggccagagctgca

SEQ ID NO: 396 - DNA, 5364 bp, synthetic construct
  source 1..5364: mol_type = other DNA
tcgcgcgtttcggtgatgacggtcgaggtgagccccacgttctgcttcactctccccatctcccccccctccccacccccaattttgtatttatttattttttaattattttgtgcagcgatgggggcggggggggggggggggcgcgcgccaggcggggcggggcggggcgaggggcggggcggggcgaggcggagaggtgcggcggcagccaatcagagcggcgcgctccgaaagtttccttttatggcgaggcggcggcggcggcggccctataaaaagcgaagcgcgcggcgggcgggagtcgctgcgacgctgccttcgccccgtgccccgctccgccgccgcctcgcgccgcccgccccggctctgactgaccgcgttactcccacaggtgagcgggcgggacggcccttctcctccgggctgtaattagcgcttggtttaatgacggcttgtttcttttctgtggctgcgtgaaagccttgaggggctccgggagggccctttgtgcgggggggagcggctcggggggtgcgtgcgtgtgtgtgtgcgtggggagcgccgcgtgcggcccgcgctgcccggcggctgtgagcgctgcgggcgcggcgcggggctttgtgcgctccgcagtgtgcgcgaggggagcgcggccgggggcggtgccccgcggtgcggggggggctgcgaggggaacaaaggctgcgtgcggggtgtgtgcgtgggggggtgagcagggggtgtgggcgcggcggtcgggctgtaacccccccctgcacccccctccccgagttgctgagcacggcccggcttcgggtgcggggctccgtacggggcgtggcgcggggctcgccgtgccgggcggggggtggcggcaggtgggggtgccgggcggggcggggccgcctcgggccggggagggctcgggggaggggcgcggcggcccccggagcgccggcggctgtcgaggcgcggcgagccgcagccattgccttttatggtaatcgtgcgagagggcgcagggacttcctttgtcccaaatctgtgcggagccgaaatctgggaggcgccgccgcaccccctctagcgggcgcggggcgaagcggtgcggcgccggcaggaaggaaatgggcggggagggccttcgtgcgtcgccgcgccgccgtccccttctccctctccagcctcggggctgtccgcggggggacggctgccttcgggggggacggggcagggcggggttcggcttctggcgtgtgaccggcggctctagagcctctgctaaccatgttcatgccttcttctttttcctacagctcctgggcaacgtgctggttattgtgctgtctcatcattttggcaaaaccggtctcgaaggcctgcaggcggccgccgccaccgccaccatggaagacgccaaaaacataaagaaaggcccggcgccattctatccgctggaagatggaaccgctggagagcaactgcataaggctatgaagagatacgccctggttcctggaacaattgcttttacagatgcacatatcgaggtggacatcacttacgctgagtacttcgaaatgtccgttcggttggcagaagctatgaaacgatatgggctgaatacaaatcacagaatcgtcgtatgcagtgaaaactctcttcaattctttatgccggtgttgggcgcgttatttatcggagttgcagttgcgcccgcgaacgacatttataatgaacgtgaattgctcaacagtatgggcatttcgcagcctaccgtggtgttcgtttccaaaaaggggttgcaaaaaattttgaacgtgcaaaaaaagctcccaatcatccaaaaaattattatcatggattctaaaacggattaccagggatttcagtcgatgtacacgttcgtcacatctcatctacctcccggttttaatgaatacgattttgtgccagagtccttcgatagggacaagacaattgcactgatcatgaactcctctggatctactggtctgcctaaaggtgtcgctctgcctcatagaactgcctgcgtgagattctcgcatgccagagatcctatttttggcaatcaaatcattccggatactgcgattttaagtgttgttccattccatcacggttttggaatgtttactacactcggatatttgatatgtggatttcgagtcgtcttaatgtatagatttgaagaagagctgtttctgaggagccttcaggattacaagattcaaagtgcgctgctggtgccaaccctattctccttcttcgccaaaagcactctgattgacaaatacgatttatctaatttacacgaaattgcttctggtggcgctcccctctctaaggaagtcggggaagcggttgccaagaggttccatctgccaggtatcaggcaaggatatgggctcactgagactacatcagctattctgattacacccgagggggatgataaaccgggcgcggtcggtaaagttgttccattttttgaagcgaaggttgtggatctggataccgggaaaacgctgggcgttaatcaaagaggcgaactgtgtgtgagaggtcctatgattatgtccggttatgtaaacaatccggaagcgaccaacgccttgattgacaaggatggatggctacattctggagacatagcttactgggacgaagacgaacacttcttcatcgttgaccgcctgaagtctctgattaagtacaaaggctatcaggtggctcccgctgaattggaatccatcttgctccaacaccccaacatcttcgacgcaggtgtcgcaggtcttcccgacgatgacgccggtgaacttcccgccgccgttgttgttttggagcacggaaagacgatgacggaaaaagagatcgtggattacgtcgccagtcaagtaacaaccgcgaaaaagttgcgcggaggagttgtgtttgtggacgaagtaccgaaaggtcttaccggaaaactcgacgcaagaaaaatcagagagatcctcataaaggccaagaagggcggaaagatcgccgtgtaatccatggcccaacttgtttattgcagcttataatggttacaaataaagcaatagcatcacaaatttcacaaataaagcatttttttcactgcattctagttgtggtttgtccaaactcatcaatgtatcttatcatgtctggatctcgttaactcgagggatccatcgatgtcgactgcagaggcctgcatgcaagcttggtgtaatcatggtcatagctgtttcctgtgtgaaattgttatccgctcacaattccacacaacatacgagccggaagcataaagtgtaaagcctggggtgcctaatgagtgagctaactcacattaattgcgttgcgctcactgcccgctttccagtcgggaaacctgtcgtgccagctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaagcccaatctgaataatgttacaaccaattaaccaattctgattagaaaaactcatcgagcatcaaatgaaactgcaatttattcatatcaggattatcaataccatatttttgaaaaagccgtttctgtaatgaaggagaaaactcaccgaggcagttccataggatggcaagatcctggtatcggtctgcgattccgactcgtccaacatcaatacaacctattaatttcccctcgtcaaaaataaggttatcaagtgagaaatcaccatgagtgacgactgaatccggtgagaatggcaaaagtttatgcatttctttccagacttgttcaacaggccagccattacgctcgtcatcaaaatcactcgcatcaaccaaaccgttattcattcgtgattgcgcctgagcgagacgaaatacgcgatcgctgttaaaaggacaattacaaacaggaatcgaatgcaaccggcgcaggaacactgccagcgcatcaacaatattttcacctgaatcaggatattcttctaatacctggaatgctgtttttccggggatcgcagtggtgagtaaccatgcatcatcaggagtacggataaaatgcttgatggtcggaagaggcataaattccgtcagccagtttagtctgaccatctcatctgtaacatcattggcaacgctacctttgccatgtttcagaaacaactctggcgcatcgggcttcccatacaagcgatagattgtcgcacctgattgcccgacattatcgcgagcccatttatacccatataaatcagcatccatgttggaatttaatcgcggcctcgacgtttcccgttgaatatggctcataacaccccttgtattactgtttatgtaagcagacagttttattgttcatgatgatatatttttatcttgtgcaatgtaacatcagagattttgagacacgggccagagctgca

SEQ ID NO: 397 - protein, 32 aa, synthetic construct
KKRHRKYPKKSRRSRLRNFRGDYNQYTRRRRR

SEQ ID NO: 398 - protein, 33 aa, synthetic construct
KRKKRHRKRIRGRDVKYSYARKRHRKFQKWNYK

SEQ ID NO: 399 - protein, 32 aa, synthetic construct
KKRHRKARRVTALREGRRHRKGERRRRRPPSY

SEQ ID NO: 400 - protein, 32 aa, synthetic construct
KKRHRKALGSSDSLLARKRHRKKRKRKKRHRK

SEQ ID NO: 401 - protein, 34 aa, synthetic construct
KKRHRKGSSKKRPKPRKKRHRKKRHRKKRHRKLL

SEQ ID NO: 402 - protein, 36 aa, synthetic construct
KKRHRKRIQRRSRRGSSKHKGRDVILKKDVRKRHRK

SEQ ID NO: 403 - protein, 36 aa, synthetic construct
KKRHRKKKDGKKRKRLLRKKHARALYIGSRKRGRKP

SEQ ID NO: 404 - protein, 33 aa, synthetic construct
KKRHRKPPKDGEAQPKRHRKRRRRRKRHRKLRA

SEQ ID NO: 405 - protein, 34 aa, synthetic construct
KKRKKRHRKLARGPRVARKRHRKRRRRRDRYQRL

SEQ ID NO: 406 - protein, 38 aa, synthetic construct
KKRHRKRGFRRVSRRRGKKKEQRRERNARGKKGKRHRK

SEQ ID NO: 407 - protein, 39 aa, synthetic construct
KKRHRKRRQPPRSISSHPLRKKRKGKTRRLRGDLRNSRR

SEQ ID NO: 408 - protein, 34 aa, synthetic construct
KKRHRKRLRKKRKGKGSRPGSGFVKKTKQRRRRR

SEQ ID NO: 409 - protein, 39 aa, synthetic construct
KKRHRKHRTKSGRSRIRKKRKGKRHARKKRRQRRRPPSY

SEQ ID NO: 410 - protein, 32 aa, synthetic construct
KKRHRKKPVNRWSARNRRKKRALLRRRHYQRL

SEQ ID NO: 411 - protein, 36 aa, synthetic construct
KKRHRKRKYKQCHKKGGHCFPKEKARRKKRKGKNEI

SEQ ID NO: 412 - protein, 35 aa, synthetic construct
KKRHRKRIKKYRYYLKPLKKKRKKRKGKRHYLIIR

SEQ ID NO: 413 - protein, 34 aa, synthetic construct
KKRHRKDRGRKKRRQRRRPQKPRKKRRQRRFQQI

SEQ ID NO: 414 - protein, 36 aa, synthetic construct
KKRHRKGSSDPFRDDPFHRKRHRKKRHRKKRHRGRR

SEQ ID NO: 415 - protein, 37 aa, synthetic construct
KKRHRKARSKTFNTHPQSTPYKRHRKRKKRKGKKRPK

SEQ ID NO: 416 - protein, 36 aa, synthetic construct
RKKRKGKRAKRHRKKRHRKKPKNMTPYRSPPPYVPP

SEQ ID NO: 417 - protein, 30 aa, synthetic construct
KKKRKRGDKRKRKRHRKKKRRRRLSIPPKA

SEQ ID NO: 418 - protein, 30 aa, synthetic construct
KKKRKRGDKRKRKRHRKKKRRRRFQTPPQL

SEQ ID NO: 419 - protein, 30 aa, synthetic construct
KKKRKRGDKRKRKRHRKKKRRRRLTPATAI

SEQ ID NO: 420 - protein, 30 aa, synthetic construct
KKKRKRGDKRKRKRHRKKKRRRRSIGYPLP

SEQ ID NO: 421 - protein, 32 aa, synthetic construct
KKKRKRGDKRKRKRHRKKKRRRRCLIRRTSIC

SEQ ID NO: 422 - protein, 33 aa, synthetic construct
KKKRKRGDKRKRKRHRKKKRRRRCFFWKFRWMC

SEQ ID NO: 423 - protein, 33 aa, synthetic construct
KKKRKRGDKRKRKRHRKKKRRRREYYLSIPPKA

SEQ ID NO: 424 - protein, 32 aa, synthetic construct
KKKRKRGDKRKRKRHRKKKRRRDYRFQTPPQL

SEQ ID NO: 425 - protein, 32 aa, synthetic construct
KKKRKRGDKRKRKRHRKKKRRRHYRLTPATAI

SEQ ID NO: 426 - protein, 33 aa, synthetic construct
KKKRKRGDKRKRKRHRKKKRRRRVYQSIGYPLP

SEQ ID NO: 427 - protein, 35 aa, synthetic construct
KKKRKSYRRGDKRKRKRHRKKKRRRRCLIRRTSIC

SEQ ID NO: 428 - protein, 35 aa, synthetic construct
KKKRKEYRGDKRKRKRHRKKKRRRRCFFWKFRWMC

SEQ ID NO: 429 - protein, 35 aa, synthetic construct
KKRHRKARARKKAAKARIKKAAPAKKAANRARKKH

SEQ ID NO: 430 - protein, 40 aa, synthetic construct
KKRHRKGSSRRPRPGTGPGRRPRPRPRPRKKRNRSRQRRR

SEQ ID NO: 431 - protein, 34 aa, synthetic construct
KKRHRKKYKQKIKHVVKLKKHRKRKRNRSIKVAV

SEQ ID NO: 432 - protein, 34 aa, synthetic construct
KKRHRKSSSRTLQAHHDRQSNKRKRKNRSRRRRR

SEQ ID NO: 433 - protein, 36 aa, synthetic construct
KKRHRKRNRSIHFNPRHRRRRRRDVARARAEKSKKK

SEQ ID NO: 434 - protein, 38 aa, synthetic construct
KKRHRKNRSKKQRFRHRNRKGYRSQRGHSRGRNQNSRR

SEQ ID NO: 435 - protein, 38 aa, synthetic construct
KKRHRKARARKKAAKARIKKAAPAKKAARACIILRKKH

SEQ ID NO: 436 - protein, 41 aa, synthetic construct
KKRHRKGSSRRPRPGTGPGRRPRPRPRPRKKRCASERQRRR

SEQ ID NO: 437 - protein, 35 aa, synthetic construct
KKRHRKCIIEKYKQKIKHVVKLKKHRKRKRIKVAV

SEQ ID NO: 438 - protein, 35 aa, synthetic construct
KKRHRKCQALSSSRTLQAHHDRQSNKRKRKRRRRR

SEQ ID NO: 439 - protein, 37 aa, synthetic construct
KKRHRKRIHFNPRHRRRRRRCIAEDVARARAEKSKKK

SEQ ID NO: 440 - protein, 39 aa, synthetic construct
KRHRKKKQRFRHRNRKGYRSQRGHSRGRNQNSRRCIILR

SEQ ID NO: 441 - protein, 34 aa, synthetic construct
KKRHRKARARKKAAKARIKKAAPAKKAARARKKH

SEQ ID NO: 442 - protein, 37 aa, synthetic construct
KKRHRKGSSRRPRPGTGPGRRPRPRPRPRKKRRQRRR

SEQ ID NO: 443 - protein, 31 aa, synthetic construct
KKRHRKKYKQKIKHVVKLKKHRKRKRIKVAV

SEQ ID NO: 444 - protein, 31 aa, synthetic construct
KKRHRKSSSRTLQAHHDRQSNKRKRKRRRRR

SEQ ID NO: 445 - protein, 33 aa, synthetic construct
KKRHRKRIHFNPRHRRRRRRDVARARAEKSKKK

SEQ ID NO: 446 - protein, 34 aa, synthetic construct
KRHRKKKQRFRHRNRKGYRSQRGHSRGRNQNSRR

SEQ ID NO: 447 - protein, 32 aa, synthetic construct
KKRHRKGGSLLRGEKELKRPPRRRRRKYIGSR

SEQ ID NO: 448 - protein, 36 aa, synthetic construct
KKKRKLRGDLKRKPLISRRLIDRYQKKKRKRGDKRK

SEQ ID NO: 449 - protein, 35 aa, synthetic construct
KKKRKLRGDLKRKSSSVRKKPGGSKKKRKRGDKRK

SEQ ID NO: 450 - protein, 35 aa, synthetic construct
KKKRKLRGDLKRKGTQPEHSSTDHKKKRKRGDKRK

SEQ ID NO: 451 - protein, 30 aa, synthetic construct; identical to SEQ ID NO: 417

SEQ ID NO: 452 - protein, 30 aa, synthetic construct; identical to SEQ ID NO: 418

SEQ ID NO: 453 - protein, 33 aa, synthetic construct
KKKRKRGDKRKRKRHRKKKRRRRNRSLTPATAI

SEQ ID NO: 454 - protein, 30 aa, synthetic construct; identical to SEQ ID NO: 420

SEQ ID NO: 455 - protein, 35 aa, synthetic construct
KKKRKRGDKRKRKRHRKKKRRRRNRSCLIRRTSIC

SEQ ID NO: 456 - protein, 36 aa, synthetic construct
KKKRKRGDKRKRKRHRKKKRRRRNRSCFFWKFRWMC

SEQ ID NO: 457 - protein, 35 aa, synthetic construct
KKKRKNRSRGDKRKRKRHRKKKRRRRIELLQARGC

SEQ ID NO: 458 - protein, 30 aa, synthetic construct
KKKRKRGDKRKRKRHRKKKNRSRRRRREDV

SEQ ID NO: 459 - protein, 36 aa, synthetic construct
KKKRKRGDKRKRKRHRKKKRRRRVHPKQHRGGSKGC

SEQ ID NO: 460 - DNA, 10664 bp, synthetic construct
  source 1..10664: mol_type = other DNA
tcgcgcgtttcggtgatcgaggtgagccccacgttctgcttcactctccccatctcccccccctccccacccccaattttgtatttatttattttttaattattttgtgcagcgatgggggcggggggggggggggggcgcgcgccaggcggggcggggcggggcgaggggcggggcggggcgaggcggagaggtgcggcggcagccaatcagagcggcgcgctccgaaagtttccttttatggcgaggcggcggcggcggcggccctataaaaagcgaagcgcgcggcgggcgggagtcgctgcgacgctgccttcgccccgtgccccgctccgccgccgcctcgcgccgcccgccccggctctgactgaccgcgttactcccacaggtgagcgggcgggacggcccttctcctccgggctgtaattagcgcttggtttaatgacggcttgtttcttttctgtggctgcgtgaaagccttgaggggctccgggagggccctttgtgcgggggggagcggctcggggggtgcgtgcgtgtgtgtgtgcgtggggagcgccgcgtgcggcccgcgctgcccggcggctgtgagcgctgcgggcgcggcgcggggctttgtgcgctccgcagtgtgcgcgaggggagcgcggccgggggcggtgccccgcggtgcggggggggctgcgaggggaacaaaggctgcgtgcggggtgtgtgcgtgggggggtgagcagggggtgtgggcgcggcggtcgggctgtaacccccccctgcacccccctccccgagttgctgagcacggcccggcttcgggtgcggggctccgtacggggcgtggcgcggggctcgccgtgccgggcggggggtggcggcaggtgggggtgccgggcggggcggggccgcctcgggccggggagggctcgggggaggggcgcggcggcccccggagcgccggcggctgtcgaggcgcggcgagccgcagccattgccttttatggtaatcgtgcgagagggcgcagggacttcctttgtcccaaatctgtgcggagccgaaatctgggaggcgccgccgcaccccctctagcgggcgcggggcgaagcggtgcggcgccggcaggaaggaaatgggcggggagggccttcgtgcgtcgccgcgccgccgtccccttctccctctccagcctcggggctgtccgcggggggacggctgccttcgggggggacggggcagggcggggttcggcttctggcgtgtgaccggcggctctagagcctctgctaaccatgttcatgccttcttctttttcctacagctcctgggcaacgtgctggttattgtgctgtctcatcattttggcaaaaccggtctcgaaggcctgcaggcggccgccgccaccgccaccatgcaaatagcactcttcgcttgcttctttctgagccttttcaatttctgctctagtgccatcagaagatactaccttggtgcagtggaattgtcctggaactatattcagagtgatctgctcagtgtgctgcatacagactcaagatttcttcctagaatgtcaacatcttttccattcaacacctccatcatgtataaaaagactgtgtttgtagagtacaaggaccagcttttcaacattgccaagcccaggccaccctggatgggtttgctaggtcctaccatttggactgaggttcatgacacagtggtcattacacttaaaaacatggcttctcatcctgtcagtcttcatgctgttggtgtgtcctactggaaagcttctgagggagatgaatatgaagatcagacaagccaaatggagaaggaagatgataaagttttccctggtgaaagtcatacttatgtttggcaagtcctgaaagagaatggtccaatggcctctgaccctccatgtctcacttactcatatatgtctcatgtggatctggtgaaagatttgaattcaggcctcattggagctctgctagtatgtaaagaaggcagtctctccaaagaaagaacacagatgttgtaccaatttgtactgctttttgctgtatttgatgaagggaagagctggcactcagaaacaaacgactcttatacacagtctatggattctgcatctgctagagactggcctaaaatgcacacagtcaatggctatgtaaacaggtctcttccaggtctgattggatgccataggaaatcagtctactggcacgtgattggaatgggcaccactcctgaaatacactcaatattcctcgaaggtcacacattttttgtgaggaaccaccgtcaagcttcattggagatatcaccaataactttccttactgctcaaacactcttgatagatcttgggcagttcctactattttgtcatatctcttcccataaacatgatggcatggaagcttatgtcaaagtagatagctgccctgaggaatcccaatggcaaaagaaaaataataatgaggaaatggaagattatgatgatgatctttattcagaaatggatatgttcacattggattatgacagctctccttttatccaaattcgctcggttgctaaaaagtaccctaaaacttggatacattatatttctgctgaggaggaagactgggactatgcaccttcagttcctacctcggataatggaagttataaaagccagtatctgagcaatggtcctcatcggattggtaggaaatataaaaaagtcagatttatagcatacacagatgaaacctttaagactcgtgaaactattcagcatgaatcaggactcttgggacctttactttatggagaagttggagacacactgttgattatttttaagaatcaagcaagccgaccatataacatttaccctcatggaatcactgatgtcagtcctctacatgcaaggagattgccaagaggtataaagcacgtgaaggatttgccaattcatccaggagagatattcaagtacaagtggacagttacagtagaagatggaccaactaaatcagatccacggtgcctgacccgctattattcaagtttcattaaccctgagagagatctagcttcaggactgattggccctcttctcatctgctacaaagaatctgtagatcaaaggggaaaccagatgatgtcagacaaaagaaatgtcatcctgttttctatatttgatgagaaccaaagctggtacatcacagagaacatgcaacgcttcctccccaatgcagctaaaacacagccccaggaccctgggttccaggcctccaacatcatgcacagcatcaatggctatgtttttgatagcttggagttgacagtttgtttgcatgaggtggcatactggcacattctcagtgttggagcacagacagacttcttatctatcttcttctctggatatactttcaaacacaaaatggtctatgaagatacacttaccctgttcccattctcaggagaaactgtctttatgtcgatggaaaacccaggtctatgggtcttggggtgtcataattcagactttcggaagagaggtatgacagcattgctgaaagtttctagttgtgacaagagcactagtgattattatgaagaaatatatgaagatattccaacacagttggtgaatgagaacaatgtcattgatcccagaagcttcttccagaatacaaatcatcctaatactaggaaaaagaaattcaaagattccacaattccaaaaaatgatatggagaagattgagcctcagtttgaagagatagcagagatgcttaaagtacagagtgtctcagttagtgacatgttgatgctcttgggacagagtcatcctactccacatggcttatttttatcagatggccaagaagccatctatgaggctattcatgatgatcattcaccaaatgcaatagacagcaatgaaggcccatctaaagtgacccaactcaggccagaatcccatcacagtgagaaaatagtatttactcctcagcccggcctccagttaagatccaataaaagtttggagacaactatagaagtaaagtggaagaaacttggtttgcaagtttctagtttgccaagtaatctaatgactacaacaattctgtcagacaatttgaaagcaacttttgaaaagacagattcttcaggatttccagatatgccagttcactctagtagtaaattaagtactactgcatttggtaagaaagcatattcccttgttgggtctcatgtacctttaaacgtgagtgaagaaaatagtgattccaacatattggattcaactttaatgtatagtcaagaaagtttaccaagagataatatattatcaatggagaatgatagattactcagagagaagaggtttcatggaattgctttattgaccaaagataatactttattcaaagacaatgtctccttaatgaaaacaaacaaaacatataatcattcaacaactaatgaaaaactacacactgagagcccaacatcaattgagaatagtacaacagacttgcaagatgccatattaaaggtcaatagtgagattcaagaagtaacagctttgattcatgatggaacacttttaggcaaaaattctacatatttgagactaaaccatatgctaaatagaactacctcaacaaaaaataaagacatatttcatagaaaagatgaagatcctattccacaagatgaagagaatacaatcatgccattttccaagatgttgttcttgtcagaatcttcaaattggtttaaaaagaccaatggaaataattccttgaactctgagcaagaacatagtccaaagcaattagtatatttaatgtttaaaaaatatgtaaaaaatcaaagtttcttgtcagagaaaaataaagtcacagtagaacaggatggatttacaaagaacataggacttaaagacatggcttttccacataatatgagcatatttcttaccactttgtctaacgtacatgaaaatggtaggcacaatcaagaaaaaaatattcaggaagagatagagaaggaagcactaattgaagagaaagtagttttgccccaggtgcacgaagcaactggctctaagaatttcttgaaagacatattgatactaggcactaggcaaaatataagtttatatgaagtacatgtaccagtacttcaaaacatcacatcaataaacaattcaacaaatacagtacagattcacatggagcatttctttaaaagaaggaaggacaaggaaacaaattcagaaggcttggtaaataaaaccagagaaatggtaaaaaactatccaagccagaagaatattactactcaacgtagtaaacgggctttgggacaattcagactgtcaactcaatggcttaaaaccataaactgttcaacacagtgtatcattaaacagatagaccacagcaaggaaatgaaaaagttcattactaaatcttccttatcagattcttctgtgattaaaagcaccactcagacaaatagttctgactcacacattgtaaaaacatcagcatttccaccaatagatctcaaaaggagtccattccaaaacaaattttctcatgttcaagcatcatcctacatttatgactttaagacaaaaagttcaagaattcaagaaagcaataatttcttaaaagaaaccaaaataaataacccttctttagccattctaccatggaatatgttcatagatcaaggaaaatttacctccccagggaaaagtaacacaaactcagtcacatataagaaacgtgagaacattattttcttgaaaccaactttgcctgaagaatctggcaaaattgaattgcttcctcaagtttccattcaagaggaagaaattttacctacagaaactagccatggatctcctggacacttgaatctcatgaaagaggtctttcttcagaaaatacaggggcctactaaatggaataaagcaaagaggcatggagaaagtataaaaggtaaaacagagagctctaaaaatactcgctcaaaactgctaaatcatcatgcttgggattatcattatgctgcacagataccaaaagatatgtggaaatccaaagagaagtcaccagaaattatatccattaagcaagaggacaccattttgtctctgaggcctcatggaaacagtcattcaataggggcaaatgagaaacaaaattggcctcaaagagaaaccacttgggtaaagcaaggccaaactcaaaggacatgctctcaaatcccaccagtgttgaaacgacatcaaagggaacttagtgcttttcaatcagaacaagaagcaactgactatgatgatgccatcaccattgaaacaatcgaggattttgacatttacagtgaggacataaagcaaggtccccgcagctttcaacagaaaacaaggcactattttattgcagctgtggaacgactctgggactatgggatgagtacatctcatgttctacgaaataggtatcaaagtgacaatgtacctcagttcaagaaagtagttttccaggaatttactgatggctcctttagtcagcccttatatcgtggagaattaaatgaacacctggggttgttgggcccatatataagagcagaagttgaagacaacattatggtaactttcaaaaaccaggcctcccgtccctactccttctattctagcctcatttcttataaagaagatcagagaggagaagaacctagaagaaactttgtcaagcctaatgaaaccaaaatttatttttggaaagtacaacatcatatggcacccacagaagatgagtttgactgcaaggcctgggcttatttctctgatgttgatcttgaaagagatatgcactcgggattaattggaccccttctgatttgccacgcgaacacactgaatcctgctcatgggagacaagtgtcagtacaggaatttgctctgcttttcactatctttgatgagaccaagagctggtacttcactgaaaacgtgaaaaggaactgcaagacaccctgcaatttccagatggaagaccccactttgaaagagaattatcgcttccatgcaatcaatggttatgtaatggataccctaccaggcttagtaatggctcaagatcaaaggattcgatggtatcttctcagcatgggcaacaatgagaacatccaatctattcatttcagtggacatgttttcactgtacggaaaaaagaggagtataaaatggcagtgtacaacctctacccaggtgtttttgagactctggaaatgataccatccagagctggaatatggcgagtagaatgccttattggcgagcacttacaggctgggatgagcactctttttctggtgtacagcaagcagtgtcagattcctcttggaatggcttctggaagcatccgtgatttccagattacagcttcaggacattatggacagtgggccccaaacctggcaagacttcattattccggatcaatcaatgcctggagtaccaaggagcccttttcttggatcaaggtagatctgttggcaccaatgattgttcatggcatcaagactcagggtgctcgtcagaaattttccagcctttatatctctcaatttatcatcatgtatagcctggatgggaagaagtggctgagttatcaaggaaattccactggaaccttaatggttttctttggcaatgtggactcatctgggattaagcataatagttttaatcctccaattattgctcgatatatccgtttgcaccccactcattctagcatccgtagtactcttcgcatggagttgatgggctgtgatttaaacagttgcagcataccattgggaatggaaagtaaagtaatatcagatacacaaatcactgcctcatcctacttcaccaacatgtttgctacttggtctccttcacaagctcgacttcacctccagggaaggactaatgcctggcgacctcaggtgaatgatccaaaacaatggttgcaagtggacttacaaaagacaatgaaagtcactggaataataacccagggagtgaaatctctctttaccagcatgtttgtgaaagagttccttatttccagcagtcaagatggccatcactggactcaaattttatacaatggcaaggtaaaggtttttcaggggaatcaggactcatccacacctatgatgaattctctagacccaccattactcactcgctatcttcgaattcacccccagatctgggagcaccaaattgctctgaggcttgagattctaggatgtgaggcccagcagcaatactgaccatggcccaacttgtttattgcagcttataatggttacaaataaagcaatagcatcacaaatttcacaaataaagcatttttttcactgcattctagttgtggtttgtccaaactcatcaatgtatcttatcatgtctggatctcgttaactcgagggatccatcgatgtcgactgcagaggcctgcatgcaagcttggtgtaatcatggtcatagctgtttcctgtgtgaaattgttatccgctcacaattccacacaacatacgagccggaagcataaagtgtaaagcctggggtgcctaatgagtgagctaactcacattaattgcgttgcgctcactgcccgctttccagtcgggaaacctgtcgtgccagctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaagcccaatctgaataatgttacaaccaattaaccaattctgattagaaaaactcatcgagcatcaaatgaaactgcaatttattcatatcaggattatcaataccatatttttgaaaaagccgtttctgtaatgaaggagaaaactcaccgaggcagttccataggatggcaagatcctggtatcggtctgcgattccgactcgtccaacatcaatacaacctattaatttcccctcgtcaaaaataaggttatcaagtgagaaatcaccatgagtgacgactgaatccggtgagaatggcaaaagtttatgcatttctttccagacttgttcaacaggccagccattacgctcgtcatcaaaatcactcgcatcaaccaaaccgttattcattcgtgattgcgcctgagcgagacgaaatacgcgatcgctgttaaaaggacaattacaaacaggaatcgaatgcaaccggcgcaggaacactgccagcgcatcaacaatattttcacctgaatcaggatattcttctaatacctggaatgctgtttttccggggatcgcagtggtgagtaaccatgcatcatcaggagtacggataaaatgcttgatggtcggaagaggcataaattccgtcagccagtttagtctgaccatctcatctgtaacatcattggcaacgctacctttgccatgtttcagaaacaactctggcgcatcgggcttcccatacaagcgatagattgtcgcacctgattgcccgacattatcgcgagcccatttatacccatataaatcagcatccatgttggaatttaatcgcggcctcgacgtttcccgttgaatatggctcataacaccccttgtattactgtttatgtaagcagacagttttattgttcatgatgatatatttttatcttgtgcaatgtaacatcagagattttgagacacgggccagagctgca

SEQ ID NO: 461 - protein, 5 aa, synthetic construct
RRRRR

SEQ ID NO: 462 - protein, 4 aa, synthetic construct
NRDN

SEQ ID NO: 463 - protein, 9 aa, synthetic construct
KLKIKRPVK

SEQ ID NO: 464 - protein, 7 aa, synthetic construct
PKKKRKV

SEQ ID NO: 465 - skipped

SEQ ID NO: 466 - protein, 5 aa, synthetic construct
GTALL

SEQ ID NO: 467 - protein, 4 aa, synthetic construct
DSLL

SEQ ID NO: 468 - protein, 4 aa, synthetic construct
NPTY

SEQ ID NO: 469 - skipped

SEQ ID NO: 470 - protein, 5 aa, synthetic construct
FYQPL

SEQ ID NO: 471 - protein, 6 aa, synthetic construct
IKVAVA

SEQ ID NO: 472 - protein, 20 aa, synthetic construct
KRHRKKRHRKKRHRKKRHRK

SEQ ID NO: 473 - protein, 32 aa, synthetic construct
KKRHRKGGSLLRGDKELKRPPRRRRRHYNKSR

SEQ ID NO: 474 - protein, 32 aa, synthetic construct
KKRHRKGGVLLRGDKELKRPPRRRRRHYNKSR

SEQ ID NO: 475 - protein, 32 aa, synthetic construct
KKRHRKGGALLRGDKELKRPPRRRRRHYNKSR

SEQ ID NO: 476 - protein, 33 aa, synthetic construct
KKRHRKGGSLLRGDKELKRPPRRRRRHYCAILR

SEQ ID NO: 477 - protein, 33 aa, synthetic construct
KKRHRKKGGSLLRGDKELKRPPRRRRRHYNKSR

SEQ ID NO: 478 - protein, 4 aa, synthetic construct
FQVT

SEQ ID NO: 479 - protein, 30 aa, synthetic construct
  VARIANT 5..30: note = Residues may be deleted
RRRRRRRRRRRRRRRRRRRRRRRRRRRRRR

SEQ ID NO: 480 - protein, 6 aa, synthetic construct
KKRHRK

SEQ ID NO: 481 - protein, 22 aa, synthetic construct
KRHRKLRRRRRLKRHRKKRHRK

SEQ ID NO: 482 - protein, 20 aa, synthetic construct
KKKRHRKRKRKRKRRRRKKK

SEQ ID NO: 483 - protein, 8 aa, synthetic construct
ASSLNIAK

SEQ ID NO: 484 - protein, 5 aa, synthetic construct
KKKRK

SEQ ID NO: 485 - protein, 22 aa, synthetic construct
KRKRKKRHRKKKKRRRRKRHRK

SEQ ID NO: 486 - protein, 11 aa, synthetic construct
KKKRHRKKKKK

SEQ ID NO: 487 - protein, 4 aa, synthetic construct
KKKK

SEQ ID NO: 488 - protein, 17 aa, synthetic construct
KKGKKKKRRRRKAAPKK